(19) United States
(12) Patent Application Publication
(10) Pub. No.: US 2022/0281866 A1
(43) Pub. Date: Sep. 8, 2022

(54) 5,8-DISUBSTITUTED-[1,2,4]TRIAZOLO[1,5-A]PYRIDINYL AND 5,8-DISUBSTITUTED-IMIDAZO[1,2-A]PYRIDINE DERIVATIVES USEFUL AS INHIBITORS OF ENTEROPEPTIDASE

(71) Applicant: Janssen Pharmaceutica NV, Beerse (BE)

(72) Inventors: Xuqing Zhang, Audubon, PA (US); Weimei Sun, Lower Gwynedd, PA (US); James C. Lanter, Audubon, PA (US); Brahmananda Ghosh, North Wales, PA (US)

(21) Appl. No.: 17/626,830

(22) PCT Filed: Jul. 17, 2020
(86) PCT No.: PCT/EP2020/070337

§ 371 Date: Jan. 13, 2022

Related U.S. Application Data
(60) Provisional application No. 62/875,989, filed on Jul. 19, 2019.

(51) Int. Cl. C07D 471/04

(52) U.S. Cl. CPC C07D 471/04

(57) ABSTRACT

The present invention is directed to 5,8-disubstituted-[1,2,4]triazolo[1,5-a]pyridinyl and 5,8-disubstituted-imidazo[1,2-a]pyridine derivatives, pharmaceutical compositions containing them and their use in the treatment of disorders and conditions modulated by the enteropeptidase enzyme.

### CROSS REFERENCE TO RELATED APPLICATIONS

This application claims priority to U.S. Provisional Patent Application No. 62/875,989, filed Jul. 19, 2019, the disclosure of which is hereby incorporated by reference in its entirety.

### FIELD OF THE INVENTION

The present invention is directed to 5,8-disubstituted-[1,2,4]triazolo[1,5-a]pyridinyl and 5,8-disubstituted-imidazo[1,2-a]pyridine derivatives, pharmaceutical compositions containing them and their use in the treatment of disorders and conditions modulated by the enteropeptidase enzyme. More particularly, the compounds of the present invention are inhibitors of the enteropeptidase enzyme, useful in the treatment of obesity, Type II diabetes mellitus, Syndrome X, kidney disorders such as chronic kidney disease, NASH, NAFLD, etc.

### BACKGROUND OF THE INVENTION

Obesity is a complex metabolic disorder in which many environmental factors and numerous genes are implicated. It is a multi-faceted chronic condition and is the most prevalent nutritional problem in the United States today. Considerable effort has been devoted to developing drugs which are involved, directly or indirectly, in energy management; control of appetite, satiety, or thermogenesis; fatty acid, carbohydrate and protein metabolisms; energy generation etc. Obesity, a condition caused by an excess of energy intake as compared to energy expenditure, contributes to the pathogenesis of hypertension, type II or non-insulin dependent diabetes mellitus, hypercholesterolemia, hyperlipidemia, hypertriglyceridemia, heart disease, pancreatitis, and such common forms of cancer such as breast cancer, prostate cancer, uterine cancer and colon cancer.

At present, only a limited number of drugs for treating obesity are commercially available. Unfortunately, while some of these drugs may bring short-term relief to the patient, a long-term successful treatment has not yet been achieved. The presently known targets for the treatment of obesity and related disorders can be divided into four main classes: (i) appetite blockers; (ii) satiety stimulators; (iii) energy or fatty acid burning agents; (iv) fat absorption inhibitors. The use of these targets is highly limited by their redundancy, their multiple targeting and/or their lack of tissue specificity. There is thus a widely recognized need for, and it would be highly advantageous to have compositions and methods for treating obesity and related diseases and disorders devoid of the above limitations.

Serine proteases are involved in a large number of important physiological processes. Selective inhibition of a given serine protease is one of the strategies for the treatment of pathological conditions associated with the activity or overactivity of these serine proteases. Enteropeptidase (EP, also termed enterokinase) is a serine protease localized on the brush border of the duodenum. This enzyme catalyzes the conversion of inactive trypsinogen into active trypsin via the cleavage of the acidic propeptide from trypsinogen. In turn, the activation of trypsin initiates a cascade of proteolytic reactions leading to the activation of many pancreatic zymogens, such as chymotrypsinogen, proelastase, procarboxypeptidases A and B, and pro-lipase. Those active forms of such digestive enzymes are required for the processing and ultimate absorption of protein and fat matter in the gastrointestinal (GI) tract. Congenital EP deficiency is an extremely rare, recessive inherited disorder that results in failure to thrive, diarrhea, anemia, hypoproteinemia and edema. This condition is usually successfully treated by pancreatic enzyme replacement or by dietary administration of protein hydrolysate. In vitro studies of small intestinal mucosal biopsy specimens suggest that EP deficiency is due to the formation of structurally altered enzymes with no EP activity. On the other hand, because enteropeptidase is located in the intestinal lumen, inhibition of this enzyme requires that the compounds selectively inhibit enteropeptidase without interfering with circulating serine proteases, such as thrombin, kalikrein, and the like. Thus, there is a need for compounds to treat obesity, excess overweight as well as diseases associated with an abnormal fat metabolism on a long term basis that have a specific target. It is challenging to discover compounds as serine protease inhibitors with a specific and selective inhibition of a unique target. Moreover, very few compounds have been disclosed or suggested, to selectively and specifically inhibit the enteropeptidase, and to be used in the treatment of obesity, excess weight or diseases associated with an abnormal fat metabolism.

Kidneys are bean-shaped organs, located near the middle of the back. Inside each kidney about a million tiny structures called nephrons filter blood. They remove waste products and extra water, which become urine. Damage to the nephrons represents an important form of kidney disease. This damage may leave kidneys unable to remove wastes. Some damage, e.g. damage related to hyperfiltration can occur slowly over years, initially often without obvious symptoms.

The ‘hyperfiltrative hypothesis’ implies that the excess demand on a limited renal reserve produces adaptive and ultimately pathologic changes in the kidney which finally lead to ‘nephron exhaustion’. At the single-nephron level, hyperfiltration is hypothesized to be an early link in the chain of events that lead from intraglomerular hypertension to albuminuria and, subsequently, to reduced Glomerular Filtration Rate (GFR). Based on this hyperfiltration therefore represents a risk for subsequent renal injury and could be classified as an early manifestation of renal pathology often referred to as the hyperfiltrative stage. Such renal hyperfiltration can lead to early glomerular lesions and to microalbuminuria, which itself can lead to macroalbuminuria and to end-stage renal disease.

The influence of hyperfiltration on renal function decline has been most thoroughly evaluated in kidney transplant recipients and donors, and in patients with a single kidney removed for acquired renal disease, but also in patients with diabetes mellitus (MAGEE, et al. Diabetologia 2009; 52: 691-697). In theory, any reduction in functional nephron number will lead to adaptive glomerular hyperfiltration whether induced genetically, surgically, or by acquired renal disease. Moreover, hyperfiltration has been shown to occur in certain pathophysiologic conditions even when renal mass is intact, e.g. in diabetes. Therefore, there is a medical need for interventions with a good efficacy with regard to renal hyperfiltrative injury.

Creatinine is a breakdown product of creatine phosphate in muscle tissue and is usually produced at a constant rate in the body. Serum creatinine is an important indicator of renal health, because it is an easily measured byproduct of muscle metabolism that is excreted unchanged by the kidneys. Creatinine is removed from the blood chiefly by the kidneys, primarily by glomerular filtration, but also by proximal tubular secretion. Little or no tubular reabsorption of creatinine occurs. If the filtration in the kidney is deficient, creatinine blood levels rise. Therefore, creatinine levels in blood and urine may be used to calculate the creatinine clearance (CrCl), which correlates with the glomerular filtration rate (GFR). Blood creatinine levels may also be used alone to estimate the GFR (eGFR). The GFR is clinically important because it is a measurement of renal function. An alternate estimation of renal function can be made when interpreting the blood (plasma) concentration of creatinine along with that of urea. The BUN-to-creatinine ratio (the ratio of blood urea to creatinine) can indicate other problems besides those intrinsic to the kidney; for example, a urea level raised out of proportion to the creatinine may indicate a pre-renal problem such as volume depletion.

A rise in blood creatinine level is observed only with marked damage to functioning nephrons. An estimation of kidney function is given by calculating the estimated glomerular filtration rate (eGFR). eGFR can be accurately calculated using serum creatinine concentration. The typical human reference ranges for serum creatinine are 0.5 to 1.0 mg/dl (about 45-90 μmol/l) for women and 0.7 to 1.2 mg/dl (60-110 μmol/l) for men. The trend of serum creatinine levels over time is generally more important than absolute creatinine level.

Creatinine levels may increase modestly when an ACE inhibitor (ACEi) or angiotensin II receptor antagonist (or angiotensin receptor blocker, ARB) is taken. Using both an ACE inhibitor and ARB concomitantly will increase creatinine levels to a greater degree than either of the two drugs would individually. An increase of <30% is to be expected with ACE inhibitor or ARB use.

Albuminuria is a condition, where albumin is present in the urine. In healthy individuals, albumin is filtered by the kidneys. When the kidneys do not properly filter large molecules (such as albumin) from the urine, albumin is excreted in urine and is typically a sign of kidney damage or excessive salt intake. Albuminuria can also occur in patients with long-standing diabetes mellitus, either Type I (1) or Type II (2) diabetes mellitus. Urine albumin may be measured by dipstick or as direct measure of the amount of protein excreted in total volume of urine collected over a 24 hour period

Microalbuminuria, occurs when the kidney leaks small amounts of albumin into the urine, as a result of an abnormally high permeability for albumin in the renal glomerulus. Microalbuminuria as a condition of diabetic nephropathy is indicated when urine albumin levels are in the range of 30 mg to 300 mg in a 24 hour period.

An alternate measure of microalbuminuria is creatinine levels and the ratio of albumin to creatinine in serum. The albumin/creatinine ratio (ACR) and microalbuminuria are defined as ACR ≥3.5 mg/mmol (female) or ≥2.5 mg/mmol (male), or, with both substances measured by mass, as an ACR between 30 μg albumin/mg creatinine and 300 μg albumin/mg creatinine.

Microalbuminuria may be an important prognostic marker for the development and progression of kidney disease, particularly in patients with diabetes mellitus or hypertension. Microalbuminuria is also an indicator of subclinical cardiovascular disease, a marker of vascular endothelial dysfunction and a risk factor for venous thrombosis.

Diabetic nephropathy is one of the microvascular complications of diabetes mellitus and is characterized by persistent albuminuria and a progressive decline in renal function. Hyperglycemia is an important contributor to the onset and progression of diabetic nephropathy.

The clinical progression of diabetic nephropathy in patients with T1 DM (Type 1 Diabetes Mellitus) is well characterized. Initially, hyperfiltration accompanied by increases in glomerular filtration rate (GFR) and increased renal plasma flow is seen. A meta-analysis found that the presence of hyperfiltration in patients with T1 DM more than doubled the risk of developing micro- or macroalbuminuria. This phase is followed by reductions in GFR and the development of microalbuminuria, defined as urinary albumin excretion of >30 mg/day (or 20 μg/min) and <300 mg/24 h (or <200 μg/min), which may be accompanied by increases in blood pressure. Later in the progression of the disease as GFR continues to decline, overt proteinuria (i.e., macroalbuminuria), defined as urinary albumin excretion of >300 mg/day ensues and is associated with worsening hypertension. Eventually, ESKD (End Stage Kidney Disease) progresses, leading to the need for renal replacement therapy.

In patients with Type 2 Diabetes Mellitus (T2DM), the clinical progression is variable, primarily due to multiple renal insults, including not only hyperglycemia, but also vascular pathology resulting in ischemic renal injury. However, other common features are likely to contribute to renal injury in patients with T2DM include hyperfiltration at the level of the single nephron, proximal tubular glucotoxicity, and a stimulus for tubular cell growth as a result of enhanced sodium coupled glucose transport into tubular cells.

Studies have demonstrated that albuminuria is a biomarker for predicting progression of diabetic nephropathy and is a cardiovascular (CV) risk factor. When compared with patients with normo-albuminuria and estimated glomerular filtration rate (eGFR) ≥90 mL/min/1.73 m², patients with both macroalbuminuria and eGFR <60 mL/min/1.73 m² were at 5.9-fold higher risk (95% Cl 3.5 to 10.2) for cardiovascular death and 22.2-fold higher risk (95% Cl 7.6 to 64.7) for experiencing ESKD, and subjects with macroalbuminuria and reduced eGFR (ie, <60 mL/min/1.73 m²) were nearly 6 times more likely to experience a composite renal event (i.e., death as a result of kidney disease, requirement for dialysis or transplantation, or doubling of serum creatinine (See, e.g., NINOMIYA, T., et al., J. Am. Soc. Nephrol., 2009, pp 1813-1821, Vol. 20(8)). A close link between the degree of albuminuria and CV disease has also been demonstrated in the RENAAL study, showing that patients with high baseline urinary albumin/creatinine ratio (ACR) (≥3 g/g) had a 1.2-fold (95% Cl, 1.54 to 2.38) higher risk of a composite of myocardial infarction (MI), stroke, first hospitalization for heart failure or unstable angina, coronary or peripheral revascularization, or CV death, and a 2.7-fold (95% Cl, 1.94 to 3.75) higher risk of heart failure compared with patients with an ACR <1.5 g/g.

Increased urinary albumin excretion and reduced eGFR are also independently associated with the risk for both cardiovascular and kidney outcomes in patients with T2DM, without evidence for an interaction between these risk factors. Moderately increased albuminuria also has been associated with an increase in renal disease progression.

In summary, the magnitude of albuminuria positively correlates with the development of ESKD and adverse CV outcomes. Treatment-related reductions in albuminuria in patients with T2DM and albuminuria using agents acting by a hemodynamic mechanism (i.e., ACEi and ARBs) are correlated with reductions in the progression of diabetic nephropathy and in the incidence of adverse CV outcomes. Thus, agents acting by a unique hemodynamic mechanism to reduce albuminuria beyond that seen with other antihypertensive or antihyperglycemic agents and which are additive to agents disrupting the renin-angiotensin system may exert reno-protective effects and possibly reduce adverse CV outcomes in diabetic nephropathy.

Non-alcoholic fatty liver disease (NAFLD) is one cause of a fatty liver, occurring when fat is deposited (steatosis) in the liver. NAFLD is considered to cover a spectrum of disease activity. This spectrum begins as fatty accumulation in the liver (hepatic steatosis). A liver can remain fatty without disturbing liver function, but by varying mechanisms and possible insults to the liver may also progress to become NASH, a state in which steatosis is combined with inflammation and fibrosis. Non-alcoholic steatohepatitis (NASH) is a progressive, severe form of NAFLD. Over a 10-year period, up to 20% of patients with NASH will develop cirrhosis of the liver, and 10% will suffer death related to liver disease. The exact cause of NAFLD is still unknown, however, both obesity and insulin resistance are thought to play a strong role in the disease process. The exact reasons and mechanisms by which the disease progresses from one stage to the next are not known.

NAFLD has been linked to insulin resistance (IR) and the metabolic syndrome (MS). As the renin-angiotensin system (RAS) plays a central role in insulin resistance, and subsequently in NAFLD and NASH, an attempt to block the deleterious effects of RAS overexpression has been proposed a target for treatment. While many potential therapies tested in NASH target only the consequences of this condition, or try to “get rid” of excessive fat, angiotensin receptor blockers (ARBs) may act as a tool for correction of the various imbalances that act in harmony in NASH/NAFLD. Indeed, by inhibiting RAS the intracellular insulin signaling pathway may be improved, resulting in better control of adipose tissue proliferation and adipokine production, as well as more balanced local and systemic levels of various cytokines. At the same time, by controlling the local RAS in the liver fibrosis may be prevented and the cycle that links steatosis to necroinflammation slowed down. (GEORGESCU, E. F., in Advances in Therapy, 2008, pp 1141-1174, Vol. 25, Issue 11)

HAROSH, I., et al., in PCT Publication WO2009071601 A1, published Jun. 11, 2009 describe derivatives of boroarginine, boroornithine and borolysine that selectively modulate, regulate and/or inhibit enteropeptidase; as well as methods to treat excess weight, obesity and diseases associated with an abnormal fat metabolism.

KOSHIBA, T., et al., in US Patent Publication 20130338132 A1, published Dec. 19, 2013 describe heteroarylcarboxylic acid ester derivatives, which exhibit a serine protease (particularly trypsin and enteropeptidase) inhibitory activity; for the treatment or prophylaxis of diabetes.

IKEDA, Z., et al., in US Patent Publication 20150225354 A1, published Aug. 13, 2015 describe fused heterocyclic compounds having an enteropeptidase inhibitory activity, and use of said compounds for the treatment or prophylaxis of obesity, diabetes mellitus, etc.

IKEDA, Z., et al., in US Patent Publication US 20150225361 A1, published Aug. 13, 2015 describe fused heterocyclic compounds having an enteropeptidase inhibitory activity, and use of said compounds for the treatment or prophylaxis of obesity, diabetes mellitus, etc.

KAKEGAWA, K., et al., in U.S. Pat. No. 10,017,487 B2, Issued Jul. 10, 2018 (Equivalent of WO 2016158788 A1) describe condensed heterocyclic compounds that have an enteropeptidase inhibitory effect and the use of said compounds for the treatment or prevention of obesity, diabetes mellitus, or the like.

SASAKI, M., et al., in U.S. Pat. No. 9,975,903 B2, Issued May 22, 2018 (Equivalent of WO 2016104630 A1) describe condensed heterocyclic compounds that have an enteropeptidase inhibitory effect and the use of said compounds for the treatment or prevention of obesity, diabetes mellitus, or the like.

There remains a need for enteropeptidase inhibitor compounds (preferably selective enteropeptidase inhibitor compounds) that have pharmacokinetic and pharmacodynamic properties suitable for use as human pharmaceuticals for the treatment of, for example, obesity, Type II diabetes mellitus, NASH, NAFLD, chronic kidney disease, and other related disorders.

### SUMMARY OF THE INVENTION

The present invention is directed to compounds of formula (I)

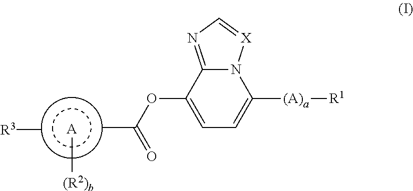

wherein

X is selected from the group consisting of N and CH;

a is an integer from 0 to 1;

A is —(C₁₋₆alkyl)-; R¹ is selected from the group consisting of 1,2,3,5-tetrazol-4-yl, imidazolidin-5-yl-2,4-dione, oxazolidin-5-yl-2,4-dione, 1,2,4-oxadiazol-5(4H)-one, thiazolidin-5-yl-2,4-dione, 1,2,4-thiadiazol-5(4H)-one, isothiazolidin-4-yl-3-one 1,1-dioxide, 2,4-dihydro-3H-1,2,4-triazol-3-one, —CO₂H, —C(O)—O—(C₁₋₄alkyl), —SO₃H, —PO₃H₂, —C(O)—NH—CH₂—CO₂H, —C(O)—NH—CH(CO₂H)—CH₂—CO₂H, —C(O)—N(CO₂H)—CH₂—CO₂H, —C(O)—N(CH₂CO₂H)—CH₂—CO₂H, —C(O)—NH—C(CH₂CH₂—CO₂H)₃, —C(O)—NH—SO₂—(C₁₋₄alkyl) and —C(O)—NH—CH(R⁵)—CO₂H and —C(O)-proline;

wherein R⁵ is selected from the group consisting of hydrogen, methyl, isopropyl, 2-methylpropyl, 3-methylpropyl, 2-methylthio-ethyl, benzyl, 4-hydroxy-benzyl, 1H-indol-3-methyl, carbamoyl-methyl, sulfanyl-methyl, 2-carbamoyl-ethyl, hydroxy-methyl, 2-hydroxy-ethyl, carboxyl-methyl, 2-carboxy-ethyl, 3-guanidino-propyl, 1H-imidazol-4-yl-methyl, 4-amino-butyl;

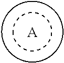

is selected from the group consisting of phen-1,4-diyl, phen-1,3-diyl, 5-6 membered heteroaryl and bicyclic 9-10 membered heteroaryl;

wherein the 5 membered heteroaryl is bound (to the —R³ and —C(O)O substituents) to the rest of the compound through two carbon atoms in a meta orientation;

wherein the 6 membered heteroaryl is bound (to the —R³ and —C(O)O substituents) to the rest of the compound through two carbon atoms in a meta or para orientation; and

wherein the bicyclic 9-10 membered heteroaryl is bound (to the —R³ and —C(O)O substituent) to the rest of the compound through two carbon atoms in a meta or para orientation;

b is an integer from 0 to 1;

R² is selected from the group consisting of halogen, hydroxy, C₁₋₄alkyl, fluorinated C₁₋₂alkyl, C₁₋₄alkoxy, fluorinated C₁₋₂alkoxy, cyano, 3-(prop-2-yn-1-yloxy)prop-1-yn-1-yl and 3-(2-(prop-2-yn-1-yloxy)ethoxy)prop-1-yn-1-yl;

provided that R² is bound to a carbon atom;

provided further that when R² is 3-(prop-2-yn-1-yloxy)prop-1-yn-1-yl or 3-(2-(prop-2-yn-1-yloxy)ethoxy)prop-1-yn-1-yl, then the R² is bound to a carbon atom at the ortho position to the —C(O)—O— group of the compound of formula (I); R³ is selected from the group consisting of —CH(═NH)—NH—NH2, —CH(═NH)—NH(C₁₋₄alkyl), —CH(═NH)—NH(OC₁₋₄alkyl), —CH(═NH)—NH—CN, —NH—CH(═NH)—NH₂, —NH—CH(═N(Boc))—NH(Boc), —NH—CH(═NH)—NH(C₁₋₄alkyl), —NH—CH(═NH)—NH(OH), —NH—CH(═NH)—NH—O(C₁₋₄alkyl), —N(C₁₋₄alkyl)-CH(═NH)—NH₂, —N(C₁₋₄alkyl)-CH(═NH)—NH(C₁₋₄alkyl), —NH—CH(═NH)—(C₁₋₄alkyl), —NH—C(═NOH)—(C₁₋₄alkyl) and imidazolidin-1-yl-2-imine;

and isotopologues, stereoisomers, tautomers and pharmaceutically acceptable salts thereof.

The present invention is further directed to processes for the preparation of the compounds of formula (I). The present invention is further directed to a compound of formula (I) prepared according to any of the process(es) described herein.

Illustrative of the invention are pharmaceutical compositions comprising a pharmaceutically acceptable carrier and a compound of formula (I) as described herein. An illustration of the invention is a pharmaceutical composition made by mixing a compound of formula (I) as described herein and a pharmaceutically acceptable carrier. Illustrating the invention is a process for making a pharmaceutical composition comprising mixing a compound of formula (I) as described herein and a pharmaceutically acceptable carrier.

Exemplifying the invention are methods of treating a disease, disorder, or condition mediated by enteropeptidase activity such as obesity, excess weight, hypertension, hypercholesterolemia, hyperlipidemia, hypertriglyceridemia, heart disease, angina, atherosclerosis, heart disease, heart attack, ischemia, stroke; pancreatitis, impaired glucose tolerance (IGT), impaired fasting glucose (IFT), gestational diabetes, Type II or non-insulin dependent diabetes mellitus, Syndrome X (also known as Metabolic Syndrome), neuropathy, retinopathy, nerve damage or poor blood flow in the feet, chronic kidney disease, microalbuminuria (elevated urine albumin levels), macroalbuminuria, elevated urine albumin levels, elevated albumin/creatinine ratio (ACR), nephropathy (including but not limited to, hyperfiltrative diabetic nephropathy), renal hyperfiltration, glomerular hyperfiltration, renal allograft hyperfiltration, compensatory hyperfiltration (e.g. after renal mass reduction by surgery), hyperfiltrative chronic kidney disease, hyperfiltrative acute renal failure, non-alcoholic steatohepatitis (NASH), non-alcoholic fatty liver disease (NAFLD), liver fibrosis, cataracts, polycystic ovarian syndrome, irritable bowel syndrome, inflammation and cancer (including breast cancer, prostate cancer, uterine cancer, colon cancer, and pancreatic cancer), comprising administering to a subject in need thereof a therapeutically effective amount of any of the compounds or pharmaceutical compositions described above.

In an embodiment, the present invention is directed to a compound of formula (I) for use as a medicament. In another embodiment, the present invention is directed to a compound of formula (I) for use in the treatment of a disorder mediated enteropeptidase activity such as obesity, excess weight, hypertension, hypercholesterolemia, hyperlipidemia, hypertriglyceridemia, heart disease, angina, atherosclerosis, heart disease, heart attack, ischemia, stroke, pancreatitis, impaired glucose tolerance (IGT), impaired fasting glucose (IFT), gestational diabetes, Type II or non-insulin dependent diabetes mellitus, Syndrome X (also known as Metabolic Syndrome), neuropathy, retinopathy, nerve damage or poor blood flow in the feet, chronic kidney disease, microalbuminuria (elevated urine albumin levels), macroalbuminuria, elevated urine albumin levels, elevated albumin/creatinine ratio (ACR), nephropathy (including but not limited to, hyperfiltrative diabetic nephropathy), renal hyperfiltration, glomerular hyperfiltration, renal allograft hyperfiltration, compensatory hyperfiltration (e.g. after renal mass reduction by surgery), hyperfiltrative chronic kidney disease, hyperfiltrative acute renal failure, non-alcoholic steatohepatitis (NASH), non-alcoholic fatty liver disease (NAFLD), liver fibrosis, cataracts, polycystic ovarian syndrome, irritable bowel syndrome, inflammation and cancer (including breast cancer, prostate cancer, uterine cancer, colon cancer, and pancreatic cancer). In another embodiment, the present invention is directed to a composition comprising a compound of formula (I) for the treatment of a disorder mediated by enteropeptidase activity such as obesity, excess weight, hypertension, hypercholesterolemia, hyperlipidemia, hypertriglyceridemia, heart disease, angina, atherosclerosis, heart disease, heart attack, ischemia, stroke, pancreatitis), impaired glucose tolerance (IGT), impaired fasting glucose (IFT), gestational diabetes, Type II or non-insulin dependent diabetes mellitus, Syndrome X (also known as Metabolic Syndrome), neuropathy, retinopathy, nerve damage or poor blood flow in the feet, chronic kidney disease, microalbuminuria (elevated urine albumin levels), macroalbuminuria, elevated urine albumin levels, elevated albumin/creatinine ratio (ACR), nephropathy (including but not limited to, hyperfiltrative diabetic nephropathy), renal hyperfiltration, glomerular hyperfiltration, renal allograft hyperfiltration, compensatory hyperfiltration (e.g. after renal mass reduction by surgery), hyperfiltrative chronic kidney disease, hyperfiltrative acute renal failure, non-alcoholic steatohepatitis (NASH), non-alcoholic fatty liver disease (NAFLD), liver fibrosis, cataracts, polycystic ovarian syndrome, irritable bowel syndrome, inflammation and cancer (including breast cancer, prostate cancer, uterine cancer, colon cancer, and pancreatic cancer).

Another example of the invention is the use of any of the compounds described herein in the preparation of a medicament for treating: (a) obesity, (b) excess weight, (c) hypertension, (d) hypercholesterolemia, (e) hyperlipidemia, (f) hypertriglyceridemia, (g) heart disease, (h) angina, (i) atherosclerosis, (j) heart disease, (k) heart attack, (l) ischemia, (m) stroke, (n) pancreatitis, (o) impaired glucose tolerance (IGT), (p) impaired fasting glucose (IFT), (q) gestational diabetes, (r) Type II or non-insulin dependent diabetes mellitus, (s) Syndrome X (also known as Metabolic Syndrome), (t) neuropathy, (u) retinopathy, (v) nerve damage or poor blood flow in the feet, (w) chronic kidney disease, (x) microalbuminuria (elevated urine albumin levels), (y) macroalbuminuria, (z) elevated urine albumin levels, (aa) elevated albumin/creatinine ratio (ACR), (ab) nephropathy (including but not limited to, hyperfiltrative diabetic nephropathy), (ac) renal hyperfiltration, (ad) glomerular hyperfiltration, (ae) renal allograft hyperfiltration, (af) compensatory hyperfiltration (e.g. after renal mass reduction by surgery), (ag) hyperfiltrative chronic kidney disease, (ah) hyperfiltrative acute renal failure, (ai) non-alcoholic steatohepatitis (NASH), (aj) non-alcoholic fatty liver disease (NAFLD), (ak) liver fibrosis, (al) cataracts, (am) polycystic ovarian syndrome, (an) irritable bowel syndrome, (ao) inflammation and (ap) cancer (including breast cancer, prostate cancer, uterine cancer, colon cancer, and pancreatic cancer), in a subject in need thereof.

In another example, the present invention is directed to a compound as described herein for use in a method for treating a disorder selected from the group consisting of obesity, excess weight, hypertension, hypercholesterolemia, hyperlipidemia, hypertriglyceridemia, heart disease, angina, atherosclerosis, heart disease, heart attack, ischemia, stroke, pancreatitis, impaired glucose tolerance (IGT), impaired fasting glucose (IFT), gestational diabetes, Type II or non-insulin dependent diabetes mellitus, Syndrome X (also known as Metabolic Syndrome), neuropathy, retinopathy, nerve damage or poor blood flow in the feet, chronic kidney disease, microalbuminuria (elevated urine albumin levels), macroalbuminuria, elevated urine albumin levels, elevated albumin/creatinine ratio (ACR), nephropathy (including but not limited to, hyperfiltrative diabetic nephropathy), renal hyperfiltration, glomerular hyperfiltration, renal allograft hyperfiltration, compensatory hyperfiltration (e.g. after renal mass reduction by surgery), hyperfiltrative chronic kidney disease, hyperfiltrative acute renal failure, non-alcoholic steatohepatitis (NASH), non-alcoholic fatty liver disease (NAFLD), liver fibrosis, cataracts, polycystic ovarian syndrome, irritable bowel syndrome, inflammation and cancer (including breast cancer, prostate cancer, uterine cancer, colon cancer, and pancreatic cancer), in a subject in need thereof.

### DETAILED DESCRIPTION OF THE INVENTION

The present invention is directed to compounds of formula (I)

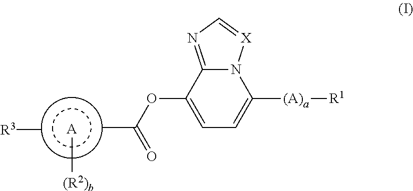

wherein a, b, A, R¹, R², R³, X, and

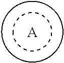

are as herein defined; and isotopologues and pharmaceutically acceptable salts thereof.

The compounds formula (I) of the present invention are useful as inhibitors of enteropeptidase enzyme. As such, the compounds of formula (I) of the present invention are useful in the treatment of diseases, disorders and complications associated with enteropeptidase activity selected from the group consisting of obesity, excess weight, hypertension, hypercholesterolemia, hyperlipidemia, hypertriglyceridemia, heart disease, angina, atherosclerosis, heart disease, heart attack, ischemia, stroke, pancreatitis, impaired glucose tolerance (IGT), impaired fasting glucose (IFT), gestational diabetes, Type II or non-insulin dependent diabetes mellitus, Syndrome X (also known as Metabolic Syndrome), neuropathy, retinopathy, nerve damage or poor blood flow in the feet, chronic kidney disease, microalbuminuria (elevated urine albumin levels), macroalbuminuria, elevated urine albumin levels, elevated albumin/creatinine ratio (ACR), nephropathy (including but not limited to, hyperfiltrative diabetic nephropathy), renal hyperfiltration, glomerular hyperfiltration, renal allograft hyperfiltration, compensatory hyperfiltration (e.g. after renal mass reduction by surgery), hyperfiltrative chronic kidney disease, hyperfiltrative acute renal failure, non-alcoholic steatohepatitis (NASH), non-alcoholic fatty liver disease (NAFLD), liver fibrosis, cataracts, polycystic ovarian syndrome, irritable bowel syndrome, inflammation and cancer (including breast cancer, prostate cancer, uterine cancer, colon cancer, and pancreatic cancer).

In an embodiment, the compounds of formula (I) of the present invention inhibit enteropeptidase, preferably, the compounds of formula (I) of the present invention selectively inhibit enteropeptidase. In particular, the compounds of formula (I) of the present invention are non-absorbable inhibitors of enteropeptidase (wherein the compounds of formula (I) do not pass from the intestine into the blood stream).

In another embodiment, the present invention is directed to compounds which are derivatives of carboxylic ester incorporating a highly hydrophilic side chain, such as an amino acid, PEG, quaternary ammonium salt, and the like, and are further non-absorbable inhibitors of enteropeptidase.

In an embodiment, the present invention is directed to methods for the treatment of diseases, disorders and complications associated with enteropeptidase activity selected from the group consisting of obesity, excess weight, hypertension, hypercholesterolemia, hyperlipidemia, hypertriglyceridemia, heart disease, angina, atherosclerosis, heart disease, heart attack, ischemia, stroke, pancreatitis, impaired glucose tolerance (IGT), impaired fasting glucose (IFT), gestational diabetes, Type II or non-insulin dependent diabetes mellitus, Syndrome X (also known as Metabolic Syndrome), neuropathy, retinopathy, nerve damage or poor blood flow in the feet, chronic kidney disease, microalbuminuria (elevated urine albumin levels), macroalbuminuria, elevated urine albumin levels, elevated albumin/creatinine ratio (ACR), nephropathy (including but not limited to, hyperfiltrative diabetic nephropathy), renal hyperfiltration, glomerular hyperfiltration, renal allograft hyperfiltration, compensatory hyperfiltration (e.g. after renal mass reduction by surgery), hyperfiltrative chronic kidney disease, hyperfiltrative acute renal failure, non-alcoholic steatohepatitis (NASH), non-alcoholic fatty liver disease (NAFLD), liver fibrosis, cataracts, polycystic ovarian syndrome, irritable bowel syndrome, inflammation and cancer (including breast cancer, prostate cancer, uterine cancer, colon cancer, and pancreatic cancer), comprising administrating, to a subject in need thereof, a compound of formula (I) or a composition comprising a compound of formula (I), as herein described.

In another embodiment, the present invention is directed to the use of a compound of formula (I) or a composition comprising a compound of formula (I) for the treatment of diseases, disorders and complications associated with enteropeptidase activity selected from the group consisting of obesity, excess weight, hypertension, hypercholesterolemia, hyperlipidemia, hypertriglyceridemia, heart disease, angina, atherosclerosis, heart disease, heart attack, ischemia, stroke, pancreatitis, impaired glucose tolerance (IGT), impaired fasting glucose (IFT), gestational diabetes, Type II or non-insulin dependent diabetes mellitus, Syndrome X (also known as Metabolic Syndrome), neuropathy, retinopathy, nerve damage or poor blood flow in the feet, chronic kidney disease, microalbuminuria (elevated urine albumin levels), macroalbuminuria, elevated urine albumin levels, elevated albumin/creatinine ratio (ACR), nephropathy (including but not limited to, hyperfiltrative diabetic nephropathy), renal hyperfiltration, glomerular hyperfiltration, renal allograft hyperfiltration, compensatory hyperfiltration (e.g. after renal mass reduction by surgery), hyperfiltrative chronic kidney disease, hyperfiltrative acute renal failure, non-alcoholic steatohepatitis (NASH), non-alcoholic fatty liver disease (NAFLD), liver fibrosis, cataracts, polycystic ovarian syndrome, irritable bowel syndrome, inflammation and cancer (including breast cancer, prostate cancer, uterine cancer, colon cancer, and pancreatic cancer).

One skilled in the art will recognize that certain compounds of formula (I) may be further useful as intermediates in the synthesis of other compounds of formula (I) (for example, compounds of formula (I) wherein R³ is —NH—CH(═N(Boc))—NH(Boc) may be useful as N-protected intermediates for the synthesis of compounds of formula (I) wherein R³ is —NH—CH(NH)—NH₂).

In an embodiment, the present invention is directed to compounds of formula (I-A)

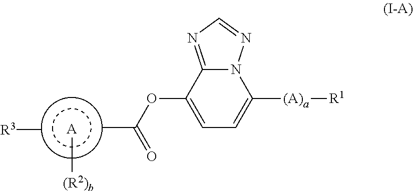

compounds of formula (I) wherein X is N. In another embodiment, the present invention is directed to compounds of formula (I-B)

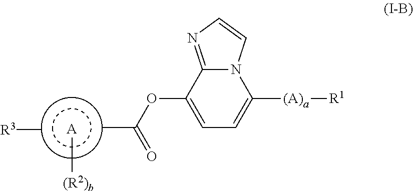

compounds of formula (I) wherein X is CH.

In an embodiment, the present invention is directed to compounds of formula (I) wherein a is an integer from 0 to 1. In another embodiment, the present invention is directed to compounds of formula (I) wherein a is 0. In another embodiment, the present invention is directed to compounds of formula (I) wherein a is 1.

In an embodiment, the present invention is directed to compounds of formula (I) wherein A is C₁₋₄alkyl. In another embodiment, the present invention is directed to compounds of formula (I) wherein A is selected from the group consisting of —CH₂—, —CH₂CH₂—, —CH₂CH₂CH₂— and —CH₂CH₂CH₂CH₂—. In another embodiment, the present invention is directed to compounds of formula (I) wherein A is selected from the group consisting of —CH₂—, —CH₂CH₂—, —CH₂CH₂CH₂—, —CH(CH₂)CH₂— and —CH₂CH(CH₂)—. In another embodiment, the present invention is directed to compounds of formula (I) wherein A is selected from the group consisting of —CH₂— and —CH₂CH₂—. In another embodiment, the present invention is directed to compounds of formula (I) wherein A is —CH₂—. In another embodiment, the present invention is directed to compounds of formula (I) wherein A is —CH₂CH₂—.

In an embodiment, the present invention is directed to compounds of formula (I) wherein R¹ is selected from the group consisting of 1,2,3,5-tetrazol-4-yl, imidazolidin-5-yl-2,4-dione, oxazolidin-yl-2,4-dione, 1,2,4-oxadiazol-5(4H)-one, thiazolidin-5-yl-2,4-dione, —CO₂H, —C(O)—O—(C₁₋₄alkyl), —SO₃H, —C(O)—NH—CH₂—CO₂H, —C(O)—NH—CH(CO₂H)—CH₂—CO₂H, —C(O)—N(CO₂H)—CH₂—CO₂H, —C(O)—N(CH₂CO₂H)—CH₂—CO₂H, —C(O)—NH—C(CH₂CH₂—CO₂H)₃, —C(O)—NH—SO₂—(C₁₋₄alkyl), —C(O)—NH—CH(R⁵)—CO₂H and —C(O)-proline; and wherein R⁵ is selected from the group consisting of hydrogen, methyl, isopropyl, 2-methylpropyl, 3-methylpropyl, benzyl, 4-hydroxy-benzyl, hydroxy-methyl, 2-hydroxy-ethyl, carboxyl-methyl and 2-carboxy-ethyl. In another embodiment, the present invention is directed to compounds of formula (I) wherein R¹ is selected from the group consisting of 1,2,3,5-tetrazol-4-yl, oxazolidin-5-yl-2,4-dione, —C(O)OH, —C(O)—O—(C₁₋₄alkyl), —C(O)—NH—CH₂—CO₂H, —C(O)—NH—CH(CO₂H)—CH₂—CO₂H, —C(O)—N(CH₂CO₂H)—CH₂—CO₂H, —C(O)—NH—C(CH₂CH₂—CO₂H)₃ and —C(O)—NH—SO₂—(C₁₋₂alkyl).

In another embodiment, the present invention is directed to compounds of formula (I) wherein R¹ is selected from the group consisting of 1,2,3,5-tetrazol-4-yl, oxazolidin-5-yl-2,4-dione, —C(O)OH, 2-(C(O)OH), —C(O)—NH—CH₂—CO₂H, —C(O)—O—C(CH₃)₃, —C(O)—N(CH₂CO₂H)—CH₂—CO₂H, —C(O)—NH—CH(CO₂H)—CH₂—CO₂H, —C(O)—NH—C(CH₂CH₂—CO₂H)₃ and —C(O)—NH—SO₂—CH₃. In another embodiment, the present invention is directed to compounds of formula (I) wherein R¹ is selected from the group consisting of 1,2,3,5-tetrazol-4-yl, oxazolidin-5-yl-2,4-dione, —C(O)OH, 2-(C(O)OH), —C(O)—O—C(CH₃)₃, —C(O)—NH—CH₂—CO₂H, —C(O)—NH—CH(CO₂H)—CH₂—CO₂H, —C(O)—NH—C(CH₂CH₂—CO₂H)₃ and —C(O)—NH—SO₂—CH₃. In another embodiment, the present invention is directed to compounds of formula (I) wherein R¹ is selected from the group consisting of 1,2,3,5-tetrazol-4-yl, oxazolidin-5-yl-2,4-dione, —C(O)OH, —C(O)—NH—CH(CO₂H)—CH₂—CO₂H and —C(O)—NH—SO₂—CH₃.

In another embodiment, the present invention is directed to compounds of formula (I) wherein R¹ is selected from the group consisting of —C(O)OH, 2-(C(O)OH) and —C(O)—NH—CH(CO₂H)—CH₂—CO₂H. In another embodiment, the present invention is directed to compounds of formula (I) wherein R¹ is selected from the group consisting of —C(O)OH, —C(O)—NH—CH(CO₂H)—CH₂—CO₂H and —C(O)—NH—C(CH₂CH₂—CO₂H)₃. In another embodiment, the present invention is directed to compounds of formula (I) wherein R¹ is selected from the group consisting of —C(O)OH and —C(O)—NH—SO₂—CH₃.

In an embodiment, the present invention is directed to compounds of formula (I) wherein

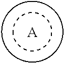

is selected from the group consisting of phen-1,4-diyl, phen-1,3-diyl, 6 membered heteroaryl and 9 membered heteroaryl; wherein the 6 membered heteroaryl is bound to the compound through two carbon atoms in para orientation; and wherein the 9 membered heteroaryl is bound to the compound through two carbon atoms in a para orientation.

In another embodiment, the present invention is directed to compounds of formula (I) wherein

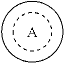

is selected from the group consisting of phen-1,4-diyl, thiophen-2,5-diyl, pyrimidin-2,5-diyl and benzofur-4,7-diyl. In another embodiment, the present invention is directed to compounds of formula (I) wherein is

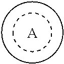

selected from the group consisting of phen-1,4-diyl, thiophen-2,5-diyl and pyrimidin-2,5-diyl. In another embodiment, the present invention is directed to compounds of formula (I) wherein

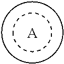

is selected from the group consisting of phen-1,4-diyl and pyrimidin-2,5-diyl. In another embodiment, the present invention is directed to compounds of formula (I) wherein

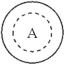

is pheny-1,4-diyl.

In an embodiment, the present invention is directed to compounds of formula (I) wherein b is an integer from 0 to 1. In another embodiment, the present invention is directed to compounds of formula (I) wherein b is 0. In another embodiment, the present invention is directed to compounds of formula (I) wherein b is 1.

In an embodiment, the present invention is directed to compounds of formula (I) wherein R² is selected from the group consisting of halogen, hydroxy, C₁₋₄alkyl, fluorinated C₁₋₂alkyl, C₁₋₄alkoxy, fluorinated C₁₋₂alkoxy, cyano, 3-(prop-2-yn-1-yloxy)prop-1-yn-1-yl and 3-(2-(prop-2-yn-1-yloxy)ethoxy)prop-1-yn-1-yl; provided that R² is bound to a carbon atom; and provided further that when R² is 3-(prop-2-yn-1-yloxy)prop-1-yn-1-yl or 3-(2-(prop-2-yn-1-yloxy)ethoxy)prop-1-yn-1-yl, then the R² is bound to a carbon atom at the ortho position to the —C(O)—O— group of the compound of formula (I).

In another embodiment, the present invention is directed to compounds of formula (I) wherein R² is selected from the group consisting of halogen, hydroxy, C₁₋₂alkyl, fluorinated C₁₋₂alkyl, C₁₋₂alkoxy, fluorinated C₁₋₂alkoxy, cyano, 3-(prop-2-yn-1-yloxy) prop-1-yn-1-yl and 3-(2-(prop-2-yn-1-yloxy) ethoxy) prop-1-yn-1-yl; provided that R² is bound to a carbon atom; and provided further that when R² is 3-(prop-2-yn-1-yloxy)prop-1-yn-1-yl or 3-(2-(prop-2-yn-1-yloxy)ethoxy)prop-1-yn-1-yl, then the R² is bound to a carbon atom at the ortho position to the —C(O)—O— group of the compound of formula (I).

In another embodiment, the present invention is directed to compounds of formula (I) wherein R² is selected from the group consisting of 2-chloro, 2-fluoro, 2-iodo, 3-chloro, 3-fluoro, 3-iodo, 3-hydroxy, 2-methyl, 3-methyl, 2-trifluoromethyl, 3-methoxy, 3-cyano, 3-(prop-2-yn-1-yloxy) prop-1-yn-1-yl and 3-(2-(prop-2-yn-1-yloxy) ethoxy) prop-1-yn-1-yl.

In another embodiment, the present invention is directed to compounds of formula (I) wherein R² is selected from the group consisting of 2-chloro, 2-fluoro, 3-fluoro, 3-hydroxy, 3-(prop-2-yn-1-yloxy) prop-1-yn-1-yl and 3-(2-(prop-2-yn-1-yloxy) ethoxy) prop-1-yn-1-yl. In another embodiment, the present invention is directed to compounds of formula (I) wherein R² is selected from the group consisting of 2-chloro, 2-fluoro and 3-fluoro. In another embodiment, the present invention is directed to compounds of formula (I) wherein R² is selected from the group consisting of 2-fluoro and 3-fluoro.

In an embodiment, the present invention is directed to compounds of formula (I) wherein R³ is selected from the group consisting —CH(═NH)—NH—NH₂, —CH(═NH)—NH(C₁₋₄alkyl), —CH(═NH)—NH(OC₁₋₄alkyl), —CH(═NH)—NH—CN, —NH—CH(═NH)—NH₂, —NH—CH(═N(Boc))—NH(Boc), —NH—CH(═NH)—NH(C₁₋₄alkyl), —NH—CH(═NH)—NH(OH), —NH—CH(═NH)—NH—O(C₁₋₄alkyl), —N(C₁₋₄alkyl)-CH(═NH)—NH₂, —N(C₁₋₂alkyl)-CH(═NH)—NH(C₁₋₂alkyl), —NH—CH(═NH)—(C₁₋₄alkyl), —NH—C(═NOH)—(C₁₋₄alkyl) and imidazolidin-1-yl-2-imine.

In another embodiment, the present invention is directed to compounds of formula (I) wherein R³ is selected from the group consisting of —CH(═NH)—NH—NH₂, —CH(═NH)—NH(C₁₋₂alkyl), —CH(═NH)—NH(OC₁₋₂alkyl), —CH(═NH)—NH—CN, —NH—CH(═NH)—NH₂, —NH—CH(═N(Boc))—NH(Boc), —NH—CH(═NH)—NH(C₁₋₂alkyl), —N(C₁₋₂alkyl)-CH(═NH)—NH₂, —NH—CH(═NH)—(C₁₋₂alkyl), —NH—C(═NOH)—(C₁₋₂alkyl) and imidazolidin-1-yl-2-imine.

In another embodiment, the present invention is directed to compounds of formula (I) wherein R³ is selected from the group consisting of —CH(═NH)—NH—NH₂, —CH(═NH)—NH(CH₃), —CH(═NH)—NH(OCH₃), —CH(═NH)—NH—CN, —NH—CH(═NH)—NH₂, —NH—CH(═N(Boc))—NH(Boc), —NH—CH(═NH)—NH(CH₃), —N(CH₃)—CH(═NH)—NH₂, —NH—CH(═NH)—CH₃, —NH—C(═NOH)—CH₃ and imidazolidin-1-yl-2-imine.

In another embodiment, the present invention is directed to compounds of formula (I) wherein R³ is selected from the group consisting of —NH—CH(═NH)—NH₂ and —N(CH₃)—CH(═NH)—NH₂. In another embodiment, the present invention is directed to compounds of formula (I) wherein R³ is —NH—CH(═NH)—NH₂.

In an embodiment, the present invention is directed to compound of formula (I) selected from the group consisting of
2-[8-(4-guanidinobenzoyl)oxy-[1,2,4]triazolo[1,5-a]pyridin-5-yl]acetic acid;
3-[8-(4-guanidinobenzoyl)oxy-[1,2,4]triazolo[1,5-a]pyridin-5-yl]propanoic acid;
2-[[2-[8-(4-guanidinobenzoyl)oxy-[1,2,4]triazolo[1,5-a]pyridin-5-yl]acetyl]amino]butanedioic acid;
4-(2-carboxyethyl)-4-[[2-[8-(4-guanidinobenzoyl)oxy-[1,2,4]triazolo[1,5-a]pyridin-5-yl]acetyl]amino]heptanedioic acid;
2-[8-(4-guanidinobenzoyl)oxyimidazo[1,2-a]pyridin-5-yl]acetic acid;

and pharmaceutically acceptable salts thereof.

Additional embodiments of the present invention, include those wherein the substituents selected for one or more of the variables defined herein (i.e. a, b, A, R¹, R², R³, X,

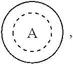

etc.) are independently selected to be any individual substituent or any subset of substituents selected from the complete list as defined herein. Additional embodiments of the present invention, include those wherein the substituents selected for one or more of the variables defined herein (i.e. a, b, A, R¹, R², R³, X,

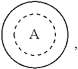

etc.) are independently selected to correspond to any of the embodiments as defined herein.

In another embodiment of the present invention is any single compound or subset of compounds selected from the representative compounds listed in Table 1, below.

Representative compounds of the present invention are as listed in Table 1, below. Unless otherwise noted, wherein a stereogenic center is present in the listed compound, the compound was prepared as a mixture of stereo-configurations.

TABLE 1
Representative Compounds of Formula (I)

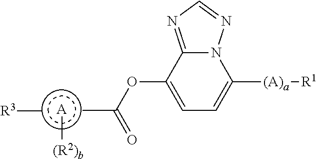

| ID No | —(A)ₐ— | —R¹ | 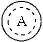 | (R²)_{b} | R³ |
|---|---|---|---|---|---|
| 1 | —CH₂— | —C(O)OH | phen-1,4- | b = 0 | —NH—CH(═NH)—NH₂ |
| | | | diyl | | |
| 3 | —CH₂—CH₂— | —C(O)OH— | phen-1,4- | b = 0 | —NH—CH(═NH)—NH₂ |
| | | | diyl | | |
| 4 | —CH₂— | —C(O)—NH—CH₂—CO₂H | phen-1,4- | b = 0 | —NH—CH(═NH)—NH₂ |
| | | | diyl | | |
| 5 | —CH₂— | —C(O)—NH—CH(CO₂H)—CH₂—CO₂H | phen-1,4- | b = 0 | —NH—CH(═NH)—NH₂ |
| | | | diyl | | |
| 6 | —CH₂— | —C(O)—N(CH₂CO₂H)—CH₂—CO₂H | phen-1,4- | b = 0 | —NH—CH(═NH)—NH₂ |
| | | | diyl | | |
| 7 | —CH₂— | —C(O)—NH—C(CH₂CH₂—CO₂H)₃ | phen-1,4- | b = 0 | —NH—CH(═NH)—NH₂ |
| | | | diyl | | |
| 8 | —CH₂— | —C(O)—NH—SO₂—CH₃ | phen-1,4- | b = 0 | —NH—CH(═NH)—NH₂ |
| | | | diyl | | |
| 10 | —CH₂— | —C(O)OH | thiophen- | b = 0 | —NH—CH(═NH)—NH₂ |
| | | | 2,5-diyl | | |
| 11 | —CH₂— | 1,2,3,5-tetrazol-4-yl | phen-1,4- | b = 0 | —NH—CH(═NH)—NH₂ |
| | | | diyl | | |
| 12 | —CH₂— | —C(O)OH | phen-1,4- | 3-methoxy | —NH—CH(═NH)—NH₂ |
| | | | diyl | | |
| 13 | —CH₂— | —C(O)OH | phen-1,4- | 3-iodo | —NH—CH(═NH)—NH₂ |
| | | | diyl | | |
| 14 | —CH₂— | —C(O)OH | phen-1,4- | 3-fluoro | —NH—CH(═NH)—NH₂ |
| | | | diyl | | |
| 15 | —CH₂— | —C(O)OH | phen-1,4- | 3-methyl | —NH—CH(═NH)—NH₂ |
| | | | diyl | | |
| 16 | —CH₂— | —C(O)OH | phen-1,4- | 3-chloro | —NH—CH(═NH)—NH₂ |
| | | | diyl | | |
| 17 | a = 0 | oxazolidin-5-yl-2,4-dione | phen-1,4- | b = 0 | —NH—CH(═NH)—NH₂ |
| | | | diyl | | |
| 18 | —CH₂— | —C(O)OH | phen-1,4- | 2-chloro | —NH—CH(═NH)—NH₂ |
| | | | diyl | | |
| 19 | —CH₂— | —C(O)OH | phen-1,4- | 2-methyl | —NH—CH(═NH)—NH₂ |
| | | | diyl | | |
| 20 | —CH₂— | —C(O)OH | phen-1,4- | 2- | —NH—CH(═NH)—NH₂ |
| | | | diyl | trifluoro- | |
| | | | | methyl | |
| 21 | —CH₂— | —C(O)OH | phen-1,4- | 2-fluoro | —NH—CH(═NH)—NH₂ |
| | | | diyl | | |
| 22 | —CH₂— | —C(O)OH | phen-1,4- | 3-cyano | —NH—CH(═NH)—NH₂ |
| | | | diyl | | |
| 23 | —CH₂— | —C(O)OH | phen-1,4- | 3-hydroxy | —NH—CH(═NH)—NH₂ |
| | | | diyl | | |
| 24 | —CH₂— | —C(O)OH | benzofur- | b = 0 | —NH—CH(═NH)—NH₂ |
| | | | 4,7-diyl | | |
| 25 | —CH₂—CH₂— | 2—C(O)OH | phen-1,4- | b = 0 | —NH—CH(═NH)—NH₂ |
| | | | diyl | | |
| 26 | —CH₂— | —C(O)OH | phen-1,4- | b = 0 | —NH—CH(═NH)—NH₂ |
| | | | diyl | | |
| 28 | —CH₂— | —C(O)—NH—CH(CO₂H)—CH₂—CO₂H | pyrimidin- | b = 0 | —NH—CH(═NH)—NH₂ |
| | | | 2,5-diyl | | |
| 30 | —CH₂— | —C(O)—NH—SO₂—CH₃ | pyrimidin- | b = 0 | —NH—CH(═NH)—NH₂ |
| | | | 2,5-diyl | | |
| 32 | —CH₂— | —C(O)OH | phen-1,4- | b = 0 | imidazolidin-1-yl- |
| | | | yl | | 2-imine |
| 33 | —CH₂— | —C(O)OH | phen-1,4- | b = 0 | —CH(═NH)—NH(CH₃) |
| | | | diyl | | |
| 34 | —CH₂— | —C(O)OH | phen-1,4- | b = 0 | —NH—CH(═NH)—NH(CH₃) |
| | | | diyl | | |
| 35 | —CH₂— | —C(O)OH | phen-1,4- | b = 0 | —N(CH₃)—CH(═NH)—NH₂ |
| | | | diyl | | |
| 36 | —CH₂— | —C(O)OH | phen-1,4- | b = 0 | —CH(═NH)—NH(OCH₃) |
| | | | diyl | | |
| 37 | —CH₂— | —C(O)OH | phen-1,4- | b = 0 | —NH—CH(═NH)—CH₃ |
| | | | diyl | | |
| 38 | —CH₂— | —C(O)OH | phen-1,4- | b = 0 | —CH(═NH)—NH—CN |
| | | | diyl | | |
| 39 | —CH₂— | —C(O)OH | phen-1,4- | b = 0 | —CH(═NH)—NH—NH₂ |
| | | | diyl | | |
| 40 | —CH₂— | —C(O)OH | phen-1,4- | b = 0 | —NH—C(═NOH)—CH₃ |
| | | | diyl | | |
| 41 | —CH₂— | —C(O)—O—C(CH₃)₃ | phen-1,4- | 2-iodo | —NH—CH(═NBoc)—NH(Boc) |
| | | | diyl | | |
| 42 | —CH₂— | —C(O)OH | phen-1,4- | 3-(2-(prop-2- | —NH—CH(═NH)—NH₂ |
| | | | diyl | yn-1-yloxy) | |
| | | | | ethoxy) | |
| | | | | prop- | |
| | | | | 1-yn-1-yl | |
| 43 | —CH_{2—} | —C(O)OH | phen-1,4- | 3-(prop-2- | —NH—CH(═NH)—NH₂ |
| | | | | yn- | |
| | | | diyl | 1-yloxy) | |
| | | | | prop- | |
| | | | | 1-yn-1-yl | |
| 2 | —CH₂—CH₂— | 2—C(O)OH— | phen-1,4- | b = 0 | —NH—CH(═NH)—NH₂ |
| | | | diyl | | |
| 9 | —CH₂— | —C(O)OH | phen-1,4- | b = 0 | —NH—CH(═NH)—NH₂ |
| | | | diyl | | |
| 27 | —CH₂— | —C(O)—NH—CH(CO₂H)—CH₂—CO₂H | phen-1,4- | b = 0 | —NH—CH(═NH)—NH₂ |
| | | | diyl | | |
| 29 | —CH₂— | —C(O)OH | pyrimidin- | b = 0 | —NH—CH(═NH)—NH₂ |
| | | | 2,5-diyl | | |
| 31 | —CH₂— | —C(O)—NH—CH(CO₂H)—CH₂—CO₂H | pyrimidin- | b = 0 | —NH—CH(═NH)—NH₂ |
| | | | 2,5-diyl | | |

In an embodiment, the present invention is directed to a compound of formula (I); wherein the compound of formula exhibits a % Inhibition at 0.1 μM against enteropeptidase, measured as described in Biological Example 1, which follows hereinafter, of greater than or equal to about 20%, preferably greater than or equal to about 50%, more preferably greater than or equal to about 75%, more preferably greater than or equal to about 80%, more preferably greater than or equal to about 90%, more preferably greater than or equal to about 95%.

In an embodiment, the present invention is directed to a compound of formula (I); wherein the compound of formula exhibits a % Inhibition at 0.1 μM against trypsin, measured as described in Biological Example 2, which follows hereinafter, of greater than or equal to about 20%, preferably greater than or equal to about 30%, more preferably greater than or equal to about 50%, more preferably greater than or equal to about 75%, more preferably greater than or equal to about 80%, more preferably greater than or equal to about 90%, more preferably greater than or equal to about 95%.

### Definitions

As used herein, “halogen” shall mean chloro, bromo, fluoro and iodo.

As used herein, the term “C_{X-Y}alkyl” wherein X and Y are integers, whether used alone or as part of a substituent group, include straight and branched chains containing between X and Y carbon atoms. For example, C₁₋₄alkyl radicals include straight and branched chains of between 1 and 4 carbon atoms, including methyl, ethyl, propyl, isopropyl, butyl, isobutyl, sec-butyl and t-butyl.

One skilled in the art will recognize that when the term “C_{X-Y}alkyl” is used as part of a larger substituent group, in the form of “—(C_{X-Y}alkyl)- and —C_{X-Y}alkyl-” wherein X and Y are integers, then said term shall denote any C_{X-Y}alkyl carbon chain as herein defined, wherein said C_{X-Y}alkyl chain is divalent and is further bound through two points of attachment, preferably through two terminal carbon atoms.

As used herein, unless otherwise noted, the terms “fluorinated C_{X-Y}alkyl” and “fluoro substituted C_{X-Y}alkyl” shall mean any C_{X-Y}alkyl group as defined above substituted with at least one fluorine atom, preferably one to three fluorine atoms. In an example, “fluorinated C₁₋₄alkyl” include, but are not limited, to —CH₂F, —CF₂H, —CF₃, —CH₂—CF₃, —CF₂—CF₂—CF₂—CF₃, and the like.

As used herein, unless otherwise noted, “C₁₋₄alkoxy” shall denote an oxygen ether radical of the above described straight or branched chain alkyl groups containing one to four carbon atoms. For example, methoxy, ethoxy, n-propoxy, isopropoxy, sec-butoxy, t-butoxy, and the like.

As used herein, unless otherwise noted, the terms “fluorinated C_{X-Y}alkoxy” and “fluoro substituted C_{X-Y}alkoxy”, shall mean any C_{X-Y}alkoxy group as defined above substituted with at least one fluorine atom, preferably one to three fluorine atoms. In an example, “fluorinated C₁₋₄alkoxy” include, but are not limited, —OCH₂F, —OCF₂H, —OCF₃, —OCH₂—CF₃, —OCF₂—CF₂—CF₂—CF₃, and the like.

As used herein, unless otherwise noted, “5-6 membered heteroaryl” shall denote any five or six membered monocyclic aromatic ring structure containing at least one heteroatom selected from the group consisting of O, N and S, optionally containing one to three additional heteroatoms independently selected from the group consisting of O, N and S. Suitable examples include, but are not limited to, pyrrolyl, furyl, thienyl, oxazolyl, imidazolyl, purazolyl, isoxazolyl, isothiazolyl, triazolyl, thiadiazolyl, pyridyl, pyridazinyl, pyrimidinyl, pyrazinyl, pyranyl, furazanyl, and the like.

As used herein, unless otherwise noted, the term “bicyclic 9-10 membered heteroaryl” shall denote any nine or ten membered bicyclic aromatic ring structure containing at least one heteroatom selected from the group consisting of O, N and S, optionally containing one to four additional heteroatoms independently selected from the group consisting of O, N and S. Suitably examples include, but are not limited to, indolyl, isoindolinyl, indazolyl, benzofuryl, benzothienyl, benzimidazolyl, benzthiazolyl, purinyl, quinolizinyl, quinolinyl, isoquinolinyl, isothiazolyl, cinnolinyl, phthalazinyl, quinazolinyl, quinoxalinyl, naphthyridinyl, pteridinyl, and the like.

When a particular group is “substituted” (e.g. C_{X-Y}alkyl, C_{X-Y}alkoxy, C_{X-Y}cycloalkyl, etc.), that group may have one or more substituents, preferably from one to five substituents, more preferably from one to three substituents, most preferably from one to two substituents, independently selected from the list of substituents.

With reference to substituents, the term “independently” means that when more than one of such substituents is possible, such substituents may be the same or different from each other.

In an embodiment of the present invention, wherein any substituent or part or a substituent group is an amino acid, said amino acid is preferably present in a racemic mixture or in an enantiomeric excess of the L-configuration.

As used herein, the notation “*” shall denote the presence of a stereogenic center.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Preferably, wherein the compound is present as an enantiomer, the enantiomer is present at an enantiomeric excess of greater than or equal to about 80%, more preferably, at an enantiomeric excess of greater than or equal to about 90%, more preferably still, at an enantiomeric excess of greater than or equal to about 95%, more preferably still, at an enantiomeric excess of greater than or equal to about 98%, most preferably, at an enantiomeric excess of greater than or equal to about 99%. Similarly, wherein the compound is present as a diastereomer, the diastereomer is present at an diastereomeric excess of greater than or equal to about 80%, more preferably, at an diastereomeric excess of greater than or equal to about 90%, more preferably still, at an diastereomeric excess of greater than or equal to about 95%, more preferably still, at an diastereomeric excess of greater than or equal to about 98%, most preferably, at an diastereomeric excess of greater than or equal to about 99%.

Furthermore, some of the crystalline forms for the compounds of the present invention may exist as polymorphs and as such are intended to be included in the present invention. In addition, some of the compounds of the present invention may form solvates with water (i.e., hydrates) or common organic solvents, and such solvates are also intended to be encompassed within the scope of this invention.

As used herein, unless otherwise noted, the term “isotopologues” shall mean molecules that differ only in their isotopic composition. More particularly, an isotopologue of a molecule differs from the parent molecule in that it contains at least one atom which is an isotope (i.e. has a different number of neutrons from its parent atom).

For example, isotopologues of water include, but are not limited to, “light water” (HOH or H₂O), “semi-heavy water” with the deuterium isotope in equal proportion to protium (HDO or ¹H²HO), “heavy water” with two deuterium isotopes of hydrogen per molecule (D₂O or ²H₂O), “super-heavy water” or tritiated water (T₂O or ³H₂O), where the hydrogen atoms are replaced with tritium (³H) isotopes, two heavy-oxygen water isotopologues (H₂¹⁸O and H₂¹⁷O) and isotopologues where the hydrogen and oxygen atoms may each independently be replaced by isotopes, for example the doubly labeled water isotopologue D₂¹⁸O.

It is intended that within the scope of the present invention, any one or more element(s), in particular when mentioned in relation to a compound of formula (I), shall comprise all isotopes and isotopic mixtures of said element(s), either naturally occurring or synthetically produced, either with natural abundance or in an isotopically enriched form. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O. The isotopes may be radioactive or non-radioactive.

Radiolabeled compounds of formula (I) may comprise one or more radioactive isotope(s) selected from the group of ³H, ¹¹C, ¹⁸F, ¹²²I, ¹²³I, ¹²⁵I, ¹³¹I, ⁷⁵Br, ⁷⁶Br, ⁷⁷Br and ⁸²Br. Preferably, the radioactive isotope is selected from the group of ³H ¹¹C and ¹⁸F.

It is further intended that the present invention includes the compounds described herein, including all isomers thereof (including, but not limited to stereoisomers, enantiomers, diastereomers, tautomers, isotopologues, isotopomers, and the like).

Under standard nomenclature used throughout this disclosure, the terminal portion of the designated side chain is described first, followed by the adjacent functionality toward the point of attachment. Thus, for example, a “phenylC₁-C₆alkylaminocarbonylC₁-C₆alkyl” substituent refers to a group of the formula

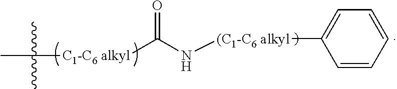

Abbreviations used in the specification, particularly the Schemes and Examples, are as listed in Table A, below.

TABLE A
Abbreviations

| | | |
|---|---|---|
| ACE | = | Angiotensin Converting Enzyme |
| EtOAc or EA | = | Ethyl Acetate |
| ACR | = | Albumin/Creatnine Ratio |
| ARB | = | Angiotensin receptor blockers |
| ARF | = | Acute Renal Failure |
| ASH | = | Alcoholic Steatohepatitis |
| BF₃•Et₂O | = | Boron trifluoride diethyl etherate |
| BMI | = | Body Mass Index |
| Bn or Bz | = | Benzyl |
| Boc or BOC | = | tert-Butoxycarbonyl |
| Boc₂O | = | tert-Butoxidecarbonyl Anhydride |
| t-BuOK | = | Potassium tert-Butoxide |
| CBz | = | Benzyloxycarbonyl |
| CBzCl | = | Benzyloxycarbonyl Chloride |
| CDI | = | Carbonyldiimidazole |
| CHD | = | Coronary Heart Disease |
| CKD | = | Chronic Kidney Disease |
| CPhos | = | 2-Dicyclohexylphosphine-2’,6’-bis(N,N- |
| | | dimethylamino)biphenyl |
| CSA | = | Camphorsulfonic Acid |
| DBP | = | Diastolic Blood Pressure |
| DCC | = | N,N’-Dicyclohexylcarbodiimide |
| DCM | = | Dichloromethane |
| Dess-Martin Reagent | = | 1,1,1-Triacetoxy-1,1-dihydro-1,2-benziodoxol- |
| | | 3(1H)-one |
| DIBAL or DIBAL-H | = | Diidobutylaluminum hydride |
| DIPEA or DIEA | = | Diisopropylethylamine |
| DMAP | = | 4-Dimethylaminopyridine |
| DMF | = | N,N-Dimethylformamide |
| DMF-DMA | = | N,N-Dimethylformamide Dimethylacetal |
| DMSO | = | Dimethylsulfoxide |
| dppf | = | 1,1’-Bis(diphenylphosphino)ferrocene |
| EDCI | = | 1-Ethyl-3-(3-dimetylaminopropyl)carbodiimide |
| eGFR | = | Estimated Glomerular Filtration Rate |
| EP | = | Enteropeptidase (enzyme) |
| EtOH | = | Ethanol |
| Et₃N or TEA | = | Triethylamine |
| EtOAc or EA | = | Ethyl acetate |
| FI | = | Fluorescence Intensity |
| GFR | = | Glomerular Filtration Rate |
| HATU | = | O-(7-Azabenzotriazol-1-yl)-N,N,N”,N”-Tetramethyl |
| | | Uronium Hexafluorophosphate |
| HDL | = | High density lipoprotein |
| HOBT or HOBt | = | 1-Hydroxybenzotriazole |
| HPLC | = | High Pressure Liquid Chromatography |
| IFG | = | Impaired fasting glucose |
| IGT | = | Impaired glucose tolerance |
| LAH or LiAlH₄ | = | Lithium aluminum hydride |
| LBH | = | Lithium Borohydride |
| LC-MS or LCMS | = | Liquid chromatography-mass spectrometry |
| LDL | = | Low Density Lipoprotein |
| LiHMDS | = | Lithium hexamethyldisilazide or Lithium |
| | | bis(trimethylsilyl)amide |
| MACE | = | Major Adverse Cardiac Event |
| mCPBA | | meta-Chloroperoxybenzoic Acid |
| Me | = | Methyl (i.e.-CH₃) |
| MeOH | = | Methanol |
| MOM | = | Methoxy methyl |
| Ms | = | Mesyl (methane sulfonyl) |
| MsCl | = | Mesyl Chloride (methanesulfonyl chloride) |
| MTBE | = | Methyl t-butyl ether |
| NAFLD | = | Non-alcoholic fatty liver disease |
| NaOAc | = | Sodium Acetate |
| NaOMe | = | Sodium Methoxide |
| NaOEt | = | Sodium Ethoxide |
| NASH | = | Non-alcoholic steatohepatitis |
| NMR | = | Nuclear magnetic resonance |
| OGTT | = | Oral Glucose Tolerance Test |
| OTf | = | Triflate (trifluoromethanesulfonate) |
| Pd/C | = | Palladium on Carbon (catalyst) |
| Pd₂(dba)₃ | = | Tris(dibenzylideneacetone) dipalladium (0) |
| Pd(PPh₃)₄ or | = | Tetrakistriphenylphosphine palladium (0) |
| Pd(Ph₃P)₄ | | |
| Pd(Ph₃P)₂Cl₂ or | | Bis(triphenylphosphine) palladium(II) dichloride |
| Pd(PPh₃)₂Cl₂ | | |
| PE | = | Petroleum ether |
| PPh₃ | = | Triphenylphosphine |
| i-PrOH | = | Isopropanol |
| PyBrop | = | Bromortripyrrolidinophosphonium |
| | | hexafluorophosphate |
| Q-Phos | = | 1,2,3,4,5-Pentaphenyl-1’-(Di-tert- |
| | | butylphosphino)ferrocene |
| SBP | = | Systolic Blood Pressure |
| −Sn(Bu)₃ | = | Tributyl Tin |
| TBAF | = | Tetra-n-butylammonium fluoride |
| TEA | = | Triethylamine |
| TFA | = | Trifluoroacetic Acid |
| TFAA | = | Trifluoroacetic acid anhydride |
| THF | = | Tetrahydrofuran |
| THP | = | Tetrahydropyran |
| TLC | = | Thin Layer Chromatography |
| TMS | = | Trimethylsilyl |
| TMSCN | = | Trimethylsilyl Cyanide |
| TMSN₃ | | Trimethylsilyl Azide |
| (o-Tol)₃P | = | Tris(o-tolyl)phosphine |
| Tris HCl or Tris-Cl | = | Tris[hydroxymethyl]aminomethyl hydrochloride |
| or Tris base | | |
| Triton X-100 | = | Non-ionic surfactant (C₁₄H₂O(C₂H₄O)ₙ |
| Ts | = | Tosyl (p-toluenesulfonyl) |
| pTSA | = | para-Toluenesulfonic Acid |
| TsCl | = | Tosyl chloride (p-toluenesulfonyl chloride) |
| X-Phos or XPhos | = | 2-Dicyclohexylphosphino-2’,4’,6’- |
| | | triisopropylphenyl |

As used herein, unless otherwise noted, the term “isolated form” shall mean that the compound is present in a form which is separate from any solid mixture with another compound(s), solvent system or biological environment. In an embodiment of the present invention, the compound of formula (I) is present in an isolated form.

As used herein, unless otherwise noted, the term “substantially pure form” shall mean that the mole percent of impurities in the isolated compound is less than about 5 mole percent, preferably less than about 2 mole percent, more preferably, less than about 0.5 mole percent, most preferably, less than about 0.1 mole percent. In an embodiment of the present invention, the compound of formula (I) is present as a substantially pure form.

As used herein, unless otherwise noted, the term “substantially free of a corresponding salt form(s)” when used to described the compound of formula (I) shall mean that mole percent of the corresponding salt form(s) in the isolated base of formula (I) is less than about 5 mole percent, preferably less than about 2 mole percent, more preferably, less than about 0.5 mole percent, most preferably less than about 0.1 mole percent. In an embodiment of the present invention, the compound of formula (I) is present in a form which is substantially free of corresponding salt form(s).

In an embodiment of the present invention, the disease, disorder, or condition mediated by enteropeptidase activity is selected from the group consisting of obesity, excess weight, hypertension, hypercholesterolemia, hyperlipidemia, hypertriglyceridemia, heart disease, angina, atherosclerosis, heart disease, heart attack, ischemia, stroke and pancreatitis.

In another embodiment of the present invention, the disease, disorder, or condition mediated by enteropeptidase activity is selected from the group consisting of impaired glucose tolerance (IGT), impaired fasting glucose (IFT), gestational diabetes, Type II or non-insulin dependent diabetes mellitus, Syndrome X (also known as Metabolic Syndrome), neuropathy, retinopathy, nerve damage or poor blood flow in the feet.

In another embodiment of the present invention, the disease, disorder, or condition mediated by enteropeptidase activity is selected from the group consisting of chronic kidney disease, microalbuminuria (elevated urine albumin levels), macroalbuminuria; elevated urine albumin levels, elevated albumin/creatinine ratio (ACR). In another embodiment of the present invention, the disease, disorder, or condition mediated by enteropeptidase activity is selected from the group consisting of nephropathy (including but not limited to, hyperfiltrative diabetic nephropathy), renal hyperfiltration, glomerular hyperfiltration, renal allograft hyperfiltration, compensatory hyperfiltration (e.g. after renal mass reduction by surgery), hyperfiltrative chronic kidney disease, hyperfiltrative acute renal failure.

In another embodiment of the present invention, the disease, disorder, or condition mediated by enteropeptidase activity is selected from the group consisting of non-alcoholic steatohepatitis (NASH), non-alcoholic fatty liver disease (NAFLD), liver fibrosis.

In an embodiment of the present invention, the disease, disorder, or condition mediated by enteropeptidase activity is selected from the group consisting of hypertension, hypercholesterolemia, hyperlipidemia, hypertriglyceridemia, heart disease, angina, atherosclerosis, heart disease, heart attack, ischemia, stroke and pancreatitis.

In another embodiment of the present invention, the disease, disorder, or condition mediated by enteropeptidase activity is selected from the group consisting of cataracts, polycystic ovarian syndrome, irritable bowel syndrome, inflammation and cancer (including breast cancer, prostate cancer, uterine cancer, colon cancer, and pancreatic cancer).

In an embodiment of the present invention, the disease, disorder, or condition mediated by enteropeptidase activity is selected from the group consisting of obesity, excess weight, hypertension, hypercholesterolemia, hyperlipidemia, hypertriglyceridemia, heart disease, angina, atherosclerosis, heart disease, heart attack, ischemia, stroke, pancreatitis, impaired glucose tolerance (IGT), impaired fasting glucose (IFT), gestational diabetes, Type II or non-insulin dependent diabetes mellitus, Syndrome X (also known as Metabolic Syndrome), neuropathy, retinopathy, nerve damage or poor blood flow in the feet, chronic kidney disease, microalbuminuria (elevated urine albumin levels), macroalbuminuria, elevated urine albumin levels, elevated albumin/creatinine ratio (ACR), nephropathy (including but not limited to, hyperfiltrative diabetic nephropathy), non-alcoholic steatohepatitis (NASH), non-alcoholic fatty liver disease (NAFLD) and liver fibrosis.

In another embodiment of the present invention, the disease, disorder, or condition mediated by enteropeptidase activity is selected from the group consisting of obesity, excess weight, impaired glucose tolerance (IGT), impaired fasting glucose (IFT), gestational diabetes, Type II or non-insulin dependent diabetes mellitus, Syndrome X (also known as Metabolic Syndrome), neuropathy, retinopathy, nerve damage or poor blood flow in the feet, chronic kidney disease, microalbuminuria (elevated urine albumin levels), macroalbuminuria, elevated urine albumin levels, elevated albumin/creatinine ratio (ACR), hyperfiltrative diabetic nephropathy, non-alcoholic steatohepatitis (NASH) and non-alcoholic fatty liver disease (NAFLD).

In another embodiment of the present invention, the disease, disorder, or condition mediated by enteropeptidase activity is selected from the group consisting of obesity, impaired glucose tolerance (IGT), impaired fasting glucose (IFT), gestational diabetes, Type II or non-insulin dependent diabetes mellitus, Syndrome X (also known as Metabolic Syndrome), chronic kidney disease, microalbuminuria (elevated urine albumin levels), macroalbuminuria, elevated urine albumin levels, elevated albumin/creatinine ratio (ACR), hyperfiltrative diabetic nephropathy, non-alcoholic steatohepatitis (NASH) and non-alcoholic fatty liver disease (NAFLD).

In another embodiment of the present invention, the disease, disorder, or condition mediated by enteropeptidase activity is selected from the group consisting of obesity, impaired glucose tolerance (IGT), impaired fasting glucose (IFT), gestational diabetes, Type II or non-insulin dependent diabetes mellitus and Syndrome X (also known as Metabolic Syndrome).

In another embodiment of the present invention, the disease, disorder, or condition mediated by enteropeptidase activity is selected from the group consisting of chronic kidney disease, microalbuminuria (elevated urine albumin levels), macroalbuminuria, elevated urine albumin levels, elevated albumin/creatinine ratio (ACR) and hyperfiltrative diabetic nephropathy. In another embodiment of the present invention, the disease, disorder, or condition mediated by enteropeptidase activity is selected from the group consisting of non-alcoholic steatohepatitis (NASH) and non-alcoholic fatty liver disease (NAFLD).

As used herein, unless otherwise noted, the terms “treating”, “treatment” and the like, shall include the management and care of a subject or patient (preferably mammal, more preferably human) for the purpose of combating a disease, condition, or disorder and includes the administration of a compound of the present invention to prevent the onset of the symptoms or complications, alleviate the symptoms or complications, slow the progression of the disease or disorder, or eliminate the disease, condition, or disorder.

As used herein, unless otherwise noted, the term “prevention” shall include (a) reduction in the frequency of one or more symptoms; (b) reduction in the severity of one or more symptoms; (c) the delay or avoidance of the development of additional symptoms; and/or (d) delay or avoidance of the development of the disorder or condition.

One skilled in the art will recognize that wherein the present invention is directed to methods of prevention, a subject in need of thereof (i.e. a subject in need of prevention) shall include any subject or patient (preferably a mammal, more preferably a human) who has experienced or exhibited at least one symptom of the disorder, disease or condition to be prevented. Further, a subject in need thereof may additionally be a subject (preferably a mammal, more preferably a human) who has not exhibited any symptoms of the disorder, disease or condition to be prevented, but who has been deemed by a physician, clinician or other medical profession to be at risk of developing said disorder, disease or condition. For example, the subject may be deemed at risk of developing a disorder, disease or condition (and therefore in need of prevention or preventive treatment) as a consequence of the subject's medical history, including, but not limited to, family history, pre-disposition, co-existing (comorbid) disorders or conditions, genetic testing, and the like.

The term “body mass index” or “BMI” of a human patient is defined as the weight in kilograms divided by the square of the height in meters, such that BMI has units of kg/m². The term “overweight” is defined as the condition wherein the adult individual of Europid origin has a BMI equal to or greater than 25 kg/m² and less than 30 kg/m². In subjects of Asian origin the term “overweight” is defined as the condition wherein the adult individual has a BMI equal to or greater than 23 kg/m² and less than 25 kg/m². The terms “overweight” and “pre-obese” are used interchangeably.

The term “obesity” is defined as the condition wherein the adult individual of Europid origin has a BMI equal to or greater than 30 kg/m². According to a WHO definition the term obesity may be categorized as follows: the term “class I obesity” is the condition wherein the BMI is equal to or greater than 30 kg/m² but lower than 35 kg/m²; the term “class II obesity” is the condition wherein the BMI is equal to or greater than 35 kg/m² but lower than 40 kg/m²; the terms “class Ill obesity” is the condition wherein the BMI is equal to or greater than 40 kg/m². In subjects of Asian origin the term “obesity” is defined as the condition wherein the adult individual has a BMI equal or greater than 25 kg/m². Obesity in Asians may be categorized further as follows: the term “class I obesity” is the condition wherein the BMI is equal to or greater than 25 kg/m² but lower than 30 kg/m²; the term “class II obesity” is the condition wherein the BMI is equal to or greater than 30 kg/m².

The term “visceral obesity” is defined as the condition wherein a waist-to-hip ratio of greater than or equal to 1.0 in men and 0.8 in women is measured. It defines the risk for insulin resistance and the development of pre-diabetes. The term “abdominal obesity” is usually defined as the condition wherein the waist circumference is >40 inches or 102 cm in men, and is >35 inches or 94 cm in women (for normal ranges of populations, see for example “Joint scientific statement (IDF, NHLB1, AHA, WHO, IAS, IASO). Circulation 2009; 120:1640-1645”).

The term “morbid obesity” is defined herein as a condition in which the individual of Europid origin has a BMI >40 or has a BMI >35 and a comorbidity such as diabetes mellitus or hypertension (see World Health Organization. Obesity: Preventing and Managing the Global Epidemic: Report on a WHO Consultation. World Health Organ Tech Rep Ser. 2000; 894: i-xii, 1-253).

The term “euglycemia” is defined as the condition in which a subject has a fasting blood glucose concentration within the normal range, greater than 70 mg/dL (3.89 mmol/L) and less than 100 mg/dL (5.6 mmol/L), and a 2 h postprandial glucose concentration less than 140 mg/dl.

The term “hyperglycemia” is defined as the condition in which a subject has a fasting blood glucose concentration above the normal range, greater than 100 mg/dL (5.6 mmol/L).

The term “hypoglycemia” is defined as the condition in which a subject has a blood glucose concentration below the normal range, in particular below 70 mg/dL (3.89 mmol/L).

The term “postprandial hyperglycemia” is defined as the condition in which a subject has a 2 hour postprandial blood glucose or serum glucose concentration greater than 200 mg/dL (11.11 mmol/L).

The term “impaired fasting blood glucose” or “IFG” is defined as the condition in which a subject has a fasting blood glucose concentration or fasting serum glucose concentration in a range from 100 to 125 mg/dl (i.e. from 5.6 to 6.9 mmol/1. A subject with “normal fasting glucose” has a fasting glucose concentration smaller than 100 mg/dl, i.e. smaller than 5.6 mmol/1.

The term “impaired glucose tolerance” or “IGT” is defined as the condition in which a subject has a 2 hour postprandial blood glucose or serum glucose concentration greater than 140 mg/dl (7.78 mmol/L) and less than 200 mg/dL (11.11 mmol/L). The abnormal glucose tolerance, i.e. the 2 hour postprandial blood glucose or serum glucose concentration can be measured as the blood sugar level in mg of glucose per dL of plasma 2 hours after taking 75 g of glucose after a fast. A subject with “normal glucose tolerance” has a 2 hour postprandial blood glucose or serum glucose concentration smaller than 140 mg/dl (7.78 mmol/L).

The term “hyperinsulinemia” is defined as the condition in which a subject with insulin resistance, with or without euglycemia, has fasting or postprandial serum or plasma insulin concentration elevated above that of normal, lean individuals without insulin resistance, having a waist-to-hip ratio <1.0 (for men) or <0.8 (for women).

The term “insulin resistance” is defined as a state in which circulating insulin levels in excess of the normal response to a glucose load are required to maintain the euglycemic state (FORD, E. S., et al., JAMA, (2002), pp 356-9, Vol, 287). A method of determining insulin resistance is the euglycaemic-hyperinsulinaemic clamp test. The ratio of insulin to glucose is determined within the scope of a combined insulin-glucose infusion technique. There is found to be insulin resistance if the glucose absorption is below the 25th percentile of the background population investigated (WHO definition). Rather less laborious than the clamp test are so called minimal models in which, during an intravenous glucose tolerance test, the insulin and glucose concentrations in the blood are measured at fixed time intervals and from these the insulin resistance is calculated. With this method, it is not possible to distinguish between hepatic and peripheral insulin resistance.

As a rule, other parameters are used in everyday clinical practice to assess insulin resistance. Preferably, the patient's triglyceride concentration is used, for example, as increased triglyceride levels correlate significantly with the presence of insulin resistance.

Patients with a predisposition for the development of IGT or IFG or Type 2 diabetes are those having euglycemia with hyperinsulinemia and are by definition, insulin resistant. A typical patient with insulin resistance is usually overweight or obese. If insulin resistance can be detected, this is a particularly strong indication of the presence of pre-diabetes. Thus, it may be that in order to maintain glucose homoeostasis a person needs 2-3 times as much insulin as a healthy person, without this resulting in any clinical symptoms.

The term “pre-diabetes” is the condition wherein an individual is pre-disposed to the development of type 2 diabetes. Pre-diabetes extends the definition of impaired glucose tolerance to include individuals with a fasting blood glucose within the high normal range 100 mg/dL (MEIGS, J. B., et al. Diabetes, 2003, pp 1475-1484, Vol. 52) and fasting hyperinsulinemia (elevated plasma insulin concentration). The scientific and medical basis for identifying pre-diabetes as a serious health threat is laid out in a Position Statement entitled “The Prevention or Delay of Type 2 Diabetes” issued jointly by the American Diabetes Association and the National Institute of Diabetes and Digestive and Kidney Diseases (Diabetes Care 2002; 25:742-749). Individuals likely to have insulin resistance are those who have two or more of the following attributes: 1) overweight or obese, 2) high blood pressure, 3) hyperlipidemia, 4) one or more 1^{st} degree relative with a diagnosis of IGT or IFG or type 2 diabetes.

The term “Type 2 diabetes of Type II diabetes mellitus” is defined as the condition in which a subject has a fasting (i.e., no caloric intake for 8 hours) blood glucose or serum glucose concentration greater than 125 mg/dL (6.94 mmol/L), when measured at minimum two independent occasions. The measurement of blood glucose values is a standard procedure in routine medical analysis. Type 2 diabetes is also defined as the condition in which a subject has HbA1c equal to, or greater than 6.5%, a two hour plasma glucose equal to, or greater than 200 mg/dL (11.1 mmol/L) during an oral glucose tolerance test (OGTT) or a random glucose concentration equal to, or greater than 200 mg/dL (11.1 mmol/L) in conjunction with classic symptoms of hyperglycaemia or hyperglycaemic crisis. In the absence of unequicoval hyperglycaemia, as with most diagnostic tests, a test result diagnostic of diabetes should be repeated to rule out laboratory error. The assessment of HbA1c should be performed using a method certified by the National Glycohemoglobin Standardization Program (NGSP) and standardized or traceable to the Diabetes Control and Complications Trial (DCCT) reference assay. If a OGTT is carried out, the blood sugar level of a diabetic will be in excess of 200 mg of glucose per dL (11.1 mmol/l) of plasma 2 hours after 75 g of glucose have been taken on an empty stomach. In a glucose tolerance test 75 g of glucose are administered orally to the patient being tested after a minimum of 8 hours, typically after 10-12 hours, of fasting and the blood sugar level is recorded immediately before taking the glucose and 1 and 2 hours after taking it. In a healthy subject, the blood sugar level before taking the glucose will be between 60 and 110 mg per dL of plasma, less than 200 mg per dL 1 hour after taking the glucose and less than 140 mg per dL after 2 hours. If after 2 hours the value is between 140 and 200 mg, this is regarded as abnormal glucose tolerance.

The term “late stage Type 2 diabetes mellitus” includes patients with a long-standing duration of diabetes, secondary drug failure, indication for insulin therapy and potentially progression to micro- and macrovascular complications e.g. diabetic nephropathy, or coronary heart disease (CHD).

The term “MODY” (“maturity onset diabetes of the youth”) describes a monogenic form for diabetes that, according to gene affects, is split into MODY variants, e.g., MODY 1,2.3.4 etc.

The term “LADA” (“latent autoimmune diabetes of adults”) refers to patients that has a clinical diagnosis of Type 2 Diabetes Mellitus, but who is being detected to have autoimmunity towards the pancreatic beta cell.

The term “HbA1c” refers to the product of a non-enzymatic glycation of the haemoglobin B chain. Its determination is well known to one skilled in the art. In monitoring the treatment of diabetes mellitus the HbA1c value is of exceptional importance. As its production depends essentially on the blood sugar level and the life of the erythrocytes, the HbA1c in the sense of a “blood sugar memory” reflects the average blood sugar levels of the preceding 4-6 weeks. Diabetic patients whose HbA1c value is consistently well adjusted by intensive diabetes treatment (i.e. <6.5% of the total haemoglobin in the sample), are significantly better protected against diabetic microangiopathy. For example, metformin on its own achieves an average improvement in the HbA1c value in the diabetic of the order of 1.0-1.5%. This reduction of the HbA1C value is not sufficient in all diabetics to achieve the desired target range of <6.5% and preferably <6% HbA1c.

The term “insufficient glycemic control” or “inadequate glycemic control” in the scope of the present invention means a condition wherein patients show HbA1c values above 6.5%, in particular above 7.0%, even more preferably above 7.5%, especially above 8%.

The “metabolic syndrome”, also called “syndrome X” (when used in the context of a metabolic disorder), also called the “dysmetabolic syndrome” is a syndrome complex with the cardinal feature being insulin resistance (LAAKSONEN, D. E., et al. Am J Epidemiol, 2002, pp 1070-7, Vol 156). According to the ATP III/NCEP guidelines (Executive Summary of the Third Report of the National Cholesterol Education Program (NCEP) Expert Panel on Detection, Evaluation, and Treatment of High Blood Cholesterol in Adults (Adult Treatment Panel III) JAMA: Journal of the American Medical Association, (2001), pp 2486-2497, Vol 285), diagnosis of the metabolic syndrome is made when three or more of the following risk factors are present:
1. Abdominal obesity, defined as waist circumference greater than about 40 inches or 102 cm in men, and greater than about 35 inches or 94 cm in women;
2. Triglycerides equal to or greater than about 150 mg/dL;
3. HDL-cholesterol less than about 40 mg/dL in men and less than about 50 in women;
4. Blood pressure equal to or greater than about 130/85 mm Hg (SBP equal to or greater than about 130 or DBP equal to or greater than about 85);
5. Fasting blood glucose equal to or greater than about 100 mg/dL.

According to a commonly used definition, hypertension is diagnosed if the systolic blood pressure (SBP) exceeds a value of 140 mm Hg and diastolic blood pressure (DBP) exceeds a value of 90 mm Hg. If a patient is suffering from manifest diabetes it is currently recommended that the systolic blood pressure be reduced to a level below 130 mm Hg and the diastolic blood pressure be lowered to below 80 mm Hg.

The definitions of NODAT (new onset diabetes after transplantation) and PTMS (post-transplant metabolic syndrome) follow closely that of the American Diabetes Association diagnostic criteria for type 2 diabetes, and that of the International Diabetes Federation (IDF) and the American Heart Association/National Heart, Lung, and Blood Institute, for the metabolic syndrome. NODAT and/or PTMS are associated with an increased risk of micro- and macrovascular disease and events, graft rejection, infection, and death. A number of predictors have been identified as potential risk factors related to NODAT and/or PTMS including a higher age at transplant, male gender, the pre-transplant body mass index, pre-transplant diabetes, and immunosuppression.

The term “gestational diabetes” (diabetes of pregnancy) denotes a form of the diabetes which develops during pregnancy and usually ceases again immediately after the birth. Gestational diabetes is diagnosed by a screening test which often is carried out between the 24th and 28th weeks of pregnancy, but could be conducted at any time during pregnancy, in particular if previous gestational diabetes has been diagnosed. It is usually a simple test in which the blood sugar level is measured e.g., one hour after the administration of 50 g of glucose solution. If this 1 h level is above 140 mg/dl, gestational diabetes is suspected. Final confirmation may be obtained by a standard glucose tolerance test, for example with 75 g of glucose, which also serve as a diagnostic test in the absence of the 50 g challenge.

The term “subject” as used herein, refers to an animal, preferably a mammal, most preferably a human, who has been the object of treatment, observation or experiment. Preferably, the subject has experienced and/or exhibited at least one symptom of the disease or disorder to be treated and/or prevented.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

As used herein, the term “composition” is intended to encompass a product comprising the specified ingredients in the specified amounts, as well as any product which results, directly or indirectly, from combinations of the specified ingredients in the specified amounts.

As more extensively provided in this written description, terms such as “reacting” and “reacted” are used herein in reference to a chemical entity that is any one of: (a) the actually recited form of such chemical entity, and (b) any of the forms of such chemical entity in the medium in which the compound is being considered when named.

One skilled in the art will recognize that, where not otherwise specified, the reaction step(s) is performed under suitable conditions, according to known methods, to provide the desired product. One skilled in the art will further recognize that, in the specification and claims as presented herein, wherein a reagent or reagent class/type (e.g. base, solvent, etc.) is recited in more than one step of a process, the individual reagents are independently selected for each reaction step and may be the same of different from each other. For example wherein two steps of a process recite an organic or inorganic base as a reagent, the organic or inorganic base selected for the first step may be the same or different than the organic or inorganic base of the second step. Further, one skilled in the art will recognize that wherein a reaction step of the present invention may be carried out in a variety of solvents or solvent systems, said reaction step may also be carried out in a mixture of the suitable solvents or solvent systems.

One skilled in the art will recognize that wherein a reaction step of the present invention may be carried out in a variety of solvents or solvent systems, said reaction step may also be carried out in a mixture of the suitable solvents or solvent systems.

One skilled in the art will further recognize that the reaction or process step(s) as herein described are allowed to proceed for a sufficient period of time until the reaction is complete, as determined by any method known to one skilled in the art, for example, chromatography (e.g. HPLC). In this context a “completed reaction or process step” shall mean that the reaction mixture contains a significantly diminished amount of the starting material(s)/reagent(s) and a significantly reduced amount of the desired product(s), as compared to the amounts of each present at the beginning of the reaction.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

To provide a more concise description, some of the quantitative expressions herein are recited as a range from about amount X to about amount Y. It is understood that wherein a range is recited, the range is not limited to the recited upper and lower bounds, but rather includes the full range from about amount X through about amount Y, or any amount or range therein.

Examples of suitable solvents, bases, reaction temperatures, and other reaction parameters and components are provided in the detailed descriptions which follow herein. One skilled in the art will recognize that the listing of said examples is not intended, and should not be construed, as limiting in any way the invention set forth in the claims which follow thereafter.

As used herein, unless otherwise noted, the term “aprotic solvent” shall mean any solvent that does not yield a proton. Suitable examples include, but are not limited to DMF, 1,4-dioxane, THF, acetonitrile, pyridine, dichloroethane, dichloromethane, MTBE, toluene, acetone, and the like.

As used herein, unless otherwise noted, the term “leaving group” shall mean a charged or uncharged atom or group which departs during a substitution or displacement reaction. Suitable examples include, but are not limited to, Br, Cl, I, mesylate, tosylate, and the like.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

As used herein, unless otherwise noted, the term “nitrogen protecting group” shall mean a group which may be attached to a nitrogen atom to protect said nitrogen atom from participating in a reaction and which may be readily removed following the reaction. Suitable nitrogen protecting groups include, but are not limited to carbamates—groups of the formula —C(O)O—R wherein R is for example methyl, ethyl, t-butyl, benzyl, phenylethyl, CH₂═CH—CH₂—, and the like; amides—groups of the formula —C(O)—R′ wherein R′ is for example methyl, phenyl, trifluoromethyl, and the like; N-sulfonyl derivatives—groups of the formula —SO₂—R″ wherein R″ is for example tolyl, phenyl, trifluoromethyl, 2,2,5,7,8-pentamethylchroman-6-yl-, 2,3,6-trimethyl-4-methoxybenzene, and the like. Other suitable nitrogen protecting groups may be found in texts such as T. W. Greene & P. G. M. Wuts, Protective Groups in Organic Synthesis, John Wiley & Sons, 1991.

As used herein, unless otherwise noted, the term “oxygen protecting group” shall mean a group which may be attached to an oxygen atom to protect said oxygen atom from participating in a reaction and which may be readily removed following the reaction. Suitable oxygen protecting groups include, but are not limited to, acetyl, benzoyl, t-butyl-dimethylsilyl, trimethylsilyl (TMS), MOM, THP, and the like. Other suitable oxygen protecting groups may be found in texts such as T. W. Greene & P. G. M. Wuts, Protective Groups in Organic Synthesis, John Wiley & Sons, 1991.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

Additionally, chiral HPLC against a standard may be used to determine percent enantiomeric excess (% ee). The enantiomeric excess may be calculated as follows

[(Rmoles−Smoles)/(Rmoles+Smoles)]×100%

where Rmoles and Smoles are the R and S mole fractions in the mixture such that Rmoles+Smoles=1. The enantiomeric excess may alternatively be calculated from the specific rotations of the desired enantiomer and the prepared mixture as follows:

ee=([α−obs]/[α−max])×100.

The present invention includes within its scope prodrugs of the compounds of this invention. In general, such prodrugs will be functional derivatives of the compounds which are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs”, ed. H. Bundgaard, Elsevier, 1985.

For use in medicine, the salts of the compounds of this invention refer to non-toxic “pharmaceutically acceptable salts.” Other salts may, however, be useful in the preparation of compounds according to this invention or of their pharmaceutically acceptable salts. Suitable pharmaceutically acceptable salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. Thus, representative pharmaceutically acceptable salts include, but are not limited to, the following: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

Representative acids which may be used in the preparation of pharmaceutically acceptable salts include, but are not limited to, the following: acids including acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucoronic acid, L-glutamic acid, α-oxo-glutaric acid, glycolic acid, hipuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, phosphoric acid, L-pyroglutamic acid, salicylic acid, 4-amino-salicylic acid, sebaic acid, stearic acid, succinic acid, sulfuric acid, tannic acid, (+)-L-tartaric acid, thiocyanic acid, p-toluenesulfonic acid and undecylenic acid.

Representative bases which may be used in the preparation of pharmaceutically acceptable salts include, but are not limited to, the following: bases including ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

Compounds of formula (I) of the present invention may be synthesized according to the general synthesis schemes described below. The preparation of the various starting materials used in the synthesis schemes which follow hereinafter is well within the skill of persons versed in the art.

### General Synthesis Schemes:

Compounds of formula (I) wherein X is N may be prepared as described in Scheme (A), below.

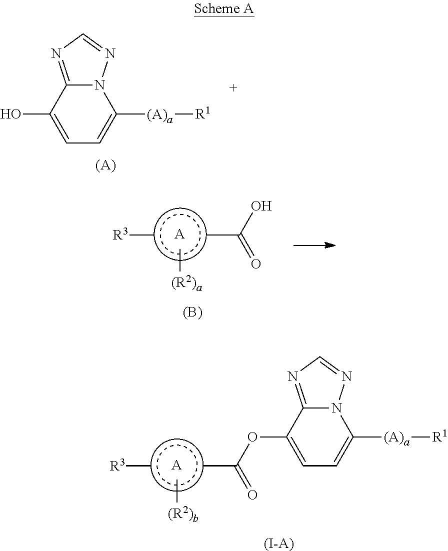

Accordingly, a suitably substituted compound of formula (A) is reacted with a suitably substituted compound of formula (B), in the presence of a suitably selected coupling agent such as EDCI, PyBrop, HATU, and the like; in the presence of a suitably selected co-coupling agent such as DMAP, HOBt, 2-hydroxypyridine-N-oxide, and the like; in a suitably selected solvent such as DCM, THF, 1,2-dichloroethane, and the like; to yield the corresponding compound of formula (I-A).

One skilled in the art will recognize that wherein the compound of formula (A) and/or the compound of formula (B), a terminal functional group is reactive under the coupling conditions described above, said group is preferably protected prior to the coupling reaction, with a suitably selected protecting group, according to known methods. Said protecting group is then removed after the coupling reaction, according to known method.

Intermediates in the synthesis of the compounds of formula (I) wherein X is N include the intermediate compound of formula (XII)

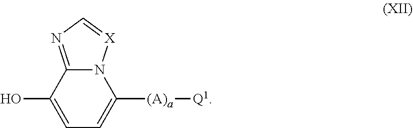

wherein Q¹ is selected from the group consisting of —C(O)O—PG² and R¹ (such that when Q¹ is R¹, then the compound of formula (XII) corresponds to the compound of formula (A) in Scheme A, above), prepared as described in Schemes 1-6, below; and the intermediate compound of formula (XXX)

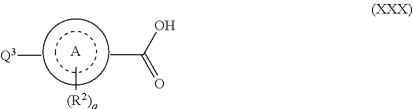

wherein Q² is R³ or a suitably protected R³, prepared as described in Schemes 7-8, below.

One skilled in the art will recognize that compounds of formula (A) wherein Q¹ is —C(O)O—PG¹ may be reacted, according to methods known to those skilled in the art, to convert the protected carboxylic group (—C(O)O—PG¹) to the desired R¹ group. One skilled in the art will further recognize that said conversion may be completed either before or after the coupling reaction with the compound of formula (B). For example, the compound of formula (A) wherein Q¹ is —C(O)O—PG¹ may be de-protected (to yield the corresponding —C(O)OH group), and then reacted with a suitably substituted amine, sulfonamide or amino acid; in the presence of a suitably selected coupling agent such as EDCI, PyBrop, HATU, and the like; in the presence of a suitably selected base such as TEA, DIPEA, pyridine, and the like; in a suitably selected solvent such as DCM, chloroform, THF, and the like; to yield the corresponding compound wherein Q¹ is a substituted amide such as —C(O)—NH—CH₂—CO₂H, —C(O)—NH—CH(CO₂H)—CH₂—CO₂H, —C(O)—N(CO₂H)—CH₂—CO₂H, —C(O)—N(CH₂CO₂H)—CH₂—CO₂H, —C(O)—NH—C(CH₂CH₂—CO₂H)₃—C(O)—NH—SO₂—(C₁₋₄alkyl), —C(O)—NH—CH(R⁵)—CO₂H or —C(O)-proline.

One skilled in the art will recognize that compounds of formula (A) wherein Q¹ is SO₂—OPG² or —P(O)—(OPG²)₂ (which compounds may be commercially available) may be reacted via a coupling reaction with the compound of formula (B), in the presence of a suitably selected coupling agent such as EDCI, PyBrop, HATU, and the like; in the presence of a suitably selected base such as TEA, DIPEA, pyridine, and the like; in a suitably selected solvent such as DCM, chloroform, THF, and the like; to yield the corresponding compound wherein Q¹ is SO₂—OPG² or —P(O)—(OPG²)₂. The compound of formula (A) wherein Q¹ is SO₂—OPG² or —P(O)—(OPG²)₂ may then be de-protected, according to known methods, to yield the corresponding compound wherein Q¹ is SO₃H or —PO₃H₂, respectively.

Compounds of formula (XII) wherein X is N and a is 1 (such that A is —C₁₋₆alkyl-) may be prepared as described in Scheme 1, below.

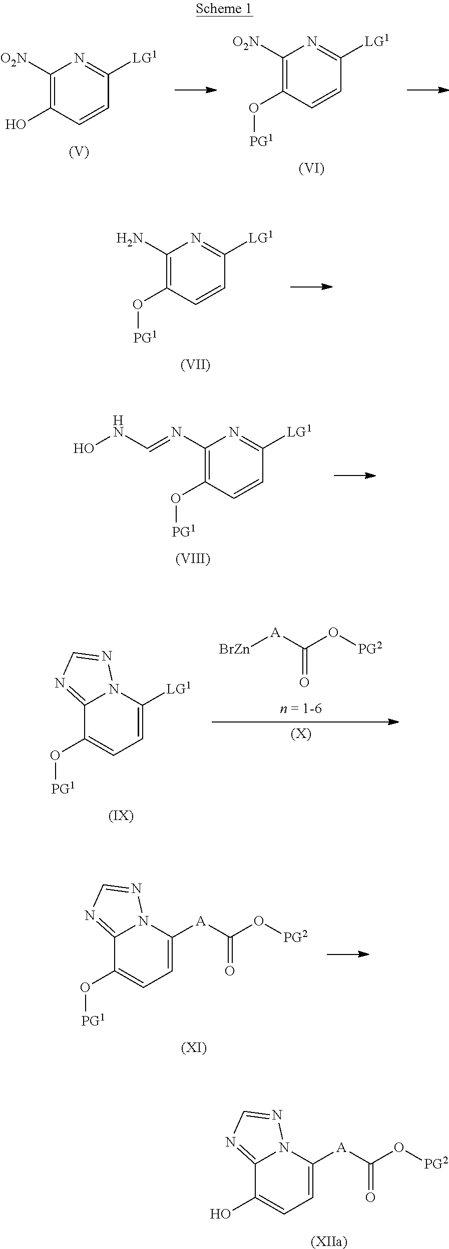

Accordingly, a suitably substituted compound of formula (V), wherein LG¹ is a suitably selected leaving group such as Br, I, OTf, and the like, a known compound or compound prepared by known methods, is reacted with a suitably selected protecting reagent, according to known methods, to yield the corresponding compound of formula (VI), wherein PG¹ is the corresponding oxygen protecting group. For example, the compound of formula (V) may be reacted with benzylbromide, a known compound, in the presence of a suitably selected inorganic base such as K₂CO₃, in a suitably selected solvent such as DMF, at about 70° C., to yield the corresponding compound of formula (VI), wherein PG¹ is benzyl.

The compound of formula (VI) is reacted to convert the nitro group to the corresponding amino group, according to known methods; to yield the corresponding compound of formula (VII). For example, the compound of formula (VI) may be reacted with ammonium chloride in the presence of iron, in a suitably selected solvent or mixture of solvents such as ethanol/water, at about 80° C.; to yield the corresponding compound of formula (VII).

The compound of formula (VII) is reacted with a suitably selected formylation agent such as DMF-DMA, N-hydroxyimidoformamide, a mixture if formic acid/hydroxyamine, and the like; in a suitably selected solvent such as isopropanol, EtOH, 1,4-dioxane, and the like; at a temperature in the range of from about 60° C. to about 100° C.; to yield the corresponding compound of formula (VIII).

The compound of formula (VIII) is subjected to ring closure condition, for example, reacting with a suitably selected acylating agent such as TFAA, TsCl, MsCl, and the like; in a suitably selected solvent such as THF, DCM, 1,4-dioxane, and the like; a temperature in the range of from about 0° C. to about 50° C., for example at about 20° C.; to yield the corresponding compound of formula (IX).

The compound of formula (IX) is reacted with a suitably substituted compound of formula (X) wherein PG² is a suitably selected protecting group such as t-butyl, di-methyl-t-butyl-silyl, benzyl, p-methoxy-benzyl, and the like, but not methyl, ethyl or isopropyl, a known compound or compound prepared by known methods, through reaction of the corresponding alkyl bromide or alkyl iodide with ZnCl2 or ZnBr2; in the presence of a suitably selected coupling agent such as Pd₂(dba)₃, Pd(Ph₃P)₄, Pd(Ph₃P)₂Cl₂, and the like; in the presence of a suitably selected phosphine ligand such as XPhos, CPhos, dppf, and the like; in a suitably selected solvent such as THF, 1,4-dioxane, DMF, and the like; a temperature in the range of from about 20° C. to about 120° C., for example at about 115° C.; to yield the corresponding compound of formula (XI).

The compound of formula (XI) is deprotected, according to known methods. For example, wherein the PG¹ protecting group is benzyl, the compound of formula (XI) is reacted with H2 in the presence of a suitably selected catalyst such as Pd/C; in a suitably selected solvent such as ethyl acetate; to yield the corresponding compound of formula (XIIa).

Alternatively, compounds of formula (XII) wherein X is N, a is 1 and A is —CH₂CH₂—(C₁₋₄alkyl)- may be prepared as described in Scheme 2, below.

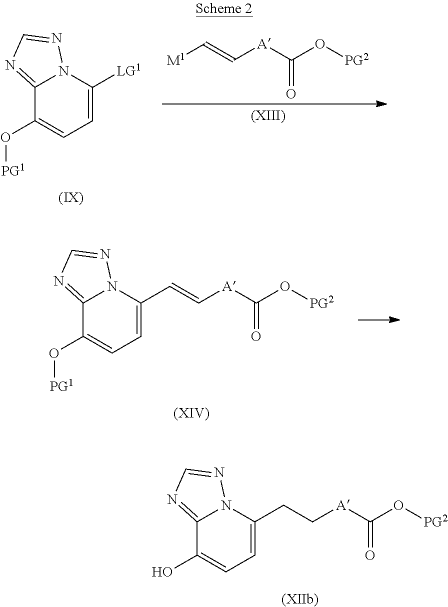

Accordingly, a suitably substituted compound of formula (IX) is reacted with a suitably substituted compound of formula (XIII), wherein M¹ is hydrogen or a suitably selected metal species such as —B(OH)₂, —Sn(Bu)₃, and the like, and wherein A′ is —(C₁₋₄alkyl)- a known compound or compound prepared by known methods, for example by coupling reactions between corresponding bromide or iodide with boron or tin reagent; in the presence of a suitably selected coupling agent such as Pd₂(dba)₃, Pd(Ph₃P)₄, Pd(Ph₃P)₂Cl₂, and the like; in the presence of a suitably selected phosphine ligand such as XPhos, CPhos, dppf, and the like; in a suitably selected solvent such as THF, 1,4-dioxane, DMF, and the like; a temperature in the range of from about 20° C. to about 120° C., for example at about 115° C.; to yield the corresponding compound of formula (XIV).

The compound of formula (XIV) reduced (reacted with a suitably selected reducing agent) and de-protected in one step, according to known methods. For example, wherein the PG¹ protecting group is benzyl, the compound of formula (XIV) is reacted with H2 in the presence of a suitably selected catalyst such as Pd/C; in a suitably selected solvent such as ethyl acetate; to yield the corresponding compound of formula (XIIb).

Compounds of formula (XII), wherein X is N and a is 0 (such that A is absent), may be prepared as described in Scheme 3, below.

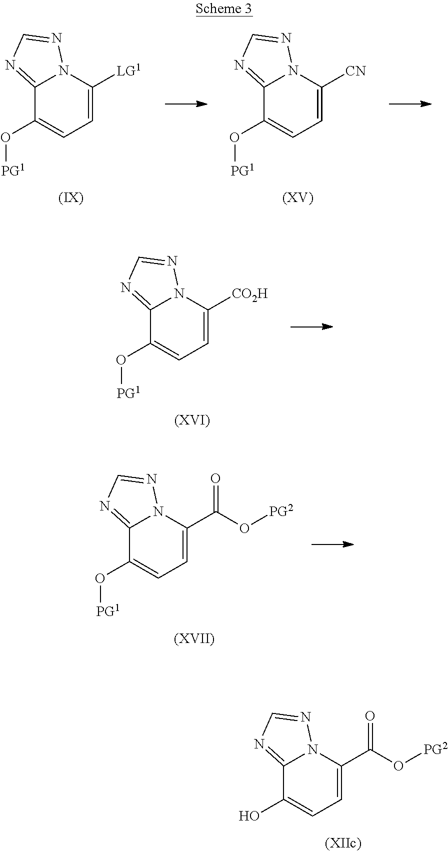

Accordingly, a suitably substituted compound of formula (IX) is reacted with a suitably selected cyanation agent such as KCN, Zn(CN)₂, CuCN, and the like’ in the presence of a suitably selected catalyst such as Pd(Ph₃P)₄, Pd(Ph₃P)₂Cl₂, CuI, and the like; in a suitably selected solvent such as THF, 1,4-dioxane, DMF, and the like; at a temperature in the range of from about 25° C. to about 120° C., for example at about 115° C.; to yield the corresponding compound of formula (XV).

The compound of formula (XV) is hydrolyzed in the presence of a suitably base such as KOH, NaOH, Ba(OH)₂, and the like; in a suitably selected solvent such as THF, MeOH, water, and the like; at a temperature in the range of from about 25° C. to about 100° C., for example at about 80° C.; to yield the corresponding compound of formula (XVI).

The compound of formula (XVI) is protected, according to known methods. For example, wherein the PG² protecting group is t-butyl, the compound of formula (XVI) is reacted with Boc₂O; in the presence of a suitably selected base such as DIPEA; in the presence of a suitably selected catalyst such as DMAP; in a suitably selected solvent such as DCM; at a temperature in the range of from about 25° C. to about 60° C., for example at about 30° C. to yield the corresponding compound of formula (XVII).

The compound of formula (XVII) is selectively de-protected, according to known methods. For example, wherein the PG¹ protecting group is benzyl, the compound of formula (XI) is reacted with H2 in the presence of a suitably selected catalyst such as Pd/C; in a suitably selected solvent such as ethyl acetate; to yield the corresponding compound of formula (XIIc).

Compounds of formula (XII) wherein X is N, a is an integer from 0 to 1, A is —(C₁₋₆alky)-, and R¹ is selected from the group consisting of —C(O)—NH—CH₂—CO₂H, —C(O)—NH—CH(CO₂H)—CH₂—CO₂H, —C(O)—N(CO₂H)—CH₂—CO₂H, —C(O)—NH—SO₂—(C₁₋₄alkyl) and —C(O)—NH—CH(R⁵)—CO₂H and —C(O)-proline; may be prepared as described in Scheme 4, below.

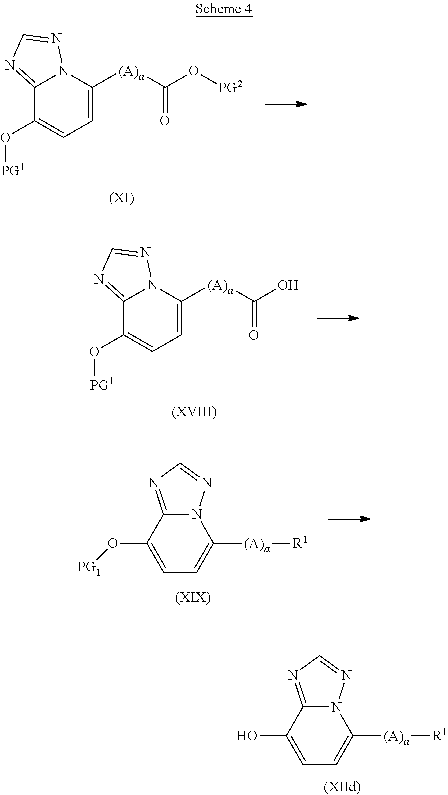

The compound of formula (XI) is selectively de-protected, according to known methods. For example, wherein the PG² protecting group is t-butyl, the compound of formula (XI) is reacted in the presence of a suitably selected acid such as TFA, HCl in 1,4-dioxane, H₂SO₄, and the like; in a suitably selected solvent such as THF, DCM, 1,4-dioxane, and the like; at a temperature in the range of from about 0° C. to about 80° C., for example at about 30° C. to yield the corresponding compound of formula (XVIII).

The compound of formula (XVIII) is reacted with

(a) a suitably substituted primary amine, a compound of the formula NH₂—R¹;

(b) a suitably substituted secondary amine, a compound of the formula R¹—NH—R, wherein R is a suitably selected substituent group such as C₁₋₄alkyl, C₁₋₄carboxylic acid, and the like;

(c) a suitably substituted sulfonamide, a compound of the formula R¹—SO₂—NH₂, wherein R¹ is C₁₋₄alkyl;

(d) a suitably substituted amino acid, a compound of the formula NH₂—CH(R⁵)—CO₂H, wherein R⁵ is selected from the group consisting of hydrogen, methyl, isopropyl, 2-methylpropyl, 3-methylpropyl, 2-methylthio-ethyl, benzyl, 4-hydroxy-benzyl, 1H-indol-3-methyl, carbamoyl-methyl, sulfanyl-methyl, 2-carbamoyl-ethyl, hydroxy-methyl, 2-hydroxy-ethyl, carboxyl-methyl, 2-carboxy-ethyl, 3-guanidino-propyl, 1H-imidazol-4-yl-methyl and 4-amino-butyl,); or

(e) proline (preferably L-proline);

a known compound or compound prepared by known methods; in the presence of a suitably selected coupling agent such as EDCI, PyBrop, HATU, and the like; in the presence of a suitably selected co-coupling agent such as DMAP, HOBt, 2-hydroxypyridine-N-oxide, and the like; in a suitably selected solvent such as DCM, THF, 1,2-dichloroethane, and the like; at a temperature in the range of from about 0° C. to about 80° C., for example at about 30° C.; to yield the corresponding compound of formula (XIX).

The compound of formula (XIX) is deprotected, according to known methods. For example, wherein the PG¹ protecting group is benzyl, the compound of formula (XIV) is reacted with H2 in the presence of a suitably selected catalyst such as Pd/C; in a suitably selected solvent such as ethyl acetate; to yield the corresponding compound of formula (XIId).

Compounds of formula (XII) wherein X is N, R¹ is

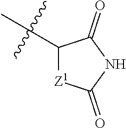

and Z¹ is O, S or NH, may be prepared as described in Scheme 5, below.

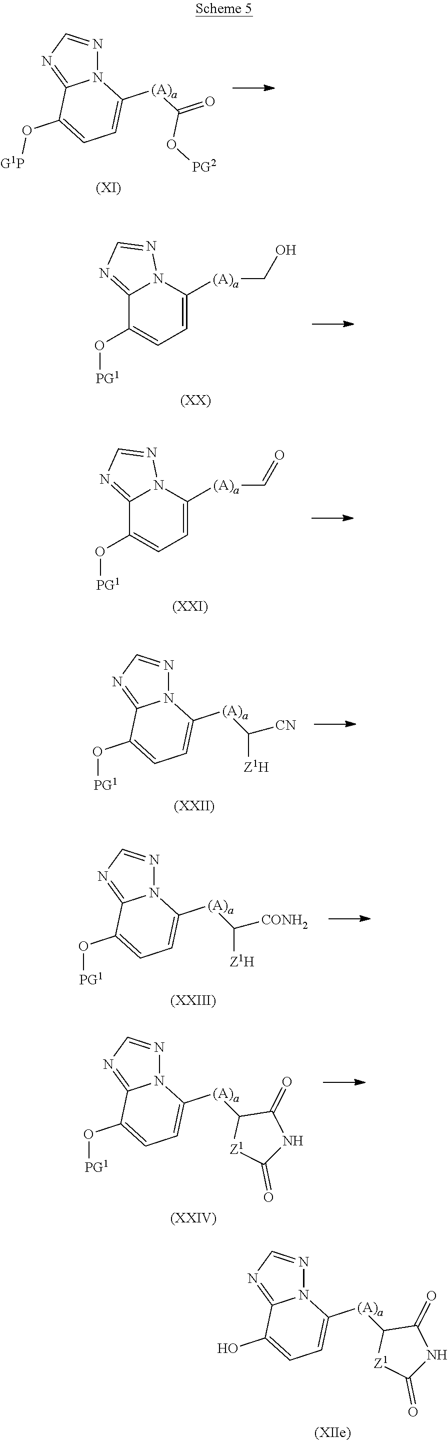

Accordingly, the compound of formula (XI) is reacted with a suitably selected reducing agent such as LAH, LBH, DIBAL and the like; in a suitably selected solvent such as diethyl ether, THF, toluene, and the like; at a temperature in the range of from about −78° C. to about 30° C., for example at about 0° C.; to yield the corresponding compound of formula (XX).

The compound of formula (XX) is reacted with a suitably selected oxidant such as Dess-Martin agent, MnO₂, oxalyl chloride/DMSO and the like; in a suitably selected solvent such as DCM, THF, 1,2-dichloroethane, and the like; at a temperature in the range of from about −78° C. to about 30° C., for example at about 0° C.; to yield the corresponding compound of formula (XXI).

The compound of formula (XXI) is reacted with a suitably selected source of CN such as TMSCN, Zn(CN)₂, KCN and the like; in the presence of Lewis acid such as AlCl₃, ZnCl₂, BF₃Et₂O, and the like; in the presence of a suitably selected nucleophilic agent such as H₂O, NH₃, Na₂S and the like; wherein the nucleophilic agent is selected to provide the desired Z¹ functionality; in a suitably selected solvent such as DCM, THF, 1,2-dichloroethane, and the like; at a temperature in the range of from about −10° C. to about 80° C., for example at about 30° C.; to yield the corresponding compound of formula (XXII), wherein Z¹ is O, S or NH.

The compound of formula (XXII) is reacted with a suitably selected oxidant such as H₂O₂, mCPBA, CH₃CO₃H and the like; in the presence of a base such as K₂CO₃, Cs₂CO₃, BaCO₃, and the like; in a suitably selected solvent such as DMSO, THF, DMF, and the like; at a temperature in the range of from about 0° C. to about 80° C., for example at about 30° C.; to yield the corresponding compound of formula (XXIII).

The compound of formula (XXIII) is reacted with a suitably selected coupling agent such as CDI, triphosgene, 2,2,-trichloroacetyl isocynate, and the like; in the presence of a base such as TEA, DIPEA, N-methylmorpholine, and the like; in a suitably selected solvent such as DCM, THF, toluene, and the like; at a temperature in the range of from about 0° C. to about 80° C., for example at about 30° C.; to yield the corresponding compound of formula (XXIV).

The compound of formula (XXIV) is de-protected, according to known methods. For example, wherein the PG¹ protecting group is benzyl, the compound of formula (XXIV) is reacted with H2 in the presence of a suitably selected catalyst such as Pd/C; in a suitably selected solvent such as ethyl acetate; to yield the corresponding compound of formula (XIIe), wherein Z¹ is O, S or NH.

Compounds of formula (XII) wherein X is N, R¹ is

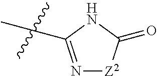

and Z² is O, S or NH, may be prepared as described in Scheme 6, below.

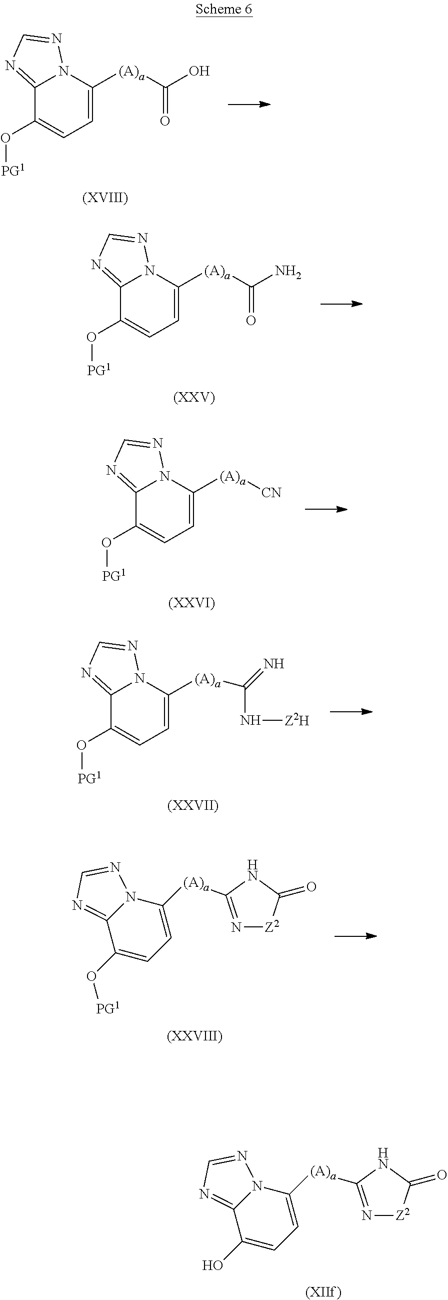

Accordingly, the compound of formula (XVIII) is treated with a suitably selected acylating agent such as SOCl₂, (COCl)₂ and the like; in a suitably selected solvent such as diethylether, THF, toluene, and the like; at a temperature in the range of from about 0° C. to about 80° C., for example at about 30° C.; followed by reaction with a suitably selected source of ammonia such as ammonium hydroxide, ammonium chloride, ammonia in MeOH, and the like; at a temperature in the range of from about 0° C. to about 80° C., to yield the corresponding compound of formula (XXV).

The compound of formula (XXV) is reacted with a suitably selected second acylating agent such as TFAA, acetic anhydride and the like; in the presence of a base such as TEA, DIPEA, pyridine, and the like; in a suitably selected solvent such as diethyl ether, THF, toluene, and the like; at a temperature in the range of from about 0° C. to about 80° C., for example at about 30° C.; to yield the corresponding compound of formula (XXVI).

The compound of formula (XXVI) is reacted with a suitably selected nucleophile such as NH₂OH, hydrazine and the like; wherein the nucleophile is selected to provide the desired Z² functionality; in the presence of a base such as NaOMe, NaOEt, t-BuOK, and the like; in a suitably selected solvent such as MeOH, EtOH, THF, and the like; at a temperature in the range of from about 20° C. to about 80° C., for example at about 30° C.; to yield the corresponding compound of formula (XXVII), wherein Z² is O, S or NH.

The compound of formula (XXVII) is reacted with a suitably selected coupling agent such as CDI, triphosgene and the like; in the presence of a base such as TEA, DIPEA, pyridine, and the like; in a suitably selected solvent such as DCM, THF, toluene, and the like; at a temperature in the range of from about 0° C. to about 60° C., for example at about 30° C.; to yield the corresponding compound of formula (XXVIII).

The compound of formula (XXVIII) is de-protected, according to known methods. For example, wherein the PG¹ protecting group is benzyl, the compound of formula (XXVIII) is reacted with H2 in the presence of a suitably selected catalyst such as Pd/C; in a suitably selected solvent such as ethyl acetate; to yield the corresponding compound of formula (XIIf) wherein Z² is O, S or NH.

Compounds of formula (XXX) wherein R³ is protected guanidine, substituted guanidine or substituted imidamide may be prepared as described in Scheme 7 below.

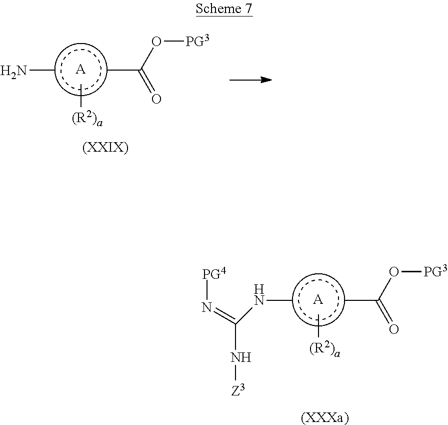

Accordingly, a suitably substituted compound of formula (XXIX), wherein PG³ is a suitably selected oxygen protecting group such as benzyl, t-butyl, p-methoxybenzyl, and the like, a known compound or compound prepared by known methods, is reacted with a suitably selected electrophilic agent such as cyanamide, N-[[(methoxycarbonyl)amino](methylthio)methylene]-methyl carbamic acid ester, N,N′-[[(trifluoromethyl)sulfonyl]carbonimidoyl]bis-C,C′-bis(1,1-dimethylethyl) carbamic acid ester, N-[[[(1,1-dimethylethoxy)carbonyl]amino]-1H-pyrazol-1-ylmethylene]-1,1-dimethylethyl carbamic acid ester, benzyl ethanimidothioate, and the like; optionally in the presence of a suitably selected Lewis acid or protonic acid such as HgCl₂, pTSA, CSA, and the like; optionally in the presence of a suitably selected base such as TEA, DIPEA, N-methyl-morpholine, and the like; in a suitably selected solvent such as THF, 1,4-dioxane, DMF, and the like; at a temperature in the range of from about 0° C. to about 100° C., for example at about 50° C.; to yield the corresponding compound of formula (XXXa), wherein PG⁴ is the corresponding nitrogen protecting group (selected on the basis of the choice of electrophilic agent) and wherein Z³ is the corresponding group (based on the choice of electrophilic agent) selected from PG⁴, OH, NH₂ and C₁₋₄alkyl.

Compounds of formula (XXX) wherein R³ is a protected amidine may be prepared as described in Scheme 8, below.

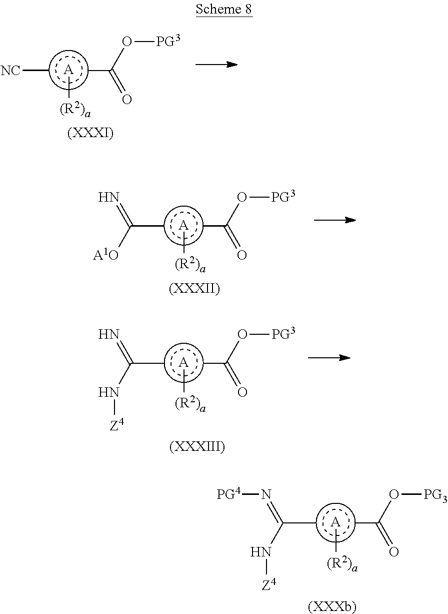

Accordingly, a suitably substituted compound of formula (XXXI), a known compound or compound prepared by known methods (e.g. cyanation of the corresponding chloro, bromo or iodo-containing derivatives), is reacted with a suitably selected acid such as HCl gas, concentrated sulfuric acid, trifluoroacetic acid, and the like; in a suitably selected alcohol of formula A¹-OH, wherein A¹ is C₁₋₄alkyl, such as EtOH, MeOH, i-PrOH, and the like; at a temperature in the range of from about 0° C. to about 60° C., for example at about 30° C.; to yield the corresponding compound of formula (XXXII).

The compound of formula (XXXII) is reacted with a suitably selected nucleophilic agent such as cyanamide, hydroxyamine, C₁₋₄alkylamine, C₁₋₄alkoxyamine, and the like; in the presence of a suitably selected base such as TEA, DIPEA, pyridine, and the like; in a suitably selected solvent such as THF, DMF, MeOH, and the like; at a temperature in the range of from about 0° C. to about 100° C., for example at about 30° C., to yield the corresponding compound of formula (XXXIII), wherein Z⁴ is selected from the group consisting of CN, OH, NH₂, —O—(C₁₋₄alky) and —NH—(C₁₋₄alkyl).

The compound of formula (XXXIII) is protected, as needed or desired with a suitably selected protecting agent such as Boc₂O, CbzCl and the like; in the presence of a base such as TEA, DIPEA, pyridine, and the like; in a suitably selected solvent such as DCM, THF, toluene, and the like; at a temperature in the range of from about 0° C. to about 60° C., for example at about 30° C.; to yield the corresponding compound of formula (XIIIb), wherein PG⁴ is the corresponding suitably selected protecting agent.

Compounds of formula (I) wherein X is CH may be prepared as described in Schemes B, below.

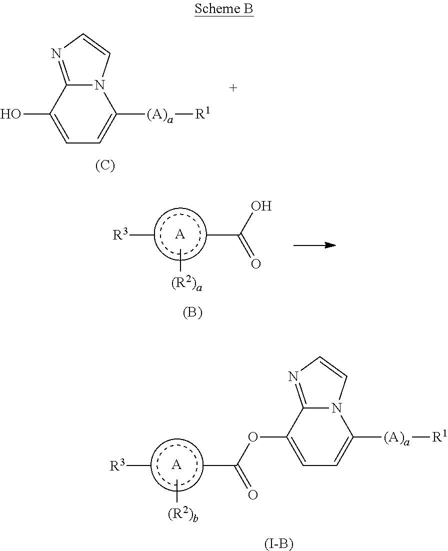

Accordingly, a suitably substituted compound of formula (C) is reacted with a suitably substituted compound of formula (B), in the presence of a suitably selected coupling agent such as EDCI, PyBrop, HATU, and the like; in the presence of a suitably selected co-coupling agent such as DMAP, HOBt, 2-hydroxypyridine-N-oxide, and the like; in a suitably selected solvent such as DCM, THF, 1,2-dichloroethane, and the like; to yield the corresponding compound of formula (I-B).

One skilled in the art will recognize that wherein the compound of formula (C) and/or the compound of formula (B), a terminal functional group is reactive under the coupling conditions described above, said group is preferably protected prior to the coupling reaction, with a suitably selected protecting group, according to known methods. Said protecting group is removed after the coupling reaction described above, according to known method.

One skilled in the art will recognize that compounds of formula (C) wherein Q¹ is —C(O)O—PG¹ may be reacted, according to methods known to those skilled in the art, to convert the protected carboxylic group (—C(O)O—PG¹) to the desired R¹ group. One skilled in the art will further recognize that said conversion may be completed either before or after the coupling reaction with the compound of formula (B). For example, the compound of formula (C) wherein Q¹ is —C(O)O—PG¹ may be de-protected (to yield the corresponding —C(O)OH group), and then reacted with a suitably substituted amine, sulfonamide or amino acid; in the presence of a suitably selected coupling agent such as EDCI, PyBrop, HATU, and the like; in the presence of a suitably selected base such as TEA, DIPEA, pyridine, and the like; in a suitably selected solvent such as DCM, chloroform, THF, and the like; to yield the corresponding compound wherein Q¹ is a substituted amide such as —C(O)—NH—CH₂—CO₂H, —C(O)—NH—CH(CO₂H)—CH₂—CO₂H, —C(O)—N(CO₂H)—CH₂—CO₂H, —C(O)—N(CH₂CO₂H)—CH₂—CO₂H, —C(O)—NH—C(CH₂CH₂—CO₂H)₃—C(O)—NH—SO₂—(C₁₋₄alkyl), —C(O)—NH—CH(R⁵)—CO₂H or —C(O)-proline.

One skilled in the art will recognize that compounds of formula (C) wherein Q¹ is SO₂—OPG² or —P(O)—(OPG²)₂ (which compounds may be commercially available) may be reacted via a coupling reaction with the compound of formula (B), in the presence of a suitably selected coupling agent such as EDCI, PyBrop, HATU, and the like; in the presence of a suitably selected base such as TEA, DIPEA, pyridine, and the like; in a suitably selected solvent such as DCM, chloroform, THF, and the like; to yield the corresponding compound wherein Q¹ is SO₂—OPG² or —P(O)—(OPG²)₂. The compound of formula (C) wherein Q¹ is SO₂—OPG² or —P(O)—(OPG²)₂ may then be de-protected, according to known methods, to yield the corresponding compound wherein Q¹ is SO₃H or —PO₃H₂, respectively.

The intermediate compound of formula (C) may be prepared as described in Scheme 9, below.

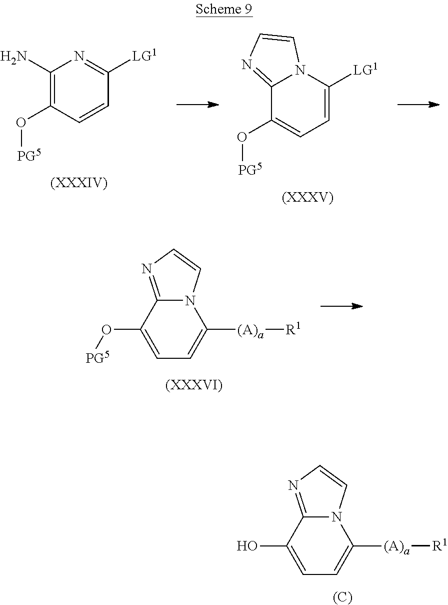

Accordingly, a suitably substituted compound of formula (XXXIV), wherein PG⁵ is a suitably selected oxygen protecting group such as benzyl, t-butyl, 4-methoxy-benzyl, and the like, and wherein LG¹ is a suitably selected leaving group such as Br, I, OTf, and the like, a known compound or compound prepared by known methods, is reacted with a suitably selected electrophilic agent such as 2-chloroacetaldehyde, 2-bromo-1,1-dimethoxyethane and the like; in the presence of an acid such as TFA, pTSA, HCl, and the like; in a suitably selected solvent such as EtOH, THF, toluene, and the like; at a temperature in the range of from about 60° C. to about 100° C., for example at about 80° C., to yield the corresponding compound of formula (XXXV).

The compound of formula (XXXV) is substituted for the compound of formula (IX) and reacted as described in Schemes 1-6, above (which series of substitutions and reaction steps would be readily understood by those skilled in the art); to yield the corresponding compound of formula (XXXVI).

The compound of formula (XXXVI) is de-protected, according to known methods, to yield the corresponding compound of formula (C). For example, wherein the PG⁵ protecting group is benzyl, the compound of formula (XXXVI) is reacted with H2 in the presence of a suitably selected catalyst such as Pd/C; in a suitably selected solvent such as ethyl acetate; to yield the corresponding compound of formula (C).

### Pharmaceutical Compositions

The present invention further comprises pharmaceutical compositions containing one or more compounds of formula (I) with a pharmaceutically acceptable carrier. Pharmaceutical compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

To prepare the pharmaceutical compositions of this invention, one or more compounds of the present invention as the active ingredient is intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like, for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, through other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, of from about 0.01 mg to about 1000 mg or any amount or range therein, and may be given at a dosage of from about 0.05 mg/day to about 300 mg/day, or any amount or range therein, preferably from about 0.1 mg/day to about 100 mg/day, or any amount or range therein, preferably from about 1 mg/day to about 50 mg/day, or any amount or range therein. The dosages, however, may be varied depending upon the requirement of the patients, the severity of the condition being treated and the compound being employed. The use of either daily administration or post-periodic dosing may be employed.

Preferably these compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from about 0.01 mg to about 1,000 mg, or any amount or range therein, of the active ingredient of the present invention. The tablets or pills of the novel composition can be coated or otherwise compounded to provide a dosage form yielding the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the novel compositions of the present invention may be incorporated for administration orally or by injection include, aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The method of treating disorders mediated by enteropeptidase activity, described in the present invention may also be carried out using a pharmaceutical composition comprising any of the compounds as defined herein and a pharmaceutically acceptable carrier. The pharmaceutical composition may contain between about 0.01 mg and about 1000 mg of the compound, or any amount or range therein, preferably from about 0.05 mg to about 300 mg of the compound, or any amount or range therein, more preferably from about 0.1 mg to about 100 mg of the compound, or any amount or range therein, more preferably from about 0.1 mg to about 50 mg of the compound, or any amount or range therein, and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixirs, emulsions, and suspensions. Forms useful for parenteral administration include sterile solutions, emulsions and suspensions.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitably flavored suspending or dispersing agents such as the synthetic and natural gums, for example, tragacanth, acacia, methylcellulose and the like. For parenteral administration, sterile suspensions and solutions are desired. Isotonic preparations which generally contain suitable preservatives are employed when intravenous administration is desired.

To prepare a pharmaceutical composition of the present invention, a compound of formula (I) as the active ingredient is intimately admixed with a pharmaceutical carrier according to conventional pharmaceutical compounding techniques, which carrier may take a wide variety of forms depending of the form of preparation desired for administration (e.g. oral or parenteral). Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

Compounds of this invention may be administered in any of the foregoing compositions and according to dosage regimens established in the art whenever treatment of disorders mediated by enteropeptidase activity, is required.

The daily dosage of the products may be varied over a wide range from about 0.01 mg to about 1,000 mg per adult human per day, or any amount or range therein. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250 and 500 milligrams of the active ingredient for the symptomatic adjustment of the dosage to the patient to be treated. An effective amount of the drug may be ordinarily supplied at a dosage level of from about 0.005 mg/kg to about 10 mg/kg of body weight per day, or any amount or range therein. Preferably, the range is from about 0.01 to about 5.0 mg/kg of body weight per day, or any amount or range therein, more preferably, from about 0.1 to about 1.0 mg/kg of body weight per day, or any amount or range therein, more preferably, from about 0.1 to about 0.5 mg/kg of body weight per day, or any amount or range therein. The compounds may be administered on a regimen of 1 to 4 times per day.

Optimal dosages to be administered may be readily determined by those skilled in the art, and will vary with the particular compound used, the mode of administration, the strength of the preparation, the mode of administration, and the advancement of the disease condition. In addition, factors associated with the particular patient being treated, including patient age, weight, diet and time of administration, will result in the need to adjust dosages.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trails including first-in-human, dose ranging and efficacy trials, in healthy patients and/or those suffering from a given disorder, may be completed according to methods well known in the clinical and medical arts.

The following Examples are set forth to aid in the understanding of the invention, and are not intended and should not be construed to limit in any way the invention set forth in the claims which follow thereafter.

In the Examples which follow, some synthesis products are listed as having been isolated as a residue. It will be understood by one of ordinary skill in the art that the term “residue” does not limit the physical state in which the product was isolated and may include, for example, a solid, an oil, a foam, a gum, a syrup, and the like.

### Example 1: Compound #1

### 2-{8-[(4-carbamimidamidophenyl)carbonyloxy]-[1,2,4]triazolo[1,5-a]pyridin-5-yl}acetic acid

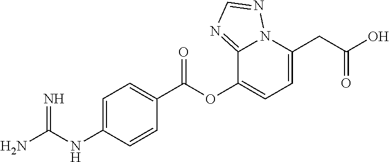 [SMILES: N=C(N)Nc1ccc(C(=O)Oc2ccc(CC(=O)O)n3ncnc23)cc1]

### Step 1: 3-(benzyloxy)-6-bromo-2-nitropyridine

Into a 250-mL round bottle was placed 6-bromo-2-nitropyridin-3-ol (2.00 g, 9.133 mmol, 1.00 equiv), K2CO₃ (1.90 g, 14.729 mmol, 1.61 equiv), DMF (100 mL). (Bromomethyl)benzene (1.87 g, 10.933 mmol, 1.20 equiv) was then added. The resulting solution was stirred 16 h at 70° C. The reaction was then quenched by H₂O (100 mL). The resulting solution was extracted with EtOAc (50 ml×3) and the organic layers combined and concentrated under vacuum. The residue was purified by silica gel chromatography (0-50% EtOAc/petroleum ether) to yield 3-(benzyloxy)-6-bromo-2-nitropyridine as a brown solid. Mass spectrum (ESI, m/z): Calculated for C₁₂H₉BrN₂O₃, 309.0 [M+H], Measured: 308.9.

### Step 2: 3-(benzyloxy)-6-bromopyridin-2-amine

Into a 250-mL round bottle was placed 3-(benzyloxy)-6-bromo-2-nitropyridine (2.57 g, 8.314 mmol, 1.00 equiv), Fe (1.87 g, 33.393 mmol, 4.02 equiv), NH₄Cl (1.80 g, 33.333 mmol, 4.01 equiv), EtOH (100 mL), H₂O (20 ml). The resulting solution was stirred for 2 h at 80° C. The solid was filtered out. The resulting solution was concentrated under vacuum quenched and quenched by H₂O (100 ml). The aqueous phase was extracted with EA (3×50 mL). The combined organic phases were washed with brine, dried (Na₂SO₄), the mixture filtered and the filtrate concentrated under vacuum to yield 3-(benzyloxy)-6-bromopyridin-2-amine as a brown solid.

### Step 3: (E)-N′-(3-(benzyloxy)-6-bromopyridin-2-yl)-N-hydroxyform imidamide

Into a 100-mL round bottle was placed 3-(benzyloxy)-6-bromopyridin-2-amine (1.20 g, 4.299 mmol, 1.00 equiv), DMF-DMA (1.03 g, 8.655 mmol, 2.01 equiv), i-PrOH (50 mL). The resulting solution was stirred 5 h at 100° C. and then cooled down to 55° C. NH₂OH.HCl (0.87 g, 12.609 mmol, 2.93 equiv) and NaHCO₃ (1.16 g, 13.810 mmol, 3.21 equiv) were added. The resulting solution was stirred 16 h at 55° C. The resulting solution was concentrated under vacuum. The residue was purified by silica gel chromatography (0-50% EtOAc/petroleum ether) to yield (E)-N′-(3-(benzyloxy)-6-bromopyridin-2-yl)-N-hydroxyformimidamide as a yellow solid. Mass spectrum (ESI, m/z): Calculated for C₁₃H₁₂BrN₃O₂:322.0 [M+H], Measured: 323.8.

### Step 4: 8-(benzyloxy)-5-bromo-[1,2,4]triazolo[1,5-a]pyridine

Into a 100-mL round bottle was placed (E)-N′-(3-(benzyloxy)-6-bromopyridin-2-yl)-N-hydroxyformimidamide (1.13 g, 3.508 mmol, 1.00 equiv), THE (50 ml). TFAA (2 ml) was added at 0° C. The resulting solution was stirred for 16 h at 20° C. The resulting solution was concentrated under vacuum. The residue was purified by silica gel chromatography (0-50% EtOAc/petroleum ether) to yield 8-(benzyloxy)-5-bromo-[1,2,4]triazolo[1,5-a]pyridine as a yellow solid. Mass spectrum (ESI, m/z): Calculated for C₁₃H₁₀BrN₃O, 304.0[M+H], Measured: 306.0.

### Step 5: tert-butyl 2-(8-(benzyloxy)-[1,2,4]triazolo[1,5-a]pyridin-5-yl)acetate

Into a 20-mL microwave tube was placed 8-(benzyloxy)-5-bromo-[1,2,4]triazolo[1,5-a]pyridine (1.00 g, 3.288 mmol, 1.00 equiv), (2-tert-butoxy-2-oxoethyl)zinc(II) bromide (1.27 g, 4.876 mmol, 1.48 equiv), Pd₂(dba)₃ (0.34 g, 0.329 mmol, 0.10 equiv), XPhos (0.32 mg, 0.671 mmol, 0.20 equiv), THE (10 ml). The resulting solution was stirred for 1.5 h at 110° C. under microwave. The resulting solution was concentrated under vacuum. The residue was purified by silica gel chromatography (0-30% EtOAc/petroleum ether) to yield tert-butyl 2-(8-(benzyloxy)-[1,2,4]triazolo[1,5-a]pyridin-5-yl)acetate as a yellow solid. Mass spectrum (ESI, m/z): Calculated for C₁₉H₂₁N₃O₃: 340.2[M+H], Measured: 340.1.

### Step 6: tert-butyl 2-(8-hydroxy-[1,2,4]triazolo[1,5-a]pyridin-5-yl)acetate

Into a 100-mL round bottle was placed tert-butyl 2-(8-(benzyloxy)-[1,2,4]triazolo[1,5-a]pyridin-5-yl)acetate (1.10 g, 3.241 mmol, 1.00 equiv), Pd/C (500 mg), EA (50 mL). H₂ was introduced. The resulting solution was stirred 3 h at 20° C. The solid was filtered out. The resulting solution was concentrated under vacuum. The residue was purified by silica gel chromatography (0-50% EtOAc/petroleum ether) to yield tert-butyl 2-(8-hydroxy-[1,2,4]triazolo [1,5-a]pyridin-5-yl)acetate as a yellow oil. Mass spectrum (ESI, m/z): Calculated for C₁₂H₁₅N₃O₃: 250.1 [M+H], Measured: 250.1.

### Step 7. 5-[2-(tert-butoxy)-2-oxoethyl]-[1,2,4]triazolo[1,5-a]pyridin-8-yl 4-{[(1Z)-{[(tert-butoxy)carbonyl]amino}({[(tert-butoxy)carbonyl]imino}) methyl]amino}benzoate

Into a 50-mL round bottle was placed tert-butyl 2-(8-hydroxy-[1,2,4]triazolo[1,5-a]pyridin-5-yl)acetate (300 mg, 1.204 mmol, 1.00 equiv), (Z)-4-(2,3-bis(tert-butoxycarbonyl)guanidino)benzoic acid (550 mg, 1.450 mmol, 1.20 equiv), EDCI (345 mg, 1.806 mmol, 1.50 equiv), DMAP (220 mg, 1.803 mmol, 1.46 equiv), DMF (20 mL). The resulting solution was stirred 4 h at 20° C. The reaction was then quenched by H₂O (100 mL). The resulting solution was extracted with EtOAc (50 ml×3) and the organic layers combined and concentrated under vacuum. The residue was purified by silica gel chromatography (0-50% EtOAc/petroleum ether) to yield 5-[2-(tert-butoxy)-2-oxoethyl]-[1,2,4]triazolo[1,5-a]pyridin-8-yl 4-{[(1Z)-{[(tert-butoxy)carbonyl]amino}({[(tert-butoxy)carbonyl]imino})methyl]amino} benzoate as a yellow solid. Mass spectrum (ESI, m/z): Calculated for C₃H₃₈N₆O₈: 611.3 [M+H], Measured: 611.4.

### Step 8. 2-{8-[(4-carbamimidamidophenyl)carbonyloxy]-[1,2,4]triazolo[1,5-a]pyridin-5-yl}acetic acid

Into a 50-mL round bottle was added 5-[2-(tert-butoxy)-2-oxoethyl]-[1,2,4]triazolo[1,5-a]pyridin-8-yl 4-{[(1Z)-{[(tert-butoxy)carbonyl]amino}({[(tert-butoxy)carbonyl]imino})methyl]amino}benzoate (100 mg, 0.164 mmol, 1.00 equiv), TFA (0.5 ml), DCM (20 mL). The resulting solution was stirred for 2 h at 20° C. The resulting solution was concentrated under vacuum. The residue was purified by Prep-HPLC with the following conditions (1#-Waters 2767-5): Column, X Bridge C18, 5 um, 19*100 mm; mobile phase, Water with 0.05% TFA and CH₃CN (8% CH₃CN up to 18% in 10 min, up to 95% in 0.1 min, hold 95% in 0.9 min, down to 8% in 0.1 min, hold 8% in 1.4 min); Detector, UV 220&254 nm to yield 2-{8-[(4-carbamimidamidophenyl)carbonyloxy]-[1,2,4]triazolo[1,5-a]pyridin-5-yl}acetic acid as a white solid.

¹H NMR (300 MHz, DMSO-d6) δ: 10.15 (s, 1H), 8.52 (s, 1H), 8.25 (d, J=8.4 Hz, 2H), 7.73-7.80 (m, 5H), 7.49 (d, J=8.4 Hz, 2H), 7.31 (d, J=8.1 Hz, 1H), 4.25 (s, 2H). Mass spectrum (ESI, m/z): Calculated for C₁₆H₁₄N₆O₄:355.1 [M+H]. Measured: 355.0.

### Example 2: Compound #2

### 3-(8-(4-guanidinobenzoyloxy)imidazo[1,2-a]pyridin-5-yl)propanoic acid

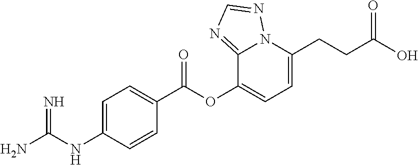 [SMILES: N=C(N)Nc1ccc(C(=O)Oc2ccc(CCC(=O)O)n3ncnc23)cc1]

### Step 1: 3-(benzyloxy)-6-bromo-2-nitropyridine

To a solution of 6-bromo-2-nitropyridin-3-ol (1 g, 45.664 mmol) in DMF (20 ml), was added di-tert-butyl dicarbonate (9.372 g, 54.797 mmol) and K2CO₃ (10.1 g, 91.328 mmol). The resulting solution was stirred for 1 h at 60° C. The reaction was monitored by TLC. Water was added, and the resulting solution was extracted with EA (100 mL×4), then dried over Na₂SO₄ and evaporated in vacuo. The residue was purified by silica gel chromatography (40-60% EtOAc/petroleum ether) to yield 3-(benzyloxy)-6-bromo-2-nitropyridine as a white solid.

### Step 2: 3-(benzyloxy)-6-bromopyridin-2-amine

To a solution of 3-(benzyloxy)-6-bromo-2-nitropyridine (14.000 g, 45.291 mmol) in EtOH (100 ml) and water (25 mL) was added iron (10.117 g, 181.162 mmol) and ammonium chloride (9.691 g, 181.162 mmol). The solution was stirred for 2 h at 80° C. The reaction was monitored by TLC. The reaction was filtrated and evaporated in vacuo. Water was added, and the resulting solution was extracted with EA (100 mL×4), dried over Na₂SO₄ and evaporated in vacuo to yield 3-(benzyloxy)-6-bromopyridin-2-amine as a brown solid. Mass spectrum (ESI, m/z): Calculated for C₁₂H₁₁BrN₂O, 281.1 [M+H], Measured: 281.0.

### Step 3: 8-(benzyloxy)-5-bromoimidazo[1,2-a]pyridine

To a solution of 3-(benzyloxy)-6-bromopyridin-2-amine (5.000 g, 17.913 mmol) in EtOH (50 mL), 2-chloroacetaldehyde (4.218 g, 53.738 mmol) was added. The solution was stirred for 3 h at 80° C. The reaction was monitored by TLC. The reaction was evaporated in vacuo and the residue was purified by silica gel chromatography (40-60% EtOAc/petroleum ether) to yield 8-(benzyloxy)-5-bromoimidazo[1,2-a]pyridine as a brown solid. Mass spectrum (ESI, m/z): Calculated for C₁₄H₁₁BrN₂O, 305.2 [M+H], Measured: 305.0.

### Step 4: (E)-tert-butyl 3-(8-(benzyloxy)imidazo[1,2-a]pyridin-5-yl)acrylate

Into a 100-mL round bottle and maintain with N₂ was placed 8-(benzyloxy)-5-bromoimidazo[1,2-a]pyridine (650 mg, 2.144 mmol, 1.00 equiv), tert-butyl acrylate (330 mg, 2.575 mmol, 1.20 equiv), Pd(dppf)Cl₂.CH₂Cl₂ (149 mg, 0.214 mmol, 0.10 equiv), NaOAc (352 mg, 4.293 mmol, 2.00 equiv), DMF (30 ml). The resulting solution was stirred for 16 h at 100° C. The reaction was then quenched by H₂O (100 mL). The resulting solution was extracted with EtOAc (50 mL×3) and the organic layers combined and concentrated under vacuum. The residue was purified by silica gel chromatography (0-50% EtOAc/petroleum ether) to yield (E)-tert-butyl 3-(8-(benzyloxy)imidazo [1,2-a]pyridin-5-yl)acrylate as a yellow oil. Mass spectrum (ESI, m/z): Calculated for C₂₁H₂₂N₂O₃:351.2[M+H], Measured: 351.1.

### Step 5: tert-butyl 2-(8-hydroxy-[1,2,4]triazolo[1,5-a]pyridin-5-yl)acetate

Into a 100-mL round bottle was placed (E)-tert-butyl 3-(8-(benzyloxy)imidazo[1,2-a]pyridin-5-yl)acrylate (500 mg, 1.427 mmol, 1.00 equiv), Pd/C (250 mg), EA (50 mL). H2 was introduced in. The resulting solution was stirred 3 h at 20° C. The solid was filtered out. The resulting solution was concentrated under vacuum. The residue was purified by silica gel chromatography (0-50% EtOAc/petroleum ether) to yield tert-butyl 3-(8-hydroxyimidazo[1,2-a]pyridin-5-yl)propanoate as a yellow oil. Mass spectrum (ESI, m/z): Calculated for C₁₄H₁₈N₂O₃: 263.1[M+H], Measured: 263.0.

### Step 6: 5-[3-(tert-butoxy)-3-oxopropyl]imidazo[1,2-a]pyridin-8-yl-4-{[(1Z)-{[(tert-butoxy)carbonyl]amino}({[(tert-butoxy)carbonyl]imino})methyl] amino}benzoate

Into a 50-mL round bottle was placed tert-butyl 3-(8-hydroxyimidazo[1,2-a]pyridin-5-yl)propanoate (200 mg, 0.762 mmol, 1.00 equiv), (Z)-4-(2,3-bis(tert-butoxycarbonyl)guanidino)benzoic acid (432 mg, 1.139 mmol, 1.49 equiv), EDCI (210 mg, 1.099 mmol, 1.44 equiv), DMAP (140 mg, 1.148 mmol, 1.51 equiv), DMF (20 mL). The resulting solution was stirred 4 h at 20° C. The reaction was then quenched by H₂O (100 mL). The resulting solution was extracted with EtOAc (50 ml×3) and the organic layers combined and concentrated under vacuum. The residue was purified by silica gel chromatography (0-50% EtOAc/petroleum ether) to yield 5-[3-(tert-butoxy)-3-oxopropyl]imidazo[1,2-a]pyridin-8-yl-4-{[(1Z)-{[(tert-butoxy)carbonyl]amino}({[(tert-butoxy) carbonyl]imino})methyl]amino}benzoate as a yellow solid. Mass spectrum (ESI, m/z): Calculated for C₃₂H₄₁N₅O₈: 624.3 [M+H], Measured: 624.6.

### Step 7: 3-(8-(4-guanidinobenzoyloxy)imidazo[1,2-a]pyridin-5-yl)propanoic acid

Into a 50-mL round bottle was added 5-[3-(tert-butoxy)-3-oxopropyl]imidazo[1,2-a]pyridin-8-yl 4-{[(1Z)-{[(tert-butoxy)carbonyl]amino}({[(tert-butoxy)carbonyl]imino})methyl]amino}benzoate (230 mg, 0.369 mmol, 1.00 equiv), TFA (2 ml), DCM (30 mL). The resulting solution was stirred for 2 h at 20° C. The resulting solution was concentrated under vacuum. The residue was purified by Prep-HPLC with the following conditions (1#-Waters 2767-5): Column, X Bridge C18, 5 um, 19*100 mm; mobile phase, Water with 0.05% TFA and CH₃CN (8% CH₃CN up to 18% in 10 min, up to 95% in 0.1 min, hold 95% in 0.9 min, down to 8% in 0.1 min, hold 8% in 1.4 min); Detector, UV 220&254 nm to yield 3-(8-(4-guanidinobenzoyloxy)imidazo[1,2-a]pyridin-5-yl)propanoic acid as a white solid.

¹H NMR (300 MHz, DMSO-d6) δ: 10.20-10.35 (m, 1H), 7.72-7.35 (m, 8H), 7.33-7.66 (m, 3H), 7.02-7.15 (m, 1H), 3.11-3.29 (m, 2H), 2.74-2.85 (m, 2H). Mass spectrum (ESI, m/z): Calculated for C₁₈H₁₇N₅O₄:368.1 [M+H]. Measured: 368.1.

### Example 3: Compound #3

### 3-(8-(4-guanidinobenzoyloxy)-[1,2,4]triazolo[1,5-a]pyridin-5-yl)propanoic acid

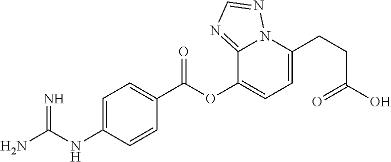 [SMILES: N=C(N)Nc1ccc(C(=O)Oc2ccc(CCC(=O)O)n3ncnc23)cc1]

### Step 1: (E)-tert-butyl 3-(8-(benzyloxy)-[1,2,4]triazolo[1,5-a]pyridin-5-yl)acrylate

Into a 100 ml round bottom-flask, 8-(benzyloxy)-5-bromo-[1,2,4]triazolo[1,5-a]pyridine (50 mg, 0.164 mmol, 1.00 equiv) and tert-butyl acrylate (42 mg, 0.328 mmol, 2.00 equiv) was dissolved in DMF (5 ml). Pd₂(dba)₃ (17 mg, 0.016 mmol, 0.10 equiv), TEA (50 mg, 0.495 mmol, 3.00 equiv) and PPh₃ (5 mg, 0.019 mmol, 0.10 equiv) were then added to the solution. Under N2 atmosphere the resulting solution was stirred 16 h at 100° C. The reaction was monitored by LCMS. The reaction was quenched by H₂O (10 ml). The resulting solution was extracted with EtOAc (15 ml×3) and the organic layers combined and concentrated under vacuum. The residue was purified by silica gel chromatography (0-50% EtOAc/petroleum ether) to yield (E)-tert-butyl 3-(8-(benzyloxy)-[1,2,4]triazolo[1,5-a]pyridin-5-yl)acrylate as a yellow solid. Mass spectrum (ESI, m/z): Calculated for C₂₀H₂₁N₃O₃, 352.3 [M+Na], Measured: 352.2.

### Step 2: tert-butyl 3-(8-hydroxy-[1,2,4]triazolo[1,5-a]pyridin-5-yl)propanoate

To a solution of (E)-tert-butyl 3-(8-(benzyloxy)-[1,2,4]triazolo[1,5-a]pyridin-5-yl)acrylate (80 mg, 0.228 mmol, 1.0 equiv) in EA (5 mL) was added Pd/C (80 mg). The resulting solution was hydrogenated under 1 atm of H₂ at 25° C. for 3 hours. The resulting mixture was filtered. The filtrate was concentrated under vacuum. The residue was purified by silica gel chromatography (0-10% EtOAc/petroleum ether) to yield tert-butyl 3-(8-hydroxy-[1,2,4]triazolo[1,5-a]pyridin-5-yl)propanoate as a yellow oil. Mass spectrum (ESI, m/z): Calculated for C₁₃H₁₇N₃O₃, 264.2 [M+H], Measured: 264.0.

### Step 3: 5-[3-(tert-butoxy)-3-oxopropyl]-[1,2,4]triazolo[1,5-a]pyridin-8-yl 4-{[(1Z)-{[(tert-butoxy)carbonyl]amino}({[(tert-butoxy)carbonyl]imino}) methyl]amino}benzoate

To a solution of tert-butyl 3-(8-hydroxy-[1,2,4]triazolo[1,5-a]pyridin-5-yl)propanoate (55 mg, 0.209 mmol) in DCM (5 ml) was added EDCI (85 mg, 0.445 mmol, 2.00 equiv), DMAP (51 mg, 0.418 mmol, 2.00 equiv) and (Z)-4-(2,3-bis(tert-butoxycarbonyl)guanidino)benzoic acid (95 mg, 0.250 mmol, 1.20 equiv). The resulting mixture was stirred for 2 h at 25° C. The resulting solution was concentrated under vacuum. The residue was purified by silica gel chromatography (0-50% EtOAc/petroleum ether) to yield 5-[3-(tert-butoxy)-3-oxopropyl]-[1,2,4]triazolo[1,5-a]pyridin-8-yl 4-{[(1Z)-{[(tert-butoxy)carbonyl]amino}({[(tert-butoxy)carbonyl]imino})methyl]amino}benzoate as a yellow oil. Mass spectrum (ESI, m/z): Calculated for C₃₁H₄₀N₆O₈, 625.3 [M+H], Measured: 625.4.

### Step 4: 3-(8-(4-guanidinobenzoyloxy)-[1,2,4]triazolo[1,5-a]pyridin-5-yl)propanoic acid

Into a 50 ml round bottom-flask, 5-[3-(tert-butoxy)-3-oxopropyl]-[1,2,4]triazolo[1,5-a]pyridin-8-yl 4-{[(1E)-{[(tert-butoxy)carbonyl]amino}({[(tert-butoxy)carbonyl]imino})methyl]amino}benzoate (80 mg, 0.128 mmol, 1.00 equiv) was dissolved in DCM (5 ml). Then TFA (2 ml) was added to the solution. The resulting solution was stirred 1.0 h at room temperature. The reaction was monitored by LCMS. The organic layers were concentrated under vacuum. The residue was purified by Prep-HPLC with the following conditions: (1#waters2767-5): Column, SunFire Prep C18, 19*150 mm 5 umH PrepC-001(T)18600256819513816414 04; mobile phase, Phase A: water with 0.05% TFA; Phase B: CH₃CN (10% CH₃CN up to 28% in 10 min, up to 100% CH₃CN in 1 min, hold 100% in 1 min, down to 10% CH₃CN in 0.1 min, hold 5% in 0.9 min); Detector, UV220 & 254 nm to yield 3-(8-(4-guanidinobenzoyloxy)-[1,2,4]triazolo[1,5-a]pyridin-5-yl)propanoic acid as a white solid.

¹H NMR (400 MHz, DMSO-d6) δ 12.42 (brs, 1H), 10.34 (s, 1H), 8.55 (s, 1H), 8.23 (d, J=4.2 Hz, 2H), 7.90 (s, 4H), 7.72 (t, J=5.4 Hz, 1H), 7.48 (d, J=4.2 Hz, 2H), 7.17 (t, J=3.8 Hz, 1H), 3.24 (t, J=3.6 Hz, 2H), 2.87 (t, J=3.8 Hz, 2H). Mass spectrum (ESI, m/z): Calculated for C₁₇H₁₆N₆O₄, 369.3[M+H], Measured: 369.2.

### Example 4: Compound #4

### 2-(2-(8-(4-guanidinobenzoyloxy)-[1,2,4]triazolo[1,5-a]pyridin-5-yl) acetamido)acetic acid

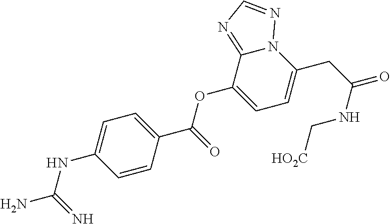 [SMILES: N=C(N)Nc1ccc(C(=O)Oc2ccc(CC(=O)NCC(=O)O)n3ncnc23)cc1]

### Step 1: 2-(8-(benzyloxy)-[1,2,4]triazolo[1,5-a]pyridin-5-yl)acetic acid

Into a 50 ml round bottom-flask, tert-butyl 2-(8-(benzyloxy)-[1,2,4]triazolo[1,5-a]pyridin-5-yl)acetate (420 mg, 1.238 mmol, 1.00 equiv) was dissolved in DCM (10 ml). Then TFA (4 ml) was added to the solution. The resulting solution was stirred 2.0 h at room temperature. The reaction was monitored by LCMS. The organic layers were concentrated under vacuum to yield 2-(8-(benzyloxy)-[1,2,4]triazolo[1,5-a]pyridin-5-yl)acetic acid. Mass spectrum (ESI, m/z): Calculated for C₁₅H₁₃N₃O₃, 284.2 [M+H], Measured: 284.3.

### Step 2: tert-butyl 2-(2-(8-(benzyloxy)-[1,2,4]triazolo[1,5-a]pyridin-5-yl)acetamido)acetate

To a solution of 2-(8-(benzyloxy)-[1,2,4]triazolo[1,5-a]pyridin-5-yl)acetic acid (100 mg, 0.353 mmol) in DCM (5 ml) were added HATU (270 mg, 0.711 mmol, 2.0 equiv), TEA (177 mg, 1.752 mmol, 5 equiv) and di-tert-butyl 2,2′-azanediyldiacetate (130 mg, 0.530 mmol, 1.50 equiv). The resulting mixture was stirred for 2 h at 25° C. The resulting solution was concentrated under vacuum. The residue was purified by silica gel chromatography (0-50% EtOAc/petroleum ether) to yield tert-butyl 2-(2-(8-(benzyloxy)-[1,2,4]triazolo[1,5-a]pyridin-5-yl)acetamido)acetate as a yellow oil. Mass spectrum (ESI, m/z): Calculated for C₂₁H₂₄N₄O₄: 397.2 [M+H], Measured: 397.4.

### Step 3: tert-butyl 2-(2-(8-hydroxy-[1,2,4]triazolo[1,5-a]pyridin-5-yl)acetamido)acetate

To a solution of tert-butyl 2-(2-(8-(benzyloxy)-[1,2,4]triazolo[1,5-a]pyridin-5-yl)acetamido)acetate (150 mg, 0.378 mmol 1.00 equiv) in EA (10 mL) was added Pd/C (150 mg). The resulting solution was hydrogenated under 1 atm of H2 at 25° C. for 3 hours. The reaction was monitored by TLC (PE/EA=1/1). The resulting mixture was filtered. The filtrate was concentrated under vacuum. The residue was purified by silica gel chromatography (0-10% EtOAc/petroleum ether) to yield tert-butyl 2-(2-(8-hydroxy-[1,2,4] triazolo[1,5-a]pyridin-5-yl)acetamido)acetate as a yellow oil.

### Step 4: 5-({[2-(tert-butoxy)-2-oxoethyl]carbamoyl}methyl)-[1,2,4] triazolo[1,5-a]pyridin-8-yl 4-{[(1Z)-{[(tert-butoxy)carbonyl]amino}({[(tert-butoxy)carbonyl]imino})methyl]amino}benzoate

To a solution of tert-butyl 2-(2-(8-hydroxy-[1,2,4]triazolo[1,5-a]pyridin-5-yl)acetamido)acetate (85 mg, 0.277 mmol) in DCM (5 ml) were added EDCI (90 mg, 0.471 mmol, 2.00 equiv), DMAP (52 mg, 0.426 mmol, 2.00 equiv) and (Z)-4-(2,3-bis(tert-butoxycarbonyl)guanidino)benzoic acid (100 mg, 0.264 mmol, 1.00 equiv). The resulting mixture was stirred for 2 h at 25° C. The resulting solution was concentrated under vacuum. The residue was purified by silica gel chromatography (0-50% EtOAc/petroleum ether) to yield 5-({[2-(tert-butoxy)-2-oxoethyl]carbamoyl}methyl)-[1,2,4]triazolo[1,5-a]pyridin-8-yl 4-{[(1Z)-{[(tert-butoxy)carbonyl]amino}({[(tert-butoxy)carbonyl]imino})methyl] amino}benzoate as a yellow oil. Mass spectrum (ESI, m/z): Calculated for C₃₂H₄₁N₇O₉, 667.7[M+H], Measured: 668.4.

### Step 5: 2-(2-(8-(4-guanidinobenzoyloxy)-[1,2,4]triazolo[1,5-a]pyridin-5-yl)acetamido)acetic acid

Into a 50 ml round bottom-flask, 5-({[2-(tert-butoxy)-2-oxoethyl]carbamoyl}methyl)-[1,2,4]triazolo[1,5-a]pyridin-8-yl 4-{[(1Z)-{[(tert-butoxy) carbonyl]amino}({[(tert-butoxy)carbonyl]imino})methyl]amino}benzoate (80 mg, 0.120 mmol, 1.00 equiv) was dissolved in DCM (5 ml). Then TFA (2 ml) was added to the solution. The resulting solution was stirred 5.0 h at room temperature. The reaction was monitored by LCMS. The organic layers were concentrated under vacuum. The residue was purified by Prep-HPLC with the following conditions: (1#waters2767-5):Column, SunFire Prep C18, 19*150 mm 5 umH PrepC-001(T)18600256819513816414 04; mobile phase, Phase A: water with 0.05% TFA; Phase B: CH₃CN (10% CH₃CN up to 28% in 10 min, up to 100% CH₃CN in 1 min, hold 100% in 1 min, down to 10% CH₃CN in 0.1 min, hold 5% in 0.9 min); Detector, UV220 & 254 nm to yield 2-(2-(8-(4-guanidinobenzoyloxy)-[1,2,4]triazolo[1,5-a]pyridin-5-yl)acetamido)acetic acid as a white solid.

¹H NMR (300 MHz, DMSO-d6) δ: 10.17 (s, 1H), 8.57-8.77 (m, 1H), 8.49 (s, 1H), 8.23 (d, J=4.35 Hz, 2H), 7.81 (s, 4H), 7.63-7.77 (m, 1H), 7.35-7.57 (m, 2H), 7.28 (t, J=4.05 Hz, 1H), 4.18 (s, 2H), 3.83 (d, J=2.85 Hz, 2H). Mass spectrum (ESI, m/z): Calculated for C₁₈H₁₇N₇O₅, 412.3 [M+H], Measured: 412.2.

### Example 5: Compound #5

### 2-(2-(8-(4-guanidinobenzoyloxy)-[1,2,4]triazolo[1,5-a]pyridin-5-yl)acetamido)succinic acid

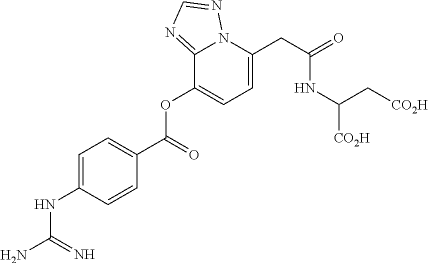 [SMILES: N=C(N)Nc1ccc(C(=O)Oc2ccc(CC(=O)NC(CC(=O)O)C(=O)O)n3ncnc23)cc1]

### Step 1: di-Tert-butyl 2-(2-(8-(benzyloxy)-[1,2,4]triazolo[1,5-a]pyridin-5-yl)acetamido)succinate

To a solution of 2-(8-(benzyloxy)-[1,2,4]triazolo[1,5-a]pyridin-5-yl)acetic acid (100 mg, 0.353 mmol) in DCM (5 ml) were added HATU (270 mg, 0.711 mmol, 2.00 equiv), TEA (177 mg, 1.752 mmol, 5.00 equiv) and (R)-di-tert-butyl 2-aminosuccinate (150 mg, 0.611 mmol, 1.50 equiv). The resulting mixture was stirred for 2 h at 25° C. The resulting solution was concentrated under vacuum. The residue was purified by silica gel chromatography (0-50% EtOAc/petroleum ether) to yield (R)-di-tert-butyl 2-(2-(8-(benzyloxy)-[1,2,4]triazolo[1,5-a]pyridin-5-yl)acetamido)succinate as a yellow oil. Mass spectrum (ESI, m/z): Calculated for C₂₇H₃₄N₄O₆, 511.5[M+H], Measured: 511.3.

### Step 2: 5(R)-di-tert-butyl 2-(2-(8-hydroxy-[1,2,4]triazolo[1,5-a]pyridin-5-yl)acetamido)succinate

To a solution of (R)-di-tert-butyl 2-(2-(8-(benzyloxy)-[1,2,4]triazolo[1,5-a]pyridin-5-yl)acetamido)succinate (130 mg, 0.255 mmol, 1.00 equiv) in EA (5 mL) was added Pd/C (130 mg). The resulting solution was hydrogenated under 1 atm of H2 at 25° C. for 3 hours. The reaction was monitored by TLC (PE/EA=1/1). The resulting mixture was filtered. The filtrate was concentrated under vacuum. The residue was purified by silica gel chromatography (0-10% EtOAc/petroleum ether) to yield (R)-di-tert-butyl 2-(2-(8-hydroxy-[1,2,4]triazolo[1,5-a]pyridin-5-yl)acetamido)succinate as a yellow oil.

### Step 3: 1,4-di-tert-butyl (2R)-2-(2-{8-[(4-{[(1Z)-{[(tert-butoxy)carbonyl] amino}({[(tert-butoxy)carbonyl]imino})methyl]amino}phenyl) carbonyloxy]-[1,2,4]triazolo[1,5-a]pyridin-5-yl}acetamido)butanedioate

To a solution of (R)-di-tert-butyl 2-(2-(8-hydroxy-[1,2,4]triazolo[1,5-a]pyridin-5-yl)acetamido)succinate (100 mg, 0.238 mmol) in DCM (5 ml) were added EDCI (100 mg, 0.524 mmol, 2.00 equiv), DMAP (60 mg, 0.492 mmol, 2.00 equiv) and (Z)-4-(2,3-bis(tert-butoxycarbonyl)guanidino)benzoic acid (110 mg, 0.290 mmol, 1.20 equiv). The resulting mixture was stirred for 2 h at 25° C. The resulting solution was concentrated under vacuum. The residue was purified by silica gel chromatography (0-50% EtOAc/petroleum ether) to yield 1,4-di-tert-butyl (2R)-2-(2-{8-[(4-{[(1Z)-{[(tert-butoxy)carbonyl]amino}({[(tert-butoxy)carbonyl]imino})methyl]amino}phenyl)carbonyloxy]-[1,2,4]triazolo[1,5-a]pyridin-5-yl}acetamido)butanedioate as a yellow oil. Mass spectrum (ESI, m/z): Calculated for C₃₈H₅₁N₇O₁₁, 782.4[M+H], Measured: 782.5.

### Step 4: 2-(2-(8-(4-guanidinobenzoyloxy)-[1,2,4]triazolo[1,5-a]pyridin-5-yl)acetamido)succinic acid

Into a 50 ml round bottom-flask, 1,4-di-tert-butyl 2-(2-{8-[(4-{[(1E)-{[(tert-butoxy)carbonyl]amino}({[(tert-butoxy)carbonyl]imino})methyl]amino}phenyl) carbonyloxy]-[1,2,4]triazolo[1,5-a]pyridin-5-yl}acetamido)butanedioate (100 mg, 0.128 mmol, 1.00 equiv) was dissolved in DCM (5 ml). Then TFA (2 ml) was added to the solution. The resulting solution was stirred 1.0 h at room temperature. The reaction was monitored by LCMS. The organic layers were concentrated under vacuum. The residue was purified by Prep-HPLC with the following conditions: (1#waters2767-5): Column, SunFire Prep C18, 19*150 mm 5 umH PrepC-001(T)18600256819513816414 04; mobile phase, Phase A: water with 0.05% TFA; Phase B: CH₃CN (10% CH₃CN up to 28% in 10 min, up to 100% CH₃CN in 1 min, hold 100% in 1 min, down to 10% CH₃CN in 0.1 min, hold 5% in 0.9 min); Detector, UV220 & 254 nm to yield 2-(2-(8-(4-guanidinobenzoyloxy)-[1,2,4]triazolo[1,5-a]pyridin-5-yl)acetamido)succinic acid as a white solid.

¹H NMR (300 MHz, DMSO-d6) δ 12.69-12.44 (m, 2H), 10.19 (s, 1H), 8.72 (d, J=3.9 Hz, 1H), 8.47 (d, J=2.1 Hz, 1H), 8.23 (d, J=4.35 Hz, 2H), 7.81 (s, 4H), 7.70 (t, J=3.9 Hz, 1H), 7.48 (d, J=4.4 Hz, 2H), 7.25 (d, J=3.9 Hz, 1H), 4.54-4.61 (m, 1H), 4.16 (s, 2H), 2.56-2.77 (m, 2H)./¹⁹F NMR (300 MHz, DMSO-d6) δ −73.31. Mass spectrum (ESI, m/z): Calculated for C₂₀H₁₉N₇O₇, 470.1 [M+H], Measured: 470.2.

### Example 6: Compound #6

### 2,2′-(2-(8-(4-guanidinobenzoyloxy)-[1,2,4]triazolo[1,5-a]pyridin-5-yl)acetamido)diacetic acid

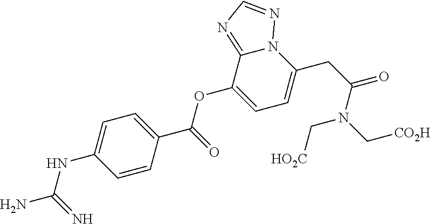 [SMILES: N=C(N)Nc1ccc(C(=O)Oc2ccc(CC(=O)N(CC(=O)O)CC(=O)O)n3ncnc23)cc1]

### Step 1: di-tert-butyl 2,2′-(2-(8-(benzyloxy)-[1,2,4]triazolo[1,5-a]pyridin-5-yl)acetamido)diacetate

To a solution of 2-(8-(benzyloxy)-[1,2,4]triazolo[1,5-a]pyridin-5-yl)acetic acid (100 mg, 0.353 mmol) in DCM (10 ml) were added HATU (270 mg, 0.711 mmol, 2.00 equiv), TEA (177 mg, 1.752 mmol, 5.00 equiv) and di-tert-butyl 2,2′-azanediyldiacetate (130 mg, 0.530 mmol, 1.50 equiv). The resulting mixture was stirred for 2 h at 25° C. The resulting solution was concentrated under vacuum. The residue was purified by silica gel chromatography (0-50% EtOAc/petroleum ether) to yield di-tert-butyl 2,2′-(2-(8-(benzyloxy)-[1,2,4]triazolo[1,5-a]pyridin-5-yl)acetamido)diacetate as a yellow oil. Mass spectrum (ESI, m/z): Calculated for C₂₇H₃₄N₄O₆: 511.2 [M+H], Measured: 511.4.

### Step 2: (R)-di-tert-butyl 2-(2-(8-hydroxy-[1,2,4]triazolo[1,5-a]pyridin-5-yl)acetamido)succinate

To a solution of (R)-di-tert-butyl 2-(2-(8-(benzyloxy)-[1,2,4]triazolo[1,5-a]pyridin-5-yl)acetamido)succinate (150 mg, 0.294 mmol, 1.00 equiv) in EA (10 mL) was added Pd/C (150 mg). The resulting solution was hydrogenated under 1 atm of H2 at 25° C. for 3 hours. The reaction was monitored by TLC (PE/EA=1/1). The resulting mixture was filtered. The filtrate was concentrated under vacuum. The residue was purified by silica gel chromatography (0-10% EtOAc/petroleum ether) to yield (R)-di-tert-butyl 2-(2-(8-hydroxy-[1,2,4]triazolo[1,5-a]pyridin-5-yl)acetamido)succinate as a yellow oil.

### Step 3: 5-({bis[2-(tert-butoxy)-2-oxoethyl]carbamoyl}methyl)-[1,2,4]triazolo [1,5-a]pyridin-8-yl 4-{[(1Z)-{[(tert-butoxy)carbonyl]amino}({[(tert-butoxy) carbonyl]imino})methyl]amino}benzoate

To a solution of di-tert-butyl 2,2′-(2-(8-hydroxy-[1,2,4]triazolo[1,5-a]pyridin-5-yl)acetamido)diacetate (85 mg, 0.202 mmol) in DCM (5 ml) were added EDCI (70 mg, 0.336 mmol, 2.00 equiv), DMAP (40 mg, 0.328 mmol, 2.00 equiv) and (Z)-4-(2,3-bis(tert-butoxycarbonyl)guanidino)benzoic acid (76 mg, 0.200 mmol, 1.00 equiv). The resulting mixture was stirred for 2 h at 25° C. The resulting solution was concentrated under vacuum. The residue was purified by silica gel chromatography (0-50% EtOAc/petroleum ether) to yield 5-({bis[2-(tert-butoxy)-2-oxoethyl]carbamoyl}methyl)-[1,2,4]triazolo[1,5-a]pyridin-8-yl 4-{[(1Z)-{[(tert-butoxy)carbonyl]amino}({[(tert-butoxy)carbonyl]imino})methyl]amino}benzoate as a yellow oil. Mass spectrum (ESI, m/z): Calculated for C₃₈H₅₁N₇O₁₁, 782.4 [M+H], Measured: 782.5.

### Step 4: 2,2′-(2-(8-(4-guanidinobenzoyloxy)-[1,2,4]triazolo[1,5-a]pyridin-5-yl)acetamido)diacetic acid

Into a 50 ml round bottom-flask, 5-({bis[2-(tert-butoxy)-2-oxoethyl]carbamoyl}methyl)-[1,2,4]triazolo[1,5-a]pyridin-8-yl 4-{[(1E)-{[(tert-butoxy)carbonyl]amino}({[(tert-butoxy)carbonyl]imino})methyl]amino} benzoate (80 mg, 0.102 mmol, 1.00 equiv) was dissolved in DCM (5 ml). Then TFA (2 ml) was added to the solution. The resulting solution was stirred 5.0 h at room temperature. The reaction was monitored by LCMS. The organic layers were concentrated under vacuum. The residue was purified by Prep-HPLC with the following conditions: (1#waters2767-5): Column, SunFire Prep C18, 19*150 mm 5 umH PrepC-001(T)1 8600256819513816414 04; mobile phase, Phase A: water with 0.05% TFA; Phase B: CH₃CN (10% CH₃CN up to 28% in 10 min, up to 100% CH₃CN in 1 min, hold 100% in 1 min, down to 10% CH₃CN in 0.1 min, hold 5% in 0.9 min); Detector, UV220 & 254 nm to yield 2,2′-(2-(8-(4-guanidino benzoyloxy)-[1,2,4]triazolo[1,5-a]pyridin-5-yl)acetamido)diacetic acid as a white solid.

¹H NMR (300 MHz, DMSO-d⁶) δ 12.97 (s, 1H), 10.21 (s, 1H), 8.47 (s, 1H), 8.24 (d, J=4.35 Hz, 2H), 7.82 (s, 4H), 7.70 (d, J=3.9 Hz, 1H), 7.48 (d, J=4.35 Hz, 2H), 7.21 (d, J=3.9 Hz, 1H), 4.36 (d, J=10.0 Hz, 4H), 4.03 (s, 2H). Mass spectrum (ESI, m/z): Calculated for C₂₀H₁₉N₇O₇, 470.1 [M+H], Measured: 470.2.

### Example 7: Compound #7

### 4-(2-carboxyethyl)-4-(2-(8-(4-guanidinobenzoyloxy)-[1,2,4]triazolo[1,5-a]pyridin-5-yl)acetamido)heptanedioic acid

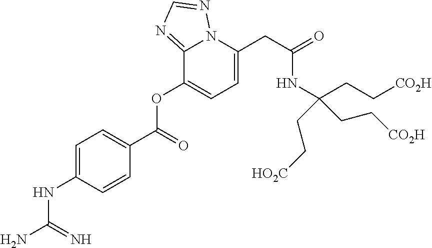 [SMILES: N=C(N)Nc1ccc(C(=O)Oc2ccc(CC(=O)NC(CCC(=O)O)(CCC(=O)O)CCC(=O)O)n3ncnc23)cc1]

### Step 1: 4-[2-(benzyloxy)-2-oxoethyl]phenyl 5-{[(1Z)-{[(tertbutoxy)carbonyl]amino}({[(tert-butoxy)carbonyl]imino})methyl]amino}indazole-1-carboxylate

To a solution of 2-(8-(benzyloxy)-[1,2,4]triazolo[1,5-a]pyridin-5-yl)acetic acid (60 mg, 0.212 mmol) in DCM (5.0 ml), was added HATU (96.582 mg, 0.254 mmol), Et₃N (42.784 mg, 0.424 mmol), di-tert-butyl 4-amino-4-(3-tert-butoxy-3-oxopropyl)heptanedioate (96.820 mg, 0.233 mmol). The resulting solution was stirred overnight at room temperature. The reaction was monitored by LCMS. The resulting mixture was concentrated under vacuum. The residue was purified by silica gel chromatography (40-60% EtOAc/petroleum ether) to yield 4-[2-(benzyloxy)-2-oxoethyl]phenyl 5-{[(1Z)-{[(tert-butoxy) carbonyl]amino}({[(tert-butoxy)carbonyl]imino})methyl]amino}indazole-1-carboxylate as a yellow oil. Mass spectrum (ESI, m/z): Calculated for C₃₇H₅₂N₄O₈, 681.4[M+H], Measured: 681.7.

### Step 2: di-tert-butyl 4-(3-tert-butoxy-3-oxopropyl)-4-(2-(8-hydroxy-[1,2,4]triazolo[1,5-a]pyridin-5-yl)acetamido)heptanedioate

Into a 30-mL round-bottom flask purged and maintained with atmosphere of hydric, was placed 1,7-di-tert-butyl 4-{2-[8-(benzyloxy)-[1,2,4]triazolo[1,5-a]pyridin-5-yl]acetamido}-4-[3-(tert-butoxy)-3-oxopropyl]heptanedioate (110 mg, 0.162 mmol), EA (10 ml), Pd/C (110 mg). The resulting solution was stirred 2.0 h at room temperature. The reaction progress was monitored by LCMS. The mixture was filtered through a CELITE® pad. The resulting mixture was concentrated under vacuum to yield di-tert-butyl 4-(3-tert-butoxy-3-oxopropyl)-4-(2-(8-hydroxy-[1,2,4]triazolo[1,5-a]pyridin-5-yl)acetamido)heptanedioate as a white solid. Mass spectrum (ESI, m/z): Calculated for C₃₀H₄₆N₄O₈, 591.3[M+H], Measured: 591.6.

### Step 3: 1,7-di-tert-butyl 4-[3-(tert-butoxy)-3-oxopropyl]-4-(2-{8-[(4-{[(1Z)-{[(tert-butoxy)carbonyl]amino}({[(tert-butoxy)carbonyl]imino})methyl]amino}phenyl)carbonyloxy]-[1,2,4]triazolo[1,5-a]pyridin-5-yl}acetamido)heptanedioate

To a solution of di-tert-butyl 4-(3-tert-butoxy-3-oxopropyl)-4-(2-(8-hydroxy-[1,2,4]triazolo[1,5-a]pyridin-5-yl)acetamido)heptanedioate (70 mg, 0.119 mmol) in DCM (5.0 ml), was added EDCI (45.268 mg, 0.237 mmol), DMAP (28.914 mg, 0.237 mmol), (E)-4-(2,3-bis(tert-butoxycarbonyl)guanidino) benzoic acid (53.953 mg, 0.142 mmol). The resulting solution was stirred overnight at room temperature. The reaction was monitored by LCMS. The resulting mixture was concentrated under vacuum. The residue was purified by silica gel chromatography (40-60% EtOAc/petroleum ether) to yield 1,7-di-tert-butyl 4-[3-(tert-butoxy)-3-oxopropyl]-4-(2-{8-[(4-{[(1Z)-{[(tert-butoxy)carbonyl]amino}({[(tert-butoxy)carbonyl]imino})methyl]amino}phenyl) carbonyloxy]-[1,2,4]triazolo[1,5-a]pyridin-5-yl}acetamido)heptanedioate as a yellow oil. Mass spectrum (ESI, m/z): Calculated for C₄₈H₆₉N₇O₁₃, 952.5[M+H], Measured: 952.8.

### Step 4: 4-(2-carboxyethyl)-4-(2-(8-(4-guanidinobenzoyloxy)-[1,2,4]triazolo[1,5-a]pyridin-5-yl)acetamido)heptanedioic acid

To a solution of 1,7-di-tert-butyl 4-[3-(tert-butoxy)-3-oxopropyl]-4-(2-{8-[(4-{[(1Z)-{[(tert-butoxy)carbonyl]amino}({[(tert-butoxy)carbonyl]imino})methyl]amino}phenyl) carbonyloxy]-[1,2,4]triazolo[1,5-a]pyridin-5-yl}acetamido) heptanedioate (120 mg, 0.126 mmol) in DCM (5.0 ml), was added TFA (1.2 ml). The resulting solution was stirred 2.0 h at room temperature. The reaction was monitored by LCMS. The resulting mixture was concentrated under vacuum. The residue was purified by Prep-HPLC with the following conditions (1#-Waters 2767-5): Column, SunFire Prep C18, 5 um, 19*100 mm; mobile phase, Water with 0.05% TFA and CH₃CN (5% CH₃CN up to 30% in 10 min, up to 100% in 0.1 min, hold 100% in 0.9 min, down to 5% in 0.1 min, hold 5% in 1.4 min); Detector, UV 220&254 nm to yield 4-(2-carboxyethyl)-4-(2-(8-(4-guanidinobenzoyloxy)-[1,2,4]triazolo[1,5-a]pyridin-5-yl)acetamido)heptanedioic acid as a white solid.

¹H NMR (300 MHz, DMSO-d⁶) δ: 12.08 (s, 3H), 10.28 (s, 1H), 8.35 (s, 1H), 8.31 (d, J=8.7 Hz, 2H), 7.86-7.99 (m, 5H), 7.71-7.79 (m, 1H), 7.46-7.49 (m, 2H), 7.19-7.23 (m, 1H), 4.11 (s, 2H), 2.15-2.21 (m, 6H), 1.74-1.87 (m, 6H). Mass spectrum (ESI, m/z): Calculated for C₂₈H₃₀F₃N₇O₁₁, 584.2[M+H], Measured: 584.2.

### Example 8: Compound #8

### 5-(2-(methylsulfonamido)-2-oxoethyl)-[1,2,4]triazolo[1,5-a]pyridin-8-yl4-guanidinobenzoate

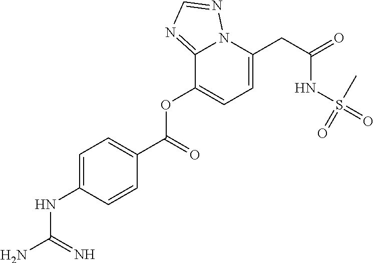 [SMILES: CS(=O)(=O)NC(=O)Cc1ccc(OC(=O)c2ccc(NC(=N)N)cc2)c2ncnn12]

### Step 1: 2-(8-(benzyloxy)-[1,2,4]triazolo[1,5-a]pyridin-5-yl)-N-(methylsulfonyl)acetamide

To a solution of 2-(8-(benzyloxy)-[1,2,4]triazolo[1,5-a]pyridin-5-yl)acetic acid (200 mg, 0.706 mmol, 1.00 equiv) in DCM (10 ml) were added EDCI (200 mg, 1.047 mmol, 1.50 equiv), DMAP (130 mg, 1.057 mmol, 1.50 equiv) and methanesulfonamide (80 mg, 0.841 mmol, 1.20 equiv). The resulting mixture was stirred for 2 h at 25° C. The reaction was monitored by TLC (MeOH/DCM=1/2). The resulting solution was concentrated under vacuum. The residue was purified by silica gel chromatography (0-50% EtOAc/petroleum ether) to yield 2-(8-(benzyloxy)-[1,2,4]triazolo[1,5-a]pyridin-5-yl)-N-(methylsulfonyl)acetamide as a yellow oil. Mass spectrum (ESI, m/z): Calculated for C₁₆H₁₆N₄O₄S, 361.1 [M+H], Measured: 360.9.

### Step 2: 2-(8-hydroxy-[1,2,4]triazolo[1,5-a]pyridin-5-yl)-N-(methylsulfonyl) acetamide

To a solution of 2-(8-(benzyloxy)-[1,2,4]triazolo[1,5-a]pyridin-5-yl)-N-(methylsulfonyl)acetamide (150 mg, 0.416 mmol, 1.00 equiv) in EA (5 mL) was added Pd/C (150 mg). The resulting solution was hydrogenated under 1 atm of H2 at 25° C. for 3 hours. The reaction was monitored by TLC (MeOH/DCM=1/2). The resulting mixture was filtered. The filtrate was concentrated under vacuum. The residue was purified by silica gel chromatography (0-10% EtOAc/petroleum ether) to yield 2-(8-hydroxy-[1,2,4]triazolo[1,5-a]pyridin-5-yl)-N-(methylsulfonyl)acetamide as a yellow oil. Mass spectrum (ESI, m/z): Calculated for C₉H₁₀N₄O₄S, 271.2 [M+H], Measured: 271.2.

### Step 3: 5-[(methanesulfonylcarbamoyl)methyl]-[1,2,4]triazolo[1,5-a]pyridin-8-yl 4-{[(1Z)-{[(tert-butoxy)carbonyl]amino}({[(tert-butoxy) carbonyl]imino})methyl]amino}benzoate

To a solution of 2-(8-(benzyloxy)-[1,2,4]triazolo[1,5-a]pyridin-5-yl)-N-(methylsulfonyl)acetamide (90 mg, 0.333 mmol, 1.00 equiv) in DCM (5 ml) were added EDCI (95 mg, 0.497 mmol, 1.50 equiv), DMAP (60 mg, 0.492 mmol, 1.50 equiv) and (Z)-4-(2,3-bis(tert-butoxycarbonyl)guanidino)benzoic acid (125 mg, 0.833 mmol, 1.20 equiv). The resulting mixture was stirred for 2 h at 25° C. The reaction was monitored by LCMS. The resulting solution was concentrated under vacuum. The residue was purified by silica gel chromatography (0-50% EtOAc/petroleum ether) to yield 5-[(methanesulfonylcarbamoyl)methyl]-[1,2,4] triazolo[1,5-a]pyridin-8-yl 4-{[(1Z)-{[(tert-butoxy)carbonyl]amino}({[(tert-butoxy)carbonyl]imino})methyl]amino}benzoate as a yellow oil. Mass spectrum (ESI, m/z): Calculated for C₂₇H₃₃N₇O₉S, 631.6 [M+H], Measured: 632.2.

### Step 4: 5-(2-(methylsulfonamido)-2-oxoethyl)-[1,2,4]triazolo[1,5-a]pyridin-8-yl 4-guanidinobenzoate

Into a 50 ml round bottom-flask, 5-[(methanesulfonylcarbamoyl)methyl]-[1,2,4]triazolo[1,5-a]pyridin-8-yl 4-{[(1Z)-{[(tert-butoxy)carbonyl]amino}({[(tert-butoxy)carbonyl]imino})methyl]amino}benzoate (100 mg, 0.158 mmol, 1.00 equiv) was dissolved in DCM (6 ml). Then TFA (2 ml) was added to the solution. The resulting solution was stirred 3.0 h at room temperature. The reaction was monitored by LCMS. The organic layers were concentrated under vacuum. The residue was purified by Prep-HPLC with the following conditions: (1#waters2767-5): Column, SunFire Prep C18, 19*150 mm 5 umH PrepC-001(T)18600256819513816414 04; mobile phase, Phase A: water with 0.05% TFA; Phase B: CH₃CN (5% CH₃CN up to 14% in 10 min, up to 100% CH₃CN in 1 min, hold 100% in 1 min, down to 14% CH₃CN in 0.1 min, hold 5% in 0.9 min); Detector, UV220 & 254 nm to yield 5-(2-(methylsulfonamido)-2-oxoethyl)-[1,2,4]triazolo[1,5-a]pyridin-8-yl 4-guanidinobenzoate as a white solid.

¹H NMR (400 MHz, DMSO-d⁶) δ: 12.30 (s, 1H), 10.19 (s, 1H), 8.53 (s, 1H), 8.24 (d, J=3.3 Hz, 2H), 7.81 (s, 4H), 7.74 (d, J=2.8 Hz, 1H), 7.48 (d, J=3.3 Hz, 2H), 7.31 (d, J=3.0 Hz, 1H), 4.33 (s, 2H), 3.26 (s, 3H). Mass spectrum (ESI, m/z): Calculated for C₁₇H₁₇N₇O₅S, 432.4 [M+H], Measured: 432.2.

### Example 9: Compound #9

### 2-(8-(4-guanidinobenzoyloxy)imidazo[1,2-a]pyridin-5-yl)acetic acid

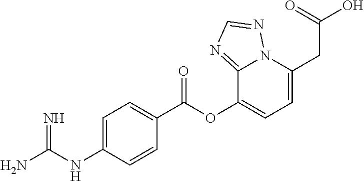 [SMILES: N=C(N)Nc1ccc(C(=O)Oc2ccc(CC(=O)O)n3ncnc23)cc1]

### Step 1: tert-butyl 2-(8-(benzyloxy)imidazo[1,2-a]pyridin-5-yl)acetate

Into a microwave tube were added 8-(benzyloxy)-5-bromoimidazo[1,2-a]pyridine (200 mg, 0.660 mmol 1.0 equiv) Pd₂(dba)₃ (136 mg, 0.131 mmol, 0.20 equiv), XPhos (125 mg, 0.263 mmol, 0.40 equiv) in THE (5 ml). (2-tert-butoxy-2-oxoethyl)zinc(II) bromide (1.00 g, 3.839 mmol, 6.00 equiv) was then added. The resulting mixture was stirred for 1 h at 110° C. The reaction was monitored by LCMS. The reaction was added H₂O (10 ml). The resulting solution was extracted with EtOAc (20 ml×3) and the organic layers combined and concentrated under vacuum. The residue was purified by silica gel chromatography (0-50% EtOAc/petroleum ether) to yield tert-butyl 2-(8-(benzyloxy)imidazo[1,2-a]pyridin-5-yl)acetate as a yellow oil. Mass spectrum (ESI, m/z): Calculated for C₂₀H₂₂N₂O₃, 339.4[M+H], Measured: 339.3.

### Step 2: tert-butyl-2-(8-hydroxyimidazo[1,2-a]pyridin-5-yl)acetate

To a solution of tert-butyl 2-(8-(benzyloxy)imidazo[1,2-a]pyridin-5-yl)acetate (150 mg, 0.443 mmol 1.0 equiv) in EA (5 mL) was added Pd/C (150 mg). The resulting solution was hydrogenated under 1 atm of H2 at 25° C. for 3 hours. The reaction was monitored by LCMS. The resulting mixture was filtered. The filtrate was concentrated under vacuum. The residue was purified by silica gel chromatography (0-10% EtOAc/petroleum ether) to yield tert-butyl 2-(8-hydroxyimidazo[1,2-a]pyridin-5-yl)acetate as a yellow oil. Mass spectrum (ESI, m/z): Calculated for C₁₃H₁N₂O₃, 249.2 [M+H], Measured: 249.2.

### Step 3: 5-[2-(tert-butoxy)-2-oxoethyl]imidazo[1,2-a]pyridin-8-yl 4-{[(1E)-{[(tert-butoxy)carbonyl]amino}({[(tert-butoxy)carbonyl]imino})methyl] amino}benzoate

To a solution of tert-butyl 2-(8-hydroxyimidazo[1,2-a]pyridin-5-yl)acetate (70 mg, 0.282 mmol, 1.0 equiv) in DCM (10 ml) were added EDCI (114 mg, 0.597 mmol, 2.00 equiv), DMAP (70 mg, 0.574 mmol, 2.00 equiv) and (Z)-4-(2,3-bis(tert-butoxycarbonyl)guanidino)benzoic acid (130 mg, 0.343 mmol, 1.00 equiv). The resulting mixture was stirred for 4 h at 25° C. The resulting solution was concentrated under vacuum. The reaction was monitored by LCMS. The residue was purified by silica gel chromatography (0-50% EtOAc/petroleum ether) to yield 5-[2-(tert-butoxy)-2-oxoethyl]imidazo[1,2-a]pyridin-8-yl 4-{[(1E)-{[(tert-butoxy)carbonyl]amino}({[(tert-butoxy)carbonyl]imino})methyl]amino}benzoate as a yellow oil. Mass spectrum (ESI, m/z): Calculated for C₃₁H₃₉N₅O, 610.3[M+H], Measured: 610.3.

### Step 4: 2-(8-(4-guanidinobenzoyloxy)imidazo[1,2-a]pyridin-5-yl)acetic acid

Into a 50 ml round bottom-flask, 5-[2-(tert-butoxy)-2-oxoethyl]imidazo[1,2-a]pyridin-8-yl 4-{[(1E)-{[(tert-butoxy)carbonyl]amino}({[(tert-butoxy)carbonyl] imino})methyl]amino}benzoate (100 mg, 0.164 mmol, 1.00 equiv) was dissolved in DCM (6 ml). Then TFA (2 ml) was added to the solution. The resulting solution was stirred 2.0 h at room temperature. The reaction was monitored by LCMS. The organic layers were concentrated under vacuum. The residue was purified by Prep-HPLC with the following conditions: (1#waters27675: Column, SunFire Prep C18, 19*150 mm 5 umH PrepC-001(T) 18600256819513816414 04; mobile phase, Phase A: water with 0.05% TFA; Phase B: CH₃CN (5% CH₃CN up to 14% in 10 min, up to 100% CH₃CN in 1 min, hold 100% in 1 min, down to 14% CH₃CN in 0.1 min, hold 5% in 0.9 min); Detector, UV220 & 254 nm to yield 2-(8-(4-guanidinobenzoyloxy)imidazo[1,2-a]pyridin-5-yl)acetic acid as an off-white solid.

¹H NMR (300 MHz, DMSO-d⁶) δ: 12.89 (brs, 1H), 10.41 (s, 1H), 8.11-8.32 (m, 3H), 7.78-8.04 (m, 5H), 7.42-7.68 (m, 3H), 7.01-7.39 (m, 1H), 4.25 (s, 2H). Mass spectrum (ESI, m/z): Calculated for C₁₇H₁₅N₅O₄, 354.3[M+H], Measured: 354.0.

### Example 10: Compound #10

### 2-(8-(5-guanidinothiophene-2-carbonyloxy)-[1,2,4]triazolo[1,5-a]pyridin-5-yl)acetic acid

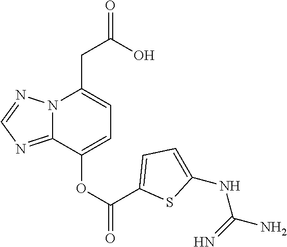 [SMILES: N=C(N)Nc1ccc(C(=O)Oc2ccc(CC(=O)O)n3ncnc23)s1]

### Step 1: Methyl 5-aminothiophene-2-carboxylate

Into a 30-mL round-bottom flask purged and maintained with atmosphere of hydric, was placed methyl 5-nitrothiophene-2-carboxylate (500 mg, 1.630 mmol), EA (10 ml), Pd/C (500 mg). The resulting solution was stirred 2.0 h at room temperature. The reaction progress was monitored by LCMS. The mixture was filtered through a CELITE® pad. The resulting mixture was concentrated under vacuum to yield methyl 5-aminothiophene-2-carboxylate as a white solid. Mass spectrum (ESI, m/z): Calculated for C₆H₇NO₂S, 158.0[M+H], Measured: 158.3.

### Step 2: methyl 5-{[(1E)-{[(tert-butoxy)carbonyl]amino}({[(tert-butoxy) carbonyl]imino})methyl]amino}thiophene-2-carboxylate

To a solution of methyl 5-aminothiophene-2-carboxylate (420 mg, 2.672 mmol) in DMF (20 mL) was added tert-butyl N-[(1E)-{[(tert-butoxy)carbonyl]amino}(methylsulfanyl)methylidene]carbamate (931.045 mg, 3.206 mmol), Et₃N (539.729 mg, 5.344 mmol), HgCl₂ (726.764 mg, 2.672 mmol). The resulting mixture was stirred at room temperature overnight. The reaction progress was monitored by LCMS. The reaction was quenched with H₂O (20 mL). The resulting mixture was extracted with EtOAc (3×20 mL). The organic layers were combined, dried over Na₂SO₄, filtered and concentrated. The residue was purified by TLC (PE:EA=3:1) to yield methyl 5-{[(1E)-{[(tert-butoxy)carbonyl]amino}({[(tert-butoxy)carbonyl]imino})methyl]amino}thiophene-2-carboxylate as a yellow solid. Mass spectrum (ESI, m/z): Calculated for C₁₇H₂₅N₃O₆S, 400.1 [M+H], Measured: 400.4.

### Step 3: 5-(3-(tert-butoxycarbonyl)guanidino)thiophene-2-carboxylic acid

To a solution of methyl 5-{[(1E)-{[(tert-butoxy)carbonyl]amino}({[(tert-butoxy)carbonyl]imino})methyl]amino}thiophene-2-carboxylate (200 mg, 0.501 mmol) in THE (4.0 ml) with H₂O (2.0 ml), was added LiOH (60.081 mg, 2.503 mmol). The resulting solution was stirred 1.0 h at 50° C. The reaction was monitored by LCMS. The reaction was quenched with H₂O (5 ml) and with 2 N HCl adjusted pH to 6. The mixture was extracted with EA (3×5 ml), the organic layers were combined, dried over Na₂SO₄ and concentrated under vacuum to yield 5-(3-(tert-butoxycarbonyl)guanidino)thiophene-2-carboxylic acid as a yellow oil. Mass spectrum (ESI, m/z): Calculated for C₁₁H₁₅N₃O₄S, 286.1[M+H], Measured: 286.4.

### Step 4: 5-[2-(tert-butoxy)-2-oxoethyl]-[1,2,4]triazolo[1,5-a]pyridin-8-yl 5-[({[(tert-butoxy)carbonyl]amino}methanimidoyl)amino]thiophene-2-carboxylate

To a solution of tert-butyl 2-(8-hydroxy-[1,2,4]triazolo[1,5-a]pyridin-5-yl)acetate (50 mg, 0.201 mmol) in DCM (5.0 ml), was added EDCI (76.625 mg, 0.401 mmol), DMAP (48.944 mg, 0.401 mmol), 5-(3-(tert-butoxycarbonyl) guanidino) thiophene-2-carboxylic acid (57.232 mg, 0.201 mmol). The resulting solution was stirred overnight at room temperature. The reaction was monitored by LCMS. The resulting mixture was concentrated under vacuum. The residue was purified by TLC (PE:EA=2:1) to yield 5-[2-(tert-butoxy)-2-oxoethyl]-[1,2,4]triazolo[1,5-a]pyridin-8-yl 5-[({[(tert-butoxy)carbonyl]amino}methanimidoyl)amino]thiophene-2-carboxylate as a yellow oil. Mass spectrum (ESI, m/z): Calculated for C₂₃H₂₈N₆O₆S, 517.2[M+H], Measured: 517.5.

### Step 5: 2-(8-(5-guanidinothiophene-2-carbonyloxy)-[1,2,4]triazolo[1,5-a]pyridin-5-yl)acetic acid

To a solution of 5-[2-(tert-butoxy)-2-oxoethyl]-[1,2,4]triazolo[1,5-a]pyridin-8-yl 5-[({[(tert-butoxy)carbonyl]amino}methanimidoyl)amino]thiophene-2-carboxylate (60 mg, 0.116 mmol) in DCM (4.0 ml), was added TFA (1.0 ml). The resulting solution was stirred 2.0 h at room temperature. The reaction was monitored by LCMS. The resulting mixture was concentrated under vacuum. The residue was purified by Prep-HPLC with the following conditions (1#-Waters 2767-5): Column, SunFire Prep C18, 5 um, 19*100 mm; mobile phase, Water with 0.05% TFA and CH₃CN (5% CH₃CN up to 30% in 10 min, up to 100% in 0.1 min, hold 100% in 0.9 min, down to 5% in 0.1 min, hold 5% in 1.4 min); Detector, UV 220&254 nm to yield 2-(8-(5-guanidinothiophene-2-carbonyloxy)-[1,2,4]triazolo[1,5-a]pyridin-5-yl)acetic acid as a white solid.

¹H NMR (400 MHz, DMSO-d⁶) δ: 12.81 (s, 1H), 10.52-10.62 (m, 1H), 8.53 (s, 1H), 8.03-8.04 (m, 1H), 7.81-7.89 (m, 4H), 7.71-7.79 (m, 1H), 7.22-7.29 (m, 1H), 7.15-7.16 (m, 1H), 4.23 (s, 2H). Mass spectrum (ESI, m/z): Calculated for C₁₄H₁₂N₆O₄S, 361.1 [M+H], Measured: 361.1.

### Example 11: Compound #11

### 5-((2H-tetrazol-5-yl)methyl)-[1,2,4]triazolo[1,5-a]pyridin-8-yl 4-guanidinobenzoate

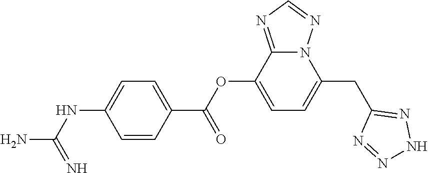 [SMILES: N=C(N)Nc1ccc(C(=O)Oc2ccc(Cc3nn[nH]n3)n3ncnc23)cc1]

### Step 1: (8-(benzyloxy)-[1,2,4]triazolo[1,5-a]pyridin-5-yl)methanol

Into a 100-mL round-bottom flask, was placed methyl 8-(benzyloxy)-[1,2,4]triazolo[1,5-a]pyridine-5-carboxylate (500 mg, 1.765 mmol, 1.00 equiv), THE (10 ml), lithiumaluminiumtetrahydride (134 mg, 3.530 mmol, 2.00 equiv). The resulting solution was stirred for 2 h at 0° C. The reaction progress was monitored by TLC (PE:EA=1:1). The reaction was then quenched by the addition Na₂SO₄.10H₂O. The resulting mixture was concentrated under vacuum. The residue was applied onto a silica gel column with ethyl acetate/petroleum ether (1:1) to yield (8-(benzyloxy)-[1,2,4]triazolo[1,5-a]pyridin-5-yl)methanol as light yellow oil. Mass spectrum (ESI, m/z): Calculated for C₁₄H₁₃N₃O₂, 256.0 [M+H], Measured: 256.0.

### Step 2: 8-(benzyloxy)-5-(chloromethyl)-[1,2,4]triazolo[1,5-a]pyridine

Into a 100-mL round-bottom flask, to a solution of (8-(benzyloxy)-[1,2,4]triazolo[1,5-a]pyridin-5-yl)methanol (100 mg, 0.392 mmol, 1.00 equiv) in DCM (5 ml) was added SOCl₂ (233, 1.595 ml, 5.00 equiv). The mixture was stirred at 25° C. 2 h. The reaction progress was monitored by TLC (PE:EA=3:1). The resulting solution was concentrated to yield 8-(benzyloxy)-5-(chloromethyl)-[1,2,4]triazolo[1,5-a]pyridine as a light yellow solid, Mass spectrum (ESI, m/z): Calculated for C₁₄H₁₂CIN₃O, 274.1 [M+H], Measured: 274.3.

### Step 3: 2-(8-(benzyloxy)-[1,2,4]triazolo[1,5-a]pyridin-5-yl)-2-hydroxyacetonitrile

Into a 100-mL round-bottom flask, to a solution of 8-(benzyloxy)-5-(chloromethyl)-[1,2,4]triazolo[1,5-a]pyridine (100 mg, 0.365 mmol, 1.00 equiv) in DMF (8 ml) was added KCN (90 mg, 1.827 mmol, 5.00 equiv). The resulting mixture was stirred overnight at 25° C. The reaction progress was monitored by TLC (PE:EA=3:1). The reaction was quenched with H₂O (10 mL). The resulting mixture was extracted with DCM (3×10 mL). The organic layers were combined, dried over Na₂SO₄, filtered and concentrated. The residue was purified by silica gel chromatography (0-50% EtOAc/petroleum ether) to yield 2-(8-(benzyloxy)-[1,2,4]triazolo[1,5-a]pyridin-5-yl)-2-hydroxy acetonitrile as a light yellow oil (80 mg, 95% yield). Mass spectrum (ESI, m/z): Calculated for C₁₅H₁₂N₄O, 265.1 [M+H], Measured: 265.0.

### Step 4: 5-((2H-tetrazol-5-yl)methyl)-8-(benzyloxy)-[1,2,4]triazolo[1,5-a]pyridine

Into a 100-mL round-bottom flask, to a solution of 2-(8-(benzyloxy)-[1,2,4]triazolo[1,5-a]pyridin-5-yl)acetonitrile (80 mg, 0.303 mmol, 1.00 equiv) in toluene (8 ml) was added TMSN₃ (105 mg, 0.908 mmol, 3.00 equiv), di(n-butyl)tin oxide ((75 mg, 0.303 mmol, 1.00 equiv). The resulting mixture was stirred overnight at 120° C. The reaction progress was monitored by LCMS. The residue was purified by silica gel chromatography (0-50% EtOAc/petroleum ether) to yield 5-((2H-tetrazol-5-yl)methyl)-8-(benzyloxy)-[1,2,4]triazolo[1,5-a]pyridine as a light yellow solid (80 mg, 82% yield). Calculated for C₁₅H₁₃N₇O, 308.1 [M+H], Measured: 308.2.

### Step 5. 5-((2H-tetrazol-5-yl)methyl)-[1,2,4]triazolo[1,5-a]pyridin-8-ol

Into a 50-mL round-bottom flask, was placed 5-((2H-tetrazol-5-yl)methyl)-8-(benzyloxy)-[1,2,4]triazolo[1,5-a]pyridine (80 mg, 0.260 mmol, 1.00 equiv), Pd/C 10% (80 mg), EA (5 ml). The reaction was purged with an inert atmosphere of hydrogen and was stirred 2 h at 25° C. The reaction progress was monitored by LCMS. A filtration was performed. The filtrate was concentrated under vacuum to yield 5-((2H-tetrazol-5-yl)methyl)-[1,2,4]triazolo[1,5-a]pyridin-8-ol as a light yellow solid. Mass spectrum (ESI, m/z): Calculated for C₈H₇N₇O, 218.1 [M+H], Measured: 218.2.

### Step 6: 5-(2H-1,2,3,4-tetrazol-5-ylmethyl)-[1,2,4]triazolo[1,5-a]pyridin-8-yl 4-[({[(tert-butoxy)carbonyl]amino}({[(tert-butoxy)carbonyl]imino}) methyl)amino]benzoate

Into a 100-mL round-bottom flask, was placed 5-((2H-tetrazol-5-yl)methyl)-[1,2,4]triazolo[1,5-a]pyridin-8-ol (40 mg, 0.184 mmol, 1.00 equiv), 4-{[(1E)-{[(tert-butoxy)carbonyl]amino}({[(tert-butoxy)carbonyl]imino})methyl]amino} benzoic acid (77 mg, 0.203 mmol, 1.00 equiv), EDCI (43 mg, 0.276 mmol, 1.50 equiv), DMAP (34 mg, 0.276 mmol, 1.50 equiv), DCM (5 ml). The reaction was stirred 2 h at 25° C. The reaction progress was monitored by LCMS. The resulting solution concentrated. The residue was purified by silica gel chromatography (0-50% PE/EA) to yield 5-(2H-1,2,3,4-tetrazol-5-ylmethyl)-[1,2,4]triazolo[1,5-a]pyridin-8-yl 4-[({[(tert-butoxy) carbonyl] amino}({[(tert-butoxy)carbonyl]imino})methyl)amino]benzoate as a light yellow oil. Mass spectrum (ESI, m/z): Calculated for C₂₆H₃₀N₁₀O₆, 579.2 [M+H], Measured: 579.3.

### Step 7: 5-((2H-tetrazol-5-yl)methyl)-[1,2,4]triazolo[1,5-a]pyridin-8-yl 4-guanidinobenzoate

Into a 50-mL round-bottom flask was placed 5-(2H-1,2,3,4-tetrazol-5-ylmethyl)-[1,2,4]triazolo[1,5-a]pyridin-8-yl 4-{[(1Z)-{[(tert-butoxy)carbonyl]amino}({[(tert-butoxy)carbonyl]imino})methyl]amino}benzoate (30 mg, 0.052 mmol, 1.00 equiv), DCM (5 ml), TFA (2 ml). The reaction was stirred 2 h at 25° C. The reaction progress was monitored by LCMS. The resulting mixture was concentrated under vacuum. The residue was purified by Prep-HPLC with the following conditions (1#-Waters 2767-5): Column, SunFire Prep C18, 5 um, 19*100 mm; mobile phase, Water of 0.05% TFA and CH₃CN (5% CH₃CN up to 20% in 10 min, up to 100% in 2 min, down to 20% in 2 min; Detector, 254 nm to yield 5-((2H-tetrazol-5-yl)methyl)-[1,2,4] triazolo[1,5-a]pyridin-8-yl 4-guanidinobenzoate as off-white solid.

¹HNMR (300 MHz, DMSO-d⁶) δ: 10.14 (s, 1H), 8.49 (s, 1H), 8.23 (d, J=8.7 Hz, 2H), 7.77-7.79 (m, 5H), 7.48 (d, J=8.7 Hz, 2H), 7.36 (d, J=7.8 Hz, 1H), 4.91 (s, 2H). Mass spectrum (ESI, m/z): Calculated for C₁₆H₁₄N₁₀O₂, 379.1 [M+H], Measured: 379.2.

### Example 12: Compound #12

### 2-(8-(4-guanidino-3-methoxybenzoyloxy)-[1,2,4]triazolo[1,5-a]pyridin-5-yl)acetic acid

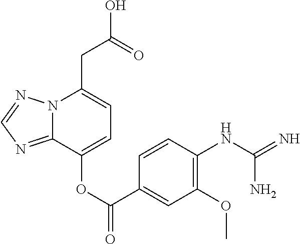 [SMILES: COc1cc(C(=O)Oc2ccc(CC(=O)O)n3ncnc23)ccc1NC(=N)N]

### Step 1: methyl 4-{[(1Z)-{[(tert-butoxy)carbonyl]amino}({[(tert-butoxy) carbonyl]imino})methyl]amino}-3-methoxybenzoate

To a solution of methyl 4-amino-3-methoxybenzoate (300 mg, 1.656 mmol) in DMF (20 mL) was added tert-butyl N-[(1E)-{[(tert-butoxy)carbonyl]amino}(methylsulfanyl)methylidene]carbamate (576.947 mg, 1.987 mmol), Et₃N (334.457 mg, 3.311 mmol), HgCl₂ (450.358 mg, 1.656 mmol). The resulting mixture was stirred at room temperature overnight. The reaction progress was monitored by LCMS. The reaction was quenched with H₂O (20 mL). The resulting mixture was extracted with EtOAc (3×20 mL). The organic layers were combined, dried over Na₂SO₄, filtered and concentrated. The residue was purified by TLC (PE:EA=3:1) to yield methyl 4-{[(1Z)-{[(tert-butoxy)carbonyl]amino}({[(tert-butoxy)carbonyl]imino})methyl]amino}-3-methoxybenzoate as a yellow solid. Mass spectrum (ESI, m/z): Calculated for C₂₀H₂₉N₃O₇, 424.2[M+H], Measured: 424.5.

### Step 2. (Z)-4-(2,3-bis(tert-butoxycarbonyl)guanidino)-3-methoxybenzoic acid

To a solution of methyl 4-{[(1Z)-{[(tert-butoxy)carbonyl]amino}({[(tert-butoxy)carbonyl]imino})methyl]amino}-3-methoxybenzoate (200 mg, 0.472 mmol) in THE (4.0 ml) with H₂O (2.0 ml), was added NaOH (94.460 mg, 2.361 mmol). The resulting solution was stirred 1.0 h at 50° C. The reaction was monitored by LCMS. The reaction was quenched by H₂O (5 ml) and with 2 N HCl adjusted pH to 6. The mixture was extracted with EA (3×5 ml), the organic layers were combined, dried over Na₂SO₄ and concentrated under vacuum to yield (Z)-4-(2,3-bis(tert-butoxycarbonyl)guanidino)-3-methoxybenzoic acid as a yellow oil. Mass spectrum (ESI, m/z): Calculated for C₁₉H₂₇N₃O₇, 410.2[M+H], Measured: 410.4.

### Step 3: 5-[2-(tert-butoxy)-2-oxoethyl]-[1,2,4]triazolo[1,5-a]pyridin-8-yl 4-{[(1Z)-{[(tert-butoxy)carbonyl]amino}({[(tert-butoxy)carbonyl]imino}) methyl]amino}-3-methoxybenzoate

To a solution of tert-butyl 2-(8-hydroxy-[1,2,4]triazolo[1,5-a]pyridin-5-yl)acetate (60 mg, 0.241 mmol) in DCM (5.0 ml), was added EDCI (91.950 mg, 0.481 mmol), DMAP (58.732 mg, 0.481 mmol), (Z)-4-(2,3-bis(tert-butoxy carbonyl)guanidino)-3-methoxybenzoic acid (74.455 mg, 0.241 mmol). The resulting solution was stirred overnight at room temperature. The reaction was monitored by LCMS. The resulting mixture was concentrated under vacuum. The residue was purified by TLC (PE:EA=2:1) to yield 5-[2-(tert-butoxy)-2-oxoethyl]-[1,2,4]triazolo[1,5-a]pyridin-8-yl 4-{[(1Z)-{[(tert-butoxy)carbonyl]amino}({[(tert-butoxy)carbonyl]imino})methyl]amino}-3-methoxybenzoate as a yellow oil. Mass spectrum (ESI, m/z): Calculated for C₃₁H₄₀N₆O₉, 641.3[M+H], Measured: 641.6.

### Step 4: 2-(8-(4-guanidino-3-methoxybenzoyloxy)-[1,2,4]triazolo[1,5-a]pyridin-5-yl)acetic acid

To a solution of 5-[2-(tert-butoxy)-2-oxoethyl]-[1,2,4]triazolo[1,5-a]pyridin-8-yl 4-{[(1Z)-{[(tert-butoxy)carbonyl]amino}({[(tert-butoxy) carbonyl]imino})methyl]amino}-3-methoxybenzoate (55 mg, 0.076 mmol) in DCM (4.0 ml), was added TFA (1.0 ml). The resulting solution was stirred 2.0 h at room temperature. The reaction was monitored by LCMS. The resulting mixture was concentrated under vacuum. The residue was purified by Prep-HPLC with the following conditions (1#-Waters 2767-5): Column, SunFire Prep C18, 5 um, 19*100 mm; mobile phase, Water with 0.05% TFA and CH₃CN (5% CH₃CN up to 30% in 10 min, up to 100% in 0.1 min, hold 100% in 0.9 min, down to 5% in 0.1 min, hold 5% in 1.4 min); Detector, UV 220&254 nm to yield 2-(8-(4-guanidino-3-methoxybenzoyloxy)-[1,2,4]triazolo[1,5-a]pyridin-5-yl)acetic acid as a white solid.

¹H NMR (300 MHz, DMSO-d⁶) δ: 12.78-12.92 (m, 1H), 9.55 (s, 1H), 8.53 (s, 1H), 7.73-7.91 (m, 3H), 7.51-7.61 (m, 5H), 7.26-7.31 (m, 1H), 4.25 (s, 2H), 3.98 (s, 3H). Mass spectrum (ESI, m/z): Calculated for C₁₇H₆N₆O₅, 385.1[M+H], Measured: 385.2.

### Example 13: Compound #13

### 2-(8-(4-guanidino-3-iodobenzoyloxy)-[1,2,4]triazolo[1,5-a]pyridin-5-yl)acetic acid

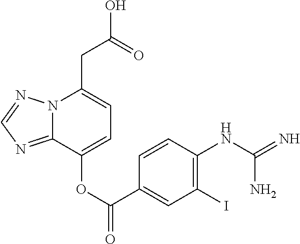 [SMILES: N=C(N)Nc1ccc(C(=O)Oc2ccc(CC(=O)O)n3ncnc23)cc1I]

### Step 1: methyl 4-{[(1Z)-{[(tert-butoxy)carbonyl]amino}({[(tert-butoxy) carbonyl]imino})methyl]amino}-3-iodobenzoate

To a solution of methyl 4-amino-3-iodobenzoate (300 mg, 1.083 mmol) in DMF (20 mL) was added tert-butyl N-[(1E)-{[(tert-butoxy)carbonyl]amino}(methylsulfanyl)methylidene]carbamate (377.308 mg, 1.299 mmol), Et₃N (218.726 mg, 2.166 mmol), HgCl₂ (294.522 mg, 1.083 mmol). The resulting mixture was stirred at room temperature overnight. The reaction progress was monitored by LCMS. The reaction was quenched with H₂O (20 mL). The resulting mixture was extracted with EtOAc (3×20 mL). The organic layers were combined, dried over Na₂SO₄, filtered and concentrated. The residue was purified by TLC (PE:EA=3:1) to yield methyl 4-{[(1Z)-{[(tert-butoxy)carbonyl]amino}({[(tert-butoxy)carbonyl]imino})methyl]amino}-3-iodobenzoate as a yellow solid. Mass spectrum (ESI, m/z): Calculated for C₁₉H₂₆IN₃O₆, 520.1 [M+H], Measured: 520.1.

### Step 2: (E)-4-(2-(tert-butoxycarbonyl)guanidino)-3-fluorobenzoic acid

To a solution of methyl 4-{[(1Z)-{[(tert-butoxy)carbonyl]amino}({[(tert-butoxy)carbonyl]imino})methyl]amino}-3-iodobenzoate (150 mg, 0.289 mmol) in THE (4.0 ml) with H₂O (2.0 ml), was added NaOH (57.767 mg, 1.444 mmol). The resulting solution was stirred 1.0 h at 50° C. The reaction was monitored by LCMS. The reaction was quenched by H₂O (5 ml) and with 2 N HCl adjusted pH to 6. The mixture was extracted with EA (3*5 ml), the organic layers were combined, dried over Na₂SO₄ and concentrated under vacuum to yield (E)-4-(2-(tert-butoxycarbonyl)guanidino)-3-fluorobenzoic acid as a yellow oil. Mass spectrum (ESI, m/z): Calculated for C₁₃H₁₆IN₃O₄, 406.0[M+H], Measured: 406.2.

### Step 3: (E)-5-(2-tert-butoxy-2-oxoethyl)-[1,2,4]triazolo[1,5-a]pyridin-8-yl 4-(2-(tert-butoxycarbonyl)guanidino)-3-iodobenzoate

To a solution of tert-butyl 2-(8-hydroxy-[1,2,4]triazolo[1,5-a]pyridin-5-yl)acetate (60 mg, 0.241 mmol) in DCM (5.0 ml), was added EDCI (91.950 mg, 0.481 mmol), DMAP (58.732 mg, 0.481 mmol), (E)-4-(2-(tert-butoxycarbonyl) guanidino)-3-fluorobenzoic acid (97.531 mg, 0.241 mmol). The resulting solution was stirred overnight at room temperature. The reaction was monitored by LCMS. The resulting mixture was concentrated under vacuum. The residue was purified by TLC (PE:EA=2:1) to yield (E)-5-(2-tert-butoxy-2-oxoethyl)-[1,2,4]triazolo[1,5-a]pyridin-8-yl 4-(2-(tert-butoxycarbonyl)guanidino)-3-iodobenzoate as a yellow oil. Mass spectrum (ESI, m/z): Calculated for C₂₅H₂₉IN₆O₆, 637.1[M+H], Measured: 637.5.

### Step 4: 2-(8-(4-guanidino-3-iodobenzoyloxy)-[1,2,4]triazolo[1,5-a]pyridin-5-yl)acetic acid

To a solution of 5-[2-(tert-butoxy)-2-oxoethyl]-[1,2,4]triazolo[1,5-a]pyridin-8-yl 4-{[(1Z)-{[(tert-butoxy)carbonyl]amino}({[(tert-butoxy)carbonyl]imino})methyl]amino}-3-methoxybenzoate (55 mg, 0.078 mmol) in DCM (4.0 ml) was added TFA (1.0 ml). The resulting solution was stirred 2.0 h at room temperature. The reaction was monitored by LCMS. The resulting mixture was concentrated under vacuum. The residue was purified by Prep-HPLC with the following conditions (1#-Waters 2767-5): Column, SunFire Prep C18, 5 um, 19*100 mm; mobile phase, Water with 0.05% TFA and CH₃CN (5% CH₃CN up to 30% in 10 min, up to 100% in 0.1 min, hold 100% in 0.9 min, down to 5% in 0.1 min, hold 5% in 1.4 min); Detector, UV 220&254 nm to yield 2-(8-(4-guanidino-3-iodobenzoyloxy)-[1,2,4]triazolo[1,5-a]pyridin-5-yl)acetic acid as a white solid.

¹H NMR (300 MHz, DMSO-d⁶) δ: 8.65-8.66 (m, 1H), 8.53 (s, 1H), 8.21-8.28 (m, 1H), 7.72-7.76 (m, 1H), 7.41-7.67 (m, 5H), 7.31-7.35 (m, 1H), 4.25 (s, 2H). Mass spectrum (ESI, m/z): Calculated for C₁₆H₁₃IN₆O₄, 481.0[M+H], Measured: 481.1.

### Example 14: Compound #14

### 2-(8-(3-fluoro-4-guanidinobenzoyloxy)-[1,2,4]triazolo[1,5-a]pyridin-5-yl)acetic acid

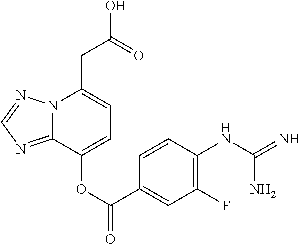 [SMILES: N=C(N)Nc1ccc(C(=O)Oc2ccc(CC(=O)O)n3ncnc23)cc1F]

### Step 1: Methyl 4-{[(1Z)-{[(tert-butoxy)carbonyl]amino}({[(tert-butoxy) carbonyl]imino})methyl]amino}-3-fluorobenzoate

To a solution of methyl 4-amino-3-fluorobenzoate (300 mg, 1.774 mmol) in DMF (20 mL) was added tert-butyl N-[(1E)-{[(tert-butoxy)carbonyl]amino}(methylsulfanyl)methylidene]carbamate (617.999 mg, 2.128 mmol), Et₃N (358.256 mg, 3.547 mmol), HgCl₂ (482.404 mg, 1.774 mmol). The resulting mixture was stirred at room temperature overnight. The reaction progress was monitored by LCMS. The reaction was quenched with H₂O (20 mL). The resulting mixture was extracted with EtOAc (3×20 mL). The organic layers were combined, dried over Na₂SO₄, filtered and concentrated. The residue was purified by TLC (PE:EA=3:1) to yield methyl 4-{[(1Z)-{[(tert-butoxy)carbonyl]amino}({[(tert-butoxy)carbonyl]imino})methyl]amino}-3-fluorobenzoate as a yellow solid. Mass spectrum (ESI, m/z): Calculated for C₁₉H₂₆FN₃O₆, 412.2[M+H], Measured: 412.2.

### Step 2: 4-(3-(tert-butoxycarbonyl)guanidino)-3-fluorobenzoic acid

To a solution of methyl 4-{[(1Z)-{[(tert-butoxy)carbonyl]amino}({[(tert-butoxy)carbonyl]imino})methyl]amino}-3-fluorobenzoate (200 mg, 0.486 mmol) in THE (4.0 ml) with H₂O (2.0 ml), was added NaOH (97.223 mg, 2.431 mmol). The resulting solution was stirred 1.0 h at 50° C. The reaction was monitored by LCMS. The reaction was quenched by H₂O (5 ml) and with 2 N HCl adjusted pH to 6. The mixture was extracted with EA (3×5 ml), the organic layers were combined, dried over Na₂SO₄ and concentrated under vacuum to yield 4-(3-(tert-butoxycarbonyl)guanidino)-3-fluorobenzoic acid as a yellow oil. Mass spectrum (ESI, m/z): Calculated for C₁₃H₁₆FN₃O₄, 298.1 [M+H], Measured: 298.1.

### Step 3: 5-[2-(tert-butoxy)-2-oxoethyl]-[1,2,4]triazolo[1,5-a]pyridin-8-yl 4-[({[(tert-butoxy)carbonyl]amino}methanimidoyl)amino]-3-fluorobenzoate

To a solution of tert-butyl 2-(8-hydroxy-[1,2,4]triazolo[1,5-a]pyridin-5-yl)acetate (60 mg, 0.241 mmol) in DCM (5.0 ml), was added EDCI (91.950 mg, 0.481 mmol), DMAP (58.732 mg, 0.481 mmol), 4-(3-(tert-butoxycarbonyl) guanidino)-3-fluorobenzoic acid (71.558 mg, 0.241 mmol). The resulting solution was stirred 3.0 h at room temperature. The reaction was monitored by LCMS. The resulting mixture was concentrated under vacuum. The residue was purified by TLC (PE:EA=2:1) to yield 5-[2-(tert-butoxy)-2-oxoethyl]-[1,2,4]triazolo[1,5-a]pyridin-8-yl 4-[({[(tert-butoxy)carbonyl] amino}methanimidoyl)amino]-3-fluorobenzoate as a yellow oil. Mass spectrum (ESI, m/z): Calculated for C₂₅H₂₉FN₆O₆, 529.2[M+H], Measured: 529.5.

### Step 4: 2-(8-(3-fluoro-4-guanidinobenzoyloxy)-[1,2,4]triazolo[1,5-a]pyridin-5-yl)acetic acid

To a solution of 5-[2-(tert-butoxy)-2-oxoethyl]-[1,2,4]triazolo[1,5-a]pyridin-8-yl 4-[({[(tert-butoxy)carbonyl]amino}methanimidoyl)amino]-3-fluorobenzoate (80 mg, 0.107 mmol) in DCM (4.0 ml), was added TFA (1.0 ml). The resulting solution was stirred 2.0 h at room temperature. The reaction was monitored by LCMS. The resulting mixture was concentrated under vacuum. The residue was purified by Prep-HPLC with the following conditions (1#-Waters 2767-5): Column, SunFire Prep C18, 5 um, 19*100 mm; mobile phase, Water with 0.05% TFA and CH₃CN (5% CH₃CN up to 30% in 10 min, up to 100% in 0.1 min, hold 100% in 0.9 min, down to 5% in 0.1 min, hold 5% in 1.4 min); Detector, UV 220&254 nm to yield 2-(8-(3-fluoro-4-guanidinobenzoyloxy)-[1,2,4]triazolo[1,5-a]pyridin-5-yl)acetic acid as a white solid.

¹H NMR (300 MHz, DMSO-d⁶) δ: 8.53 (s, 1H), 8.08-8.11 (m, 2H), 7.60-7.76 (m, 6H), 7.29-7.32 (m, 1H), 4.24 (s, 2H). Mass spectrum (ESI, m/z): Calculated for C₁₆H₁₃FN₆O₄, 373.1 [M+H], Measured: 373.2.

### Example 15: Compound #15

### 2-(8-(4-guanidino-3-methylbenzoyloxy)-[1,2,4]triazolo[1,5-a]pyridin-5-yl)acetic acid

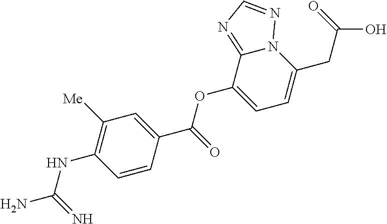 [SMILES: Cc1cc(C(=O)Oc2ccc(CC(=O)O)n3ncnc23)ccc1NC(=N)N]

### Step 1: Methyl 4-{[(1Z)-{[(tert-butoxy)carbonyl]amino}({[(tert-butoxy)carbonyl]imino})methyl]amino}benzoate

Into a 500-mL round bottle was placed methyl 4-amino-3-methyl benzoate (500 mg, 3.027 mmol, 1.00 equiv) DMF (20 mL). Then tert-butyl N-[(1E)-{[(tert-butoxy) carbonyl]imino}(methylsulfanyl)methyl]carbamate (880 mg, 3.031 mmol, 1.00 equiv), TEA (610 mg, 6.04 mmol, 2.00 equiv) and HgCl₂ (820 mg, 3.026 mmol, 1.00 equiv) was added. The resulting solution was stirred 16 h at 25° C. The reaction was monitored by LCMS. The reaction was quenched by H₂O and extracted with EtOAc (20 ml×3). The organic layers were washed by brine (10 ml×3). The resulting solution was concentrated under vacuum. The residue was purified by silica gel chromatography (0-50% EtOAc/petroleum ether) to yield methyl 4-{[(1Z)-{[(tert-butoxy)carbonyl]amino}({[(tert-butoxy)carbonyl]imino})methyl]amino}benzoate as a yellow oil. Mass spectrum (ESI, m/z): Calculated for C₂₀H₂₉N₃O₆, 408.4 [M+H], Measured: 408.2.

### Step 2: (Z)-4-(2-(tert-butoxycarbonyl)guanidino)-3-methylbenzoic acid

To a solution of methyl 4-{[(1Z)-{[(tert-butoxy)carbonyl]amino}({[(tert-butoxy)carbonyl]imino})methyl]amino}benzoate (1700 mg, 0.417 mmol, 1.00 equiv) in THE (4 ml) and H₂O (4 ml) and MeOH (1 ml). NaOH (100 mg, 2.5 mmol, 6.00 equiv) was added. The resulting mixture was stirred for 3 h at 50° C. The reaction was monitored by LCMS. The reaction was adjusted pH to 6 with 2N HCl. The solution was extracted with EtOAc (20 ml×3) and the organic layers combined and concentrated under vacuum. The residue was washed by brine and dried by Na₂SO₄ to yield (Z)-4-(2-(tert-butoxycarbonyl)guanidino)-3-methylbenzoic acid as a yellow oil. Mass spectrum (ESI, m/z): Calculated for C₁₄H₁₉N₃O₄, 294.4 [M+H], Measured: 294.2.

### Step 3: (Z)-5-(2-tert-butoxy-2-oxoethyl)-[1,2,4]triazolo[1,5-a]pyridin-8-yl 4-(2-(tert-butoxycarbonyl)guanidino)-3-methylbenzoate

To a solution of tert-butyl 2-(8-hydroxy-[1,2,4]triazolo[1,5-a]pyridin-5-yl)acetate (40 mg, 0.16 mmol, 1.0 equiv) in DCM (10 ml) were added EDCI (60 mg, 0.314 mmol, 1.50 equiv), DMAP (40 mg, 0.328 mmol, 2.00 equiv) and (Z)-4-(2-(tert-butoxycarbonyl)guanidino)-3-methylbenzoic acid (40 mg, 0.136 mmol, 0.8 equiv). The resulting mixture was stirred for 2 h at 25° C. The reaction was monitored by LCMS. The solution was concentrated under vacuum. The residue was washed by brine and dried by Na₂SO₄ to yield (Z)-5-(2-tert-butoxy-2-oxoethyl)-[1,2,4]triazolo[1,5-a]pyridin-8-yl4-(2-(tert-butoxycarbonyl) guanidino)-3-methylbenzoate as a yellow oil. Mass spectrum (ESI, m/z): Calculated for C₂₆H₃₂N₆O₆, 525.6 [M+H], Measured: 525.7.

### Step 4: 2-(8-(4-guanidino-3-methylbenzoyloxy)-[1,2,4]triazolo[1,5-a]pyridin-5-yl)acetic acid

Into a 50 ml round bottom-flask, 5-[2-(tert-butoxy)-2-oxoethyl]-[1,2,4]triazolo[1,5-a]pyridin-8-yl 4-{[(1E)-{[(tert-butoxy)carbonyl]amino}({[(tert-butoxy)carbonyl]imino})methyl]amino}benzoate (30 mg, 0.048 mmol, 1.00 equiv) was dissolved in DCM (5 ml). Then TFA (1.5 ml) was added to the solution. The resulting solution was stirred 3.0 h at room temperature. The reaction was monitored by LCMS. The organic layers were concentrated under vacuum. The residue was purified by Prep-HPLC with the following conditions: (1#waters2767-5): Column, SunFire Prep C18, 19*150 mm 5 umH PrepC-001(T)18600256819513816414 04; mobile phase, Phase A: water with 0.05% TFA; Phase B: CH₃CN (5% CH₃CN up to 14% in 10 min, up to 100% CH₃CN in 1 min, hold 100% in 1 min, down to 14% CH₃CN in 0.1 min, hold 5% in 0.9 min); Detector, UV220 & 254 nm to yield 2-(8-(4-guanidino-3-methylbenzoyloxy)-[1,2,4]triazolo[1,5-a]pyridin-5-yl)acetic acid as a white solid.

¹HNMR (400 MHz, DMSO-d⁶) δ 12.78 (brs, 1H), 9.70 (brs, 1H), 8.51 (s, 1H), 8.17 (s, 1H), 8.09 (t, J=10.4 Hz, 1H), 7.22 (d, J=7.6 Hz, 1H), 7.49 (d, J=8.4 Hz, 5H), 7.29 (d, J=8.0 Hz, 1H), 4.22 (s, 2H), 2.35 (s, 3H). Mass spectrum (ESI, m/z): Calculated for C₁₇H₁₆N₆O₄, 369.3 [M+H], Measured: 369.1.

### Example 16: Compound #16

### 2-(8-(3-chloro-4-guanidinobenzoyloxy)-[1,2,4]triazolo[1,5-a]pyridin-5-yl)acetic

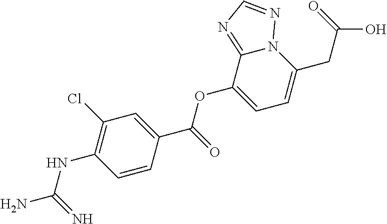 [SMILES: N=C(N)Nc1ccc(C(=O)Oc2ccc(CC(=O)O)n3ncnc23)cc1Cl]

### Step 1: Methyl 4-{[(1Z)-{[(tert-butoxy)carbonyl]amino}({[(tert-butoxy)carbonyl]imino})methyl]amino}-3-chlorobenzoate

Into a 50-mL round bottle was placed methyl 4-amino-3-chlorobenzoate (300 mg, 1.616 mmol, 1.00 equiv) DMF (20 mL) were added tert-butyl N-[(1E)-{[(tert-butoxy) carbonyl]imino}(methylsulfanyl)methyl]carbamate (470 mg, 1.619 mmol, 1.00 equiv), TEA (330 mg, 3.267 mmol, 2.00 equiv) and HgCl₂ (440 mg, 1.624 mmol, 1.00 equiv). The resulting solution was stirred 16 h at 25° C. The reaction was monitored by LCMS. The reaction was quenched by H₂O and extracted by EtOAc (20 ml×3). The organic layers was washed by brine (10 ml×3). The resulting solution was concentrated under vacuum. The residue was purified by silica gel chromatography (0-50% EtOAc/petroleum ether) to yield methyl 4-{[(1Z)-{[(tert-butoxy) carbonyl]amino}({[(tert-butoxy)carbonyl]imino})methyl]amino}-3-chlorobenzoate as a yellow oil. Mass spectrum (ESI, m/z): Calculated for C₁₉H₂₆CIN₃O₆, 428.2 [M+H], Measured: 428.2.

### Step 2: (Z)-4-(2,3-bis(tert-butoxycarbonyl)guanidino)-3-chlorobenzoic acid

To a solution of methyl 4-{[(1Z)-{[(tert-butoxy) carbonyl]amino}({[(tert-butoxy)carbonyl]imino})methyl]amino}-3-chlorobenzoate (200 mg, 0.467 mmol, 1.00 equiv) in THE (4 ml) and H₂O (4 ml) and MeOH (1 ml) was added NaOH (100 mg, 2.5 mmol, 5.00 equiv). The resulting mixture was stirred for 3 h at 50° C. The reaction was monitored by LCMS. The reaction was adjusted pH to 6 with 2N HCl. The solution was extracted with EtOAc (20 ml×3) and the organic layers combined and concentrated under vacuum. The residue was washed by brine and dried by Na₂SO₄ to yield (Z)-4-(2,3-bis(tert-butoxycarbonyl) guanidino)-3-chlorobenzoic acid as a yellow oil. Mass spectrum (ESI, m/z): Calculated for C₁₈H₂₄CIN₃O₆, 414.1[M+H], Measured: 414.2.

### Step 3: 5-[2-(tert-butoxy)-2-oxoethyl]-[1,2,4]triazolo[1,5-a]pyridin-8-yl 4-{[(1E)-{[(tert-butoxy)carbonyl]amino}({[(tert-butoxy)carbonyl]imino}) methyl]amino}-3-chlorobenzoate

To a solution of tert-butyl 2-(8-hydroxy-[1,2,4]triazolo[1,5-a]pyridin-5-yl)acetate (40 mg, 0.16 mmol, 1.00 equiv) in DCM (10 ml) were added EDCI (60 mg, 0.314 mmol, 1.50 equiv), DMAP (40 mg, 0.328 mmol, 2.00 equiv) and (E)-4-(2,3-bis(tert-butoxycarbonyl)guanidino)-3-chlorobenzoic acid (50 mg, 0.121 mmol, 0.75 equiv). The resulting mixture was stirred for 2 h at 25° C.

The reaction was monitored by LCMS. The solution was concentrated under vacuum. The residue was washed by brine and dried by Na₂SO₄. 5-[2-(tert-butoxy)-2-oxoethyl]-[1,2,4]triazolo[1,5-a]pyridin-8-yl 4-{[(1E)-{[(tert-butoxy) carbonyl]amino}({[(tert-butoxy)carbonyl]imino})methyl]amino}-3-chlorobenzoate as a yellow oil. Mass spectrum (ESI, m/z): Calculated for C₃₀H₃₇CIN₆O₈, 645.1[M+H], Measured: 645.6.

### Step 4: 2-(8-(3-chloro-4-guanidinobenzoyloxy)-[1,2,4]triazolo[1,5-a]pyridin-5-yl)acetic acid

Into a 50 ml round bottom-flask, 5-[2-(tert-butoxy)-2-oxoethyl]-[1,2,4]triazolo[1,5-a]pyridin-8-yl 4-{[(1E)-{[(tert-butoxy)carbonyl]amino}({[(tert-butoxy)carbonyl]imino})methyl]amino}-3-chlorobenzoate (40 mg, 0.062 mmol, 1.00 equiv) was dissolved in DCM (5 ml). Then TFA (2 ml) was added to the solution. The resulting solution was stirred 3.0 h at room temperature. The reaction was monitored by LCMS. The organic layers were concentrated under vacuum. The residue was purified by Prep-HPLC with the following conditions: (1#waters2767-5): Column, SunFire Prep C18, 19*150 mm 5 umH PrepC-001(T)18600256819513816414 04; mobile phase, Phase A: water with 0.05% TFA; Phase B: CH₃CN (5% CH₃CN up to 14% in 10 min, up to 100% CH₃CN in 1 min, hold 100% in 1 min, down to 14% CH₃CN in 0.1 min, hold 5% in 0.9 min); Detector, UV220 & 254 nm to yield 2-(8-(3-chloro-4-guanidinobenzoyloxy)-[1,2,4]triazolo[1,5-a]pyridin-5-yl)acetic acid as a white solid.

¹HNMR (300 MHz, DMSO-d⁶) δ: 12.66 (brs, 1H), 9.89 (s, 1H), 8.53 (s, 1H), 8.33 (s, 1H), 8.13-8.26 (m, 1H), 7.57-7.82 (m, 5H), 7.31 (d, J=8.1 Hz, 1H), 4.24 (s, 2H). Mass spectrum (ESI, m/z): Calculated for C₁₆H₁₃CIN₆O₄, 389.1[M+H], Measured: 389.1.

### Example 17: Compound #17

### 5-(2,4-dioxooxazolidin-5-yl)-[1,2,4]triazolo[1,5-a]pyridin-8-yl 4-guanidinobenzoate

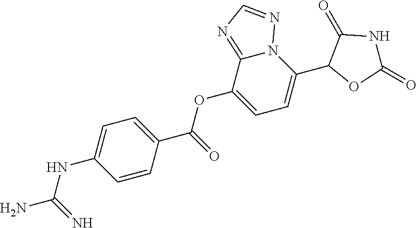 [SMILES: N=C(N)Nc1ccc(C(=O)Oc2ccc(C3OC(=O)NC3=O)n3ncnc23)cc1]

### Step 1: 8-(benzyloxy)-[1,2,4]triazolo[1,5-a]pyridine-5-carbaldehyde

Into a 100-mL round-bottom flask, to a solution of (8-(benzyloxy)-[1,2,4]triazolo[1,5-a]pyridin-5-yl)methanol (300 mg, 1.175 mmol, 1.00 equiv) in DCM (8 ml) was added DMP (1.5 g, 3.526 mmol, 3.00 equiv). The resulting mixture was stirred at 40° C. 5 h. The reaction progress was monitored by LCMS. After cooling down to room temperature, the reaction was quenched with H₂O (10 mL). The resulting mixture was extracted with DCM (3×10 mL). The organic layers were combined, dried over Na₂SO₄, filtered and concentrated. The residue was purified by silica gel chromatography (0-50% EtOAc/petroleum ether) to yield 8-(benzyloxy)-[1,2,4]triazolo[1,5-a]pyridine-5-carbaldehyde as a light yellow oil. Mass spectrum (ESI, m/z): Calculated for C₁₄H₁₁N₃O₂, 254.1 [M+H], Measured: 254.2.

### Step 2: 2-(8-(benzyloxy)-[1,2,4]triazolo[1,5-a]pyridin-5-yl)-2-hydroxyacetonitrile

Into a 100-mL round-bottom flask, to a solution of 8-(benzyloxy)-[1,2,4]triazolo[1,5-a]pyridine-5-carbaldehyde (180 mg, 0.711 mmol, 1.00 equiv) in DCM (10 ml) was added TMSCN (141 mg, 1.421 mmol, 2.00 equiv), AlCl₃ (190 mg, 1.421 mmol, 2.00 equiv). The resulting mixture was stirred at 25° C. overnight. The reaction progress was monitored by LCMS. The reaction was quenched with H₂O (10 mL). The resulting mixture was extracted with DCM (3×10 mL). The organic layers were combined, dried over Na₂SO₄, filtered and concentrated. The residue was purified by silica gel chromatography (0-50% EtOAc/petroleum ether) to yield 200 mg of 2-(8-(benzyloxy)-[1,2,4]triazolo[1,5-a]pyridin-5-yl)-2-hydroxyacetonitrile as a light yellow oil. Mass spectrum (ESI, m/z): Calculated for C₁₅H₁₂N₄O₂, 281.1 [M+H], Measured: 281.2.

### Step 3: 2-(8-(benzyloxy)-[1,2,4]triazolo[1,5-a]pyridin-5-yl)-2-hydroxyacetonitrile

Into a 100-mL round-bottom flask, to a solution of 2-(8-(benzyloxy)-[1,2,4]triazolo[1,5-a]pyridin-5-yl)-2-hydroxyacetonitrile (200 mg, 0.714 mmol, 1.00 equiv) in EtOH (8 ml) was added SOCl₂ (2 ml). The resulting mixture was stirred 3 h at 90° C. The reaction progress was monitored by TLC (PE:EA=3:1). The resulting solution concentrated to yield 200 mg of ethyl 2-(8-(benzyloxy)-[1,2,4]triazolo[1,5-a]pyridin-5-yl)-2-hydroxyacetate as a light yellow solid. Mass spectrum (ESI, m/z): Calculated for C₁₇H₁₇N₃O₄, 328.1 [M+H], Measured: 328.2.

### Step 4: 5-((2H-tetrazol-5-yl)methyl)-8-(benzyloxy)-[1,2,4]triazolo[1,5-a]pyridine

Into a 100-mL round-bottom flask, was placed ethyl 2-(8-(benzyloxy)-[1,2,4]triazolo[1,5-a]pyridin-5-yl)-2-hydroxyacetate (200 mg, 0.611 mmol, 1.00 equiv), 2,2,2-trichloroacetyl isocyanate (230 mg, 1.222 mmol, 2.00 equiv), DCM (10 ml). The reaction was stirred overnight at 25° C. The resulting solution concentrated, EtOH and Et₃N were added to the residue. The reaction was stirred 2 h at 90° C. The reaction progress was monitored by LCMS. The reaction was quenched with H₂O (10 mL). The resulting mixture was extracted with DCM (3×10 mL). The organic layers were combined, dried over Na₂SO₄, filtered and concentrated. The residue was purified by silica gel chromatography (0-50% EtOAc/petroleum ether) to yield 5-(8-(benzyloxy)-[1,2,4]triazolo[1,5-a]pyridin-5-yl)oxazolidine-2,4-dione as a light yellow oil. Calculated for C₁₆H₁₂N₄O₄, 325.1 [M+H], Measured: 325.2.

### Step 5: 5-(8-hydroxy-[1,2,4]triazolo[1,5-a]pyridin-5-yl)oxazolidine-2,4-dione

Into a 50-mL round-bottom flask, was placed 5-(8-(benzyloxy)-[1,2,4]triazolo[1,5-a]pyridin-5-yl)oxazolidine-2,4-dione (180 mg, 0.555 mmol, 1.00 equiv), Pd/C10% (30 mg), EA (8 ml). The reaction was purged with an inert atmosphere of hydrogen and was stirred 2 h at 25° C. The reaction progress was monitored by TLC (DCM:MeOH=10:1). A filtration was performed. The filtrate was concentrated under vacuum to yield 5-(8-hydroxy-[1,2,4]triazolo[1,5-a]pyridin-5-yl)oxazolidine-2,4-dione as a light yellow solid. Mass spectrum (ESI, m/z): Calculated for C₉H₆N₄O₄, 235.1 [M+H], Measured: 235.0.

### Step 6: 5-(2,4-dioxo-1,3-oxazolidin-5-yl)-[1,2,4]triazolo[1,5-a]pyridin-8-yl 4-{[(1Z)-{[(tert-butoxy)carbonyl]amino}({[(tert-butoxy)carbonyl]imino}) methyl]amino}benzoate

Into a 100-mL round-bottom flask, was placed 5-(8-hydroxy-[1,2,4]triazolo[1,5-a]pyridin-5-yl)oxazolidine-2,4-dione (30 mg, 0.128 mmol, 1.00 equiv), 4-{[(1E)-{[(tert-butoxy)carbonyl]amino}({[(tert-butoxy)carbonyl]imino}) methyl]amino}benzoic acid (49 mg, 0.128 mmol, 1.00 equiv), EDCI (30 mg, 0.192 mmol, 1.50 equiv), DMAP (24 mg, 0.192 mmol, 1.50 equiv), DCM (5 ml). The reaction was stirred 2 h at 25° C. The reaction progress was monitored by LCMS. The resulting solution concentrated. The residue was purified by silica gel chromatography (0-50% PE/EA) to yield 5-(2,4-dioxo-1,3-oxazolidin-5-yl)-[1,2,4]triazolo[1,5-a]pyridin-8-yl 4-{[(1Z)-{[(tert-butoxy)carbonyl]amino}({[(tert-butoxy)carbonyl]imino})methyl]amino}benzoat as a light yellow oil. Mass spectrum (ESI, m/z): Calculated for C₂₇H₂₉N₇O₉, 596.2 [M+H], Measured: 596.6.

### Step 7: 5-(2,4-dioxooxazolidin-5-yl)-[1,2,4]triazolo[1,5-a]pyridin-8-yl 4-guanidinobenzoate

Into a 50-mL round-bottom flask, was placed 5-(2,4-dioxo-1,3-oxazolidin-5-yl)-[1,2,4]triazolo[1,5-a]pyridin-8-yl 4-{[(1Z)-{[(tert-butoxy)carbonyl]amino}({[(tert-butoxy)carbonyl]imino})methyl]amino}benzoate (20 mg, 0.034 mmol, 1.00 equiv), DCM (5 ml), TFA (2 ml). The reaction was stirred 2 h at 25° C. The reaction progress was monitored by LCMS. The resulting mixture was concentrated under vacuum. The residue was purified by Prep-HPLC with the following conditions (1#-Waters 2767-5): Column, SunFire Prep C18, 5 um, 19*100 mm; mobile phase, Water of 0.05% TFA and CH₃CN (5% CH₃CN up to 20% in 10 min, up to 100% in 2 min, down to 20% in 2 min; Detector, 254 nm to yield 5-(2,4-dioxooxazolidin-5-yl)-[1,2,4]triazolo[1,5-a]pyridin-8-yl 4-guanidinobenzoate as white solid. ¹HNMR (300 MHz, DMSO-d⁶) δ: 12.54 (s, 1H), 10.15 (s, 1H), 8.64 (s, 1H), 8.24 (d, J=8.7 Hz, 2H), 7.76-7.89 (m, 6H), 7.48 (d, J=8.7 Hz, 2H), 6.62 (s, 1H).

Mass spectrum (ESI, m/z): Calculated for C₁₇H₁₃N₇O₅, 396.1 [M+H], Measured: 396.1.

### Example 18: Compound #18

### 2-(8-(2-chloro-4-guanidinobenzoyloxy)-[1,2,4]triazolo[1,5-a]pyridin-5-yl)acetic acid

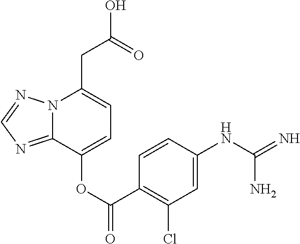 [SMILES: N=C(N)Nc1ccc(C(=O)Oc2ccc(CC(=O)O)n3ncnc23)c(Cl)c1]

### Step 1: (Z)-4-(2,3-bis(tert-butoxycarbonyl)guanidino)-2-chlorobenzoic acid

To a solution of 4-amino-2-chlorobenzoic acid (300 mg, 1.748 mmol) in 1,4-dioxane (10 ml), was added tert-butyl N-({[(tert-butoxy)carbonyl]amino}[(trifluoromethane)sulfonylimino]methyl)carbamate (752.706 mg, 1.923 mmol), Et₃N (353.186 mg, 3.497 mmol). The resulting solution was stirred overnight at 60° C. The reaction was monitored by LCMS. The resulting mixture was concentrated under vacuum. The residue was purified by silica gel chromatography (40-60% EtOAc/petroleum ether) to yield (Z)-4-(2,3-bis(tert-butoxycarbonyl)guanidino)-2-chlorobenzoic acid as a yellow solid. Mass spectrum (ESI, m/z): Calculated for C₁₈H₂₄CIN₃O₆, 414.1[M+H], Measured: 414.5.

### Step 2: 5-[2-(tert-butoxy)-2-oxoethyl]-[1,2,4]triazolo[1,5-a]pyridin-8-yl 4-{[(1Z)-{[(tert-butoxy)carbonyl]amino}({[(tert-butoxy)carbonyl]imino}) methyl] amino}-2-chlorobenzoate

To a solution of tert-butyl 2-(8-hydroxy-[1,2,4]triazolo[1,5-a]pyridin-5-yl)acetate (70 mg, 0.281 mmol) in DCM (5.0 ml), was added EDCI (107.275 mg, 0.562 mmol), DMAP (68.521 mg, 0.562 mmol), (Z)-4-(2,3-bis(tert-butoxycarbonyl)guanidino)-2-chlorobenzoic acid (127.842 mg, 0.309 mmol). The resulting solution was stirred 3.0 h at room temperature. The reaction was monitored by LCMS. The resulting mixture was concentrated under vacuum. The residue was purified by TLC (PE:EA=2:1) to yield 5-[2-(tert-butoxy)-2-oxoethyl]-[1,2,4]triazolo[1,5-a]pyridin-8-yl 4-{[(1Z)-{[(tert-butoxy) carbonyl]amino}({[(tert-butoxy)carbonyl]imino})methyl]amino}-2-chlorobenzoate as a yellow solid. Mass spectrum (ESI, m/z): Calculated for C₃₀H₃₇CIN₆O₈, 645.2[M+H], Measured: 645.3.

### Step 3: 2-(8-(2-chloro-4-guanidinobenzoyloxy)-[1,2,4]triazolo[1,5-a]pyridin-5-yl)acetic acid

To a solution of 5-[2-(tert-butoxy)-2-oxoethyl]-[1,2,4]triazolo[1,5-a]pyridin-8-yl 4-{[(1Z)-{[(tert-butoxy)carbonyl]amino}({[(tert-butoxy)carbonyl]imino}) methyl]amino}-2-chlorobenzoate (160 mg, 0.151 mmol) in DCM (5.0 ml) was added TFA (2.0 ml). The resulting solution was stirred 1.0 h at room temperature. The reaction was monitored by LCMS. The resulting mixture was concentrated under vacuum. The residue was purified by Prep-HPLC with the following conditions (1#-Waters 2767-5): Column, SunFire Prep C18, 5 um, 19*100 mm; mobile phase, Water with 0.05% TFA and CH₃CN (5% CH₃CN up to 30% in 10 min, up to 100% in 0.1 min, hold 100% in 0.9 min, down to 5% in 0.1 min, hold 5% in 1.4 min); Detector, UV 220&254 nm to yield 2-(8-(2-chloro-4-guanidinobenzoyloxy)-[1,2,4]triazolo[1,5-a]pyridin-5-yl)acetic acid as a white solid.

¹H NMR (300 MHz, DMSO-d⁶) δ: 12.84 (s, 1H), 10.37 (s, 1H), 8.55 (s, 1H), 8.31 (d, J=8.7 Hz, 1H), 7.99 (s, 4H), 7.76 (d, J=7.8 Hz, 1H), 7.56-7.57 (m, 1H), 7.43-7.47 (m, 1H), 7.31 (d, J=7.8 Hz, 1H), 4.25 (s, 2H). Mass spectrum (ESI, m/z): Calculated for C₁₆H₁₃CIN₆O₄, 389.1[M+H], Measured: 389.1.

### Example 19: Compound #19

### 2-(8-(4-guanidino-2-methylbenzoyloxy)-[1,2,4]triazolo[1,5-a]pyridin-5-yl)acetic acid

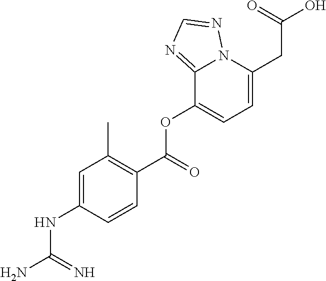 [SMILES: Cc1cc(NC(=N)N)ccc1C(=O)Oc1ccc(CC(=O)O)n2ncnc12]

### Step 1: (Z)-4-(2,3-bis(tert-butoxycarbonyl)guanidino)-2-methylbenzoic acid

To a solution of 4-amino-2-methylbenzoic acid (500 mg, 3.308 mmol, 1.0 equiv) in THE (20 ml) were added Et₃N (500 mg, 4.95 mmol, 1.50 equiv) and (E)-di-tert-butyl (1H-pyrazol-1-yl)methanediylidenedicarbamate (1.00 g, 3.222 mmol, 1.00 equiv). The resulting mixture was stirred for 16 h at 25° C. The reaction was monitored by LCMS. The resulting solution was concentrated under vacuum. The residue was purified by silica gel chromatography (0-50% CH₃CN/H₂O) to yield (Z)-4-(2,3-bis(tert-butoxycarbonyl)guanidino)-2-methylbenzoic acid as a yellow oil. Mass spectrum (ESI, m/z): Calculated for C₁₉H₂₇N₃O₆, 394.4 [M+H], Measured: 394.2.

### Step 2: 5-[2-(tert-butoxy)-2-oxoethyl]-[1,2,4]triazolo[1,5-a]pyridin-8-yl 4-{[(1Z)-{[(tert-butoxy)carbonyl]amino}({[(tert-butoxy)carbonyl]imino}) methyl]amino}-2-methylbenzoate

To a solution of tert-butyl 2-(8-hydroxy-[1,2,4]triazolo[1,5-a]pyridin-5-yl)acetate (50 mg, 0.201 mmol, 1.00 equiv) in DCM (3 ml) were added EDCI (76 mg, 0.382 mmol, 2.00 equiv), DMAP (48 mg, 0.393 mmol, 2.00 equiv) and (Z)-4-(2,3-bis(tert-butoxycarbonyl)guanidino)-2-methylbenzoic acid (87 mg, 0.221 mmol, 1.00 equiv). The resulting mixture was stirred for 2 h at 25° C. The reaction was monitored by LCMS. The resulting solution was concentrated under vacuum. The residue was purified by silica gel chromatography (0-50% EtOAc/petroleum ether) to yield 5-[2-(tert-butoxy)-2-oxoethyl]-[1,2,4]triazolo[1,5-a]pyridin-8-yl 4-{[(1Z)-{[(tert-butoxy)carbonyl]amino}({[(tert-butoxy)carbonyl]imino})methyl]amino}-2-methylbenzoate as a yellow oil. Mass spectrum (ESI, m/z): Calculated for C₃₁H₄₀N₆O₈, 625.3 [M+H], Measured: 625.7.

### Step 3: 2-(8-(4-guanidino-2-methylbenzoyloxy)-[1,2,4]triazolo[1,5-a]pyridin-5-yl)acetic acid

Into a 50 ml round bottom-flask, 5-[2-(tert-butoxy)-2-oxoethyl]-[1,2,4]triazolo[1,5-a]pyridin-8-yl 4-{[(1Z)-{[(tert-butoxy)carbonyl]amino}({[(tert-butoxy)carbonyl]imino})methyl]amino}-2-methylbenzoate (50 mg, 0.080 mmol, 1.00 equiv) was dissolved in DCM (5 ml). Then TFA (1.5 ml) was added to the solution. The resulting solution was stirred 3.0 h at room temperature. The reaction was monitored by LCMS. The organic layers were concentrated under vacuum. The residue was purified by Prep-HPLC with the following conditions: (1#waters2767-5): Column, SunFire Prep C18, 19*150 mm 5 umH PrepC-001(T)18600256819513816414 04; mobile phase, Phase A: water with 0.05% TFA; Phase B: CH₃CN (5% CH₃CN up to 14% in 10 min, up to 100% CH₃CN in 1 min, hold 100% in 1 min, down to 14% CH₃CN in 0.1 min, hold 5% in 0.9 min); Detector, UV220 & 254 nm to yield 2-(8-(4-guanidino-2-methylbenzoyloxy)-[1,2,4]triazolo[1,5-a]pyridin-5-yl)acetic acid as a white solid.

¹HNMR (400 MHz, DMSO-d⁶) δ: 12.81 (s, 1H), 10.10 (s, 1H), 8.53 (s, 1H), 8.24 (t, J=8.8 Hz, 1H), 7.73 (t, J=8.4 Hz, 4H), 7.30 (d, J=8.0 Hz, 3H), 4.24 (s, 2H), 2.63 (s, 3H). Mass spectrum (ESI, m/z): Calculated for C₁₇H₁₆N₆O₄, 369.1 [M+H], Measured: 369.1.

### Example 20: Compound #20

### 2-(8-(4-guanidino-2-(trifluoromethyl)benzoyloxy)-[1,2,4]triazolo [1,5-a] pyridin-5-yl)acetic acid

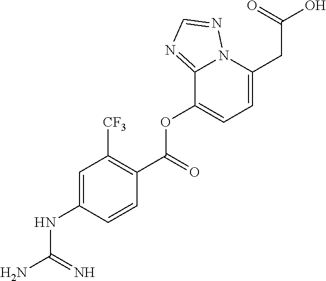 [SMILES: N=C(N)Nc1ccc(C(=O)Oc2ccc(CC(=O)O)n3ncnc23)c(C(F)(F)F)c1]

### Step 1: (Z)-4-(2,3-bis(tert-butoxycarbonyl)guanidino)-2-(trifluoromethyl) benzoic acid

Into a 250-mL round bottle was placed 4-amino-2-(trifluoromethyl) benzoic acid (500 mg, 2.437 mmol, 1.00 equiv) DMF (20 mL). Then tert-butyl N-[(1E)-{[(tert-butoxy) carbonyl]imino}(methylsulfanyl)methyl]carbamate (780 mg, 2.686 mmol, 1.10 equiv), TEA (600 mg, 5.94 mmol, 2.00 equiv) and HgCl₂ (800 mg, 2.952 mmol, 1.20 equiv) were added. The resulting solution was stirred 16 h at 25° C. The reaction was monitored by LCMS. The reaction was quenched by H₂O and extracted by EtOAc (20 ml×3). The organic layers was washed by brine (10 ml×3). The resulting solution was concentrated under vacuum. The residue was purified by silica gel chromatography (0-50% EtOAc/petroleum ether) to yield (Z)-4-(2,3-bis(tert-butoxycarbonyl) guanidino)-2-(trifluoromethyl)benzoic acid as a yellow oil. Mass spectrum (ESI, m/z): Calculated for C₁₉H₂₄F₃N₃O₆, 348.1 [M-Boc+H], Measured: 348.1.

### Step 2: (E)-5-(2-tert-butoxy-2-oxoethyl)-[1,2,4]triazolo[1,5-a]pyridin-8-yl 4-(2-(tert-butoxycarbonyl)guanidino)-2-(trifluoromethyl)benzoate

To a solution of tert-butyl 2-(8-hydroxy-[1,2,4]triazolo[1,5-a]pyridin-5-yl)acetate (50 mg, 0.201 mmol, 1.00 equiv) in DMF (3 ml) and DCM (3 ml) were added EDCI (80 mg, 0.419 mmol, 2.00 equiv), DMAP (50 mg, 0.41 mmol, 2.00 equiv) and (E)-4-(2-(tert-butoxycarbonyl) guanidino)-2-(trifluoromethyl)benzoic acid (70 mg, 0.202 mmol, 1.00 equiv). The resulting mixture was stirred for 2 h at 25° C. The reaction was monitored by LCMS. The resulting solution was quenched by H₂O and extracted with EtOAc (10 ml×3). The residue was washed by brine and dried by Na₂SO₄. The solution was concentrated under vacuum. The residue was purified by silica gel chromatography (0-50% EtOAc/petroleum ether) to yield (E)-5-(2-tert-butoxy-2-oxoethyl)-[1,2,4]triazolo[1,5-a]pyridin-8-yl 4-(2-(tert-butoxycarbonyl)guanidino)-2-(trifluoromethyl)benzoate as a yellow oil. Mass spectrum (ESI, m/z): Calculated for C₂₆H₂₉F₃N₆O₆, 579.5[M+H], Measured: 579.6.

### Step 3: 2-(8-(4-guanidino-2-(trifluoromethyl)benzoyloxy)-[1,2,4]triazolo [1,5-a]pyridin-5-yl)acetic acid

Into a 50 ml round bottom-flask, 5-[2-(tert-butoxy)-2-oxoethyl]-[1,2,4]triazolo[1,5-a]pyridin-8-yl 4-{[(1Z)-{[(tert-butoxy)carbonyl]amino}({[(tert-butoxy)carbonyl]imino})methyl]amino}-2-(trifluoromethyl)benzoate (50 mg, 0.074 mmol, 1.00 equiv) was dissolved in DCM (5 ml). Then TFA (1.5 ml) was added to the solution. The resulting solution was stirred 3.0 h at room temperature. The reaction was monitored by LCMS. The organic layers were concentrated under vacuum. The residue was purified by Prep-HPLC with the following conditions: (1#waters2767-5): Column, SunFire Prep C18, 19*150 mm 5 umH PrepC-001(T)18600256819513816414 04; mobile phase, Phase A: water with 0.05% TFA; Phase B: CH₃CN (5% CH₃CN up to 14% in 10 min, up to 100% CH₃CN in 1 min, hold 100% in 1 min, down to 14% CH₃CN in 0.1 min, hold 5% in 0.9 min); Detector, UV220 & 254 nm to yield 2-(8-(4-guanidino-2-(trifluoromethyl)benzoyloxy)-[1,2,4]triazolo[1,5-a]pyridin-5-yl)acetic acid as a white solid.

¹HNMR (400 MHz, DMSO-d⁶) δ: 8.55 (s, 1H), 8.35 (d, J=8.4 Hz, 1H), 7.84 (s, 3H), 7.79 (d, J=1.6 Hz, 1H), 7.73 (t, J=8.0 Hz, 2H), 7.30 (d, J=8.0 Hz, 1H), 4.23 (s, 2H). Mass spectrum (ESI, m/z): Calculated for C₁₇H₁₃F₃N₆O₄, 423.3[M+H], Measured: 423.05.

### Example 21: Compound #21

### 2-(8-(2-fluoro-4-guanidinobenzoyloxy)-[1,2,4]triazolo[1,5-a]pyridin-5-yl)acetic acid

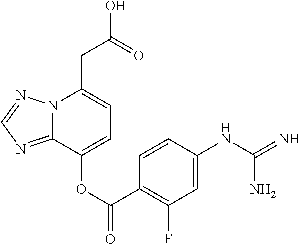 [SMILES: N=C(N)Nc1ccc(C(=O)Oc2ccc(CC(=O)O)n3ncnc23)c(F)c1]

### Step 1: 5-(2-tert-butoxy-2-oxoethyl)-[1,2,4]triazolo[1,5-a]pyridin-8-yl 2-fluoro-4-guanidinobenzoate

To a solution of tert-butyl 2-(8-hydroxy-[1,2,4]triazolo[1,5-a]pyridin-5-yl)acetate (40 mg, 0.160 mmol) in DCM (5.0 ml), was added EDCI (61.300 mg, 0.321 mmol), DMAP (39.155 mg, 0.321 mmol). Then 2-fluoro-4-guanidino benzoic acid (31.639 mg, 0.160 mmol) in DMF (2.0 ml) was added. The resulting solution was stirred 3.0 h at room temperature. The reaction was monitored by LCMS. The resulting mixture was concentrated under vacuum. The residue was purified by Prep-HPLC with the following conditions (1#-Waters 2767-5): Column, SunFire Prep C18, 5 um, 19*100 mm; mobile phase, Water with 0.05% TFA and CH₃CN (5% CH₃CN up to 30% in 10 min, up to 100% in 0.1 min, hold 100% in 0.9 min, down to 5% in 0.1 min, hold 5% in 1.4 min); Detector, UV 220&254 nm to yield 5-(2-tert-butoxy-2-oxoethyl)-[1,2,4]triazolo[1,5-a]pyridin-8-yl 2-fluoro-4-guanidinobenzoate as a white solid. Mass spectrum (ESI, m/z): Calculated for C₂₀H₂₁FN₆O₄, 429.2[M+H], Measured: 429.2.

### Step 2: 2-(8-(2-fluoro-4-guanidinobenzoyloxy)-[1,2,4]triazolo[1,5-a]pyridin-5-yl)acetic acid

To a solution of 5-(2-tert-butoxy-2-oxoethyl)-[1,2,4]triazolo[1,5-a]pyridin-8-yl 2-fluoro-4-guanidinobenzoate (30 mg, 0.070 mmol) in DCM (5.0 ml) was added TFA (1.0 ml). The resulting solution was stirred 2.0 h at room temperature. The reaction was monitored by LCMS. The resulting mixture was concentrated under vacuum. The residue was purified by Prep-HPLC with the following conditions (1#-Waters 2767-5): Column, SunFire Prep C18, 5 um, 19*100 mm; mobile phase, Water with 0.05% TFA and CH₃CN (5% CH₃CN up to 30% in 10 min, up to 100% in 0.1 min, hold 100% in 0.9 min, down to 5% in 0.1 min, hold 5% in 1.4 min); Detector, UV 220&254 nm to yield 2-(8-(2-fluoro-4-guanidinobenzoyloxy)-[1,2,4]triazolo[1,5-a]pyridin-5-yl)acetic acid as a white solid.

¹H NMR (300 MHz, DMSO-d⁶) δ: 12.82 (s, 1H), 10.45 (s, 1H), 8.52 (s, 1H), 8.18-8.24 (m, 1H), 7.95-8.01 (m, 4H), 7.71-7.75 (m, 1H), 7.27-7.38 (m, 3H), 4.24 (s, 2H). Mass spectrum (ESI, m/z): Calculated for C₁₆H₁₃N₆O₄, 373.1[M+H], Measured: 373.1.

### Example 22: Compound #22

### 2-(8-(3-cyano-4-guanidinobenzoyloxy)-[1,2,4]triazolo[1,5-a]pyridin-5-yl)acetic acid

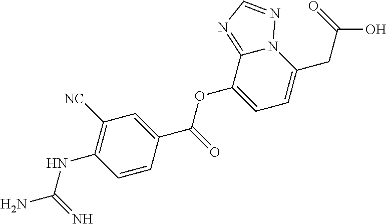 [SMILES: N#Cc1cc(C(=O)Oc2ccc(CC(=O)O)n3ncnc23)ccc1NC(=N)N]

### Step 1: Methyl 4-{[(1Z)-{[(tert-butoxy)carbonyl]amino}({[(tert-butoxy) carbonyl]imino})methyl]amino}-3-cyanobenzoate

Into a 100-mL round-bottom flask, was placed methyl 4-{[(1Z)-{[(tert-butoxy)carbonyl]amino}({[(tert-butoxy)carbonyl]imino})methyl]amino}-3-iodobenzoate (200 mg, 0.385 mmol, 1.00 equiv), dicyanozinc (90 mg, 0.770 mmol, 2.00 equiv), Pd(PPh₃)₄ (9 mg, 0.031 mmol, 0.10 equiv), DMF (8 ml). The reaction was purged with an inert atmosphere of N2 and was stirred 4 h at 80° C. The reaction progress was monitored by LCMS. The reaction was then quenched by H₂O (10 ml). The resulting mixture was extracted with EA (3×10 mL). The organic layers were combined, dried over Na₂SO₄, filtered and concentrated. The residue was purified by silica gel chromatography (0-30% EtOAc/petroleum ether) to yield methyl 4-{[(1Z)-{[(tert-butoxy)carbonyl]amino}({[(tert-butoxy)carbonyl]imino})methyl]amino}-3-cyanobenzoate as a light yellow oil. Mass spectrum (ESI, m/z): Calculated for C₂₀H₂₆N₄O₆, 419.2 [M+H], Measured: 419.2.

### Step 2: 4-{[(1Z)-{[(tert-butoxy)carbonyl]amino}({[(tert-butoxy)carbonyl]imino})methyl]amino}-3-cyanobenzoic acid

Into a 50-mL round-bottom flask, was placed methyl 4-{[(1Z)-{[(tert-butoxy)carbonyl]amino}({[(tert-butoxy)carbonyl]imino})methyl]amino}-3-cyanobenzoate (130 mg, 0.311 mmol, 1.00 equiv), NaOH (62 mg, 1.553 mmol, 5.00 equiv), THE (3 ml), H₂O (3 ml). The reaction was stirred 1 h at 50° C. The reaction progress was monitored by LCMS. The pH value of the solution was adjusted to 6 with 2N hydrogen chloride. The resulting mixture was extracted with EtOAc (3×10 mL). The organic layers were combined, dried over Na₂SO₄, filtered and concentrated to yield 4-{[(1Z)-{[(tert-butoxy)carbonyl]amino}({[(tert-butoxy)carbonyl]imino})methyl]amino}-3-cyanobenzoic acid as a light yellow oil. Mass spectrum (ESI, m/z): Calculated for C₁₉H₂₄N₄O₆, 405.2 [M+H], Measured: 405.2.

### Step 3: 5-[2-(tert-butoxy)-2-oxoethyl]-[1,2,4]triazolo[1,5-a]pyridin-8-yl 4-{[(1Z)-{[(tert-butoxy)carbonyl]amino}({[(tert-butoxy)carbonyl]imino}) methyl]amino}-3-cyanobenzoate

Into a 100-mL round-bottom flask, was placed (Z)-4-(2,3-bis(tert-butoxycarbonyl)guanidino)-3-cyanobenzoic acid (50 mg, 0.124 mmol, 1.00 equiv), tert-butyl 2-(8-hydroxy-[1,2,4]triazolo[1,5-a]pyridin-5-yl)acetate (31 mg, 0.124 mmol, 1.00 equiv), EDCI (29 mg, 0.185 mmol, 1.50 equiv), DMAP (23 mg, 0.185 mmol, 1.50 equiv), DCM (8 ml). The reaction was stirred 2 h at 25° C. The reaction progress was monitored by LCMS. The resulting solution concentrated. The residue was purified by silica gel chromatography (0-50% PE/EA) to yield 5-[2-(tert-butoxy)-2-oxoethyl]-[1,2,4]triazolo[1,5-a]pyridin-8-yl 4-{[(1Z)-{[(tert-butoxy)carbonyl]amino}({[(tert-butoxy)carbonyl]imino})methyl]amino}-3-cyanobenzoate as a light yellow oil. Mass spectrum (ESI, m/z): Calculated for C₃₁H₃₇N₇O, 636.3 [M+H], Measured: 636.3.

### Step 4: 2-(8-(3-cyano-4-guanidinobenzoyloxy)-[1,2,4]triazolo[1,5-a]pyridin-5-yl)acetic acid

Into a 50-mL round-bottom flask, was placed 5-[2-(tert-butoxy)-2-oxoethyl]-[1,2,4]triazolo[1,5-a]pyridin-8-yl 4-{[(1Z)-{[(tert-butoxy)carbonyl]amino}({[(tert-butoxy)carbonyl]imino})methyl]amino}-3-cyanobenzoat (40 mg, 0.063 mmol, 1.00 equiv), DCM (5 ml), TFA (2 ml). The reaction was stirred 2 h at 25° C. The reaction progress was monitored by LCMS. The resulting mixture was concentrated under vacuum. The residue was purified by Prep-HPLC with the following conditions (1#-Waters 2767-5): Column, SunFire Prep C18, 5 um, 19*100 mm; mobile phase, Water of 0.05% TFA and CH₃CN (5% CH₃CN up to 20% in 10 min, up to 100% in 2 min, down to 20% in 2 min; Detector, 254 nm to yield 2-(8-(3-cyano-4-guanidinobenzoyloxy)-[1,2,4]triazolo[1,5-a]pyridin-5-yl)acetic acid as white solid.

¹HNMR (300 MHz, DMSO-d6) δ: 8.98 (s, 1H), 8.52 (s, 1H), 8.17-8.20 (m, 1H), 7.80-8.00 (m, 1H), 7.72 (d, J=7.8 Hz, 1H), 7.28-7.34 (m, 2H), 6.73 (Brs, 2H), 4.24 (s, 2H). Calculated for C₁₇H₁₃N₇O₄, 380.1 [M+H], Measured: 380.1.

### Example 23: Compound #23

### 2-(8-(4-guanidino-3-hydroxybenzoyloxy)-[1,2,4]triazolo[1,5-a]pyridin-5-yl)acetic acid

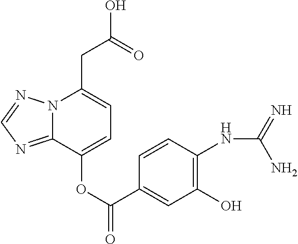 [SMILES: N=C(N)Nc1ccc(C(=O)Oc2ccc(CC(=O)O)n3ncnc23)cc1O]

### Step 1: Methyl 4-{[(1Z)-{[(tert-butoxy)carbonyl]amino}({[(tert-butoxy)carbonyl]imino})methyl]amino}-3-hydroxybenzoate

To a solution of methyl 4-amino-3-hydroxybenzoate (200 mg, 1.196 mmol) in DMF (10 mL) was added tert-butyl N-[(1E)-{[(tert-butoxy)carbonyl]amino}(methylsulfanyl)methylidene]carbamate (416.907 mg, 1.436 mmol), Et₃N (241.682 mg, 2.393 mmol), HgCl₂ (325.433 mg, 1.196 mmol). The resulting mixture was stirred at room temperature overnight. The reaction progress was monitored by LCMS. The reaction was quenched with H₂O (20 mL). The resulting mixture was extracted with EtOAc (3×20 mL). The organic layers were combined, dried over Na₂SO₄, filtered and concentrated. The residue was purified by TLC (PE:EA=3:1) to yield methyl 4-{[(1Z)-{[(tert-butoxy)carbonyl]amino}({[(tert-butoxy)carbonyl]imino})methyl]amino}-3-hydroxybenzoate as a yellow solid. Mass spectrum (ESI, m/z): Calculated for C₁₉H₂₇N₃O₇, 410.2[M+H], Measured: 410.5.

### Step 2: (E)-4-(2-(tert-butoxycarbonyl)guanidino)-3-fluorobenzoic acid

To a solution of methyl 4-{[(1Z)-{[(tert-butoxy)carbonyl]amino}({[(tert-butoxy)carbonyl]imino})methyl]amino}-3-hydroxybenzoate (280 mg, 0.684 mmol) in THE (4.0 ml) with H₂O (2.0 ml), was added NaOH (136.774 mg, 3.419 mmol). The resulting solution was stirred 1.0 h at 50° C. The reaction was monitored by LCMS. The reaction was quenched by H₂O (5 ml) and with 2 N HCl adjusted pH to 6. The mixture was extracted with EA (3*5 ml), the organic layers were combined, dried over Na₂SO₄ and concentrated under vacuum to yield (E)-4-(2-(tert-butoxycarbonyl)guanidino)-3-fluorobenzoic acid as a yellow oil. Mass spectrum (ESI, m/z): Calculated for C₁₃H₁₇N₃O₅, 296.1[M+H], Measured: 296.4.

### Step 3: 5-(2-tert-butoxy-2-oxoethyl)-[1,2,4]triazolo[1,5-a]pyridin-8-yl 4-(N-hydroxycarbamimidoyl)benzoate

To a solution of tert-butyl 2-(8-hydroxy-[1,2,4]triazolo[1,5-a]pyridin-5-yl)acetate (50 mg, 0.201 mmol) in DCM (5.0 ml), was added EDCI (76.625 mg, 0.401 mmol), DMAP (48.944 mg, 0.401 mmol), (E)-4-(2-(tert-butoxycarbonyl) guanidino)-3-hydroxybenzoic acid (65.155 mg, 0.221 mmol). The resulting solution was stirred 3.0 h at room temperature. The reaction was monitored by LCMS. The resulting mixture was concentrated under vacuum. The residue was purified by TLC (PE:EA=2:1) to yield 5-(2-tert-butoxy-2-oxoethyl)-[1,2,4]triazolo[1,5-a]pyridin-8-yl 4-(N-hydroxycarbamimidoyl)benzoate as a yellow solid. Mass spectrum (ESI, m/z): Calculated for C₂₅H₃₀N₆O₇, 527.2[M+H], Measured: 527.2.

### Step 4: 2-(8-(4-guanidino-3-hydroxybenzoyloxy)-[1,2,4]triazolo[1,5-a]pyridin-5-yl)acetic acid

To a solution of (E)-5-(2-tert-butoxy-2-oxoethyl)-[1,2,4]triazolo[1,5-a]pyridin-8-yl 4-(2-(tert-butoxycarbonyl)guanidino)-3-hydroxybenzoate (20 mg, 0.038 mmol) in DCM (4.0 ml) was added TFA (1.0 ml). The resulting solution was stirred 2.0 h at room temperature. The reaction was monitored by LCMS. The resulting mixture was concentrated under vacuum. The residue was purified by Prep-HPLC with the following conditions (1#-Waters 2767-5): Column, SunFire Prep C18, 5 um, 19*100 mm; mobile phase, Water with 0.05% TFA and CH₃CN (5% CH₃CN up to 30% in 10 min, up to 100% in 0.1 min, hold 100% in 0.9 min, down to 5% in 0.1 min, hold 5% in 1.4 min); Detector, UV 220&254 nm to yield 2-(8-(4-guanidino-3-hydroxybenzoyloxy)-[1,2,4]triazolo[1,5-a]pyridin-5-yl)acetic acid as a white solid.

¹H NMR (400 MHz, DMSO-d⁶) δ: 8.51 (s, 1H), 7.68-7.75 (m, 3H), 7.45-7.50 (m, 2H), 7.39-7.44 (m, 2H), 7.27-7.29 (m, 1H), 4.21 (s, 2H). Mass spectrum (ESI, m/z): Calculated for C₁₆H₁₄N₆O₅, 371.1 [M+H], Measured: 371.1.

### Example 24: Compound #24

### 2-(8-(4-(3-2-(8-(7-guanidinobenzofuran-4-carbonyloxy)-[1,2,4] triazolo[1,5-a]pyridin-5-yl)acetic acid

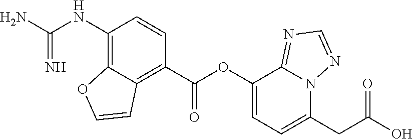 [SMILES: N=C(N)Nc1ccc(C(=O)Oc2ccc(CC(=O)O)n3ncnc23)c2ccoc12]

### Step 1: Methyl 3-(allyloxy)-4-nitrobenzoate

Into a 50 ml flask was placed a solution of methyl 3-hydroxy-4-nitrobenzoate (2.00 g, 10.14 mmol, 1.00 equiv.) in CH₃CN (30 ml). 3-Bromoprop-1-ene (1.50 g, 12.07 mmol, 1.20 equiv.), K2CO₃ (2.10 g, 15.19 mmol, 1.5 equiv.) were then added. The mixture was stirred for 3 h at 25° C. The reaction was monitored by TLC. The mixture was extracted with EA, and washed with brine, dried and concentrated under vacuum. The residue was purified by flash with PE/EA=3/1 to yield methyl 3-(allyloxy)-4-nitrobenzoate as yellow solid.

### Step 2: Methyl 2-allyl-3-hydroxy-4-nitrobenzoate

Into a 50 ml three-necked-bottom was placed methyl 3-(allyloxy)-4-nitrobenzoate (2 g, 8.431 mmol, 1 equiv.). The reaction was stirred for 4 h at 190° C. The mixture was monitored by TLC. The mixture was extracted with EA (30 ml×3) and concentrated under vacuum. The residue was purified by flash with PE/EA=3/1 yield methyl 2-allyl-3-hydroxy-4-nitrobenzoate as yellow oil. ¹H NMR (300 MHz, DMSO-d⁶) δ: 10.74 (s, 1H), 7.95 (d, J=8.7 Hz, 1H), 7.32 (d, J=8.7 Hz, 1H), 5.82-5.95 (m, 1H), 4.92-5.01 (m, 2H), 3.86 (s, 3H), 3.64-3.67 (m, 2H).

### Step 3: Methyl 2-hydroxy-7-nitro-2,3-dihydrobenzofuran-4-carboxylate

Sodium periodate (4972 mg, 23.245 mmol) was added to methyl 2-allyl-3-hydroxy-4-nitrobenzoate (1100 mq, 4.637 mmol) stirred at room temperature in THE (10 ml) and H₂O (10 ml). After 5 min., 4% wt solution of OsO₄ in H₂O (4.4 ml) was added. The resulting mixture was stirred at room temperature for 1 hour. The reaction mixture was washed sequentially with Et₂₀ (10 ml), H₂O (10 ml) then dried over Na₂SO₄. The resulting residue was purified by chromatography on silica gel using cyclohexane/ethyl acetate (3:1) to yield methyl 2-hydroxy-7-nitro-2,3-dihydrobenzofuran-4-carboxylate as a yellow oil. Mass spectrum (ESI, m/z): Calculated for C₁₀H₉NO₆, 238.0 [M−H], Measured: 237.7.

### Step 4: methyl 7-nitrobenzofuran-4-carboxylate

Methyl 2-hydroxy-7-nitro-2,3-dihydrobenzofuran-4-carboxylate (41 mg, 0.171 mmol) was suspended in 85% phosphoric acid (3 mL), stirred for 10 minutes, moved into a 100° C. oil bath and stirred for another 20 minutes. The resulting reaction mixture was added with water (5 mL), then extracted with ethyl acetate (5 mL.×.3). The combined organic phase was washed with saturated sodium carbonate solution (5 mL×3) and saturated sodium chloride solution (5 mL×3) successively, dried over anhydrous sodium sulfate, filtered and the filtrate was concentrated under reduced pressure. The resulting residue was purified by silica gel column chromatography to yield methyl 7-nitrobenzofuran-4-carboxy-late as a yellow solid.

### Step 5: Methyl 7-aminobenzofuran-4-carboxylate

To a solution of methyl 7-nitrobenzofuran-4-carboxylate (100 mg, 0.452 mmol) in EtOH (5 mL) was added Pd/C (100 mg) under H2. The resulting mixture was stirred at 25° C. overnight. The reaction was filtered and concentrated to yield methyl 7-aminobenzofuran-4-carboxylate as a yellow solid. Mass spectrum (ESI, m/z): Calculated for C₁₀H₉NO₃: 192.2 [M+H], Measured: 192.0.

### Step 6: Methyl 7-{[(1Z)-{[(tert-butoxy)carbonyl]amino}({[(tert-butoxy)carbonyl]imino})methyl]amino}-1-benzofuran-4-carboxylate

To a solution of tert-butyl N-[(1E)-[(tert-butoxycarbonyl)amino](methylsulfanyl)methylidene]carbamate (146 mg, 0.502 mmol) in DMF (10 mL) was added methyl 7-aminobenzofuran-4-carboxylate (80 mg, 0.418 mmol). TEA (51 mg, 0.503 mmol) and HgCl₂ (114 mg, 0.421 mmol) was then added. The resulting mixture was stirred at 25° C. overnight. The reaction mixture was washed sequentially with EA (10 ml), H₂O (10 ml) then dried over Na₂SO₄. The resulting residue was purified by chromatography on silica gel using cyclohexane/ethyl acetate (3:1) to yield methyl 7-{[(1Z)-{[(tert-butoxy)carbonyl]amino}({[(tert-butoxy)carbonyl]imino})methyl]amino}-1-benzofuran-4-carboxylate as a yellow oil. Mass spectrum (ESI, m/z): Calculated for C₂₁H₂₇N₃O₇: 433.5[M+H], Measured: 434.1.

### Step 7: 7-(2,3-bis(tert-butoxycarbonyl)guanidino)benzofuran-4-carboxylic acid

To a solution of methyl 7-{[(1Z)-{[(tert-butoxy)carbonyl]amino}({[(tert-butoxy)carbonyl]imino})methyl]amino}-1-benzofuran-4-carboxylate (140 mg, 0.323 mmol) in THE (5 mL) and H₂O (5 mL) was added NaOH (129 mg, 3.23 mmol). The resulting mixture was stirred at 25° C. overnight. The reaction was quenched with HCl (10 mL). The resulting mixture was extracted with EtOAc (3×10 mL). The organic layers were combined, dried over Na₂SO₄, filtered and concentrated to yield (Z)-7-(2,3-bis(tert-butoxycarbonyl)guanidino)benzofuran-4-carboxylic acid as a white solid. Mass spectrum (ESI, m/z): Calculated for C₂₀H₂₅N₃O₇: 420.4[M+H], Measured: 420.3.

### Step 8: 5-[2-(tert-butoxy)-2-oxoethyl]-[1,2,4]triazolo[1,5-a]pyridin-8-yl 7-{[(1Z)-{[(tert-butoxy)carbonyl]amino}({[(tert-butoxy)carbonyl]imino}) methyl]amino}-1-benzofuran-4-carboxylate

To a solution of (Z)-7-(2,3-bis(tert-butoxycarbonyl)guanidino)benzofuran-4-carboxylic acid (140 mg, 0.334 mmol) in DCM (5 mL) was added tert-butyl 2-(8-hydroxy-[1,2,4]triazolo[1,5-a]pyridin-5-yl)acetate (125 mg, 0.5 mmol). EDCI (130 mg, 0.676 mmol), DMAP (81 mg, 0.664 mmol) were then added. The resulting mixture was stirred at 25° C. overnight. The reaction was quenched with H₂O (10 mL). The resulting mixture was extracted with EtOAc (3×10 mL). The organic layers were combined, dried over Na₂SO₄, filtered and concentrated. The residue was purified by silica gel chromatography (0-30% EA/PE) to yield 5-[2-(tert-butoxy)-2-oxoethyl]-[1,2,4]triazolo[1,5-a]pyridin-8-yl 7-{[(1Z)-{[(tert-butoxy)carbonyl]amino}({[(tert-butoxy)carbonyl] imino}) methyl]amino}-1-benzofuran-4-carboxylate as a yellow oil. Mass spectrum (ESI, m/z): Calculated for C₃₂H₃₈N₆O₉: 650.7[M+H], Measured: 651.2.

### Step 9: 2-(8-(4-(3-2-(8-(7-guanidinobenzofuran-4-carbonyloxy)-[1,2,4]triazolo[1,5-a]pyridin-5-yl)acetic acid

Into a 25 ml round-bottom flask, was placed a solution of 5-[2-(tert-butoxy)-2-oxoethyl]-[1,2,4]triazolo[1,5-a]pyridin-8-yl 7-{[(1Z)-{[(tert-butoxy) carbonyl]amino}({[(tert-butoxy)carbonyl]imino})methyl]amino}-1-benzofuran-4-carboxylate (50 mg, 0.077 mmol) in DCM (3 mL), CF₃COOH (2 mL) was added. The resulting solution was stirred for 3 h at room temperature. The reaction progress was monitored by LCMS. The residue was dissolved in DMF. The solution was purified by Prep-HPLC with the following conditions: Column, SunFire Prep C18, 5 um, 19*100 mm; mobile phase, Water of 0.05% CF₃COOH and CH₃CN (10% CH₃CN up to 26% in 8 min, up to 100% in 1 min, down to 20% in 1 min; Detector, 220 nm, 254 nm to yield 2-(8-(7-guanidinobenzofuran-4-carbonyloxy)-[1,2,4]triazolo[1,5-a]pyridin-5-yl)acetic acid as off-white solid.

¹H NMR (400 MHz, DMSO-d⁶) 6:10.37-10.29 (m, 1H), 8.53 (s, 1H), 8.37 (s, 1H), 8.22 (s, 1H), 7.91-7.75 (m, 5H), 7.54 (s, 1H), 7.48-7.46 (m, 1H), 7.33-7.31 (m, 1H), 4.25 (s, 2H). Mass spectrum (ESI, m/z): Calculated for C₁₈H₁₄N₆O₅: 395.3[M+H], Measured: 395.1.

### Example 25: Compound #25

### 3-(8-(4-guanidinobenzoyloxy)-[1,2,4]triazolo[1,5-a]pyridin-6-yl)propanoic acid

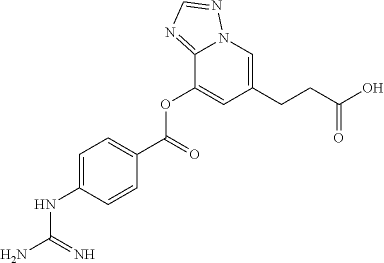 [SMILES: N=C(N)Nc1ccc(C(=O)Oc2cc(CCC(=O)O)cn3ncnc23)cc1]

### Step 1: 3-(benzyloxy)-5-bromopyridin-2-amine

Into a 500-mL round bottle was placed 2-amino-5-bromopyridin-3-ol (5.00 g, 26.454 mmol, 1.00 equiv), Cs₂CO₃ (8.6 g, 26.454 mmol, 1.00 equiv), DMF (50 mL). (Bromomethyl)benzene (4.50 g, 26.454 mmol, 1.00 equiv) was then added. Under N2 atmosphere, the resulting solution was stirred 4 h at 80° C. The reaction was then quenched by H₂O (100 mL). The resulting solution was extracted with EtOAc (150 ml×3) and the organic layers combined and concentrated under vacuum. The residue was purified by silica gel chromatography (0-50% EtOAc/petroleum ether) to yield 3-(benzyloxy)-5-bromopyridin-2-amine as a brown solid. Mass spectrum (ESI, m/z): Calculated for C₁₂H₁₁BrN₂O, 281.0 [M+H], Measured: 281.0.

### Step 2: (E)-N′-(3-(benzyloxy)-5-bromopyridin-2-yl)-N-hydroxyformimidamide

Into a 50-mL round bottle was placed 3-(benzyloxy)-5-bromopyridin-2-amine (1.5 g, 5.374 mmol, 1.00 equiv), DMF-DMA (2.6 g, 21.849 mmol, 4.0 equiv), i-PrOH (20 mL). The resulting solution was stirred 5 h at 100° C. and then cooled down to 55° C. NH₂OH.HCl (1.2 g, 17.391 mmol, 3.0 equiv) and NaHCO₃ (1.4 g, 16.667 mmol, 3.0 equiv) were added. The resulting solution was stirred 16 h at 55° C. The reaction was monitored by LCMS. The resulting solution was concentrated under vacuum. The residue was purified by silica gel chromatography (0-50% EtOAc/petroleum ether) to yield (E)-N′-(3-(benzyloxy)-5-bromopyridin-2-yl)-N-hydroxyformimidamide as a yellow oil. Mass spectrum (ESI, m/z): Calculated for C₁₃H₁₂BrN₃O₂, 322.2[M+H], Measured: 322.1.

### Step 3: 8-(benzyloxy)-5-bromo-[1,2,4]triazolo[1,5-a]pyridine

Into a 100-mL round bottle was placed (E)-N′-(3-(benzyloxy)-5-bromopyridin-2-yl)-N-hydroxyformimidamide (1.70 g, 5.277 mmol, 1.00 equiv), THE (50 ml). TFAA (10 ml) was added at 0° C. The resulting solution was stirred for 16 h at 20° C. The resulting solution was concentrated under vacuum. The residue was purified by silica gel chromatography (0-50% EtOAc/petroleum ether) to yield 8-(benzyloxy)-5-bromo-[1,2,4]triazolo[1,5-a]pyridine as a yellow solid. Mass spectrum (ESI, m/z): Calculated for C₁₃H₁₀BrN₃O, 306.1 [M+H], Measured: 306.0.

### Step 5: (E)-tert-butyl 3-(8-(benzyloxy)-[1,2,4]triazolo[1,5-a]pyridin-6-yl)acrylate

To a solution of 8-(benzyloxy)-6-bromo-[1,2,4]triazolo[1,5-a]pyridine (600 mg, 1.973 mmol, 1.0 equiv) Pd₂(dba)₃ (102 mg, 0.099 mmol, 0.05 equiv), (o-ToI)₃P (60 mg, 0.197 mmol, 0.1 equiv) and TEA (600 mg, 5.918 mmol, 3.0 equiv) in DMF (20 ml) tert-butyl acrylate (1010 mg, 7.89 mmol, 4 equiv) was added. Under N2 atmosphere, the resulting mixture was stirred for 16 h at 110° C. The reaction was monitored by LCMS. The reaction was added H₂O (10 ml). The resulting solution was extracted with EtOAc (20 ml×3) and the organic layers combined and concentrated under vacuum. The residue was purified by silica gel chromatography (0-50% EtOAc/petroleum ether) to yield (E)-tert-butyl 3-(8-(benzyloxy)-[1,2,4]triazolo[1,5-a]pyridin-6-yl)acrylate as a yellow oil. Mass spectrum (ESI, m/z): Calculated for C₂₀H₂₁N₃O₃, 352.3[M+H], Measured: 352.2.

### Step 6: tert-butyl 3-(8-hydroxy-[1,2,4]triazolo[1,5-a]pyridin-6-yl)propanoate

To a solution of (E)-tert-butyl 3-(8-(benzyloxy)-[1,2,4]triazolo[1,5-a]pyridin-6-yl)acrylate (400 mg, 1.138 mmol, 1.00 equiv) in EA (10 mL) was added Pd/C (400 mg). The resulting solution was hydrogenated under 1 atm of H2 at 25° C. for 3 hours. The reaction was monitored by LCMS. The resulting mixture was filtered. The filtrate was concentrated under vacuum. The residue was purified by silica gel chromatography (0-10% EtOAc/petroleum ether) to yield tert-butyl 3-(8-hydroxy-[1,2,4]triazolo[1,5-a]pyridin-6-yl)propanoate as a white solid. Mass spectrum (ESI, m/z): Calculated for C₁₃H₁₇N₃O₃, 264.2 [M+H], Measured: 264.1.

### Step 7: 6-[3-(tert-butoxy)-3-oxopropyl]-[1,2,4]triazolo[1,5-a]pyridin-8-yl 4-{[(1Z)-{[(tert-butoxy)carbonyl]amino}({[(tert-butoxy)carbonyl]imino}) methyl]amino}benzoate

To a solution of tert-butyl 3-(8-hydroxy-[1,2,4]triazolo[1,5-a]pyridin-6-yl)propanoate (100 mg, 0.380 mmol, 1.0 equiv) in DCM (10 ml) were added EDCI (145 mg, 0.76 mmol, 2 equiv), DMAP (92 mg, 0.76 mmol, 2 equiv) and (Z)-4-(2,3-bis(tert-butoxycarbonyl)guanidino)benzoic acid (145 mg, 0.38 mmol, 1.0 equiv). The resulting mixture was stirred for 2 h at 25° C. The reaction was monitored by LCMS. The resulting solution was concentrated under vacuum. The residue was purified by silica gel chromatography (0-50% EtOAc/petroleum ether) to yield 6-[3-(tert-butoxy)-3-oxopropyl]-[1,2,4]triazolo[1,5-a]pyridin-8-yl 4-{[(1Z)-{[(tert-butoxy)carbonyl]amino}({[(tert-butoxy)carbonyl]imino})methyl] amino} benzoate as a yellow oil. Mass spectrum (ESI, m/z): Calculated for C₃₁H₄₀N₆O₈, 624.6 [M+H], Measured: 625.3.

### Step 8: 3-(8-(4-guanidinobenzoyloxy)-[1,2,4]triazolo[1,5-a]pyridin-6-yl)propanoic acid

Into a 50 ml round bottom-flask, 6-[3-(tert-butoxy)-3-oxopropyl]-[1,2,4]triazolo[1,5-a]pyridin-8-yl 4-{[(1Z)-{[(tert-butoxy)carbonyl]amino}({[(tert-butoxy)carbonyl]imino})methyl]amino}benzoate (100 mg, 0.160 mmol, 1.00 equiv) was dissolved in DCM (5 ml). Then TFA (2 ml) was added to the solution. The resulting solution was stirred 3.0 h at room temperature. The reaction was monitored by LCMS. The organic layers were concentrated under vacuum. The residue was purified by Prep-HPLC with the following conditions: (1#waters2767-5): Column, SunFire Prep C18, 19*150 mm 5 umH PrepC-001(T)18600256819513816414 04; mobile phase, Phase A: water with 0.05% TFA; Phase B: CH₃CN (5% CH₃CN up to 14% in 10 min, up to 100% CH₃CN in 1 min, hold 100% in 1 min, down to 14% CH₃CN in 0.1 min, hold 5% in 0.9 min); Detector, UV220 & 254 nm to yield 3-(8-(4-guanidinobenzoyloxy)-[1,2,4]triazolo[1,5-a]pyridin-6-yl)propanoic acid as a white solid.

¹HNMR (400 MHz, DMSO-d⁶) δ 12.26 (brs, 1H), 10.11-10.30 (m, 1H) 8.85 (s, 1H), 8.45 (s, 1H), 8.23 (d, J=8.4 Hz, 2H), 7.98 (s, 4H), 7.72 (s, 1H), 7.48 (d, J=8.4 Hz, 2H), 2.96 (t, J=11.2 Hz, 2H), 2.68 (t, J=7.6 Hz, 2H)./Mass spectrum (ESI, m/z): Calculated for C₁₇H₁₆N₆O₄, 369.3 [M+H], Measured: 369.05.

### Example 26: Compound #26

### 2-(8-(4-guanidinobenzoyloxy)-[1,2,4]triazolo[1,5-a]pyridin-6-yl)acetic acid

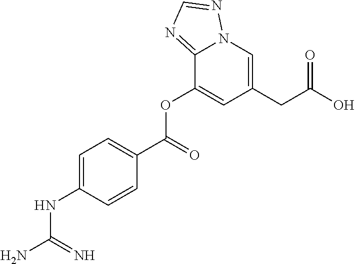 [SMILES: N=C(N)Nc1ccc(C(=O)Oc2cc(CC(=O)O)cn3ncnc23)cc1]

### Step 1: tert-butyl 2-(8-(benzyloxy)-[1,2,4]triazolo[1,5-a]pyridin-6-yl)acetate

To a microwave tube were added 8-(benzyloxy)-6-bromo-[1,2,4]triazolo[1,5-a]pyridine (800 mg, 2.63 mmol, 1.0 equiv) Pd₂(dba)₃ (544 mg, 0.52 mmol, 0.20 equiv), XPhos (500 mg, 1.052 mmol, 0.4 equiv) in THE (15 ml) and (2-tert-butoxy-2-oxoethyl)zinc(II) bromide (4.10 g, 15.8 mmol, 6.00 equiv). The resulting mixture was stirred for 0.5 h at 120° C. The reaction was monitored by LCMS. The reaction was added H₂O (50 ml). The resulting solution was extracted with EtOAc (40 ml×3) and the organic layers combined and concentrated under vacuum. The residue was purified by silica gel chromatography (0-50% EtOAc/petroleum ether) to yield tert-butyl 2-(8-(benzyloxy)-[1,2,4]triazolo[1,5-a]pyridin-6-yl)acetate as a yellow oil. Mass spectrum (ESI, m/z): Calculated for C₁₉H₂₁N₃O₃, 340.3 [M+H], Measured: 340.1.

### Step 2: tert-butyl 2-(8-hydroxy-[1,2,4]triazolo[1,5-a]pyridin-6-yl)acetate

To a solution of tert-butyl 2-(8-(benzyloxy)-[1,2,4]triazolo[1,5-a]pyridin-6-yl)acetate (400 mg, 1.179 mmol, 1.0 equiv) in EA (10 mL) was added Pd/C (400 mg). Then the resulting solution was through H₂ at 25° C. The resulting mixture was stirred for 2 h at 25° C. The reaction was monitored by LCMS. The resulting mixture was filtered. The filtrate was concentrated under vacuum. The residue was purified by silica gel chromatography (0-10% EtOAc/petroleum ether) to yield tert-butyl 2-(8-hydroxy-[1,2,4]triazolo[1,5-a]pyridin-6-yl)acetate as a white solid. Mass spectrum (ESI, m/z): Calculated for C₁₂H₁₅N₃O₃, 250.2[M+H], Measured: 250.1.

### Step 3: 6-[2-(tert-butoxy)-2-oxoethyl]-[1,2,4]triazolo[1,5-a]pyridin-8-yl 4-{[(1Z)-{[(tert-butoxy)carbonyl]amino}({[(tert-butoxy)carbonyl]imino})methyl]amino}benzoate

To a solution of tert-butyl 2-(8-hydroxy-[1,2,4]triazolo[1,5-a]pyridin-6-yl)acetate (100 mg, 0.401 mmol, 1.0 equiv) in DCM (10 ml) were added EDCI (150 mg, 0.802 mmol, 2 equiv), DMAP (100 mg, 0.802 mmol, 1.2 equiv) and (Z)-4-(2,3-bis(tert-butoxycarbonyl)guanidino)benzoic acid (150 mg, 0.41 mmol, 1.0 equiv) was added. The resulting mixture was stirred for 2 h at 25° C. The reaction was monitored by LCMS. The resulting solution was concentrated under vacuum. The residue was purified by silica gel chromatography (0-50% EtOAc/petroleum ether) to yield 6-[2-(tert-butoxy)-2-oxoethyl]-[1,2,4]triazolo[1,5-a]pyridin-8-yl 4-{[(1Z)-{[(tert-butoxy)carbonyl]amino}({[(tert-butoxy)carbonyl]imino})methyl]amino}benzoate as a yellow oil. Mass spectrum (ESI, m/z): Calculated for C₃₀H₃₈N₆O₈, 611.2[M+H], Measured: 611.3.

### Step 4: 2-(8-(4-guanidinobenzoyloxy)-[1,2,4]triazolo[1,5-a]pyridin-6-yl)acetic acid

Into a 50 ml round bottom-flask, 6-[2-(tert-butoxy)-2-oxoethyl]-[1,2,4]triazolo[1,5-a]pyridin-8-yl 4-{[(1Z)-{[(tert-butoxy)carbonyl]amino}({[(tert-butoxy)carbonyl]imino})methyl]amino}benzoate (100 mg, 0.164 mmol, 1.00 equiv) was dissolved in DCM (5 ml). Then TFA (2 ml) was added to the solution. The resulting solution was stirred 3.0 h at room temperature. The reaction was monitored by LCMS. The organic layers were concentrated under vacuum. The residue was purified by Prep-HPLC with the following conditions: (1#waters2767-5): Column, SunFire Prep C18, 19*150 mm 5 umH PrepC-001(T)18600256819513816414 04; mobile phase, Phase A: water with 0.05% TFA; Phase B: CH₃CN (5% CH₃CN up to 14% in 10 min, up to 100% CH₃CN in 1 min, hold 100% in 1 min, down to 14% CH₃CN in 0.1 min, hold 5% in 0.9 min); Detector, UV220 & 254 nm to yield 2-(8-(4-guanidinobenzoyloxy)-[1,2,4]triazolo[1,5-a]pyridin-6-yl)acetic acid as a white solid.

¹HNMR (400 MHz, DMSO-d⁶) δ 10.25 (s, 1H), 8.92 (s, 1H), 8.48 (s, 1H), 8.23 (d, J=8.8 Hz, 2H), 7.84 (s, 4H), 7.68 (s, 1H), 7.48 (d, J=8.4 Hz, 2H), 3.81 (s, 2H). Mass spectrum (ESI, m/z): Calculated for C₁₆H₁₄N₆O₄, 355.3[M+H], Measured: 355.05.

### Example 27: Compound #27

### 2-(2-(8-(4-guanidinobenzoyloxy)imidazo[1,2-a]pyridin-5-yl) acetamido)succinic acid

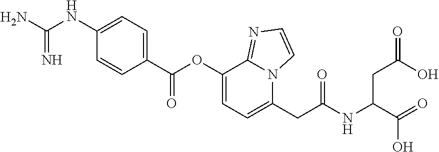 [SMILES: N=C(N)Nc1ccc(C(=O)Oc2ccc(CC(=O)NC(CC(=O)O)C(=O)O)n3ccnc23)cc1]

### Step 1: Tert-butyl 2-(8-(benzyloxy)imidazo[1,2-a]pyridin-5-yl)acetate

To a solution of 8-(benzyloxy)-5-bromoimidazo[1,2-a]pyridine (400.000 mg, 1.319 mmol), Pd₂(dba)₃ (300 mg) and Q-Phos (30 mg) in THE (12 ml) was added (2-tert-butoxy-2-oxoethyl)zinc(II) bromide (1.375 g, 5.278 mmol). The resulting solution was stirred for 40 min at 110° C. under microwave. The reaction was monitored by LCMS. Water was added to the reaction and the solution was extracted with EA (50 mL×4). The organic phases were dried by Na₂SO₄ and evaporated at reduced pressure. The residue was purified by silica gel chromatography (40-60% EtOAc/petroleum ether) to yield tert-butyl 2-(8-(benzyloxy)imidazo[1,2-a]pyridin-5-yl)acetate as a red solid. Mass spectrum (ESI, m/z): Calculated for C₂₀H₂₂N₂O₃, 399.4 [M+H], Measured: 399.2.

### Step 2: 2-(8-(benzyloxy)imidazo[1,2-a]pyridin-5-yl)acetic acid

To a solution of tert-butyl 2-(8-(benzyloxy)imidazo[1,2-a]pyridin-5-yl)acetate (180 mg, 0.532 mmol) in DCM (5 mL) was added trifluoroacetic acid (2.0 ml). The solution was stirred 4.0 h at room temperature. The reaction was monitored by LCMS. The resulting mixture was concentrated under vacuum to yield 2-(8-(benzyloxy)imidazo[1,2-a]pyridin-5-yl)acetic acid as a brown solid. Mass spectrum (ESI, m/z): Calculated for C₁₆H₁₄N₂O₃, 283.3 [M+H], Measured: 283.1.

### Step 3: Di-tert-butyl 2-(2-(8-(benzyloxy)imidazo[1,2-a]pyridin-5-yl) acetamido)succinate

To a solution of 2-(8-(benzyloxy)imidazo[1,2-a]pyridin-5-yl)acetic acid (290.000 mg, 1.027 mmol) and di-tert-butyl 2-aminosuccinate (302.414 mg, 1.233 mmol) in DCM (20 ml), was added HATU (781.219 mg, 2.055 mmol) and TEA (311.857 mg, 3.082 mmol). The resulting solution was stirred for 2 h at room temperature. The reaction was then evaporated in vacuo and the residue was purified by silica gel chromatography (40-60% EtOAc/petroleum ether) to yield di-tert-butyl 2-(2-(8-(benzyloxy)imidazo[1,2-a]pyridin-5-yl)acetamido)succinate as a yellow oil. Mass spectrum (ESI, m/z): Calculated for C₂₈H₃₅N₃O₆, 509.6 [M+H], Measured: 510.3.

### Step 4: Di-tert-butyl 2-(2-(8-hydroxyimidazo[1,2-a]pyridin-5-yl) acetamido)succinate

To a solution of di-tert-butyl 2-(2-(8-(benzyloxy)imidazo[1,2-a]pyridin-5-yl)acetamido)succinate (400 mg, 0.683 mmol) in EA (15 ml) was added Pd/C (400 mg). The resulting solution was stirred 4.0 h under hydrogen (30 atm) at 80° C. The reaction was filtrated, evaporated in vacuo to yield di-tert-butyl 2-(2-(8-hydroxyimidazo[1,2-a]pyridin-5-yl)acetamido)succinate as a colorless oil. Mass spectrum (ESI, m/z): Calculated for C₂₁H₂₉N₃O₆, 420.5 [M+H], Measured: 420.5.

### Step 5: 1,4-di-tert-butyl 2-{2-[8-(4-{[(1Z)-{[(tert-butoxy)carbonyl]amino} ({[(tert-butoxy)carbonyl]imino})methyl]amino}benzoyloxy)imidazo[1,2-a]pyridin-5-yl]acetamido}butanedioate

To a solution of di-tert-butyl 2-(2-(8-hydroxyimidazo[1,2-a]pyridin-5-yl)acetamido)succinate (180.000 mg, 0.429 mmol) and (Z)-4-(2,3-bis(tert-butoxycarbonyl)guanidino)benzoic acid (162.808 mg, 0.429 mmol) in DCM (10 ml), was added EDCI (164.523 mg, 0.858 mmol) and DMAP (104.847 mg, 0.858 mmol). The resulting solution was stirred for 2 h at room temperature. The reaction was monitored by LCMS. The reaction was then evaporated in vacuo and the residue was purified by silica gel chromatography (40-60% EtOAc/petroleum ether) to yield 1,4-di-tert-butyl 2-{2-[8-(4-{[(1Z)-{[(tert-butoxy)carbonyl]amino}({[(tert-butoxy)carbonyl]imino}) methyl]amino}benzoyloxy)imidazo[1,2-a]pyridin-5-yl]acetamido}butanedioate as a white solid. Mass spectrum (ESI, m/z): Calculated for C₃₉H₅₂N₆O₁₁, 780.9 [M+H], Measured: 781.4.

### Step 6: 2-(2-(8-(4-guanidinobenzoyloxy)imidazo[1,2-a]pyridin-5-yl) acetamido)succinic acid

To a solution of 2-(1-(4-(2,3-bis(tert-butoxycarbonyl)guanidino)benzoyl)-4,5,6,7-tetrahydro-1H-indazol-3-yl)acetic acid (140 mg, 0.179 mmol) in DCM (6 mL) was added trifluoroacetic acid (2.0 ml). The solution was stirred 2.0 h at room temperature. The reaction was monitored by LCMS. The resulting mixture was concentrated under vacuum. The residue was purified by Flash-Prep-HPLC with the following conditions (IntelFlash-1): Column, C18; mobile phase, Water with 0.05% HCl/CH₃CN=2/98 increasing to H₂O with 0.05% HCl/CH₃CN=30/70; Detector, UV 220 & 254 nm to yield 2-(2-(8-(4-guanidinobenzoyloxy)imidazo[1,2-a]pyridin-5-yl)acetamido)succinic acid as a white solid.

¹H NMR (300 MHz, DMSO-d⁶) δ: 12.81 (brs, 1H), 10.67 (s. 1H), 8.97 (s, 1H), 8.26 (d, J=6.6 Hz, 4H), 7.94-8.25 (m, 5H), 7.50 (d, J=6.6 Hz, 2H), 7.40 (s, 1H), 4.56-4.61 (m, 1H), 4.25 (s, 2H), 2.67-2.80 (m, 2H). Mass spectrum (ESI, m/z): Calculated for C₂₁H₂₀N₆O₇, 469.4 [M+H], Measured: 469.1.

### Example 28: Compound #28

### 2-(2-(8-(2-guanidinopyrimidine-5-carbonyloxy)-[1,2,4]triazolo[1,5-a]pyridin-5-yl)acetamido)succinic acid

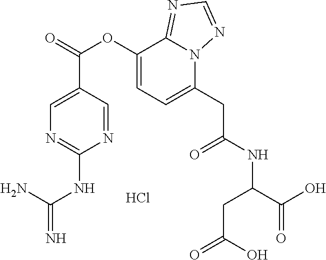 [SMILES: N=C(N)Nc1ncc(C(=O)Oc2ccc(CC(=O)NC(CC(=O)O)C(=O)O)n3ncnc23)cn1 ; Cl]

### Step 1: di-tert-butyl 2-(2-(8-(benzyloxy)-[1,2,4]triazolo[1,5-a]pyridin-5-yl)acetamido)succinate

To a solution of 2-(8-(benzyloxy)-[1,2,4]triazolo[1,5-a]pyridin-5-yl)acetic acid (400 mg, 1.412 mmol, 1.0 equiv) in DCM (10 ml) and DMF (2 ml) were added Et₃N (430 mg, 4.236 mmol, 3.0 equiv), HATU (1.1 g, 2.824 mmol, 2 equiv) and di-tert-butyl 2-aminosuccinate (415 mg, 1.694 mmol, 1.2 equiv). The resulting mixture was stirred for 2 h at 25° C. The reaction was monitored by LCMS. The reaction was washed by H₂O (10*3) and dried by N2SO₄. The organic layers were concentrated under vacuum. The residue was purified by silica gel chromatography (0-50% EtOAc/petroleum ether) to yield di-tert-butyl 2-(2-(8-(benzyloxy)-[1,2,4]triazolo[1,5-a]pyridin-5-yl)acetamido)succinate as a yellow oil. Mass spectrum (ESI, m/z): Calculated for C₂₇H₃₄N₄O₆, 510.5[M+H], Measured: 511.2.

### Step 2: di-tert-butyl 2-(2-(8-hydroxy-[1,2,4]triazolo[1,5-a]pyridin-5-yl)acetamido)succinate

To a solution of di-tert-butyl 2-(2-(8-(benzyloxy)-[1,2,4]triazolo[1,5-a]pyridin-5-yl)acetamido)succinate (500 mg, 0.979 mmol, 1.0 equiv) in EA (10 mL) was added Pd/C (500 mg). The resulting solution was through H2 at 25° C. The resulting mixture was stirred for 3 h at 25° C. The reaction was monitored by LCMS. The resulting mixture was filtered. The filtrate was concentrated under vacuum. The residue was purified by silica gel chromatography (0-10% EtOAc/petroleum ether) to yield di-tert-butyl 2-(2-(8-hydroxy-[1,2,4]triazolo[1,5-a]pyridin-5-yl)acetamido)succinate as a yellow oil. Mass spectrum (ESI, m/z): Calculated for C₂₀H₂₈N₄O₆, 421.4[M+H], Measured: 421.2.

### Step 3: 1,4-di-tert-butyl 2-{2-[8-({2-[({[(tert-butoxy)carbonyl]amino}methanimidoyl)amino]pyrimidin-5-yl}carbonyloxy)-[1,2,4]triazolo[1,5-a]pyridin-5-yl]acetamido}butanedioated

To a solution of di-tert-butyl 2-(2-(8-hydroxy-[1,2,4]triazolo[1,5-a]pyridin-5-yl)acetamido)succinate (200 mg, 0.476 mmol, 1.0 equiv) in DCM (5 ml) and DMF (3 ml) were added EDCI 180 mg, 0.951 mmol, 2.0 equiv), DMAP (120 mg, 0.951 mmol, 2 equiv) and 2-(3-(tert-butoxycarbonyl)guanidino)pyrimidine-5-carboxylic acid (135 mg, 0.476 mmol, 1.0 equiv). The resulting mixture was stirred for 2 h at 25° C. The reaction was monitored by LCMS. The reaction was washed by H₂O (10 ml×3) and dried by N2SO₄. The organic layers was concentrated under vacuum. The residue was purified by Prep-HPLC with the following conditions: (1#waters2767-5): Column, SunFire Prep C18, 19*150 mm 5 umH PrepC-001(T)18600256819513816414 04; mobile phase, Phase A: water with 0.05% TFA; Phase B: CH₃CN (28% CH₃CN up to 52% in 10 min, up to 100% CH₃CN in 1 min, hold 100% in 1 min, down to 28% CH₃CN in 0.1 min, hold 5% in 0.9 min); Detector, UV220 & 254 nm to yield 1,4-di-tert-butyl 2-{2-[8-({2-[({[(tert-butoxy)carbonyl]amino}methanimidoyl)amino]pyrimidin-5-yl}carbonyloxy)-[1,2,4]triazolo[1,5-a]pyridin-5-yl]acetamido}butanedioated as a yellow oil. Mass spectrum (ESI, m/z): Calculated for C₃₁H₄₁N₉O₉, 584.2 [M-Boc+H], Measured: 584.2.

### Step 4: 2-(2-(8-(2-guanidinopyrimidine-5-carbonyloxy)-[1,2,4]triazolo[1,5-a]pyridin-5-yl)acetamido)succinic acid

Into a 50 ml round bottom-flask, 1,4-di-tert-butyl 2-{2-[8-({2-[({[(tert-butoxy)carbonyl]amino}methanimidoyl)amino]pyrimidin-5-yl}carbonyloxy)-[1,2,4]triazolo[1,5-a]pyridin-5-yl]acetamido}butanedioate (100 mg, 0.146 mmol, 1.00 equiv) was dissolved in DCM (6 ml). Then TFA (3 ml) was added to the solution. The resulting solution was stirred 2.0 h at 25° C. The reaction was monitored by LCMS. The organic layers were concentrated under vacuum. The residue was purified by Flash-Prep-HPLC with the following conditions (IntelFlash-1): Column, C18; mobile phase, water of 0.1% HCl/CH₃CN=2/98 increasing to water of 0.1% HCl/CH₃CN=20/80 within 30 min; Detector, UV 220 & 254 nm to yield 2-(2-(8-(2-guanidinopyrimidine-5-carbonyloxy)-[1,2,4]triazolo[1,5-a]pyridin-5-yl)acetamido)succinic acid as a white solid.

¹HNMR (400 MHz, DMSO-d⁶) δ 11.77 (s, 1H), 9.38 (s, 2H), 8.73 (d, J=8.0 Hz, 1H), 8.45-8.56 (m, 5H), 7.75 (d, J=7.6 Hz 1H), 7.27 (d, J=8.0 Hz, 1H), 4.56-4.61 (m, 1H), 4.17 (s, 2H), 2.50-2.77 (m, 2H). Mass spectrum (ESI, m/z): Calculated for C₁₈H₁₇N₉O₇, 472.3 [M+H], Measured: 472.0.

### Example 29: Compound #29

### 2-(8-(2-guanidinopyrimidine-5-carbonyloxy)imidazo[1,2-a]pyridin-5-yl)acetic acid

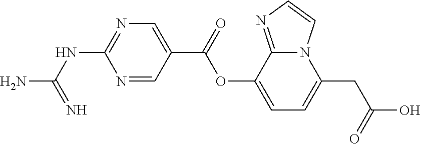 [SMILES: N=C(N)Nc1ncc(C(=O)Oc2ccc(CC(=O)O)n3ccnc23)cn1]

### Step 1: 2-(3-(tert-butoxycarbonyl)guanidino)pyrimidine-5-carboxylic acid

Into a 50 ml flask was placed a solution of tert-butyl N-carbamimidoyl carbamate (512 mg, 3.216 mmol, 1.2 equiv.) in DMF (15 ml). NaH (193 mg, 8.039 mmol, 3 equiv.) was then added. The mixture was stirred for 1 h and ethyl 2-chloropyrimidine-5-carboxylate (500 mg, 2.680 mmol, 1 equiv.) was added. The mixture was stirred for 3 h at 25° C. The reaction was monitored by LCMS. The reaction was quenched with water (10 ml) and stirred for 16 h at 25° C. The mixture was concentrated under vacuum and purified by flash with H₂O/CH₃CN=10/1 to yield 2-(3-(tert-butoxycarbonyl)guanidino) pyrimidine-5-carboxylic acid as white solid. Mass spectrum (ESI, m/z): Calculated for C₁₁H₁₅N₅O₄: 282.1 [M+H], Measured: 282.1.

### Step 2: 5-[2-(tert-butoxy)-2-oxoethyl]imidazo[1,2-a]pyridin-8-yl 2-[({[(tert-butoxy)carbonyl]amino}metha-nimidoyl)amino]pyrimidine-5-carboxylate

Into a 50 ml flask was placed a solution of tert-butyl 2-(8-hydroxy imidazo[1,2-a]pyridin-5-yl)acetate (160 mg, 0.644 mmol, 1 equiv.) in DCM (10 ml). 2-(3-(tert-Butoxycarbonyl)guanidino)pyrimidine-5-carboxylic acid (181 mg, 0.644 mmol, 1 equiv.), EDCI (247 mg, 1.289 mmol, 2 equiv.), DMAP (157 mg, 1.289 mmol, 2 equiv.) were added. The mixture was stirred for 2 h at 25° C. The reaction was monitored by LCMS. The resulting mixture was concentrated under vacuum and purified by flash with CH₃CN/H₂O (2-60%) to yield 5-[2-(tert-butoxy)-2-oxoethyl]imidazo[1,2-a]pyridin-8-yl 2-[({[(tert-butoxy)carbonyl]-amino}methanimidoyl)amino]pyrimidine-5-carboxylate as white solid. Mass spectrum (ESI, m/z): Calculated for C₂₄H₂₉N₇O₆: 512.2[M+H], Measured: 512.2.

### Step 3: 2-(8-(2-guanidinopyrimidine-5-carbonyloxy)imidazo[1,2-a]pyridin-5-yl)acetic acid

Into a 50 ml flask was placed a solution of 5-[2-(tert-butoxy)-2-oxoethyl]imidazo[1,2-a]pyridin-8-yl 2-[({[(tert-butoxy)carbonyl]amino}methanimidoyl)amino]pyrimidine-5-carboxylate (100 mg, 0.195 mmol, 1 equiv.) in DCM (3 ml). TFA (3 ml) was then added. The mixture was stirred for 3 h at 25° C. The mixture was purified by C18 reverse phase column with the following conditions: mobile phase, Water of 0.05% TFA and CH₃CN (2% CH₃CN up to 15% in 30 min, up to 100% in 5 min, down to 2% in 5 min, Detector, 280 nm, 254 nm to yield 2-(8-(2-guanidinopyrimidine-5-carbonyloxy)imidazo[1,2-a]pyridin-5-yl)acetic acid hydrochloride as white solid.

¹H NMR (400 MHz, DMSO-d⁶) δ: 11.59 (s, 1H), 9.38 (s, 2H), 8.39-8.45 (m, 5H), 8.09 (s, 1H), 7.99 (s, 1H), 7.45-7.55 (m, 1H), 7.05-7.15 (m, 1H), 4.23 (s, 2H). Mass spectrum (ESI, m/z): Calculated for C₁₅H₁₃N₇O₄: 356.1[M+H], Measured: 356.0.

### Example 30: Compound #30

### 5-(2-(methylsulfonamido)-2-oxoethyl)-[1,2,4]triazolo[1,5-a]pyridin-8-yl 2-guanidinopyrimidine-5-carboxylate

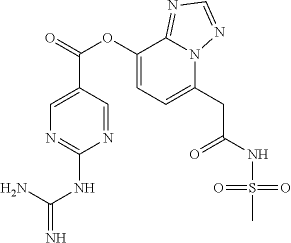 [SMILES: CS(=O)(=O)NC(=O)Cc1ccc(OC(=O)c2cnc(NC(=N)N)nc2)c2ncnn12]

### Step 1: 5-[(methanesulfonylcarbamoyl)methyl]-[1,2,4]triazolo[1,5-a]pyridin-8-yl 2-[({[(tert-butoxy)carbonyl]amino}methanimidoyl)amino] pyrimidine-5-carboxylate

To a solution of 2-(8-hydroxy-[1,2,4]triazolo[1,5-a]pyridin-5-yl)-N-(methylsulfonyl)acetamide (100 mg, 0.37 mmol, 1.0 equiv) in DCM (5 ml) and DMF (3 ml) were added EDCI (140 mg, 0.74 mmol, 2.0 equiv), DMAP (90 mg, 0.74 mmol, 2 equiv) and 2-(3-(tert-butoxycarbonyl)guanidino)pyrimidine-5-carboxylic acid (105 mg, 0.37 mmol, 1.0 equiv) was added. The resulting mixture was stirred for 2 h at 25° C. The reaction was monitored by LCMS. The reaction was washed by H₂O (10*3) and dried by N2SO₄. The organic layers was concentrated under vacuum. The residue was purified by Prep-HPLC with the following conditions: (1#waters2767-5): Column, SunFire Prep C18, 19*150 mm 5 umH PrepC-001(T)18600256819513816414 04; mobile phase, Phase A: water with 0.05% TFA; Phase B: CH₃CN (28% CH₃CN up to 52% in 10 min, up to 100% CH₃CN in 1 min, hold 100% in 1 min, down to 28% CH₃CN in 0.1 min, hold 5% in 0.9 min); Detector, UV220 & 254 nm to yield 5-[(methanesulfonylcarbamoyl) methyl]-[1,2,4]triazolo[1,5-a]pyridin-8-yl 2-[({[(tert-butoxy)carbonyl]amino} methanimidoyl)amino]pyrimidine-5-carboxylate as a yellow oil. Mass spectrum (ESI, m/z): Calculated for C₂₀H₂₃N₉O₇S, 433.5[M+H-Boc], Measured: 434.1.

### Step 2: 5-(2-(methylsulfonamido)-2-oxoethyl)-[1,2,4]triazolo[1,5-a]pyridin-8-yl 2-guanidinopyrimidine-5-carboxylate

Into a 50 ml round bottom-flask, 5-[(methanesulfonylcarbamoyl)methyl]-[1,2,4]triazolo[1,5-a]pyridin-8-yl 2-[({[(tert-butoxy)carbonyl]amino}methan imidoyl) amino]pyrimidine-5-carboxylate (80 mg, 0.150 mmol, 1.00 equiv) was dissolved in DCM (5 ml). Then TFA (2 ml) was added to the solution. The resulting solution was stirred 2.0 h at 25° C. The reaction was monitored by LCMS. The organic layers were concentrated under vacuum. The residue was purified by Flash-Prep-HPLC with the following conditions (IntelFlash-1): Column, C18; mobile phase, water of 0.1% HCl/CH₃CN=2/98 increasing to water of 0.1% HCl/CH₃CN=20/80 within 30 min; Detector, UV 220 & 254 nm to yield 5-(2-(methylsulfonamido)-2-oxoethyl)-[1,2,4]triazolo[1,5-a]pyridin-8-yl 2-guanidinopyrimidine-5-carboxylate as a white solid.

¹HNMR (400 MHz, DMSO-d⁶) δ: 9.47 (brs, 2H), 8.56 (s, 5H), 7.61-7.93 (m, 1H), 7.26-7.58 (m, 1H), 6.94-7.27 (m, 1H), 4.34 (s, 2H), 3.26 (s, 3H). Mass spectrum (ESI, m/z): Calculated for C₁₅H₁₅N₉O₅S, 434.4 [M+H], Measured: 434.0.

### Example 31: Compound #31

### 2-(2-(8-(2-guanidinopyrimidine-5-carbonyloxy)imidazo[1,2-a] pyridin-5-yl)acetamido)succinic acid

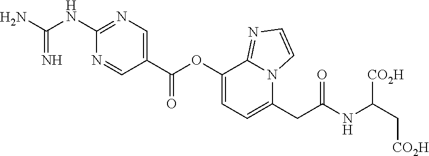 [SMILES: N=C(N)Nc1ncc(C(=O)Oc2ccc(CC(=O)NC(CC(=O)O)C(=O)O)n3ccnc23)cn1]

### Step 1: 1,4-di-tert-butyl-2-{2-[8-(2-carbamimidamidopyrimidine-5-carbonyloxy)imidazo[1,2-a]pyridin-5-yl]acetamido}butanedioate

Into a 50 ml flask was placed a solution of di-tert-butyl 2-(2-(8-hydroxyimidazo[1,2-a]pyridin-5-yl)acetamido)succinate (240 mg, 0.572 mmol, 1 equiv.) in DMF (5 ml). 2-(3-(tert-Butoxycarbonyl)guanidino)pyrimidine-5-carboxylic acid (177 mg, 0.629 mmol, 1.1 equiv.), EDCI (548 mg, 2.861 mmol, 5 equiv.), DMAP (349 mg, 2.861 mmol, 5 equiv.) were then added. The mixture was stirred for 2 h at 25° C. The mixture was extracted with DCM and washed with brine. The organic layers was concentrated under vacuum and purified by TLC with DCM/MeOH=35/1 to yield 1,4-di-tert-butyl 2-{2-[8-(2-carbamimidamidopyrimidine-5-carbonyloxy)imidazo[1,2-a]pyridin-5-yl]acetamido}-butanedioate as yellow solid. Mass spectrum (ESI, m/z): Calculated for C₂₇H₃₄N₈O₇: 583.2[M+H], Measured: 583.2.

### Step 2: 2-(2-(8-(2-guanidinopyrimidine-5-carbonyloxy)imidazo[1,2-a] pyridin-5-yl)acetamido)succinic acid

Into a 50 ml flask was placed a solution of 1,4-di-tert-butyl 2-[2-(8-{2-[({[(tert-butoxy)carbonyl]amino}methanimidoyl)amino]pyrimidine-5-carbonyloxy}imidazo[1,2-a]pyridin-5-yl)acetamido]butanedioate (150 mg, 0.220 mmol, 1 equiv.) in DCM (4 ml). TFA (3 ml) was then added. The mixture was stirred for 2 h at 25° C. The reaction was monitored by LCMS. The mixture was purified by reversed phase liquid chromatography with the following conditions: Column, C18, 120 g; mobile phase, Water of 0.1% HCl and CH₃CN (2% CH₃CN up to 15% in 30 min, up to 100% in 5 min, down to 2% in 5 min; Detector, 220 nm, 254 nm to yield 2-(2-(8-(2-guanidinopyrimidine-5-carbonyloxy)imidazo[1,2-a]pyridin-5-yl)acetamido)succinic acid hydrochloride as white solid.

¹H NMR (400 MHz, DMSO-d6) δ: 11.95 (s, 1H), 9.40 (s, 2H), 8.90-9.10 (m, 1H), 8.50-8.90 (m, 4H), 8.30-8.45 (m, 2H), 8.00-8.10 (m, 1H), 7.45-7.55 (m, 1H), 4.50-4.60 (m, 1H), 4.29 (s, 2H), 2.60-2.80 (m, 2H). Mass spectrum (ESI, m/z): Calculated for C₁₉H₁₈N₈O₇. HCl: 471.1[M+H], Measured: 471.1.

### Example 32: Compound #32

### 2-(8-(4-(2-iminoimidazolidin-1-yl)benzoyloxy)-[1,2,4]triazolo[1,5-a]pyridin-5-yl)acetic acid

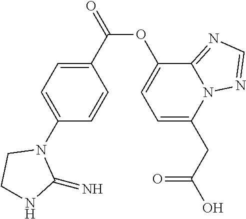 [SMILES: N=C1NCCN1c1ccc(C(=O)Oc2ccc(CC(=O)O)n3ncnc23)cc1]

### Step 1: 4-(2-(tert-butoxycarbonylimino)imidazolidin-1-yl)benzoic acid

Into a 50 ml flask was placed a solution of methyl 4-(2-(tert-butoxy carbonylimino)imidazolidin-1-yl)benzoate (380 mg, 1.190 mmol, 1.00 equiv.) in THF/H₂O (6 ml/2 ml). LiOH (570 mg, 23.798 mmol, 20 equiv.) was then added. The mixture was stirred for 16 h at 25° C. The mixture was concentrated under vacuum. The residue was purified by Prep-HPLC with the following conditions: Column, SunFire Prep C18, 5 um, 19*100 mm; mobile phase, Water of 0.05% H₂O and CH₃CN (10% CH₃CN up to 22% in 10 min, up to 100% in 2 min, down to 10% in 2 min; Detector, 220 nm, 254 nm to yield 4-(2-(tert-butoxycarbonyl imino)imidazolidin-1-yl)benzoic acid as white solid. Mass spectrum (ESI, m/z): Calculated for C₁₅H₁₉N₃O₄: 306.3 [M+H], Measured: 306.2.

### Step 2: (Z)-5-(2-tert-butoxy-2-oxoethyl)-[1,2,4]triazolo[1,5-a]pyridin-8-yl 4-(2-(tert-butoxycarbonylimino)-imidazolidin-1-yl)benzoate

Into a 50 ml flask was placed a solution of 4-(2-(tert-butoxycarbonyl imino)imidazolidin-1-yl)benzoic acid (120 mg, 0.152 mmol, 1.00 equiv.) in DCM (10 ml). tert-Butyl 2-(8-hydroxy-[1,2,4]triazolo[1,5-a]pyridin-5-yl)acetate (47 mg, 0.189 mmol, 1.30 equiv.), EDCI (63 mg, 0.328 mmol, 2.00 equiv.), DMAP (36 mg, 0.294 mmol, 2.00 equiv.) were then added. The mixture was stirred for 16 h at 25° C. The reaction was monitored by LCMS. The mixture was concentrated under vacuum and purified by TLC with PE/EA=2/1 to yield (Z)-5-(2-tert-butoxy-2-oxoethyl)-[1,2,4]triazolo[1,5-a]pyridin-8-yl 4-(2-(tert-butoxycarbonyl imino)imidazolidin-1-yl)benzoate as white solid. Mass spectrum (ESI, m/z): Calculated for C₂₇H₃₂N₆O₆: 537.2[M+H], Measured: 537.6.

### Step 3: 2-(8-(4-(2-iminoimidazolidin-1-yl)benzoyloxy)-[1,2,4]triazolo[1,5-a]pyridin-5-yl)acetic acid

Into a 50 ml flask was placed a solution of the mixture of 5-(2-tert-butoxy-2-oxoethyl)-[1,2,4]triazolo[1,5-a]pyridin-8-yl 4-(2-(tert-butoxycarbonyl imino)imidazolidin-1-yl)benzoate (70 mg, 0.130 mmol, 1.00 equiv.) in DCM (5 ml). TFA (3 ml) was then added. The mixture was stirred for 2 h at 25° C. The reaction was monitored by LCMS. The mixture was purified by Prep-HPLC with the following conditions: Column, SunFire Prep C18, 5 um, 19*100 mm; mobile phase, Water of 0.05% TFA and CH₃CN (8% CH₃CN up to 22% in 10 min, up to 100% in 2 min, down to 10% in 2 min; Detector, 220 nm, 254 nm to yield 2-(8-(4-(2-iminoimidazolidin-1-yl)benzoyloxy)-[1,2,4]triazolo[1,5-a]pyridin-5-yl)acetic acid as white solid.

¹H NMR (300 MHz, DMSO-d⁶) δ: 12.81 (s, 1H), 8.62-8.75 (m, 1H), 8.52 (s, 1H), 8.30 (d, J=8.7 Hz, 4H), 7.74 (d, J=7.8 Hz, 1H), 7.64 (d, J=8.7 Hz, 2H), 7.30 (d, J=7.8 Hz, 1H), 4.15-4.25 (m, 4H), 3.69-3.80 (m, 2H). Mass spectrum (ESI, m/z): Calculated for C₁₈H₁₆N₆O₄: 381.1 [M+H], Measured: 381.1.

### Example 33: Compound #33

### 2-(8-(4-(N-methylcarbamimidoyl)benzoyloxy)-[1,2,4]triazolo[1,5-a]pyridin-5-yl)acetic acid

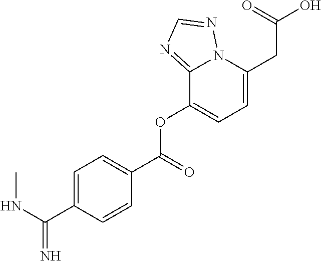 [SMILES: CNC(=N)c1ccc(C(=O)Oc2ccc(CC(=O)O)n3ncnc23)cc1]

### Step 1: Methyl 4-(ethoxy(imino)methyl)benzoate

Into a 100-mL round-bottom flask, to a solution of methyl 4-cyano benzoate (2.00 g, 12.410 mmol) was added in HCl/EtOH (20 ml). The resulting mixture was stirred at room temperature overnight. The reaction progress was monitored by LCMS. The resulting mixture was concentrated under vacuum to yield methyl 4-(ethoxy(imino)methyl)benzoate as a white solid. Mass spectrum (ESI, m/z): Calculated for C₁₁H₁₃NO₃, 208.1 [M+H], Measured: 208.2.

### Step 2: Methyl 4-(N-methylcarbamimidoyl)benzoate

Into a 100-mL round-bottom flask, was placed methyl 4-(ethoxy(imino) methyl)benzoate (200 mg, 0.965 mmol, 1.00 equiv), EtOH (5 ml), methanamine (150 mg, 4.826 mmol, 2.00 equiv). The reaction was stirred 3 h at 25° C. The reaction progress was monitored by LCMS. The mixture was concentrated to yield methyl 4-(N-methylcarbamimidoyl)benzoate as a light yellow oil. Mass spectrum (ESI, m/z): Calculated for C₁₀H₁₂N₂O₂, 193.1 [M+H], Measured: 193.0.

### Step 3: (Z)-methyl 4-(N,N′-bis(tert-butoxycarbonyl)-N-methylcarbamimidoyl)benzoate

Into a 100-mL round-bottom flask, was placed methyl 4-(N-methyl carbamimidoyl)benzoate (150 mg, 0.780 mmol, 1.00 equiv), DCM (8 ml), di-tert-butyl dicarbonate (340 mg, 1.561 mmol, 2.00 equiv) DMAP (10 mg, 0.078 mmol, 0.10 equiv), TEA (236 mg, 2.341 mmol, 3.00 equiv). The reaction was stirred overnight at 25° C. The reaction progress was monitored by LCMS. The reaction was then quenched by H₂O (10 ml). The resulting mixture was extracted with DCM (3×10 mL). The organic layers were combined, dried over Na₂SO₄, filtered and concentrated. The residue was purified by silica gel chromatography (0-30% EtOAc/petroleum ether) to yield (Z)-methyl 4-(N,N′-bis(tert-butoxycarbonyl)-N-methylcarbamimidoyl)benzoate as a light yellow oil. Mass spectrum (ESI, m/z): Calculated for C₂₀H₂₈N₂O₆, 393.1 [M+H], Measured: 393.3.

### Step 4: (Z)-4-(N,N′-bis(tert-butoxycarbonyl)-N-methylcarbamimidoyl) benzoic acid

Into a 50-mL round-bottom flask, was placed (Z)-methyl 4-(N,N′-bis(tert-butoxycarbonyl)-N-methylcarbamimidoyl)benzoate (100 mg, 0.255 mmol, 1.00 equiv), NaOH (51 mg, 1.274 mmol, 5.00 equiv), THE (3 ml), H₂O (3 ml). The reaction was stirred 1 h at 50° C. The reaction progress was monitored by LCMS. The pH value of the solution was adjusted to 6 with 2N hydrogen chloride. The resulting mixture was extracted with EtOAc (3×10 mL). The organic layers were combined, dried over Na₂SO₄, filtered and concentrated to yield (Z)-4-(N,N′-bis(tert-butoxycarbonyl)-N-methyl carbamimidoyl)benzoic acid as a light yellow oil. Mass spectrum (ESI, m/z): Calculated for C₁₉H₂₆N₂O₆, 379.1 [M+H], Measured: 379.3.

### Step 5: (Z)-5-(2-tert-butoxy-2-oxoethyl)-[1,2,4]triazolo[1,5-a]pyridin-8-yl 4-(N,N′-bis(tert-butoxycarbonyl)-N-methylcarbamimidoyl)benzoate

Into a 100-mL round-bottom flask, was placed (Z)-4-(N,N′-bis(tert-butoxycarbonyl)-N-methylcarbamimidoyl)benzoic acid (60 mg, 0.159 mmol, 1.00 equiv), tert-butyl 2-(8-hydroxy-[1,2,4]triazolo[1,5-a]pyridin-5-yl)acetate (40 mg, 0.159 mmol, 1.00 equiv), EDCI (37 mg, 0.238 mmol, 1.50 equiv), DMAP (29 mg, 0.238 mmol, 1.50 equiv), DCM (8 ml). The reaction was stirred 2 h at 25° C. The reaction progress was monitored by LCMS. The resulting solution concentrated. The residue was purified by silica gel chromatography (0-50% PE/EA) to yield (E)-5-(2-tert-butoxy-2-oxoethyl)-[1,2,4] triazolo[1,5-a]pyridin-8-yl 4-(N,N′-bis(tert-butoxycarbonyl)-N-methyl carbamimidoyl)benzoate as a light yellow oil. Mass spectrum (ESI, m/z): Calculated for C₃₁H₃₉N₅O₈, 632.2 [M+Na], Measured: 631.8.

### Step 6: 2-(8-(4-(N-methylcarbamimidoyl)benzoyloxy)-[1,2,4]triazolo[1,5-a]pyridin-5-yl)acetic acid

Into a 50-mL round-bottom flask, was placed (E)-5-(2-tert-butoxy-2-oxoethyl)-[1,2,4]triazolo[1,5-a]pyridin-8-yl 4-(N,N′-bis(tert-butoxycarbonyl)-N-methylcarbamimidoyl)benzoate (30 mg, 0.049 mmol, 1.00 equiv), DCM (5 ml), TFA (2 ml). The reaction was stirred 2 h at 25° C. The reaction progress was monitored by LCMS. The resulting mixture was concentrated under vacuum. The residue was purified by Prep-HPLC with the following conditions (1#-Waters 2767-5): Column, SunFire Prep C18, 5 um, 19*100 mm; mobile phase, Water of 0.05% TFA and CH₃CN (5% CH₃CN up to 20% in 10 min, up to 100% in 2 min, down to 20% in 2 min; Detector, 254 nm to yield 2-(8-(4-(N-methylcarbamimidoyl)benzoyloxy)-[1,2,4]triazolo[1,5-a]pyridin-5-yl)acetic acid as light yellow oil.

¹HNMR (300 MHz, DMSO-d⁶) δ: 12.80 (s, 1H), 10.04 (s, 1H), 9.68 (s, 1H), 9.15 (s, 1H), 8.53 (s, 1H), 8.40 (d, J=8.7 Hz, 2H), 8.00 (d, J=8.7 Hz, 2H), 7.78 (d, J=7.8 Hz, 1H), 7.31 (d, J=8.1 Hz, 1H), 4.25 (s, 2H), 3.04 (s, 3H). Mass spectrum (ESI, m/z): Calculated for C₁₉H₁₆F₃N₅O₆, 354.1 [M+H], Measured: 354.2.

### Example 34: Compound #34

### 2-(8-(4-(3-methylguanidino)benzoyloxy)-[1,2,4]triazolo[1,5-a]pyridin-5-yl)acetic acid

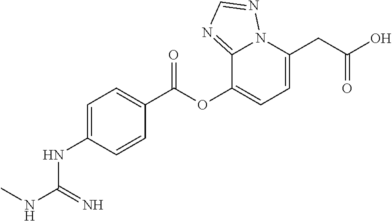 [SMILES: CNC(=N)Nc1ccc(C(=O)Oc2ccc(CC(=O)O)n3ncnc23)cc1]

### Step 1. methyl 4-[({[(tert-butoxy)carbonyl]amino}methanethioyl)amino] benzoate

To a solution of tert-butyl N-({[(tert-butoxy)carbonyl]amino}methane thioyl) carbamate (1000 mg, 3.619 mmol) in THE (20 mL) was added TFAA (840 mg, 3.999 mmol) and NaH (104 mg, 3.443 mmol) at 0° C. The resulting mixture was stirred at 25° C. overnight. After cooling down to room temperature, the reaction was quenched with H₂O (100 mL). The resulting mixture was extracted with EtOAc (3×100 mL). The organic layers were combined, dried over Na₂SO₄, filtered and concentrated. The residue was purified by silica gel chromatography (0-10% EtOAc/petroleum ether) to yield methyl 4-[({[(tert-butoxy)carbonyl]amino}methanethioyl)amino]benzoate as a white solid. Mass spectrum (ESI, m/z): Calculated for C₁₄H₁₈N₂O_{4S}: 311.4[M+H], Measured: 311.1.

### Step 2: 4-(3-(tert-butoxycarbonyl)thioureido)benzoic acid

To a solution of methyl 4-[({[(tert-butoxy)carbonyl]amino}methane thioyl)amino]benzoate (100 mg, 0.322 mmol) in THE (5 mL) and H₂O (5 mL) was added NaOH (129 mg, 3.222 mmol). The resulting mixture was stirred at 25° C. overnight. The reaction was quenched with HCl (10 mL). The resulting mixture was extracted with EtOAc (3×10 mL). The organic layers were combined, dried over Na₂SO₄, filtered and concentrated to yield 4-(3-(tert-butoxycarbonyl)thioureido)benzoic acid as a white solid. Mass spectrum (ESI, m/z): Calculated for C₁₃H₁₆N₂O_{4S}: 297.3[M+H], Measured: 297.1.

### Step 3: 4-thioureidobenzoic acid

To a solution of 4-(3-(tert-butoxycarbonyl)thioureido)benzoic acid (90 mg, 0.304 mmol) in DCM (3 mL) was added CF₃COOH (1 mL). The resulting mixture was stirred at 25° C. for 16 h. The resulting mixture was concentrated to yield 4-thioureidobenzoic acid as a yellow solid. Mass spectrum (ESI, m/z): Calculated for C₈H₈N₂O₂S: 197.2 [M+H], Measured: 196.9.

### Step 4: 4-(imino(methylthio)methylamino)benzoic acid

To a solution of 4-thioureidobenzoic acid (70 mg, 0.357 mmol) in acetone (5 mL) was added CH₃I (0.2 mL) at room temperature. The resulting mixture was stirred at 50° C. for 1 h. The resulting mixture was concentrated to yield 4-(imino(methylthio) methylamino)benzoic acid as a yellow solid. Mass spectrum (ESI, m/z): Calculated for C₉H₁₀N₂O₂S: 211.2[M+H], Measured: 211.2.

### Step 5: 4-(3-methylguanidino)benzoic acid

To a solution of 4-(imino(methylthio)methylamino)benzoic acid (70 mg, 0.333 mmol) in MeOH (5 mL) was added NH₂CH₃ in EtOH (0.2 mL). The resulting mixture was stirred at 50° C. for 1 h. The resulting mixture was concentrated to yield 4-(3-methylguanidino)benzoic acid as a yellow solid. Mass spectrum (ESI, m/z): Calculated for C₉H₁₁N₃O₂: 194.3 [M+H], Measured: 194.2.

### Step 6: (E)-5-(2-tert-butoxy-2-oxoethyl)-[1,2,4]triazolo[1,5-a]pyridin-8-yl 4-(2-(tert-butoxycarbonyl)-3-methylguanidino)benzoate

To a solution of 4-(imino(methylthio)methylamino)benzoic acid (130 mg, 0.673 mmol) in DCM (5 mL) was added tert-butyl 2-(8-hydroxy-[1,2,4]triazolo[1,5-a]pyridin-5-yl)acetate (251 mg, 1.008 mmol). EDCI (261 mg, 1.364 mmol), DMAP (13 mg, 0.103 mmol) were then added. The resulting mixture was stirred at 25° C. overnight. The reaction was quenched with H₂O (10 mL). The resulting mixture was extracted with EtOAc (3×10 mL). The organic layers were combined, dried over Na₂SO₄, filtered and concentrated. The residue was purified by silica gel chromatography (0-30% EA/PE) to yield methyl 2-(4-((5-cyano-2-(methylthio)benzamido)methyl)phenyl)acetate as a yellow oil. Mass spectrum (ESI, m/z): Calculated for C₂₁H₂₄N₆O₄: 425.5 [M+H], Measured: 425.2.

### Step 7: 2-(8-(4-(3-methylguanidino)benzoyloxy)-[1,2,4]triazolo[1,5-a]pyridin-5-yl)acetic acid

Into a 25 ml round-bottom flask, was placed a solution of 5-(2-tert-butoxy-2-oxoethyl)-[1,2,4]triazolo[1,5-a]pyridin-8-yl 4-(3-methylguanidino) benzoate (15 mg, 0.035 mmol) in DCM (3 mL). CF₃COOH (2 mL) was then added. The resulting solution was stirred for 3 h at room temperature. The reaction progress was monitored by LCMS. The resulting residue was dissolved in DMF. The solution was purified by Prep-HPLC with the following conditions: Column, SunFire Prep C18, 5 um, 19*100 mm; mobile phase, Water of 0.05% CF₃COOH and CH₃CN (5% CH₃CN up to 26% in 8 min, up to 100% in 1 min, down to 20% in 1 min; Detector, 220 nm, 254 nm to yield 2-(8-(4-(3-methylguanidino) benzoyloxy)-[1,2,4]triazolo[1,5-a]pyridin-5-yl)acetic acid as off-white solid. ¹H NMR (300 MHz, DMSO-d⁶), δ: 10.11 (s, 1H), 8.52 (s, 1H), 8.29-8.17 (m, 5H), 7.74-7.71 (m, 1H), 7.47-7.44 (m, 2H), 7.31-7.28 (m, 1H), 4.24 (s, 2H), 2.89 (s, 3H). Mass spectrum (ESI, m/z): Calculated for C₁₇H₁₆N₆O₄: 369.3[M+H], Measured: 369.1.

### Example 35: Compound #35

### 2-(8-(4-(1-methylguanidino)benzoyloxy)-[1,2,4]triazolo[1,5-a]pyridin-5-yl)acetic acid

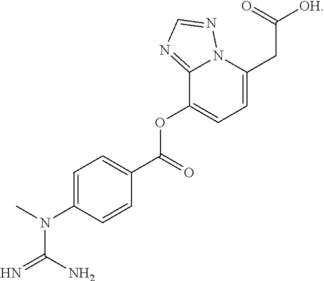 [SMILES: CN(C(=N)N)c1ccc(C(=O)Oc2ccc(CC(=O)O)n3ncnc23)cc1]

### Step 1: Methyl 4-(methylamino)benzoate

Into a 100-mL round-bottom flask, was placed methyl 4-aminobenzoate (1.00 g, 6.615 mmol, 1.00 equiv), K2CO₃ (914 mg, 6.615 mmol, 1.00 equiv), CH₃I (1.127 g, 7.938 mmol, 1.20 equiv), CH₃CN (20 ml). The reaction was stirred overnight at 80° C. The reaction progress was monitored by LCMS. The resulting solution concentrated. The residue was purified by silica gel chromatography (0-50% PE/EA) to yield methyl 4-(methylamino) benzoate as off-white solid. Mass spectrum (ESI, m/z): Calculated for C₉H₁₁NO₂, 166.0 [M+H], Measured: 166.0.

### Step 2: Methyl 4-{[(1Z)-{[(tert-butoxy)carbonyl]amino}({[(tert-butoxy) carbonyl]imino})methyl](methyl)amino}benzoate

Into a 100-mL round-bottom flask, was placed methyl 4-(methylamino) benzoate (450 mg, 2.724 mmol, 1.00 equiv), tert-butyl N-[(1E)-{[(tert-butoxy) carbonyl]amino}(methylsulfanyl)methylidene]carbamate (791 mg, 2.724 mmol, 1.00 equiv), HgCl₂ (740 mg, 2.724 mmol, 1.00 equiv), TEA (827 mg, 8.172 mmol, 3.00 equiv), DMF (10 ml). The reaction was stirred overnight at 25° C. The reaction progress was monitored by LCMS. The reaction was then quenched by H₂O (10 ml). The resulting mixture was extracted with EA (3×20 mL). The organic layers were combined, dried over Na₂SO₄, filtered and concentrated. The residue was purified by silica gel chromatography (0-30% EtOAc/petroleum ether) to yield methyl 4-{[(1Z)-{[(tert-butoxy)carbonyl]amino}({[(tert-butoxy)carbonyl]imino})methyl](methyl)amino}benzoate as a light yellow solid. Mass spectrum (ESI, m/z): Calculated for C₂₀H₂₉N₃O₆, 408.4 [M+H], Measured: 408.4.

### Step 3: 4-(2,3-bis(tert-butoxycarbonyl)-1-methylguanidino)benzoic acid

Into a 50-mL round-bottom flask, was placed methyl 4-{[(1Z)-{[(tert-butoxy)carbonyl]amino}({[(tert-butoxy)carbonyl]imino})methyl](methyl)amino}benzoate (150 mg, 0.368 mmol, 1.00 equiv), NaOH (74 mg, 1.841 mmol, 5.00 equiv), THE (4 ml), H₂O (4 ml). The reaction was stirred 30 min at 50° C. The reaction progress was monitored by LCMS. The pH value of the solution was adjusted to 6 with 2N hydrogen chloride. The resulting mixture was extracted with EtOAc (3×10 mL). The organic layers were combined, dried over Na₂SO₄, filtered and concentrated to yield 4-(2,3-bis(tert-butoxycarbonyl)-1-methylguanidino)benzoic acid as a light yellow oil. Mass spectrum (ESI, m/z): Calculated for C₁₉H₂₇N₃O₆, 394.3 [M+H], Measured: 394.3.

### Step 4: 5-[2-(tert-butoxy)-2-oxoethyl]-[1,2,4]triazolo[1,5-a]pyridin-8-yl 4-{[(1Z)-{[(tert-butoxy)carbonyl]amino}({[(tert-butoxy)carbonyl]imino})methyl](methyl)amino}benzoate

Into a 100-mL round-bottom flask, was placed (Z)-4-(2,3-bis(tert-butoxy carbonyl)-1-methylguanidino)benzoic acid (80 mg, 0.203 mmol, 1.00 equiv), tert-butyl 2-(8-hydroxy-[1,2,4]triazolo[1,5-a]pyridin-5-yl)acetate (51 mg, 0.203 mmol, 1.00 equiv), EDCI (47 mg, 0.305 mmol, 1.50 equiv), DMAP (37 mg, 0.305 mmol, 1.50 equiv), DCM (8 ml). The reaction was stirred 2 h at 25° C. The reaction progress was monitored by LCMS. The resulting solution concentrated. The residue was purified by silica gel chromatography (0-50% PE/EA) to yield 80 mg of 5-[2-(tert-butoxy)-2-oxoethyl]-[1,2,4] triazolo[1,5-a]pyridin-8-yl 4-{[(1Z)-{[(tert-butoxy)carbonyl]amino}({[(tert-butoxy)carbonyl]imino})methyl](methyl)amino}benzoate as a light yellow oil. Calculated for C₃₁H₄₀N₆O₈, 625.3 [M+H], Measured: 625.3.

### Step 5: 2-(8-(4-(1-methylguanidino)benzoyloxy)-[1,2,4]triazolo[1,5-a]pyridin-5-yl)acetic acid

Into a 50-mL round-bottom flask, was placed 5-[2-(tert-butoxy)-2-oxoethyl]-[1,2,4]triazolo[1,5-a]pyridin-8-yl 4-{[(1Z)-{[(tert-butoxy) carbonyl]amino}({[(tert-butoxy)carbonyl]imino})methyl](methyl)amino}benzoat (80 mg, 0.128 mmol, 1 equiv), DCM (5 ml), TFA (2 ml). The reaction was stirred 2 h at 25° C. The resulting mixture was concentrated under vacuum. The residue was purified by Prep-HPLC with the following conditions (1#-Waters 2767-5): Column, SunFire Prep C18, 5 um, 19*100 mm; mobile phase, Water of 0.05% TFA and CH₃CN (5% CH₃CN up to 20% in 10 min, up to 100% in 2 min, down to 20% in 2 min; Detector, 254 nm to yield 2-(8-(4-(1-methylguanidino)benzoyloxy)-[1,2,4]triazolo[1,5-a]pyridin-5-yl)acetic acid 2,2,2-trifluoroacetic acid as white solid. ¹HNMR (300 MHz, DMSO-d⁶) δ: 12.79 (brs, 1H), 8.53 (s, 1H), 8.31-8.33 (m, 2H), 7.67-7.76 (m, 3H), 7.53 (s, 4H), 7.30-7.32 (m, 1H), 4.25 (s, 2H), 3.38 (s, 3H). Mass spectrum (ESI, m/z): Calculated for C₁₉H₁₇F₃N₆O₆, 369.1 [M+H], Measured: 369.1.

### Example 36: Compound #36

### 2-(8-(4-(N-methoxycarbamimidoyl)benzoyloxy)-[1,2,4]triazolo[1,5-a]pyridin-5-yl)acetic acid

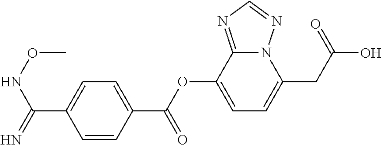 [SMILES: CONC(=N)c1ccc(C(=O)Oc2ccc(CC(=O)O)n3ncnc23)cc1]

### Step 1: Methyl 4-(N-methoxycarbamimidoyl)benzoate

Into a 100-mL round-bottom flask, was placed methyl 4-(ethoxy(imino) methyl)benzoate (200 mg, 0.965 mmol, 1.00 equiv), O-methylhydroxylamine hydrochloride (403 mg, 4.826 mmol, 5.00 equiv), pyridine (10 ml). The reaction was stirred 3 h at 25° C. The reaction progress was monitored by LCMS. The resulting mixture was concentrated under vacuum to yield methyl 4-(N-methoxycarbamimidoyl)benzoate as a light yellow solid. Mass spectrum (ESI, m/z): Calculated for C₁₀H₁₂N₂O₃, 209.1 [M+H], Measured: 209.1.

### Step 2. 4-(N-methoxycarbamimidoyl)benzoic acid

Into a 50-mL round-bottom flask, was placed methyl 4-(N-methoxy carbamimidoyl)benzoate (200 mg, 0.965 mmol, 1.00 equiv), NaOH (193 mg, 4.826 mmol, 5.00 equiv), THE (4 ml), H₂O (4 ml). The reaction was stirred 1 h at 50° C. The reaction progress was monitored by LCMS. The pH value of the solution was adjusted to 6 with 2N hydrogen chloride. The resulting mixture was extracted with EtOAc (3×10 mL). The organic layers were combined, dried over Na₂SO₄, filtered and concentrated to yield 4-(N-methoxycarbamimidoyl)benzoic acid as a light yellow oil. Mass spectrum (ESI, m/z): Calculated for C₉H₁₀N₂O₃, 195.1 [M+H], Measured: 195.1.

### Step 3: 5-(2-tert-butoxy-2-oxoethyl)-[1,2,4]triazolo[1,5-a]pyridin-8-yl 4-(N-methoxycarbamimidoyl)benzoate

Into a 100-mL round-bottom flask, was placed 4-(N-methoxycarbamimidoyl) benzoic acid (50 mg, 0.257 mmol, 1 equiv), tert-butyl 2-(8-hydroxy-[1,2,4]triazolo[1,5-a]pyridin-5-yl)acetate (64 mg, 0.257 mmol, 1 equiv), EDCI (60 mg, 0.386 mmol, 1.5 equiv), DMAP (47 mg, 0.386 mmol, 1.5 equiv), DCM (5 ml). The reaction was stirred 2 h at 25° C. The reaction progress was monitored by LCMS. The resulting solution concentrated. The residue was purified by silica gel chromatography (0-50% PE/EA) to yield 5-(2-tert-butoxy-2-oxoethyl)-[1,2,4]triazolo[1,5-a]pyridin-8-yl 4-(N-methoxy carbamimidoyl)benzoate as a light yellow oil. Mass spectrum (ESI, m/z): Calculated for C₂₁H₂₃N₅O₅, 426.1 [M+H], Measured: 426.1.

### Step 4: 2-(8-(4-(N-methoxycarbamimidoyl)benzoyloxy)-[1,2,4]triazolo[1,5-a]pyridin-5-yl)acetic acid

Into a 50-mL round-bottom flask, was placed 5-(2-tert-butoxy-2-oxoethyl)-[1,2,4]triazolo[1,5-a]pyridin-8-yl 4-(N-methoxycarbamimidoyl) benzoate (70 mg, 0.128 mmol, 1 equiv), DCM (5 ml), TFA (2 ml). The reaction was stirred 2 h at 25° C. The reaction progress was monitored by LCMS. The resulting mixture was concentrated under vacuum. The residue was purified by Prep-HPLC with the following conditions (1#-Waters 2767-5): Column, SunFire Prep C18, 5 um, 19*100 mm; mobile phase, Water of 0.05% TFA and CH₃CN (5% CH₃CN up to 20% in 10 min, up to 100% in 2 min, down to 20% in 2 min; Detector, 254 nm to yield 2-(8-(4-(N-methoxycarbamimidoyl)benzoyloxy)-[1,2,4]triazolo[1,5-a]pyridin-5-yl)acetic acid as white solid. ¹HNMR (400 MHz, DMSO-d⁶) δ: 12.83 (brs, 1H), 8.53 (s, 1H), 8.20 (d, J=8.4 Hz, 2H), 7.95 (d, J=8.8 Hz, 2H), 7.76 (d, J=7.6 Hz, 1H), 7.30 (d, J=7.6 Hz, 1H), 6.39 (s, 2H), 4.24 (s, 2H), 3.81 (s, 3H). Mass spectrum (ESI, m/z): Calculated for C₁₇H₁₅N₅O₅, 370.1 [M+H], Measured: 370.2.

### Example 37: Compound #37

### 2-(8-(4-acetimidamidobenzoyloxy)-[1,2,4]triazolo[1,5-a]pyridin-5-yl)acetic acid

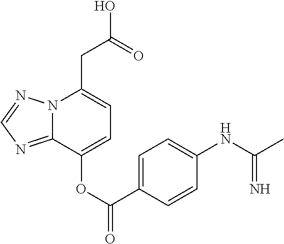 [SMILES: CC(=N)Nc1ccc(C(=O)Oc2ccc(CC(=O)O)n3ncnc23)cc1]

### Step 1: Methyl 4-acetimidamidobenzoate

Into a 250-mL round-bottom flask, was placed methyl 4-aminobenzoate (1 g, 6.615 mmol, 1 equiv), benzyl ethanimidothioate (2.186 g, 13.231 mmol, 2.00 equiv), EtOH (20 ml). The reaction was stirred overnight at 50° C. The reaction progress was monitored by LCMS. The resulting solution concentrated. The residue was purified by silica gel chromatography (0-50% PE/EA) to yield methyl 4-acetimidamidobenzoate as a light yellow oil. Mass spectrum (ESI, m/z): Calculated for C₁₀H₁₂N₂O₂, 193.1 [M+H], Measured: 193.1.

### Step 2: 4-acetimidamidobenzoic acid

Into a 50-mL round-bottom flask, was placed methyl 4-acetimidamidobenzoate (150 mg, 0.780 mmol, 1 equiv), NaOH (156 mg, 3.902 mmol, 5 equiv), THE (4 ml), H₂O (4 ml). The reaction was stirred 1 h at 50° C. The reaction progress was monitored by LCMS. The pH value of the solution was adjusted to 6 with 2N hydrogen chloride. The resulting mixture was extracted with EtOAc (3×50 mL). The organic layers were combined, dried over Na₂SO₄, filtered and concentrated to yield 4-acetimidamidobenzoic acid as a light yellow oil. Mass spectrum (ESI, m/z): Calculated for C₉H₁₀N₂O₂, 179.1 [M+H], Measured: 179.1.

### Step 3: 5-(2-tert-butoxy-2-oxoethyl)-[1,2,4]triazolo[1,5-a]pyridin-8-yl 4-acetimidamidobenzoate

Into a 100-mL round-bottom flask, was placed 4-acetimidamidobenzoic acid (60 mg, 0.337 mmol, 1 equiv), tert-butyl 2-(8-hydroxy-[1,2,4]triazolo[1,5-a]pyridin-5-yl)acetate (84 mg, 0.337 mmol, 1 equiv), EDCI (78 mg, 0.505 mmol, 1.5 equiv), DMAP (62 mg, 0.505 mmol, 1.5 equiv), DCM (8 ml). The reaction was stirred 2 h at 25° C. The reaction progress was monitored by LCMS. The resulting solution concentrated. The residue was purified by silica gel chromatography (0-50% PE/EA) to yield 5-(2-tert-butoxy-2-oxoethyl)-[1,2,4]triazolo[1,5-a]pyridin-8-yl 4-acetimidamidobenzoate as a light yellow oil. Mass spectrum (ESI, m/z): Calculated for C₂₁H₂₃N₅O₄, 410.1 [M+H], Measured: 410.1.

### Step 4: 2-(8-(4-acetimidamidobenzoyloxy)-[1,2,4]triazolo[1,5-a]pyridin-5-yl)acetic acid

Into a 50-mL round-bottom flask, was placed 5-(2-tert-butoxy-2-oxoethyl)-[1,2,4]triazolo[1,5-a]pyridin-8-yl 4-acetimidamidobenzoate (50 mg, 0.122 mmol, 1 equiv), DCM (5 ml), TFA (2 ml). The reaction was stirred 2 h at 25° C. The reaction progress was monitored by LCMS. The resulting mixture was concentrated under vacuum. The residue was purified by Prep-HPLC with the following conditions (1#-Waters 2767-5): Column, SunFire Prep C18, 5 um, 19*100 mm; mobile phase, Water of 0.05% TFA and CH₃CN (5% CH₃CN up to 20% in 10 min, up to 100% in 2 min, down to 20% in 2 min; Detector, 254 nm to yield 2-(8-(4-acetimidamidobenzoyloxy)-[1,2,4]triazolo[1,5-a]pyridin-5-yl)acetic acid as white solid.

¹HNMR (300 MHz, DMSO-d⁶) 6:12.78 (brs, 1H), 11.54 (brs, 1H), 9.73 (brs, 1H), 8.90 (brs, 1H), 8.53 (s, 1H), 8.34 (d, J=8.4 Hz, 2H), 7.75 (d, J=7.8 Hz, 1H), 7.60 (d, J=8.7 Hz, 2H), 7.31 (d, J=7.8 Hz, 1H), 4.25 (s, 2H), 2.38 (s, 3H). Mass spectrum (ESI, m/z): Calculated for C₁₉H₁₆F₃N₅O₆, 354.1 [M+H], Measured: 354.1.

### Example 38: Compound #38

### 2-(8-(4-(N-cyanocarbamimidoyl)benzoyloxy)-[1,2,4]triazolo[1,5-a]pyridin-5-yl)acetic acid

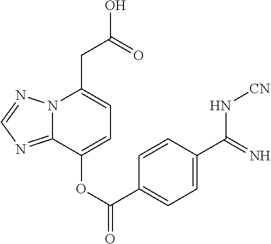 [SMILES: N#CNC(=N)c1ccc(C(=O)Oc2ccc(CC(=O)O)n3ncnc23)cc1]

### Step 1: methyl 4-(N-cyanocarbamimidoyl)benzoate

To a solution of methyl 4-(ethoxy(imino)methyl)benzoate (400 mg, 1.930 mmol) in MeOH (5.0 ml) was added Et₃N (1.0 ml). Then cyanamide (1.623 g, 38.605 mmol) in MeOH (20 ml) was added the mixture. The resulting solution was stirred overnight at 70° C. The reaction progress was monitored by LCMS. The resulting mixture was concentrated under vacuum to yield methyl 4-(N-cyanocarbamimidoyl)benzoate as an off-white solid. Mass spectrum (ESI, m/z): Calculated for C₁₀H₉N₃O₂, 204.1[M+H], Measured: 204.2.

### Step 2: 4-(N-cyanocarbamimidoyl)benzoic acid

To a solution of methyl 4-(N-cyanocarbamimidoyl)benzoate (400 mg, 1.969 mmol) in THE (5.0 ml) with H₂O (2.0 ml), was added NaOH (393.707 mg, 9.843 mmol). The resulting solution was stirred 30 min at 50° C. The reaction was monitored by LCMS. The reaction was quenched by H₂O (5 ml) and with 2 N HCl adjusted pH to 6. The mixture was extracted with EA (3×5 ml), the organic layers were combined, dried over Na₂SO₄ and concentrated under vacuum to yield 4-(N-cyanocarbamimidoyl)benzoic acid as a yellow oil. Mass spectrum (ESI, m/z): Calculated for C₉H₇N₃O₂, 190.1[M+H], Measured: 190.1.

### Step 3: 5-(2-tert-butoxy-2-oxoethyl)-[1,2,4]triazolo[1,5-a]pyridin-8-yl 4-(N-cyanocarbamimidoyl)benzoate

To a solution of tert-butyl 2-(8-hydroxy-[1,2,4]triazolo[1,5-a]pyridin-5-yl)acetate (70 mg, 0.281 mmol) in DCM (5.0 ml), was added EDCI (107.275 mg, 0.562 mmol), DMAP (68.521 mg, 0.562 mmol), 4-(N-cyanocarbamimidoyl) benzoic acid (106.248 mg, 0.562 mmol). The resulting solution was stirred 3.0 h at room temperature. The reaction was monitored by LCMS. The resulting mixture was concentrated under vacuum. The residue was purified by Prep-HPLC with the following conditions (1#-Waters 2767-5): Column, SunFire Prep C18, 5 um, 19*100 mm; mobile phase, Water with 0.05% TFA and CH₃CN (5% CH₃CN up to 30% in 10 min, up to 100% in 0.1 min, hold 100% in 0.9 min, down to 5% in 0.1 min, hold 5% in 1.4 min); Detector, UV 220&254 nm to yield 5-(2-tert-butoxy-2-oxoethyl)-[1,2,4]triazolo[1,5-a]pyridin-8-yl 4-(N-cyano carbamimidoyl)benzoate as a white solid. Mass spectrum (ESI, m/z): Calculated for C₂₁H₂₀N₆O₄, 421.2[M+H], Measured: 421.2.

### Step 4. 2-(8-(4-(N-cyanocarbamimidoyl)benzoyloxy)-[1,2,4]triazolo[1,5-a]pyridin-5-yl)acetic acid

To a solution of 5-(2-tert-butoxy-2-oxoethyl)-[1,2,4]triazolo[1,5-a]pyridin-8-yl 4-(N-cyanocarbamimidoyl)benzoate (30 mg, 0.071 mmol) in DCM (4.0 ml) was added TFA (1.0 ml). The resulting solution was stirred 3.0 h at room temperature. The resulting mixture was concentrated under vacuum. The residue was purified by Prep-HPLC with the following conditions (1#-Waters 2767-5): Column, SunFire Prep C18, 5 um, 19*100 mm; mobile phase, Water with 0.05% TFA and CH₃CN (5% CH₃CN up to 30% in 10 min, up to 100% in 0.1 min, hold 100% in 0.9 min, down to 5% in 0.1 min, hold 5% in 1.4 min); Detector, UV 220&254 nm to yield 2-(8-(4-(N-cyanocarbamimidoyl) benzoyloxy)-[1,2,4]triazolo[1,5-a]pyridin-5-yl)acetic acid as a white solid.

¹H NMR (300 MHz, DMSO-d⁶) δ: 12.73-12.80 (m, 1H), 9.34-9.39 (m, 1H), 8.92-8.99 (m, 1H), 8.53 (s, 1H), 8.29-8.32 (m, 2H), 8.16-8.18 (m, 2H), 7.78 (d, J=7.8 Hz, 1H), 7.31 (d, J=8.1 Hz, 1H), 4.24 (s, 2H). Mass spectrum (ESI, m/z): Calculated for C₁₇H₁₂N₆O₄, 365.1[M+H], Measured: 365.1.

### Example 39: Compound #39

### 2-(8-(4-(hydrazinyl(imino)methyl)benzoyloxy)-[1,2,4]triazolo[1,5-a]pyridin-5-yl)acetic acid

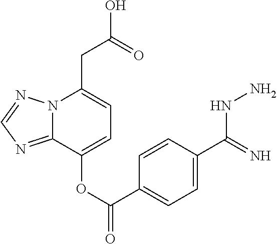 [SMILES: N=C(NN)c1ccc(C(=O)Oc2ccc(CC(=O)O)n3ncnc23)cc1]

### Step 1: Methyl 4-(hydrazinyl(imino)methyl)benzoate

Into a 100-mL round-bottom flask, was placed methyl 4-(ethoxy(imino) methyl)benzoate (200 mg, 0.965 mmol, 1.00 equiv), hydrazine hydrate ((242 mg, 4.826 mmol, 5 equiv), pyridine (8 ml). The reaction was stirred 2 h at 25° C. The reaction progress was monitored by LCMS. The resulting mixture was concentrated under vacuum to yield methyl 4-(hydrazinyl (imino)methyl)benzoate as a light yellow solid. Mass spectrum (ESI, m/z): Calculated for C₉H₁₁N₃O₂, 194.1 [M+H], Measured: 194.1.

### Step 2: 4-(hydrazinyl(imino)methyl)benzoic acid

Into a 50-mL round-bottom flask, was placed methyl 4-(hydrazinyl (imino)methyl)benzoate (150 mg, 0.776 mmol, 1 equiv), NaOH (155 mg, 3.822 mmol, 5 equiv), THE (4 ml), H₂O (4 ml). The reaction was stirred 2 h at 50° C. The reaction progress was monitored by LCMS. The pH value of the solution was adjusted to 6 with 2 N hydrogen chloride. The resulting mixture was extracted with EtOAc (3×10 mL). The organic layers were combined, dried over Na₂SO₄, filtered and concentrated to yield 4-(hydrazinyl(imino)methyl)benzoic acid as a light yellow oil. Mass spectrum (ESI, m/z): Calculated for C₈H₉N₃O₂, 180.2 [M+H], Measured: 180.2.

### Step 3: 5-(2-tert-butoxy-2-oxoethyl)-[1,2,4]triazolo[1,5-a]pyridin-8-yl 4-(hydrazinyl(imino)methyl)benzoate

Into a 100-mL round-bottom flask, was placed tert-butyl 2-(8-hydroxy-[1,2,4]triazolo[1,5-a]pyridin-5-yl)acetate (40 mg, 0.160 mmol, 1 equiv), 4-(hydrazinyl(imino)methyl)benzoic acid (29 mg, 0.160 mmol, 1 equiv), EDCI (37 mg, 0.241 mmol, 1.5 equiv), DMAP (29 mg, 0.241 mmol, 1.5 equiv), DCM (5 ml). The reaction was stirred 2 h at 25° C. The reaction progress was monitored by LCMS. The resulting solution concentrated. The residue was purified by silica gel chromatography (0-50% PE/EA) to yield 5-(2-tert-butoxy-2-oxoethyl)-[1,2,4]triazolo[1,5-a]pyridin-8-yl 4-(hydrazinyl(imino)methyl)benzoate as a light yellow oil. Mass spectrum (ESI, m/z): Calculated for C₂₀H₂₂N₆O₄, 411.2 [M+H], Measured: 411.2.

### Step 4: 2-(8-(4-(hydrazinyl(imino)methyl)benzoyloxy)-[1,2,4]triazolo[1,5-a]pyridin-5-yl)acetic acid

Into a 50-mL round-bottom flask, was placed 5-(2-tert-butoxy-2-oxoethyl)-[1,2,4]triazolo[1,5-a]pyridin-8-yl 4-(hydrazinyl(imino)methyl)benzoate (20 mg, 0.049 mmol, 1 equiv), DCM (5 ml), TFA (2 ml). The reaction was stirred 2 h at 25° C. The reaction progress was monitored by LCMS. The resulting mixture was concentrated under vacuum. The residue was purified by Prep-HPLC with the following conditions (1#-Waters 2767-5): Column, SunFire Prep C18, 5 um, 19*100 mm; mobile phase, Water of 0.05% TFA and CH₃CN (5% CH₃CN up to 20% in 10 min, up to 100% in 2 min, down to 20% in 2 min; Detector, 254 nm to yield 2-(8-(4-(hydrazinyl(imino)methyl)benzoyloxy)-[1,2,4]triazolo[1,5-a]pyridin-5-yl)acetic acid as white solid.

¹HNMR (400 MHz, DMSO-d⁶) δ: 12.80 (brs, 1H), 11.26 (brs, 1H), 9.67 (brs, 1H), 8.52 (s, 1H), 8.38 (d, J=8.4 Hz, 2H), 7.96 (d, J=8.4 Hz, 2H), 7.77 (d, J=8.4 Hz, 1H), 7.31 (d, J=8.0 Hz, 1H), 5.40 (brs, 2H), 4.25 (s, 2H). Mass spectrum (ESI, m/z): Calculated for C₁₆H₁₄N₆O₄, 355.1 [M+H], Measured: 355.1.

### Example 40: Compound #40

### (E)-2-(8-(4-(N′-hydroxyacetimidamido)benzoyloxy)-[1,2,4] triazolo[1,5-a]pyridin-5-yl)acetic acid

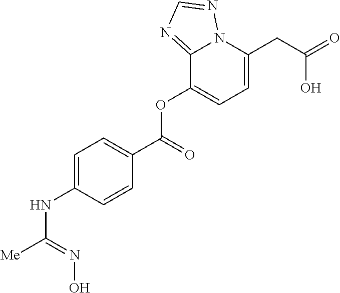 [SMILES: C/C(=N\O)Nc1ccc(C(=O)Oc2ccc(CC(=O)O)n3ncnc23)cc1]

### Step 1: (Z)-methyl 4-(N′-hydroxyacetimidamido)benzoate

Into a 100-mL round-bottom flask, was placed methyl 4-aminobenzoate (500 mg, 3.308 mmol, 1 equiv), N-hydroxyacetamide (1.241 g, 16.538 mmol, 1 equiv), PPA (10 ml). The reaction was stirred overnight h at 90° C. The reaction progress was monitored by LCMS. The reaction was then quenched by H₂O (50 ml). The resulting mixture was extracted with EA (3×50 mL). The organic layers were combined, dried over Na₂SO₄, filtered and concentrated. The residue was purified by silica gel chromatography (0-30% EtOAc/petroleum ether) to yield (Z)-methyl 4-(N′-hydroxyacetimidamido) benzoate as a light yellow oil. Mass spectrum (ESI, m/z): Calculated for C₁₀H₁₂N₂O₃, 209.1 [M+H], Measured: 209.1.

### Step 2: (Z)-4-(N′-hydroxyacetimidamido)benzoic acid

Into a 50-mL round-bottom flask, was place (Z)-methyl 4-(N′-hydroxy acetimidamido)benzoate (100 mg, 0.480 mmol, 1 equiv), NaOH (96 mg, 2.401 mmol, 5 equiv), THE (4 ml), H₂O (4 ml). The reaction was stirred 2 h at 50° C. The reaction progress was monitored by LCMS. The pH value of the solution was adjusted to 6 with 2N hydrogen chloride. The resulting mixture was extracted with EtOAc (3×50 mL). The organic layers were combined, dried over Na₂SO₄, filtered and concentrated to yield 4-acetimidamidobenzoic acid as a light yellow oil. Mass spectrum (ESI, m/z): Calculated for C₉H₁₀N₂O₃, 195.1 [M+H], Measured: 195.1.

### Step 3: (E)-5-(2-tert-butoxy-2-oxoethyl)-[1,2,4]triazolo[1,5-a]pyridin-8-yl4-(N′-hydroxyacetimidamido)benzoate

Into a 100-mL round-bottom flask, was placed (Z)-4-(N′-hydroxy acetimidamido)benzoic acid (50 mg, 0.257 mmol, 1 equiv), tert-butyl 2-(8-hydroxy-[1,2,4]triazolo[1,5-a]pyridin-5-yl)acetate (64 mg, 0.257 mmol, 1 equiv), EDCI (60 mg, 0.386 mmol, 1.5 equiv), DMAP (47 mg, 0.386 mmol, 1.5 equiv), DCM (5 ml). The reaction was stirred 2 h at 25° C. The reaction progress was monitored by LCMS. The resulting solution concentrated. The residue was purified by silica gel chromatography (0-50% PE/EA) to yield (E)-5-(2-tert-butoxy-2-oxoethyl)-[1,2,4]triazolo[1,5-a]pyridin-8-yl 4-(N′-hydroxy acetimidamido)benzoate as a light yellow oil. Mass spectrum (ESI, m/z): Calculated for C₂₁H₂₃N₅O₅, 426.1 [M+H], Measured: 426.1.

### Step 4: (E)-2-(8-(4-(N′-hydroxyacetimidamido)benzoyloxy)-[1,2,4] triazolo[1,5-a]pyridin-5-yl)acetic acid

Into a 50-mL round-bottom flask, was placed (E)-5-(2-tert-butoxy-2-oxoethyl)-[1,2,4]triazolo[1,5-a]pyridin-8-yl 4-(N′-hydroxyacetimidamido) benzoate (50 mg, 0.118 mmol, 1 equiv), DCM (5 ml), TFA (2 ml). The reaction was stirred 2 h at 25° C. The reaction progress was monitored by LCMS. The resulting mixture was concentrated under vacuum. The residue was purified by Prep-HPLC with the following conditions (1#-Waters 2767-5): Column, SunFire Prep C18, 5 um, 19*100 mm; mobile phase, Water of 0.05% TFA and CH₃CN (5% CH₃CN up to 20% in 10 min, up to 100% in 2 min, down to 20% in 2 min; Detector, 254 nm to yield (E)-2-(8-(4-(N′-hydroxyacetimidamido)benzoyloxy)-[1,2,4]triazolo[1,5-a]pyridin-5-yl)acetic acid as white solid.

¹HNMR (300 MHz, DMSO-d⁶) δ: 9.64 (brs, 1H), 8.77 (s, 1H), 8.49-8.51 (m, 1H), 8.02-8.18 (m, 3H), 7.65-7.73 (m, 3H), 7.46-7.49 (m, 1H), 7.25-7.30 (m, 1H), 4.22-4.23 (m, 2H), 2.04-2.16 (m, 3H). Mass spectrum (ESI, m/z): Calculated for C₁₇H₁₅N₅O₅, 370.1 [M+H], Measured: 370.1.

### Example 41: Compound #41

### 5-(2-(tert-butoxy)-2-oxoethyl)-[1,2,4]triazolo[1,5-a]pyridin-8-yl (Z)-4-(2,3-bis(tert-butoxycarbonyl)guanidino)-2-iodobenzoate

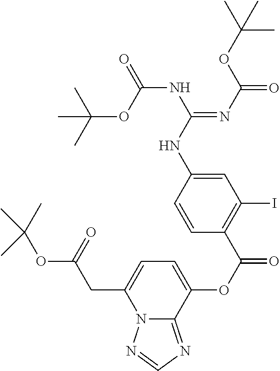 [SMILES: CC(C)(C)OC(=O)Cc1ccc(OC(=O)c2ccc(N/C(=N/C(=O)OC(C)(C)C)NC(=O)OC(C)(C)C)cc2I)c2ncnn12]

### Step 1: 2-Iodo-4-nitrobenzoic acid

Into a 250-mL 3-necked round-bottom flask purged and maintained with an inert atmosphere of nitrogen, was placed a solution of 2-amino-4-nitrobenzoic acid (10 g, 54.90 mmol, 1.00 equiv.) in 9N sulfuric acid (64 mL). This was followed by the addition of a solution of NaNO₂ (4.17 g, 60.43 mmol, 1.10 equiv.) in water (50 mL), added dropwise with stirring at 0° C., with the resulting mixture stirred for 1 h at 0° C. To the resulting mixture was then added urea (1.1 g, 18.11 mmol, 0.33 equiv.) in several batches at 0° C. To the mixture was added a solution of NaI (9.9 g, 66.00 mmol, 1.20 equiv.) in water (50 mL), dropwise with stirring at 0° C. The resulting solution was stirred for 1 h at room temperature. The reaction was then quenched by the addition of NaHSO₃ (aq. 1%, 200 mL). The solids were collected by filtration and washed with ethyl acetate (2×200 mL) to yield 2-iodo-4-nitrobenzoic acid as a brown solid.

### Step 2: 4-Amino-2-iodobenzoic acid

Into a 250-mL 3-necked round-bottom flask, was placed a solution of 2-iodo-4-nitrobenzoic acid (6 g, 20.48 mmol, 1.00 equiv.) in ethanol/H₂O (60/20 mL), NH₄Cl (760 mg, 14.21 mmol, 0.69 equiv.), Fe (6.89 g, 123.04 mmol, 6.00 equiv.). The resulting solution was heated to reflux for 5 hr. The reaction mixture was cooled to room temperature with a water/ice bath. The solids were filtered out. The resulting solution was extracted with ethyl acetate (3×100 mL) and the organic layers combined. The resulting mixture was washed with brine (2×100 mL), dried over anhydrous sodium sulfate and concentrated under vacuum to yield 4-amino-2-iodobenzoic acid as a brown solid.

### Step 3: 5-(2-(Tert-butoxy)-2-oxoethyl)-[1,2,4]triazolo[1,5-a]pyridin-8-yl 4-amino-2-iodobenzoate

Into a 50-mL round-bottom flask, was placed 4-amino-2-iodobenzoic acid (600 mg, 2.28 mmol, 1.10 equiv.), tert-butyl 2-[8-hydroxy-[1,2,4]triazolo[1,5-a]pyridin-5-yl]acetate (500 mg, 2.01 mmol, 1.00 equiv.), 4-dimethylaminopyridine (65 mg, 0.53 mmol, 0.27 equiv.), DCM (25 mL) and DCC (600 mg, 22.19 mmol, 1.4 equiv.). The resulting solution was stirred for 4 hours at room temperature. The resulting mixture was concentrated under vacuum. The residue was applied onto a silica gel column with dichloromethane/ethyl acetate (100/0˜ 90/10) to yield 5-[2-(tert-butoxy)-2-oxoethyl]-[1,2,4]triazolo[1,5-a]pyridin-8-yl 4-amino-2-iodobenzoate as a white solid. LC-MS: (ES, m/z): [M+1]⁺=495. ¹H NMR (300 MHz, DMSO-d₆, ppm) δ 8.48 (s, 1H), 8.00 (d, J=8.7 Hz, 1H), 7.60 (d, J=7.7 Hz, 1H), 7.31 (dd, J=2.2, 0.9 Hz, 1H), 7.21 (d, J=7.8 Hz, 1H), 6.66 (dd, J=8.9, 2.2 Hz, 1H), 6.39 (s, 2H), 4.18 (s, 2H), 1.37 (s, 9H).

### Step 4: 5-(2-(Tert-butoxy)-2-oxoethyl)-[1,2,4]triazolo[1,5-a]pyridin-8-yl (Z)-4-(2,3-bis(tert-butoxycarbonyl)guanidino)-2-iodobenzoate

Into a 25-mL round-bottom flask, was placed 5-[2-(tert-butoxy)-2-oxoethyl]-[1,2,4]triazolo[1,5-a]pyridin-8-yl 4-amino-2-iodobenzoate (400 mg, 0.81 mmol, 1.00 equiv.), tert-butyl N-[(1E)-[[(tert-butoxy)carbonyl]imino](1H-pyrazol-1-yl)methyl] carbamate (300 mg, 0.97 mmol, 1.20 equiv.), acetonitrile (8 mL), TFA (1 mg, 0.01 mmol, 0.01 equiv.). The resulting solution was stirred overnight at room temperature. The resulting mixture was concentrated under vacuum. The residue was applied onto a silica gel column with dichloromethane/ethyl acetate (100/0 to 90/10) to yield 5-[2-(tert-butoxy)-2-oxoethyl]-[1,2,4]triazolo[1,5-a]pyridin-8-yl 4-[[(1Z)-[[(tert-butoxy)carbonyl]amino]([[(tert-butoxy)carbonyl]imino])methyl]amino]-2-iodobenzoate as an off-white solid.

LCMS: (ES, m/z): [M+1]⁺=737. ¹H NMR: (300 MHz, DMSO-d₆, ppm) δ 11.12 (s, 1H), 10.18 (s, 1H), 8.51 (d, J=7.2 Hz, 2H), 8.20 (d, J=8.5 Hz, 1H), 7.82 (d, J=8.8 Hz, 1H), 7.71 (d, J=7.8 Hz, 1H), 7.26 (d, J=8.0 Hz, 1H), 4.21 (s, 2H)), 1.49 (s, 6H), 1.49 (s, 9H), 1.37 (s, 9H).

### Example 42: Compound #42

### 2-(8-((4-guanidino-2-(3-(2-(prop-2-yn-1-yloxy)ethoxy)prop-1-yn-1-yl)benzoyl)oxy)-[1,2,4]triazolo[1,5-a]pyridin-5-yl)acetic acid

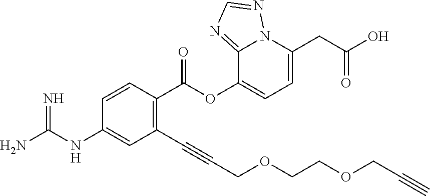 [SMILES: C#CCOCCOCC#Cc1cc(NC(=N)N)ccc1C(=O)Oc1ccc(CC(=O)O)n2ncnc12]

### Step 1: Trimethyl(3-(2-(prop-2-yn-1-yloxy)ethoxy)prop-1-yn-1-yl)silane

Into a 100-mL 3-necked round-bottom flask purged and maintained with an inert atmosphere of nitrogen, was placed a solution of 3-[2-(prop-2-yn-1-yloxy)ethoxy]prop-1-yne (2 g, 14.48 mmol, 1.00 equiv.) in THE (20 mL). This was followed by the addition of LiHMDS (17.3 mL, 17.37 mmol, 1.20 equiv. 1M). dropwise with stirring at 0° C. The resulting solution was stirred for 30 min at 0° C. To the resulting mixture was then added chlorotrimethylsilane (1.9 g, 17.49 mmol, 1.20 equiv.), dropwise with stirring at 0° C. The reaction was stirred for 1 h at 0° C. The reaction was then quenched by the addition of NH₄Cl (aq., 50 mL). The resulting solution was extracted with ethyl acetate (2×100 mL) and the organic layers combined. The resulting mixture was washed with brine (1×100 mL), dried over anhydrous sodium sulfate and concentrated under vacuum. The resulting residue was purified by Flash-Prep-HPLC with the following conditions (IntelFlash-1): Column, silica gel; mobile phase, PE/EA; Gradient: PE to 10% EA within 20 min to yield trimethyl([3-[2-(prop-2-yn-1-yloxy)ethoxy]prop-1-yn-1-yl])silane as colorless oil.

### Step 2: 5-(2-(tert-butoxy)-2-oxoethyl)-[1,2,4]triazolo[1,5-a]pyridin-8-yl 4-amino-2-(3-(2-((3-(trimethylsilyl)prop-2-yn-1-yl)oxy)ethoxy)prop-1-yn-1-yl)benzoate

Into a 25-mL sealed tube purged and maintained with an inert atmosphere of nitrogen, was placed 5-[2-(tert-butoxy)-2-oxoethyl]-[1,2,4]triazolo[1,5-a]pyridin-8-yl 4-amino-2-iodobenzoate (300 mg, 0.61 mmol, 1.00 equiv.), trimethyl([3-[2-(prop-2-yn-1-yloxy)ethoxy]prop-1-yn-1-yl])silane (510 mg, 2.42 mmol, 4.00 equiv.), DIEA (78.4 mg, 0.61 mmol, 1.00 equiv.), CuI (11.5 mg, 0.06 mmol, 0.10 equiv.), Pd(PPh₃)₂Cl₂ (42.6 mg, 0.06 mmol, 0.10 equiv.), and THE (4 mL). The reaction mixture was then irradiated with microwave radiation for 2 h at 65° C. The resulting mixture was concentrated under vacuum. The residue was applied onto a silica gel column with dichloromethane/ethyl acetate (90/10) to yield 5-[2-(tert-butoxy)-2-oxoethyl]-[1,2,4]triazolo[1,5-a]pyridin-8-yl 4-amino-2-[3-(2-[[3-(trimethylsilyl)prop-2-yn-1-yl]oxy]ethoxy)prop-1-yn-1-yl]benzoate as a light yellow solid.

### Step 3: 5-(2-(tert-butoxy)-2-oxoethyl)-[1,2,4]triazolo[1,5-a]pyridin-8-yl (Z)-4-(2,3-bis(tert-butoxycarbonyl)guanidino)-2-(3-(2-((3-(trimethylsilyl)prop-2-yn-1-yl)oxy)ethoxy)prop-1-yn-1-yl)benzoate

Into a 25-mL round-bottom flask, was placed 5-[2-(tert-butoxy)-2-oxoethyl]-[1,2,4]triazolo[1,5-a]pyridin-8-yl 4-amino-2-[3-(2-[[3-(trimethylsilyl)prop-2-yn-1-yl]oxy]ethoxy)prop-1-yn-1-yl]benzoate (700 mg, 1.21 mmol, 1.00 equiv.), tert-butyl N-[(1E)-[[(tert-butoxy)carbonyl]imino](1H-pyrazol-1-yl)methyl]carbamate (376 mg, 1.21 mmol, 1.00 equiv.), acetonitrile (12 mL), TFA (1 mg, 0.01 mmol, 0.01 equiv.). The resulting solution was stirred overnight at room temperature. The resulting mixture was concentrated under vacuum. The residue was applied onto a silica gel column with dichloromethane/ethyl acetate (100/0 to 90/10) to yield 5-[2-(tert-butoxy)-2-oxoethyl]-[1,2,4]triazolo[1,5-a]pyridin-8-yl 4-[[(1Z)-[[(tert-butoxy)carbonyl]amino]([[(tert-butoxy)carbonyl]imino])methyl]amino]-2-[3-(2-[[3-(trimethylsilyl)prop-2-yn-1-yl]oxy]ethoxy)prop-1-yn-1-yl]benzoate as a light yellow solid.

### Step 4: 5-(2-(tert-butoxy)-2-oxoethyl)-[1,2,4]triazolo[1,5-a]pyridin-8-yl (Z)-4-(2,3-bis(tert-butoxycarbonyl)guanidino)-2-(3-(2-(prop-2-yn-1-yloxy)ethoxy)prop-1-yn-1-yl)benzoate

Into a 25-mL round-bottom flask, was placed 5-[2-(tert-butoxy)-2-oxoethyl]-[1,2,4]triazolo[1,5-a]pyridin-8-yl 4-[[(1Z)-[[(tert-butoxy)carbonyl]amino]([[(tert-butoxy) carbonyl]imino])methyl]amino]-2-[3-(2-[[3-(trimethylsilyl)prop-2-yn-1-yl]oxy]ethoxy)prop-1-yn-1-yl]benzoate (120 mg, 0.15 mmol, 1.00 equiv.) and THE (12 mL). This was followed by the addition of TBAF (0.15 mL, 1 M in THF, 1.00 equiv.) at −10° C. The resulting solution was stirred for 20 min at −10° C. in a water/ice bath. The resulting mixture was washed with water (10 mL). The resulting solution was extracted with ethyl acetate (3×10 mL) and the organic layers combined and concentrated under vacuum. The residue was applied onto a silica gel column with dichloromethane/ethyl acetate (100/0 to 90/10) to yield 5-[2-(tert-butoxy)-2-oxoethyl]-[1,2,4]triazolo[1,5-a]pyridin-8-yl 4-[[(1Z)-[[(tert-butoxy)carbonyl]amino]([[(tert-butoxy)carbonyl]imino])methyl]amino]-2-[3-[2-(prop-2-yn-1-yloxy)ethoxy]prop-1-yn-1-yl]benzoate as a white solid.

### Step 5: 2-(8-((4-guanidino-2-(3-(2-(prop-2-yn-1-yloxy)ethoxy)prop-1-yn-1-yl)benzoyl)oxy)-[1,2,4]triazolo[1,5-a]pyridin-5-yl)acetic acid

Into a 50-mL round-bottom flask, was placed 5-[2-(tert-butoxy)-2-oxoethyl]-[1,2,4]triazolo[1,5-a]pyridin-8-yl 4-[[(1Z)-[[(tert-butoxy)carbonyl]amino]([[(tert-butoxy)carbonyl]imino])methyl]amino]-2-[3-[2-(prop-2-yn-1-yloxy)ethoxy]prop-1-yn-1-yl]benzoate (80 mg, 0.11 mmol, 1.00 equiv.), 1,4-dioxane (4 mL), hydrogen chloride (1.3 mL, 4M in 1,4-dioxane, 50.00 equiv.). The resulting solution was stirred for 24 h at 50° C. in an oil bath. The resulting mixture was concentrated under vacuum. The residue was purified by Flash-Prep-HPLC with the following conditions (CombiFlash-1): Column, C18; mobile phase, ACN/H₂O=5/95 increasing to ACN/H₂O=50/50 within 25 min; Detector, UV 254 nm to yield 2-[8-[(4-carbamimidamido-2-[3-[2-(prop-2-yn-1-yloxy)ethoxy]prop-1-yn-1-yl]phenyl)carbonyloxy]-[1,2,4]triazolo[1,5-a]pyridin-5-yl]acetic acid hydrochloride as a white solid.

LCMS: (ES, m/z): [M+1]⁺=491. ¹H NMR: (300 MHz, DMSO-d₆, ppm) δ 8.37 (s, 1H), 8.10 (d, J=8.8 Hz, 1H), 7.82 (s, 4H), 7.58 (d, J=7.7 Hz, 1H), 7.27-7.20 (m, 1H), 7.07 (dd, J=22.1, 8.9 Hz, 2H), 4.35 (s, 2H), 4.04 (d, J=2.4 Hz, 2H), 3.94 (s, 2H), 3.55 (t, J=4.6 Hz, 2H), 3.43 (t, J=4.6 Hz, 2H), 3.37 (d, J=2.5 Hz, 1H).

### Example 43: Compound #43

### 2-(8-((4-Guanidino-2-(3-(prop-2-yn-1-yloxy)prop-1-yn-1-yl)benzoyl)oxy)-[1,2,4]triazolo[1,5-a]pyridin-5-yl)acetic acid

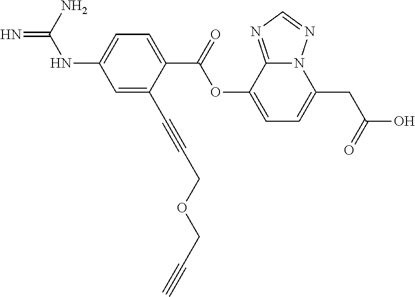 [SMILES: C#CCOCC#Cc1cc(NC(=N)N)ccc1C(=O)Oc1ccc(CC(=O)O)n2ncnc12]

The title compound was similarly prepared as a yellow solid according to the procedure described in Example 42, Steps 1-5 using 3-(prop-2-yn-1-yloxy)prop-1-yne as the starting material in Step 1.

LCMS (ES, m/z): [M+1]⁺=447.0. ¹H NMR (400 MHz, methanol-d₄) δ=8.43 (s, 1H), 8.36 (d, J=8.8 Hz, 1H), 7.65 (d, J=7.9 Hz, 1H), 7.55 (d, J=2.0 Hz, 1H), 7.45 (dd, J=2.4, 8.6 Hz, 1H), 7.24 (d, J=7.7 Hz, 1H), 4.47 (s, 2H), 4.24 (d, J=2.4 Hz, 4H), 2.85-2.64 (m, 1H).

### Biological Example 1

In vitro inhibition potencies against Enteropeptidase (EP) Recombinant full-length human EP (PeproTech, Cat #450-48C) was used in this biochemical assay, with a quenched fluorescent labeled peptide GDDDDK-naphthylamine (NA) (Sigma, Cat #G5261) as the substrate in a buffer of 50 mM Tris-HCl pH 7.4, 200 mM NaCl and 0.01% Triton X-100. The enzyme activity was measured by following the formation of the cleavage product GDDDDK-naphthylamine(NA) by monitoring the fluorescence intensity (FI) at excitation/emission wavelengths of 340/410 nm. The steady-state kinetic characterization studies yielded an average of KM to be 412±22 μM and the apparent turnover number k_{cat}^{app} of 11.8±0.4 s⁻¹ (N=2).

Compound inhibition studies were carried out under the conditions of 300 μM substrate and 1.25 nM EP. Reaction progress curves were collected by reading the FI at 340/410 nm continuously for 1 hour at an interval of 1 minute.

To screen compounds of formula (I), inhibition was studied at 4 different compound concentrations of 10 μM, 1 μM, 0.1 μM and 0.01 μM. Initial velocity of the reaction was determined, and percent of inhibition was calculated by comparing to the reaction in the absence of compound.

Accurate measurements of the inhibition potencies of selected compounds were studied in a 12-point dose-dependence fashion. Inhibition of EP by test compounds was observed to be time-dependent. Compound potency was measured by determining the inactivation rate constant (k_{obs}) following the reaction change from the initial velocity vᵢ to the steady-state velocity vₛ at various compound concentrations. The inactivation rate constants (k_{obs}), were derived by fitting the reaction progress curves to eq. 1.

$${Y}_{T} = {v}_{s} t + \frac{{v}_{i} - {v}_{s}}{{k}_{obs}} ( 1 - {e}^{- {k}_{obs} t} ) + {Y}_{0} \qquad ( 1 )$$

where Y_{T} is the detection signal of the reaction at time t, vᵢ and vₛ are the initial and the steady-state velocities of the reaction, t is the reaction time. Y₀ is the background signal (when t=0).

Compound binding mechanism was shown by plotting k_{obs}˜[compound]. Hyperbolic fitting to eq. 2

$${k}_{obs} = \frac{{k}_{3} [ I \}}{{K}_{I}^{app} + [ I ]} + {k}_{4} \qquad ( 2 )$$

conforms to a two-step binding mechanism as shown in Scheme BIO-1.

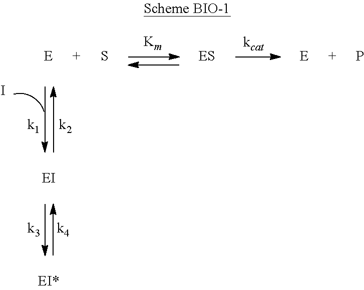

The plateau of the curve is the forward step rate constant of the isomerization step k₃, K_{I}^{app} is the inhibitor concentration that leads to half-maximal rate of inhibition of enzyme under the assay conditions. Compound inhibition potency was reported by the value of k₃/K_{I}^{app}.

A linear dependence of eq.3

k_{obs}=k₁[I]+k₂ (3)

indicates a one-step binding mechanism as shown in Scheme BIO-2

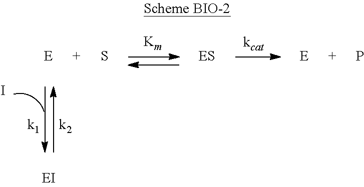

Slope of the linear fit gives the second-order rate constant of the compound binding k₁, which is used to represent the compound inhibition potency.

### Biological Example 2

### Biochemical Assay of Trypsin (T)

Bovine trypsin was purchased from Sigma-Aldrich (Cat No. T1426), and 7-amino-4-methylcoumarin (AMC) conjugated peptide H-D-cyclohexylalanine (D-CHA)-AR-AMC was used as the substrate in the assay. The protease activity of trypsin was followed by measuring AMC production by the emission fluorescence intensity of AMC at 460 nm with the excitation at 375 nm, in the same assay buffer as used in the EP assay. The enzyme kinetic characterization studies yielded an average of KM to be 3.9±0.5 μM and the apparent turnover number k_{cat}^{app} of 33.8±0.4 s⁻¹ (N=2).

Studies of compound inhibition against T were carried out under the conditions of 15 μM substrate and 0.025 nM Trypsin. Reaction progress curves were collected by reading the FI at 375/460 nm continuously for 1 hour at an interval of 1 minute. Similar to the procedure for Enteropeptidase described above, compounds of formula (I) were screened against Trypsin at 4 different concentrations to obtain the percent of inhibition at 10 μM, 1 μM, 0.1 μM and 0.01 μM, and accurate potencies of selected compounds against Trypsin were derived from detailed 12-point dose-dependence studies for time-dependent inhibitors.

Representative compounds of the present invention were tested according to the procedures as described in Biological Example 1 and Biological Example 2 above, with results as listed in Table 2 and Table 3, below. Results are reported as the %-inhibition (% Inh) or K_{inact}/K_{I} value. Variability for the functional assay was typically within 20%. For the column headed K_{inact}/K_{I} the listed measurement is the result of duplicate dilutions.

TABLE 2
Enteropeptidase Activity, Compounds of Formula (I)

| | k_{inact}/K_{I}^{App} | % Inh @ | % Inh @ | % Inh @ |
|---|---|---|---|---|
| ID No. | (1/M*s) | 0.1 μM | 1 μM | 10 μM |
| 1 | 130100 | 97.8 | 100.5 | 100.1 |
| 2 | 24380 | 27.6 | 95.1 | 100.5 |
| 3 | | 35.0 | 100.7 | 100.4 |
| 4 | | 48.1 | 101.2 | 100.2 |
| 5 | | 48.3 | 101.09 | 100.2 |
| 6 | | 20.9 | 72.9 | 101.0 |
| 7 | | 37.8 | 100.2 | 100.4 |
| 8 | 181100 | 101.0 | 100.4 | 100.1 |
| 9 | 210400 | 101.3 | 100.3 | 100.1 |
| 10 | | 82.0 | 100.3 | 100.5 |
| 11 | | 89.7 | 100.6 | 100.2 |
| 12 | | 20.8 | 87.1 | 100.8 |
| 13 | | −1.5 | 15.8 | 65.0 |
| 14 | 72800 | 96.8 | 100.3 | 100.1 |
| 15 | | 1.3 | 6.3 | 36.7 |
| 16 | | 18.7 | 84.4 | 100.7 |
| 17 | | 73.0 | 101.3 | 100.2 |
| 18 | | 81.2 | 100.5 | 100.0 |
| 19 | | 10.3 | 59.9 | 101.1 |
| 20 | | 4.1 | 38.5 | 98.9 |
| 21 | | 99.4 | 100.0 | 100.0 |
| 22 | | −3.0 | −6.3 | 32.1 |
| 23 | | 34.3 | 92.1 | 101.1 |
| 24 | | 36.3 | 98.8 | 100.2 |
| 25 | | 45.2 | 102.5 | 100.6 |
| 26 | | 13.2 | 58.8 | 101.0 |
| 27 | | 81.2 | 100.4 | 100.1 |
| 28 | | 88.6 | 98.5 | 99.9 |
| 29 | | 95.3 | 99.4 | 100.1 |
| 30 | | 92.1 | 99.5 | 100.0 |
| 31 | | 85.8 | 99.7 | 99.792 |
| 32 | | 10.5 | 57.9 | 93.2 |
| 33 | | −0.8 | 11.3 | 52.3 |
| 34 | | 15.3 | 80.1 | 97.9 |
| 35 | | 40.5 | 93.8 | 99.3 |
| 36 | | 2.4 | 9.1 | 34.0 |
| 37 | | 47.8 | 99.6 | 100.1 |
| 38 | | −1.6 | 1.9 | 13.6 |
| 39 | | 0.5 | 12.2 | 51.8 |
| 40 | | −10.7 | −10.1 | −10.8 |
| 41* | | NT | NT | NT |
| 42 | 52764 | 62.3 | 98.5 | 99.8 |
| 43 | 12260 | 56.5 | 99.9 | 99.9 |

*Compound #41 was not tested (NT)

TABLE 3
Trypsin Activity, Compounds of Formula (I)

| | K_{inact}/K_{I}^{App} | % Inh @ | % Inh @ | % Inh @ |
|---|---|---|---|---|
| ID No. | (1/M*s) | 0.1 μM | 1 μM | 10 μM |
| 1 | 158760 | 97.2 | 100.3 | 100.3 |
| 2 | | 61.0 | 100.4 | 100.0 |
| 3 | | 99.8 | 100.0 | 100.0 |
| 4 | | 99.7 | 100.0 | 100.2 |
| 5 | | 98.3 | 100.0 | 100.0 |
| 6 | | 89.6 | 99.9 | 100.1 |
| 7 | | 94.1 | 100.1 | 100.2 |
| 8 | 162610 | 98.8 | 100.5 | 99.9 |
| 9 | 111030 | 96.3 | 100.7 | 100.6 |
| 10 | | 93.8 | 99.9 | 99.8 |
| 11 | | 99.3 | 100.1 | 100.0 |
| 12 | | 13.8 | 72.4 | 99.9 |
| 13 | | 0.9 | 7.6 | 45.0 |
| 14 | 69688 | 87.6 | 100.01 | 100.1 |
| 15 | | −0.1 | 8.4 | 35.5 |
| 16 | | 6.3 | 56.6 | 99.8 |
| 17 | | 99.8 | 100.1 | 100.1 |
| 18 | | 43.5 | 97.6 | 99.9 |
| 19 | | 9.5 | 48.9 | 99.7 |
| 20 | | 2.3 | 16.6 | 88.7 |
| 21 | | 97.1 | 99.7 | 100.0 |
| 22 | | 0.9 | 12.3 | 29.9 |
| 23 | | 12.4 | 73.1 | 100.2 |
| 24 | | 14.6 | 80.0 | 99.9 |
| 25 | | 99.2 | 100.1 | 100.0 |
| 26 | | 81.7 | 100.2 | 100.0 |
| 27 | | 99.3 | 100.2 | 100.1 |
| 28 | | 97.8 | 99.9 | 100.1 |
| 29 | | 95.8 | 99.7 | 100.1 |
| 30 | | 96.5 | 100.0 | 100.0 |
| 31 | | 95.7 | 99.9 | 100.2 |
| 32 | | 10.9 | 58.3 | 93.0 |
| 33 | | 5.7 | 8.7 | 66.5 |
| 34 | | 9.6 | 57.1 | 97.7 |
| 35 | | 31.4 | 89.2 | 98.9 |
| 36 | | −3.1 | −3.3 | 18.2 |
| 37 | | 11.5 | 68.0 | 99.7 |
| 38 | | −2.1 | 2.5 | 35.8 |
| 39 | | 1.6 | 2.8 | 25.5 |
| 40 | | −8.0 | −0.4 | 10.1 |
| 41* | | NT | NT | NT |
| 42 | 59353 | 52.9 | 99.6 | 100.1 |
| 43 | 40135 | 76.1 | 99.5 | 100.1 |

*Compound #41 was not tested (NT)

### Biological Example 3—Prophetic Example

### Compound Inhibition Half-Life of Enteropeptidase

The half-life (t_{1/2}) of compound inhibition of EP is determined by measuring the rate of the hydrolysis reaction of EP-compound adduct. EP-compound adduct is made by mixing EP (0.51 μM final) with an excess amount of compound (23.3 μM final) in the assay buffer and incubated at room temperature for 50 minutes. The mixture is transferred to a pre-equilibrated Bio-Rad micro size-exclusion spin column with Bio-Gel P-6 to remove free compound. Elute of EP-compound adduct is immediately diluted 100-fold in reaction buffer, by mixing with 300 and 600 μM (final concentrations) of substrate (details see the section of Biochemical assay of EP). The rate of the EP-compound hydrolysis is determined by measuring the recovery of EP activity as a function of time. The time traces of the enzyme reactions are fit to eq. 1 (above) to derive the first-order rate constant k_{obs}. The final steady-state velocity vₛ is calculated from the control sample, where EP is incubated with only an equivalent amount of DMSO and set as a fixed parameter in the curve fitting for the derivation of the dissociation rate constant of the EP-compound adduct. The inhibition half-life is calculated by the ratio of 0.693/k_{obs}.

### Formulation Example 1

### Solid, Oral Dosage Form—Prophetic Example

As a specific embodiment of an oral composition, 100 mg of the Compound #1 (prepared as in Example 1) or Compound #9 (prepared as in Example 9) is formulated with sufficient finely divided lactose to provide a total amount of 580 to 590 mg to fill a size O hard gel capsule.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

Throughout this application, various publications are cited. The disclosure of these publications is hereby incorporated by reference into this application to describe more fully the state of the art to which this invention pertains.

1. A compound of formula (I) 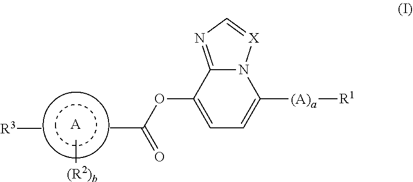
wherein
X is selected from the group consisting of N and CH;
a is an integer from 0 to 1;
A is —(C₁₋₆alkyl)-; R¹ is selected from the group consisting of 1,2,3,5-tetrazol-4-yl, imidazolidin-5-yl-2,4-dione, oxazolidin-5-yl-2,4-dione, 1,2,4-oxadiazol-5(4H)-one, thiazolidin-5-yl-2,4-dione, 1,2,4-thiadiazol-5(4H)-one, isothiazolidin-4-yl-3-one 1,1-dioxide, 2,4-dihydro-3H-1,2,4-triazol-3-one, —CO₂H, —C(O)—O—(C₁₋₄alkyl), —SO₃H, —PO₃H2, —C(O)—NH—CH₂—CO₂H, —C(O)—NH—CH(CO₂H)—CH₂—CO₂H, —C(O)—N(CO₂H)—CH₂—CO₂H, —C(O)—N(CH₂CO₂H)—CH₂—CO₂H, —C(O)—NH—C(CH₂CH₂—CO₂H)₃, —C(O)—NH—SO₂—(C₁₋₄alkyl) and —C(O)—NH—CH(R⁵)—CO₂H and —C(O)-proline;
wherein R⁵ is selected from the group consisting of hydrogen, methyl, isopropyl, 2-methylpropyl, 3-methylpropyl, 2-methylthio-ethyl, benzyl, 4-hydroxy-benzyl, 1H-indol-3-methyl, carbamoyl-methyl, sulfanyl-methyl, 2-carbamoyl-ethyl, hydroxy-methyl, 2-hydroxy-ethyl, carboxyl-methyl, 2-carboxy-ethyl, 3-guanidino-propyl, 1H-imidazol-4-yl-methyl, 4-amino-butyl; 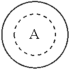
is selected from the group consisting of phen-1,4-diyl, phen-1,3-diyl, 5-6 membered heteroaryl and bicyclic 9-10 membered heteroaryl;
wherein the 5 membered heteroaryl is bound (to the —R³ and —C(O)O substituents) to the rest of the compound through two carbon atoms in a meta orientation;
wherein the 6 membered heteroaryl is bound (to the —R³ and —C(O)O substituents) to the rest of the compound through two carbon atoms in a meta or para orientation; and
wherein the bicyclic 9-10 membered heteroaryl is bound (to the —R³ and —C(O)O substituent) to the rest of the compound through two carbon atoms in a meta or para orientation;
b is an integer from 0 to 1;
R² is selected from the group consisting of halogen, hydroxy, C₁₋₄alkyl, fluorinated C₁₋₂alkyl, C₁₋₄alkoxy, fluorinated C₁₋₂alkoxy, cyano, 3-(prop-2-yn-1-yloxy)prop-1-yn-1-yl and 3-(2-(prop-2-yn-1-yloxy)ethoxy)prop-1-yn-1-yl;
provided that R² is bound to a carbon atom;
provided further that when R² is 3-(prop-2-yn-1-yloxy)prop-1-yn-1-yl or 3-(2-(prop-2-yn-1-yloxy)ethoxy)prop-1-yn-1-yl, then the R² is bound to a carbon atom at the ortho position to the —C(O)—O— group of the compound of formula (I);
R³ is selected from the group consisting of —CH(═NH)—NH—NH₂, —CH(═NH)—NH(C₁₋₄alkyl), —CH(═NH)—NH(OC₁₋₄alkyl), —CH(═NH)—NH—CN, —NH—CH(═NH)—NH₂, —NH—CH(═N(Boc))—NH(Boc), —NH—CH(═NH)—NH(C₁₋₄alkyl), —NH—CH(═NH)—NH(OH), —NH—CH(═NH)—NH—O(C₁₋₄alkyl), —N(C₁₋₄alkyl)-CH(═NH)—NH₂, —N(C₁₋₄alkyl)-CH(═NH)—NH(C₁₋₄alkyl), —NH—CH(═NH)—(C₁₋₄alkyl), —NH—C(═NOH)—(C₁₋₄alkyl) and imidazolidin-1-yl-2-imine;
or a pharmaceutically acceptable salt thereof.

2. The compound of claim 1, wherein
X is selected from the group consisting of N and CH;
a is an integer from 0 to 1;
A is —(C₁₋₄alkyl)-; R¹ is selected from the group consisting of 1,2,3,5-tetrazol-4-yl, imidazolidin-5-yl-2,4-dione, oxazolidin-yl-2,4-dione, 1,2,4-oxadiazol-5(4H)-one, thiazolidin-5-yl-2,4-dione, —CO₂H, —C(O)—O—(C₁₋₄alkyl), —SO₃H, —C(O)—NH—CH₂—CO₂H, —C(O)—NH—CH(CO₂H)—CH₂—CO₂H, —C(O)—N(CO₂H)—CH₂—CO₂H, —C(O)—N(CH₂CO₂H)—CH₂—CO₂H, —C(O)—NH—C(CH₂CH₂—CO₂H)₃, —C(O)—NH—SO₂—(C₁₋₄alkyl), —C(O)—NH—CH(R⁵)—CO₂H and —C(O)-proline;
wherein R⁵ is selected from the group consisting of hydrogen, methyl, isopropyl, 2-methylpropyl, 3-methylpropyl, benzyl, 4-hydroxy-benzyl, hydroxy-methyl, 2-hydroxy-ethyl, carboxyl-methyl and 2-carboxy-ethyl 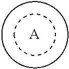
is selected from the group consisting of phen-1,4-diyl, phen-1,3-diyl, 6 membered heteroaryl and 9 membered heteroaryl;
wherein the 6 membered heteroaryl is bound to the compound through two carbon atoms in para orientation;
wherein the 9 membered heteroaryl is bound to the compound through two carbon atoms in a para orientation;
b is an integer from 0 to 1;
R² is selected from the group consisting of halogen, hydroxy, C₁₋₄alkyl, fluorinated C₁₋₂alkyl, C₁₋₄alkoxy, fluorinated C₁₋₂alkoxy, cyano, 3-(prop-2-yn-1-yloxy)prop-1-yn-1-yl and 3-(2-(prop-2-yn-1-yloxy)ethoxy)prop-1-yn-1-yl;
provided that R² is bound to a carbon atom;
provided further that when R² is 3-(prop-2-yn-1-yloxy)prop-1-yn-1-yl or 3-(2-(prop-2-yn-1-yloxy)ethoxy)prop-1-yn-1-yl, then the R² is bound to a carbon atom at the ortho position to the —C(O)—O— group of the compound of formula (I);
R³ is selected from the group consisting of —CH(═NH)—NH—NH₂, —CH(═NH)—NH(C₁₋₄alkyl), —CH(═NH)—NH(OC₁₋₄alkyl), —CH(═NH)—NH—CN, —NH—CH(═NH)—NH₂, —NH—CH(═N(Boc))—NH(Boc), —NH—CH(═NH)—NH(C₁₋₄alkyl), —NH—CH(═NH)—NH(OH), —NH—CH(═NH)—NH—O(C₁₋₄alkyl), —N(C₁₋₄alkyl)-CH(═NH)—NH₂, —N(C₁₋₂alkyl)-CH(═NH)—NH(C₁₋₂alkyl), —NH—CH(═NH)—(C₁₋₄alkyl), —NH—C(═NOH)—(C₁₋₄alkyl) and imidazolidin-1-yl-2-imine;
or a pharmaceutically acceptable salt thereof.

3. The compound of claim 2, wherein
X is selected from the group consisting of N and CH;
a is an integer from 0 to 1;
A is selected from the group consisting of —CH₂— and —CH₂CH₂—;
R¹ is selected from the group consisting of 1,2,3,5-tetrazol-4-yl, oxazolidin-5-yl-2,4-dione, —C(O)OH, —C(O)—O—(C₁₋₄alkyl), —C(O)—NH—CH₂—CO₂H, —C(O)—NH—CH(CO₂H)—CH₂—CO₂H, —C(O)—N(CH₂CO₂H)—CH₂—CO₂H, —C(O)—NH—C(CH₂CH₂—CO₂H)₃ and —C(O)—NH—SO₂—(C₁₋₂alkyl); 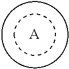
is selected from the group consisting of phen-1,4-diyl, thiophen-2,5-diyl, pyrimidin-2,5-diyl and benzofur-4,7-diyl;
b is an integer from 0 to 1;
R² is selected from the group consisting of halogen, hydroxy, C₁₋₂alkyl, fluorinated C₁₋₂alkyl, C₁₋₂alkoxy, fluorinated C₁₋₂alkoxy, cyano, 3-(prop-2-yn-1-yloxy) prop-1-yn-1-yl and 3-(2-(prop-2-yn-1-yloxy) ethoxy) prop-1-yn-1-yl;
provided that R² is bound to a carbon atom;
provided further that when R² is 3-(prop-2-yn-1-yloxy)prop-1-yn-1-yl or 3-(2-(prop-2-yn-1-yloxy)ethoxy)prop-1-yn-1-yl, then the R² is bound to a carbon atom at the ortho position to the —C(O)—O— group of the compound of formula (I);
R³ is selected from the group consisting of —CH(═NH)—NH—NH₂, —CH(═NH)—NH(C₁₋₂alkyl), —CH(═NH)—NH(OC₁₋₂alkyl), —CH(═NH)—NH—CN, —NH—CH(═NH)—NH₂, —NH—CH(═N(Boc))—NH(Boc), —NH—CH(═NH)—NH(C₁₋₂alkyl), —N(C₁₋₂alkyl)-CH(═NH)—NH₂, —NH—CH(═NH)—(C₁₋₂alkyl), —NH—C(═NOH)—(C₁₋₂alkyl) and imidazolidin-1-yl-2-imine;
or a pharmaceutically acceptable salt thereof.

4. The compound of claim 3, wherein
X is selected from the group consisting of N and CH;
a is an integer from 0 to 1;
A is selected from the group consisting of —CH₂— and —CH₂CH₂—;
R¹ is selected from the group consisting of 1,2,3,5-tetrazol-4-yl, oxazolidin-5-yl-2,4-dione, —C(O)OH, 2-(C(O)OH), —C(O)—O—C(CH₃)₃, —C(O)—NH—CH₂—CO₂H, —C(O)—N(CH₂CO₂H)—CH₂—CO₂H, —C(O)—NH—CH(CO₂H)—CH₂—CO₂H, —C(O)—NH—C(CH₂CH₂—CO₂H)₃ and —C(O)—NH—SO₂—CH₃; 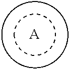
is selected from the group consisting of phen-1,4-diyl, thiophen-2,5-diyl, pyrimidin-2,5-diyl and benzofur-4,7-diyl;
b is an integer from 0 to 1;
R² is selected from the group consisting of 2-chloro, 2-fluoro, 2-iodo, 3-chloro, 3-fluoro, 3-iodo, 3-hydroxy, 2-methyl, 3-methyl, 2-trifluoromethyl, 3-methoxy, 3-cyano, 3-(prop-2-yn-1-yloxy) prop-1-yn-1-yl and 3-(2-(prop-2-yn-1-yloxy) ethoxy) prop-1-yn-1-yl;
R³ is selected from the group consisting of —CH(═NH)—NH—NH₂, —CH(═NH)—NH(CH₃), —CH(═NH)—NH(OCH₃), —CH(═NH)—NH—CN, —NH—CH(═NH)—NH₂, —NH—CH(═N(Boc))—NH(Boc), —NH—CH(═NH)—NH(CH₃), —N(CH₃)—CH(═NH)—NH₂, —NH—CH(═NH)—CH₃, —NH—C(═NOH)—CH₃ and imidazolidin-1-yl-2-imine;
or a pharmaceutically acceptable salt thereof.

5. The compound of claim 1, wherein
X is CH;
a is 1;
A is selected from the group consisting of —CH₂— and —CH₂CH₂—;
R¹ is selected from the group consisting of —C(O)OH, 2-(C(O)OH) and —C(O)—NH—CH(CO₂H)—CH₂—CO₂H; 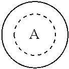
is selected from the group consisting of phen-1,4-diyl and pyrimidin-2,5-diyl;
b is 0;
R³ is —NH—CH(═NH)—NH₂;
or a pharmaceutically acceptable salt thereof.

6. The compound of claim 1, wherein
X is N;
a is an integer from 0 to 1;
A is selected from the group consisting of —CH₂— and —CH₂CH₂—;
R¹ is selected from the group consisting of 1,2,3,5-tetrazol-4-yl, oxazolidin-5-yl-2,4-dione, —C(O)OH, 2-(C(O)OH), —C(O)—O—C(CH₃)₃, —C(O)—NH—CH₂—CO₂H, —C(O)—N(CH₂CO₂H)—CH₂—CO₂H, —C(O)—NH—CH(CO₂H)—CH₂—CO₂H, —C(O)—NH—C(CH₂CH₂—CO₂H)₃ and —C(O)—NH—SO₂—CH₃; 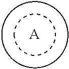
is selected from the group consisting of phen-1,4-diyl, thiophen-2,5-diyl, pyrimidin-2,5-diyl and benzofur-4,7-diyl;
b is an integer from 0 to 1;
R² is selected from the group consisting of 2-chloro, 2-fluoro, 2-iodo, 3-chloro, 3-fluoro, 3-iodo, 3-hydroxy, 2-methyl, 3-methyl, 2-trifluoromethyl, 3-methoxy, 3-cyano, 3-(prop-2-yn-1-yloxy) prop-1-yn-1-yl and 3-(2-(prop-2-yn-1-yloxy) ethoxy) prop-1-yn-1-yl;
R³ is selected from the group consisting of —CH(═NH)—NH—NH₂, —CH(═NH)—NH(CH₃), —CH(═NH)—NH(OCH₃), —CH(═NH)—NH—CN, —NH—CH(═NH)—NH₂, —NH—CH(═N(Boc))—NH(Boc), —NH—CH(═NH)—NH(CH₃), —N(CH₃)—CH(═NH)—NH₂, —NH—CH(═NH)—CH₃, —NH—C(═NOH)—CH₃ and imidazolidin-1-yl-2-imine;
or a pharmaceutically acceptable salt thereof.

7. The compound of claim 1, wherein
X is selected from the group consisting of N and CH;
a is an integer from 0 to 1;
A is selected from the group consisting of —CH₂— and —CH₂CH₂—;
R¹ is selected from the group consisting of 1,2,3,5-tetrazol-4-yl, oxazolidin-5-yl-2,4-dione, —C(O)OH, 2-(C(O)OH), —C(O)—NH—CH₂—CO₂H, —C(O)—NH—CH(CO₂H)—CH₂—CO₂H, —C(O)—NH—C(CH₂CH₂—CO₂H)₃ and —C(O)—NH—SO₂—CH₃; 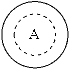
is selected from the group consisting of phen-1,4-diyl, thiophen-2,5-diyl, pyrimidin-2,5-diyl and benzofur-4,7-diyl;
b is an integer from 0 to 1;
R² is selected from the group consisting of 2-chloro, 2-fluoro, 3-fluoro, 3-hydroxy, 3-(prop-2-yn-1-yloxy) prop-1-yn-1-yl and 3-(2-(prop-2-yn-1-yloxy) ethoxy) prop-1-yn-1-yl;
R³ is selected from the group consisting of —NH—CH(═NH)—NH₂ and —N(CH₃)—CH(═NH)—NH₂;
or a pharmaceutically acceptable salt thereof.

8. The compound of claim 1, wherein
X is selected from the group consisting of N and CH;
a is an integer from 0 to 1;
A is —CH₂—;
R¹ is selected from the group consisting of 1,2,3,5-tetrazol-4-yl, oxazolidin-5-yl-2,4-dione, —C(O)OH, —C(O)—NH—CH(CO₂H)—CH₂—CO₂H and —C(O)—NH—SO₂—CH₃; 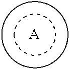
is selected from the group consisting of phen-1,4-diyl, thiophen-2,5-diyl and pyrimidin-2,5-diyl;
b is an integer from 0 to 1;
R² is selected from the group consisting of 2-chloro, 2-fluoro and 3-fluoro;
R³ is —NH—CH(═NH)—NH₂;
or a pharmaceutically acceptable salt thereof.

9. The compound of claim 1, wherein
X is selected from the group consisting of N and CH;
a is 1;
A is —CH₂—;
R¹ is selected from the group consisting of —C(O)OH and —C(O)—NH—SO₂—CH₃; 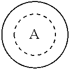
is selected from the group consisting of phen-1,4-diyl and pyrimidin-2,5-diyl;
b is an integer from 0 to 1;
R² is selected from the group consisting of 2-fluoro and 3-fluoro;
R³ is —NH—CH(═NH)—NH₂;
or a pharmaceutically acceptable salt thereof.

10. The compound of claim 1, wherein
X is selected from the group consisting of N and CH;
a is 1;
A is selected from the group consisting of —CH₂— and —CH₂CH₂—;
R¹ is selected from the group consisting of —C(O)OH, —C(O)—NH—CH(CO₂H)—CH₂—CO₂H and —O(O)—NH—C(CH₂CH₂—CO₂H)₃; 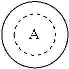
is pheny-1,4-diyl;
b is 0; and
R³ is —NH—CH(═NH)—NH₂;
or a pharmaceutically acceptable salt thereof.

11. A pharmaceutical composition comprising a pharmaceutically acceptable carrier and the compound of claim 1.

12-13. (canceled)

14. A method of treating a disorder mediated by enteropeptidase enzyme activity, comprising administering to a subject in need thereof a therapeutically effective amount of the compound of claim 1.

15. The method of claim 14, wherein the disorder mediated by enteropeptidase enzyme activity is selected from the group consisting of obesity, excess weight, hypertension, hypercholesterolemia, hyperlipidemia, hypertriglyceridemia, heart disease, angina, atherosclerosis, heart disease, heart attack, ischemia, stroke; pancreatitis, impaired glucose tolerance (IGT), impaired fasting glucose (IFT), gestational diabetes, Type II or non-insulin dependent diabetes mellitus, Syndrome X (also known as Metabolic Syndrome), neuropathy, retinopathy, nerve damage or poor blood flow in the feet, chronic kidney disease, microalbuminuria (elevated urine albumin levels), macroalbuminuria, elevated urine albumin levels, elevated albumin/creatinine ratio (ACR), nephropathy, renal hyperfiltration, glomerular hyperfiltration, renal allograft hyperfiltration, compensatory hyperfiltration, hyperfiltrative chronic kidney disease, hyperfiltrative acute renal failure, non-alcoholic steatohepatitis (NASH), non-alcoholic fatty liver disease (NAFLD), liver fibrosis, cataracts, polycystic ovarian syndrome, irritable bowel syndrome, inflammation and cancer.

16. The method of claim 14, wherein the disorder mediated by enteropeptidase enzyme activity is selected from the group consisting of obesity, impaired glucose tolerance (IGT), impaired fasting glucose (IFT), gestational diabetes, Type II or non-insulin dependent diabetes mellitus, Syndrome X (also known as Metabolic Syndrome), chronic kidney disease, microalbuminuria, macroalbuminuria, elevated urine albumin levels, elevated albumin/creatinine ratio (ACR), hyperfiltrative diabetic nephropathy, non-alcoholic steatohepatitis (NASH) and non-alcoholic fatty liver disease (NAFLD).

17. The method of claim 14, wherein the disorder mediated by enteropeptidase enzyme activity is selected from the group consisting of obesity, Type II or non-insulin dependent diabetes mellitus, chronic kidney disease, microalbuminuria, macroalbuminuria, elevated urine albumin levels, elevated albumin/creatinine ratio (ACR), non-alcoholic steatohepatitis (NASH) and non-alcoholic fatty liver disease (NAFLD).

18-25. (canceled)
